(19) United States
(12) Patent Application Publication
(10) Pub. No.: US 2018/0180619 A1
(43) Pub. Date: Jun. 28, 2018

(54) Means and Methods for Diagnosing Pancreatic Cancer in a Subject Based on a Biomarker Panel

(71) Applicant: METANOMICS HEALTH GMBH, Berlin (DE)

(72) Inventors: Beate Kamlage, Berlin (DE); Regina Reszka, Panketal (DE); Philip Schatz, Berlin (DE); Martin Dostler, Henningsdorf (DE); Susan Carvalho, Brelin (DE); Erik Peter, Potsdam (DE); Philipp Mappes, Berlin (DE); Holger Kalthoff, Kiel (DE); Bodo Schniewind, Kiel (DE); Julia Mayerle, Greifwald (DE); Marcus Lerch, Greifwald (DE); Robert Gruetzmann, Dresden (DE); Christian Pilarsky, Dresden (DE)

(73) Assignee: METANOMICS HEALTH GMBH, Berlin (DE)

(21) Appl. No.: 15/738,295

(22) PCT Filed: Jun. 24, 2016
(86) PCT No.: PCT/EP2016/064736

§ 371 Date: Dec. 20, 2017

(30) Foreign Application Priority Data
Jun. 25, 2015 (EP) 15173898.6

(51) Int. Cl. G01N 33/574

(52) U.S. Cl. CPC G01N 33/57438; G01N 2405/08

(57) ABSTRACT

The present invention relates to a method for diagnosing pancreatic cancer in a subject comprising the steps of: (a) determining in at least one sample of said subject the amounts of a group of diagnostic biomarkers comprising (i) at least one diagnostic amino acid, said diagnostic amino acid being proline, histidine or tryptophan, preferably, being proline; (ii) at least one diagnostic ceramide, said diagnostic ceramide being ceramide (d18:1,C24:0) or ceramide (d18:2,C24:0), preferably being ceramide (d18:1,C24:0); (iii) at least one diagnostic sphingomyelin, said diagnostic sphingomyelin being sphingomyelin (35:1), sphingomyelin (d17:1,C16:0), sphingomyelin (41:2) or sphingomyelin (d18:2,C17:0), preferably being sphingomyelin (35:1); and (iv) CA19-9; and (b) comparing said amounts of the diagnostic biomarkers with a reference, whereby pancreatic cancer is diagnosed. Moreover, the present invention relates to a method for determining the probability for a subject to suffer from pancreatic cancer, and to devices and uses related to said methods.

The present invention relates to a method for diagnosing pancreatic cancer in a subject comprising the steps of: (a) determining in at least one sample of said subject the amounts of a group of diagnostic biomarkers comprising (i) at least one diagnostic amino acid, said diagnostic amino acid being proline, histidine or tryptophan, preferably, being proline; (ii) at least one diagnostic ceramide, said diagnostic ceramide being ceramide (d18:1,C24:0) or ceramide (d18:2,C24:0), preferably being ceramide (d18:1,C24:0); (iii) at least one diagnostic sphingomyelin, said diagnostic sphingomyelin being sphingomyelin (35:1), sphingomyelin (d17:1,C16:0), sphingomyelin (41:2) or sphingomyelin (d18:2,C17:0), preferably being sphingomyelin (35:1); and (iv) CA19-9; and (b) comparing said amounts of the diagnostic biomarkers with a reference, whereby pancreatic cancer is diagnosed. Moreover, the present invention relates to a method for determining the probability for a subject to suffer from pancreatic cancer, and to devices and uses related to said methods.

Pancreatic cancer has the worst prognosis of all solid tumors, with 5-year survival rates of less than 5% but an increasing incidence (Everhart 2009, Gastroenterology 136:1134-11449). There is a widely acknowledged demand for the establishment of innovative tools and technologies for point-of-care utilization of specific biomarkers and novel molecular imaging tools for early diagnosis, prognostic stratification and differential diagnosis of pancreatic cancer. Advances in these areas are pivotal to improve the prognosis of this malignancy, since timely surgical resection of early stage tumors is currently the only effective means of treatment of this dismal disease.

The mortality of this cancer type is the highest of any cancer type in Europe and the western world. People die soon after diagnosis due to the lack of means for early detection. Early symptoms are rare and uncharacteristic. Thus, pancreatic ductal adenocarcinomas (PDACs) are commonly diagnosed in an advanced stage of the disease. To date, the best imaging technologies to detect PDAC are endoscopic ultrasound (EUS), spiral computer tomography (CT), magnetic resonance cholangiopancreatography (MRCP) or endoscopic retrograde cholangiopancreatography (ERCP) (Dewitt 2006, Gastroenterol Hepatol. (4):717-25). Unfortunately, the resolution of these technologies for detecting neoplastic lesions within the pancreas is in the range of 3-10 mm. Thus, they are not able to detect pancreatic neoplasia at a curable stage. The serum concentration of conventional tumor markers such as CA19-9 is increased in a subset of pancreatic cancer patients (Fry 2008, Langenbecks Arch Surg. (393): 883-90). However, so far all available markers lack sensitivity and tumor specificity (Gupta et al., 1985, Cancer 56 (277-283)). Thus, new approaches are urgently needed to increase the diagnostic sensitivity towards the detection of very small, early stage PDAC and its precursor lesions (PaniNs and IPMNs) as well as prognostic subgroups of advanced tumors.

The association between chronic inflammation and the development of malignancies has been recognized for many years. For pancreatic cancer, this association was only recently confirmed and a consensus conference agreed upon a new classification for pancreatic intraepithelial neoplasia as noninvasive precursor lesions (Hruban 2004, Am J Surg Path (28): 977-987). Chronic pancreatitis is defined as recurrent bouts of a sterile inflammatory disease characterized by often progressive and irreversible morphological changes, typically causing pain and permanent impairment of pancreatic function. With an incidence of 8.2, a prevalence of 27.4 per 100 000 population and a 0.04% to 5% frequency in unselected autopsy specimens, chronic pancreatitis represents a frequent disorder of the gastrointestinal tract. Various etiologies are responsible for the development of chronic pancreatitis. An increased risk of patients suffering from of chronic pancreatitis to die from pancreatic cancer was shown in an international cooperative investigation conducted by AB Lowenfels and coworkers as a multicenter historical cohort study of 2015 patients with chronic pancreatitis recruited from clinical centers in 6 countries in 1993. This study found a cumulative risk of pancreatic cancer in patients with chronic pancreatitis of 1.8% after 10 years and of 4% after 20 years with a standardized incidence ratio of 14.4. For patients with a minimum of two years follow up the risk of pancreatic cancer was 16.5 fold higher than that of the general population (Lowenfels 1993, N Engl J Med (328): 1433-1437). The search for an association between chronic pancreatitis and pancreatic cancer intensified when in 1996 a single point mutation in the third exon of the cationic trypsinogen gene on chromosome 7 (7q35) was found to be associated with hereditary pancreatitis and multiple kindreds were subsequently identified and reported. Only very recently the EUROPAC study group presented their work on clinical and genetic characteristics in hereditary pancreatitis. In a multilevel proportional hazard model employing data obtained from the European Registry of Hereditary Pancreatitis this group presented 112 families in 14 countries (418 affected individuals) (Howes 2004, Clinical Gastroenterology and Hepatology (2): 252-261). The cumulative risk (95% CI) of pancreatic cancer was 44.0% (8.0%-80.0%) at 70 years from symptom onset with a standardized incidence ratio of 67% (50%-82%). A previous study had also shown an estimated lifetime risk of pancreatic cancer of 40% (Lowenfels 2001, JAMA 286: 169-170, Lowenfels 1997, J Natl Cancer Inst 89: 442-44656).

In pancreatic cancer, imaging studies fail to detect early pancreatic malignancies in a curable stage. Thus, the detection of pancreatic malignancy in a high risk cohort would be highly desired.

There are a few reports of metabolic changes in patients suffering from pancreas-associated diseases. Schrader et al (Schrader 2009, Pancreas 38: 416-421) suggests that patients with pancreatic cancer and chronic pancreatitis show significant changes in serum amino acid levels. It has been suggested that sphingolipids on the cell surface of cells takes actively part in cell signaling (Pitson 2011, Trend Biochem Sci 36:97-107). Ceramides are known to induce apoptosis in cancer cells. Low levels of sphingomyelin suggest less responsiveness to gemcitabine treatment (Modrak 2009, Mol Cancer Res 7:890-896). Further single metabolic biomarkers have been reported in WO 2011/151252 and WO 2013/079594.

CA 19-9 blood levels are elevated in many patients with pancreatic cancer. The CA19-9 level is of limited value for pancreatic cancer diagnostic in terms of both sensitivity and specificity. CA19-9 sensitivity for pancreatic cancer diagnostic is impaired by false positives due to other gastrointestinal cancers such as colon cancer, gastric cancer, and liver cancer, as well as breast cancer and other gynecological cancer, lung cancer, and bronchial cancer. Benign diseases such as pancreatitis also result in false positive CA19-9 levels. CA19-9 specificity for pancreatic cancer diagnostic is further impaired by false negatives patients that are negative for Lewis a/b antigen and will therefore not express CA19-9.

In conclusion, with a 5-year survival rate of 0.5-5%, pancreatic cancer carries the most dismal prognosis of all human tumors and represents the 4th leading cause in cancer-related deaths worldwide. It is thus a disease with a major socioeconomic impact. Accurate diagnosis including its differentiation from pancreatitis and timely surgical resection of early tumors currently offer the only realistic prospect for the improvement of patient prognosis.

The technical problem underlying the present invention can be seen as the provision of means and methods for complying with the aforementioned needs. The technical problem is solved by the embodiments characterized in the claims and herein below.

Accordingly, the present invention relates to a method for diagnosing pancreatic cancer in a subject comprising the steps of:
(a) determining in at least one sample of said subject the amounts of a group of diagnostic biomarkers comprising
   (i) at least one diagnostic amino acid, said diagnostic amino acid being proline, histidine or tryptophan, preferably, being proline;
   (ii) at least one diagnostic ceramide, said diagnostic ceramide being ceramide (d18:1,C24:0) or ceramide (d18:2,C24:0), preferably being ceramide (d18:1,C24:0);
   (iii) at least one diagnostic sphingomyelin, said diagnostic sphingomyelin being sphingomyelin (35:1), sphingomyelin (d17:1,C16:0), sphingomyelin (41:2) or sphingomyelin (d18:2,C17:0), preferably being sphingomyelin (35:1); and
   (iv) CA19-9;
and
(b) comparing said amounts of the diagnostic biomarkers with a reference, whereby pancreatic cancer is diagnosed.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention. The term “about” as used herein refers to a value differing +/−20%, preferably +/−10%, more preferably +/−5%, even more preferably +/−2%, most preferably +/−1% from the value indicated.

The method of the present invention, preferably, is an in vitro method. Moreover, it may comprise steps in addition to those explicitly mentioned above. For example, further steps may relate, e.g., to sample pretreatment for step (a), calculating a value derived from the determined amounts in step b), or recommending further proceeding to the subject after step (b), in particular, in case pancreatic cancer is diagnosed. Moreover, one or more of said steps may be performed by automated equipment.

The term “pancreatic cancer” or “pancreas cancer”, as used herein, relates to a neoplasm derived from pancreatic cells, preferably, from pancreatic epithelial cells. Thus, preferably, pancreatic cancer as used herein is pancreatic ductal adenocarcinoma. The symptoms accompanying pancreatic cancer are well known from standard text books of medicine such as Stedmen or Pschyrembl and include abdominal pain, lower back pain, nausea, vomiting, and in some cases, jaundice. Preferably, the pancreatic cancer is a resectable pancreatic cancer, i.e., preferably, is a pancreatic cancer at a tumor stage permitting, preferably complete, resection of the tumor from the subject. More preferably, said pancreatic cancer is a pancreatic cancer of tumor stage IA-IIB.

The term “diagnosing” as used herein refers to assessing whether a subject suffers from pancreatic cancer, or not. As will be understood by those skilled in the art, such an assessment, although preferred to be, may usually not be correct for 100% of the investigated subjects. The term, however, requires that a statistically significant portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, or 0.05.

The term diagnosing, preferably, includes individual diagnosis of pancreatic cancer or its symptoms as well as continuous monitoring of a patient. Monitoring, i.e. diagnosing the presence or absence of pancreatic cancer or the symptoms accompanying it at various time points, includes monitoring of patients known to suffer from pancreatic cancer as well as monitoring of subjects known to be at risk of developing pancreatic cancer. Furthermore, monitoring can also be used to determine whether treatment of a patient is successful or whether at least symptoms of pancreatic cancer can be ameliorated over time by a certain therapy.

Moreover, the term diagnosing also, preferably, relates to differentially diagnosing pancreatic cancer and, more preferably, differentiating between pancreatic cancer and pancreatitis. Pancreatitis, as used herein, refers to an inflammation of the pancreas. Usually, the cause of pancreatitis is an activation of the pancreatic enzymes, e.g., trypsin, in the pancreas rather than the small intestine. Pancreatitis may occur as an acute disease which occurs suddenly and lasts a few days, or as a chronic disease which persists over many years. Preferably, pancreatitis referred to in accordance with the present invention is chronic pancreatitis. Typical symptoms of pancreatitis can be found in the aforementioned standard text books and encompass severe upper abdominal pain, often radiating to the back, nausea and vomiting. Differentiating between pancreatic cancer and chronic pancreatitis is preferably achieved by applying the methods of the present invention to at least one sample of a subject known or suspected to suffer from pancreatitis and comparing the measured amounts of the biomarkers with references, whereby pancreatic cancer is diagnosed. In a further preferred embodiment, said diagnosis of pancreatic cancer leads to the differentiation whether the person known or suspected to suffer from pancreatitis additionally suffers from pancreatic cancer.

The term “subject”, as used herein, relates to an animal, preferably, to a mammal. More preferably, the subject is a primate and, most preferably, a human. Preferably, the subject is an apparently healthy subject.

Preferably, the subject is a subject at risk of suffering from pancreatic cancer. Risk factors for developing pancreatic cancer are known in the art, e.g. from Brand R E et al., Gut. 2007; 56:1460-9, or Del Chiaro et al., World J Gastroenterol 2014; 20:12118-12131 and include genetic factors, chronic disease, new-onset diabetes and age; thus, preferably, the subject at risk of suffering from pancreatic cancer is a subject having a genetic predisposition, preferably familiar pancreatic cancer, including Peutz-Jeghers Syndrome, BRCA1 positivity, or a genetic predisposition for developing pancreatitis. Also preferably, the subject at risk of suffering from pancreatic cancer is a subject at least 40 years old, most preferably, at least 50 years old. More preferably, the subject at risk of suffering from pancreatic cancer is a subject with new-onset diabetes; and/or said subject at risk of suffering from pancreatic cancer is a subject suffering from chronic pancreatitis. The term “new-onset diabetes” is known to the skilled person and relates to diagnosis of diabetes in a subject not previously having been diagnosed with diabetes and, preferably, not having previously documented symptoms of diabetes, preferably according to WHO guidelines, more preferably an 8-h fasting blood glucose value of >125 mg/dL.

Preferably, the subject is a subject suspected to suffer from pancreatic cancer. Suspicion that a subject may suffer from pancreatic cancer, preferably, arises from at least one clinical symptom known to the skilled person to be associated with pancreatic cancer. Thus, preferably, the subject suspected to suffer from pancreatic cancer, preferably, is a subject having at least one clinical symptom of pancreatic cancer, more preferably selected from the list consisting of abdominal pain, lower back pain, nausea, vomiting, and in some cases, jaundice. Also preferably, the subject suspected to suffer from pancreatic cancer is a subject requiring differential diagnosis between pancreatic cancer and chronic pancreatitis, i.e., preferably, a subject suspected to suffer from pancreatic cancer is a subject suspected to suffer from pancreatic cancer or from chronic pancreatitis. Also preferably, the subject suspected to suffer from pancreatic cancer is a subject having an increased CA19-9 concentration in the blood as compared to a healthy control, preferably more than 37 U/mL, more preferably more than 500 U/mL, most preferably more than 1000 U/mL.

Preferably, the subject is a subject with a low CA19-9 value. Preferably, a low CA19-9 value is a blood CA19-9 value of less than 42 U/mL, preferably less than 37 U/mL. As will be appreciated by the skilled person, Lewis a/b antigen negative subjects have low CA19-9 values (Tian et al., 1992 Annals of Surgery 215 350-355) or a CA19-9 value below the detection limit, preferably, a value of zero. Thus, preferably, the subject with a low CA19-9 value is a Lewis a/b antigen negative subject.

In a preferred embodiment, the subject is a subject having an abdominal cystic lesion, preferably a subject diagnosed with an unclear abdominal expansive lesion. In another preferred embodiment, the subject is a subject having a pancreatic cystic lesion, preferably a subject diagnosed with an unclear pancreatic expansive lesion.

The term “sample” as used herein refers to a sample of a body fluid, preferably, blood, plasma, serum, saliva or urine, or a sample derived by lavage from tissues or organs, in particular from the bile duct. More preferably, the sample is a blood, plasma, serum or urine sample. Even more preferably, the sample is a blood or plasma sample or is a serum or plasma sample, most preferably, a plasma sample. Preferably, if the sample is a blood sample, the method of the present invention comprises a further step of obtaining a serum or plasma sample from said blood sample. Preferably, the sample is a citrate plasma sample, a heparin plasma sample, or an EDTA plasma sample. More preferably, the sample is an EDTA plasma sample. Biological samples can be derived from a subject as specified elsewhere herein. Techniques for obtaining the aforementioned different types of biological samples are well known in the art. For example, blood samples may be obtained by blood taking while tissue or organ samples are to be obtained, e.g., by biopsy. Preferably, the sample is a fasting sample, in particular a fasting blood, plasma or serum sample. Thus, preferably, the sample is obtained from a fasting subject. A fasting subject, in particular, is a subject who refrained from food and beverages, except for water, prior to obtaining the sample to be tested. Preferably, a fasting subject refrained from food and beverages, except for water, for at least eight hours prior to obtaining the sample to be tested. More preferably, the sample has been obtained from the subject after an overnight fast. Preferably said fasting continued up to at least one hour before sample taking, more preferably up to at least 30 min before sample taking, still more preferable up to at least 15 min before sample taking, most preferably until the sample was taken.

The term “biomarker” as used herein refers to a molecular species which serves as an indicator for a disease or effect as referred to in this specification. Said molecular species can be a metabolite itself which is found in a sample of a subject. Moreover, the biomarker may also be a molecular species which is derived from said metabolite. In such a case, the actual metabolite will be chemically modified in the sample or during the determination process and, as a result of said modification, a chemically different molecular species, i.e. an analyte, will be the determined molecular species. It is to be understood that in such a case, the analyte represents the actual metabolite and has the same potential as an indicator for the respective medical condition.

Moreover, a biomarker according to the present invention is not necessarily corresponding to one molecular species. Rather, the biomarker may comprise stereoisomers or enantiomers of a compound. Further, a biomarker can also represent the sum of isomers of a biological class of isomeric molecules, or of a subgroup thereof. Said isomers shall exhibit identical analytical characteristics in some cases and are, therefore, not distinguishable or distinguished by various analytical methods including those applied in the accompanying Examples described below. However, preferably, the isomers will share at least identical sum formula parameters and, thus, in the case of, e.g., lipids, an identical chain length and identical numbers of double bonds in the sum of the fatty acid and other long-chain aliphatic moieties, e.g., sphingobase moieties.

The term “diagnostic biomarker” is used herein as a generic term for the biomarkers of the present invention, i.e., the diagnostic amino acids, the diagnostic ceramides, the diagnostic sphingomyelins, and the diagnostic ethanolamine lipids of the present invention, as specified elsewhere herein and as shown in Table 1, and for CA19-9. As used herein, the term “small molecule diagnostic biomarker” is used as a generic term for diagnostic biomarkers as specified above except CA19-9; i.e. for the diagnostic amino acids, the diagnostic ceramides, the diagnostic sphingomyelins, and the diagnostic ethanolamine lipids of the present invention as specified elsewhere herein and as shown in Table 1. As will be understood by the skilled person, the method of the present invention may comprise determining further biomarkers. As used herein, the term “further biomarker” relates to a biomarker different from the diagnostic biomarkers of the present invention. Nonetheless, the definitions provided herein for biomarkers apply, except otherwise noted, to diagnostic biomarkers mutatis mutandis.

The term “metabolite”, as used herein, refers to a compound produced by or consumed in the metabolism of a subject. The term relates to at least one molecule of a specific metabolite up to a plurality of molecules of the said specific metabolite. It is to be understood further that a group of metabolites means a plurality of chemically different molecules, wherein for each metabolite at least one molecule up to a plurality of molecules may be present. A metabolite in accordance with the present invention encompasses all classes of organic or inorganic chemical compounds, including those being comprised by biological material, such as organisms. Preferably, the metabolite other than CA19-9 in accordance with the present invention is a small molecule compound, i.e., preferably, the metabolite other than CA19-9 is not a biological macromolecule, more preferably, the metabolite other than CA19-9 is a small organic molecule. More preferably, the metabolite other than CA19-9 is a chemical compound with a molecular mass of less than 2000 u (2000 Da; 1 u=1.66×10⁻²⁷ kg), most preferably, less than 1500 u. Thus, preferably, diagnostic biomarkers of the present invention with a molecular mass of less than 2000 u, more preferably, less than 1500 u, are small molecule diagnostic biomarkers of the present invention. In accordance with the above, for biomarkers of the present invention with a molecular mass of less than 2000 u, more preferably, less than 1500 u, the term “small molecule biomarkers” is used; and for further biomarkers of the present invention with a molecular mass of less than 2000 u, more preferably, less than 1500 u, the term “small molecule further biomarkers” is used.

The methods for diagnosing pancreatic cancer of the present invention comprise determining the amount of a diagnostic biomarker or determining the amounts of a group of diagnostic biomarkers. As will be understood by the skilled person, the diagnostic biomarkers of the present invention may be determined as single biomarkers, preferably, by determining each diagnostic biomarker in a specific assay; in such case, it is envisaged that each diagnostic biomarker of the group of diagnostic biomarkers is determined from a specific sample; more preferably, at least two, most preferably at least three diagnostic biomarkers are determined from the same sample. In particular, CA19-9 may, preferably, be determined from a sample, which may be identical or different from the sample used to determine the diagnostic amino acid, the diagnostic ceramide, and/or the diagnostic sphingomyelin. Preferably, said sample used for determining CA19-9 and said sample or samples used for determining the remaining diagnostic biomarkers are obtained within a time frame of at most one year, more preferably at most three months, even more preferably at most two months, most preferably, at most one month. Thus, preferably, the term “determining the amount of CA19-9” includes providing a concentration value for CA19-9 determined earlier for said subject, e.g., preferably, for deciding whether said subject is suspected to suffer from pancreatic cancer. Preferably, at least two, at least three, at least four, at least five, at least six, at least seven, at least eight, at least nine, or at least ten biomarkers are determined in a common assay from the same sample, i.e., an assay providing measured values for said number of biomarkers as an output. The skilled person is aware that some biomarkers are preferably determined in specific assays, e.g., a biomarker having a complex structure, in particular CA19-9 is, preferably, determined in an immunological assay, e.g. preferably, a radioimmunoassay (RIA).

In an embodiment, the diagnostic biomarkers of the group of diagnostic biomarkers of the present invention are determined from the same sample, wherein said sample is, preferably split into at least two subsamples, of which in one subsample the small molecule diagnostic biomarkers are determined and of which in a second subsample CA19-9 is determined. In a further embodiment, small molecule diagnostic biomarkers are determined in a first sample and CA19-9 is determined in a second sample, wherein, preferably, said samples are taken at the same time, or, preferably, at different times as specified above. In a further embodiment, in the method of diagnosing pancreatic cancer, CA19-9 is not determined; in such case, preferably, the subject is a subject known or suspected to be a subject with a low CA19-9 value, more preferably a subject which is Lewis a/b antigen negative, as specified elsewhere herein.

In view of the above, the method for diagnosing pancreatic cancer in a subject of the present invention includes, preferably, a method comprising the steps of

(a1) determining in at least one sample of said subject the amounts of a group of diagnostic biomarkers comprising
(i) at least one diagnostic amino acid, said diagnostic amino acid being proline, histidine or tryptophan, preferably, being proline;
(ii) at least one diagnostic ceramide, said diagnostic ceramide being ceramide (d18:1,C24:0) or ceramide (d18:2,C24:0), preferably being ceramide (d18:1,C24:0); and
(iii) at least one diagnostic sphingomyelin, said diagnostic sphingomyelin being sphingomyelin (35:1), sphingomyelin (d17:1,C16:0), sphingomyelin (41:2) or sphingomyelin (d18:2,C17:0), preferably being sphingomyelin (35:1);
   (a2) providing a value of an amount of the diagnostic biomarker CA19-9 in a sample of said subject; and
   (b) comparing said amounts of said diagnostic biomarkers of (a1) and (a2) with a reference, whereby pancreatic cancer is diagnosed.

Moreover, in view of the above, the method for diagnosing pancreatic cancer in a subject of the present invention includes, preferably, a method comprising the steps of

(a) determining in at least one sample of said subject the amounts of a group of diagnostic biomarkers comprising
(i) at least one diagnostic amino acid, said diagnostic amino acid being proline, histidine or tryptophan, preferably, being proline;
(ii) at least one diagnostic ceramide, said diagnostic ceramide being ceramide (d18:1,C24:0) or ceramide (d18:2,C24:0), preferably being ceramide (d18:1,C24:0); and
(iii) at least one diagnostic sphingomyelin, said diagnostic sphingomyelin being sphingomyelin (35:1), sphingomyelin (d17:1,C16:0), sphingomyelin (41:2) or sphingomyelin (d18:2,C17:0), preferably being sphingomyelin (35:1); and
   (b) comparing said amounts of said diagnostic biomarkers with a reference, whereby pancreatic cancer is diagnosed.

In the method according to the present invention, at least the amounts of at least four diagnostic biomarkers shall be determined. The term “at least four diagnostic biomarkers”, as used herein, means four or more than four. Accordingly, the amounts of four, five, six, seven, eight, nine, ten, eleven, or even more diagnostic biomarkers may be determined (and compared to a reference, as specified elsewhere herein). Preferably, the amounts of four to eleven diagnostic biomarkers, are determined (and compared to a reference).

According to the present invention, the diagnostic biomarkers of a group of diagnostic biomarkers are selected such that said group comprises at least one diagnostic amino acid biomarker, at least one diagnostic ceramide biomarker, at least one diagnostic sphingomyelin biomarker, and CA19-9. Preferably, said group of diagnostic biomarkers further comprises at least one diagnostic ethanolamine lipid.

As used herein, the term “diagnostic amino acid” relates to proline, histidine or tryptophan; preferably, the diagnostic amino acid is proline; in another embodiment, preferably, the diagnostic amino acid is tryptophan.

As used herein, the term “diagnostic ceramide” relates to ceramide (d18:1,C24:0) or ceramide (d18:2,C24:0); preferably, the diagnostic ceramide is ceramide (d18:1,C24:0); in another embodiment, preferably, the diagnostic ceramide is ceramide (d18:2,C24:0).

As used herein, the term “diagnostic sphingomyelin” relates to sphingomyelin (35:1), sphingomyelin (d17:1,C16:0), sphingomyelin (41:2) or sphingomyelin (d18:2,C17:0); preferably, the diagnostic sphingomyelin is sphingomyelin (35:1); in another embodiment, preferably, the diagnostic sphingomyelin is sphingomyelin (41:2). In a preferred embodiment, the diagnostic sphingomyelin is sphingomyelin (35:2).

As will be understood by the skilled person, the term “sphingomyelin (35:1)” relates to sphingomyelins wherein the sum of carbon atoms in the sphingoid moiety and the fatty acid moiety together is 35, and wherein said sphingomyelins comprise one double bond. Preferably, in case said double bond is present in the sphingoid base, said double bond is a trans double bond, and, in case said double bond is present in the fatty acid moiety, said double bond is a cis double bond. Accordingly, the diagnostic biomarker sphingomyelin (35:1) preferably represents sphingomyelin (d18:1,C17:0) and sphingomyelin (d17:1,C18:0); or represents sphingomyelin (d18:1,C17:0); or represents sphingomyelin (d17:1,C18:0). More preferably, the diagnostic biomarker sphingomyelin (35:1) represents sphingomyelin (d18:1,C17:0) and sphingomyelin (d17:1,C18:0); or represents sphingomyelin (d18:1,C17:0). Even more preferably, sphingomyelin (35:1) represents sphingomyelin (d17:1,C18:0).

Similarly, the term “sphingomyelin (41:2)” relates to a sphingomyelin wherein the sum of carbon atoms in the sphingoid moiety and the fatty acid moiety together is 41, and wherein said sphingomyelins comprise two double bonds. Preferably, in case a double bond is present in the sphingoid base, said double bond is a trans double bond, and, in case a double bond is present in the fatty acid moiety, said double bond is a cis double bond. In case two double bonds are present in the sphingoid base, one of those said double bonds is preferably a trans double bond and the second one of those double bonds can be either of trans or cis configuration; i.e. preferably, in case two double bonds are present in the sphingoid base, one thereof is a trans double bond. Accordingly, the diagnostic biomarker sphingomyelin (41:2) preferably represents sphingomyelin (d18:1,C23:1), sphingomyelin (d17:1,C24:1), and sphingomyelin (d18:2,C23:0); or represents sphingomyelin (d18:1,C23:1) and sphingomyelin (d17:1,C24:1); or represents sphingomyelin (d17:1,C24:1) and sphingomyelin (d18:2,C23:0); or represents sphingomyelin (d18:1,C23:1) and sphingomyelin (d18:2,C23:0); or represents sphingomyelin (d18:1,C23:1); or represents sphingomyelin (d17:1,C24:1); or represents sphingomyelin (d18:2,C23:0). More preferably, the diagnostic biomarker sphingomyelin (41:2) represents sphingomyelin (d17:1,C24:1) or sphingomyelin (d18:2,C23:0); even more preferably, it represents sphingomyelin (d17:1,C24:1).

In a preferred embodiment, the term “sphingomyelin (35:2)” relates to a sphingomyelin wherein the sum of carbon atoms in the sphingoid moiety and the fatty acid moiety together is 35, and wherein said sphingomyelin comprises two double bonds. Preferably, the diagnostic biomarker sphingomyelin (35:2) represents sphingomyelin (d18:2,C17:0), sphingomyelin (d17:1,C18:1), sphingomyelin (d17:2,C18:0), and/or sphingomyelin (d18:1,C17:1). More preferably, the diagnostic biomarker sphingomyelin (35:2) represents at least three, even more preferably at least two sphingomyelins selected from the list consisting of sphingomyelin (d18:2,C17:0), sphingomyelin (d17:1,C18:1), sphingomyelin (d17:2,C18:0), and sphingomyelin (d18:1,C17:1). Most preferably, sphingomyelin (35:2) represents sphingomyelin (d18:2,C17:0).

The term “CA19-9” is known to the skilled person, also as “carbohydrate antigen 19-9” or as “gastrointestinal cancer associated antigen 19-9”, e.g. from Gupta et al., 1985, Cancer 56 (277-283). Tests for specifically determining CA19-9 in, e.g., a blood derived sample, are commercially available.

As used herein, the term “diagnostic ethanolamine lipid” relates to phosphatidylethanolamine (C18:0,C22:6), lysophosphatidylethanolamine (C18:2) or lysophosphatidylethanolamine (C18:0); preferably, the diagnostic ethanolamine lipid is phosphatidylethanolamine (C18:0,C22:6); in another embodiment, preferably the diagnostic ethanolamine lipid is lysophosphatidylethanolamine (C18:0).

Preferably, in the group of diagnostic biomarkers, the diagnostic amino acid is proline and/or the diagnostic sphingomyelin is sphingomyelin (35:1) or sphingomyelin (d18:1,C17:0). More preferably, in the group of diagnostic biomarkers, the diagnostic amino acid is proline and the diagnostic sphingomyelin is sphingomyelin (35:1). Even more preferably, the group of diagnostic biomarkers comprises, preferably consists of, the diagnostic biomarkers proline, ceramide (d18:1,C24:0), sphingomyelin (35:1), and CA19-9; or the group of diagnostic biomarkers comprises, preferably consists of, the diagnostic biomarkers proline, ceramide (d18:2,C24:0), sphingomyelin (35:1), and CA19-9. In another embodiment, preferably, in the group of diagnostic biomarkers, the diagnostic amino acid is tryptophan. More preferably, in the group of diagnostic biomarkers, the diagnostic amino acid is tryptophan and the diagnostic ceramide is ceramide (d18:1,C24:0).

Preferably, the group of diagnostic biomarkers, further comprises at least one diagnostic ethanolamine lipid. More preferably, the group of diagnostic biomarkers comprises, preferably consists of, the diagnostic biomarkers proline, ceramide (d18:1,C24:0), sphingomyelin (35:1), phosphatidylethanolamine (C18:0,C22:6), and CA19-9. In another embodiment, more preferably, the group of diagnostic biomarkers comprises, preferably consists of, the diagnostic biomarkers proline, ceramide (d18:2,C24:0), sphingomyelin (35:1), phosphatidylethanolamine (C18:0,C22:6) and CA19-9.

In a preferred embodiment, the group of diagnostic biomarkers comprises, preferably consists of, the diagnostic biomarkers of at least one of the panels of Table 9, i.e., preferably, the diagnostic biomarkers of a panel selected from the list consisting of panels 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, and 104 of Table 9. In a preferred embodiment, the diagnostic biomarkers are those of a panel selected from the list consisting of panels 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, and 106 of Tables 9 and 17.

In a preferred embodiment, the group of diagnostic biomarkers comprises, preferably consists of, the diagnostic biomarkers of panel 1, 2, 6, 7, 11, 82, 9, 89, 44, 10, 58, 46, 66, 13, 31, 30, 92, 86, 48, 81 or 90 of Table 9. In another preferred embodiment, the group of diagnostic biomarker comprises, preferably consists of, the diagnostic biomarkers of panel 2, 6, 7, 11, 82, 9, 89, 44, 10, 58, 46, 66, 13, 31, 30, 92, 86, 48, 81 or 90 of Table 9; in an even more preferred embodiment, the group of diagnostic biomarker comprises, preferably consists of, the diagnostic biomarkers of panel 2, 7, 82, 89, 44, 58, 46, 66, 13, 31, 30, 92, 48 or 90 of Table 9.

In a further preferred embodiment, the subject is a subject suffering from chronic pancreatitis and said group of diagnostic biomarkers comprises, preferably consists of, the diagnostic biomarkers of panel 1, 2, 6, 7, 4, 12, 14, 43, 19, 13, 16, 41, 21, 50, 44, 47, 46, 48, 3, or 5 of Table 9. In another preferred embodiment the group of diagnostic biomarkers comprises, preferably consists of, the diagnostic biomarkers of panel 2, 6, 7, 4, 12, 14, 43, 19, 13, 16, 41, 21, 50, 44, 47, 46, 48, 3, or 5 of Table 9; in an even more preferred embodiment, the group of diagnostic biomarker comprises, preferably consists of, the diagnostic biomarkers of panel 2, 7, 4, 12, 14, 43, 19, 13, 16, 41, 21, 50, 44, 47, 46 or 48 of Table 9. In a further preferred embodiment, the group of diagnostic biomarkers comprises, preferably consists of, the diagnostic biomarkers of panel 13, 66, 46, 104, 58, 10, 44, 89, 9, 82, 2, or 11 of Table 9; in an even more preferred embodiment, the group of diagnostic biomarker comprises, preferably consists of, the diagnostic biomarkers of 13, 66, 46, 58, 44, 89, 82, or 2 of Table 9.

In a further preferred embodiment, said subject is a subject suffering from new-onset diabetes and the group of diagnostic biomarkers comprises, preferably consists of, the diagnostic biomarkers of panel 1, 2, 6, 7, 13, 9, 43, 12, 10, 11, 47, 21, 14, 49, 48, 4, 19, 46, 82 or 52 of Table 9; in another preferred embodiment the group of diagnostic biomarkers comprises, preferably consists of, the diagnostic biomarkers of panel, 2, 6, 7, 13, 9, 43, 12, 10, 11, 47, 21, 14, 49, 48, 4, 19, 46, 82 or 52 of Table 9; in an even more preferred embodiment group of diagnostic biomarkers comprises, preferably consists of, the diagnostic biomarkers of panel, 2, 7, 13, 43, 12, 47, 21, 14, 49, 48, 4, 19, 46, 82 or 52 of Table 9.

In a further preferred embodiment, said subject is a subject with a low CA19-9 value and the group of diagnostic biomarkers comprises, preferably consists of, the diagnostic biomarkers of panel 1, 2, 6, 7, 9, 13, 12, or 3 of Table 9; in another preferred embodiment the group of diagnostic biomarkers comprises, preferably consists of, the diagnostic biomarkers of panel 2, 6, 7, 9, 13, 12, or 3 of Table 9, in an even more preferred embodiment group of diagnostic biomarkers comprises, preferably consists of, the diagnostic biomarkers of panel 2, 7, 13 or 12 of Table 9.

In another preferred embodiment, the pancreatic cancer is a resectable pancreatic cancer and the group of diagnostic biomarkers comprises, preferably consists of, the diagnostic biomarkers of panel 1, 2, 6, 7, 3, 4, 5, 9, 10, 12, 13, 14, 15, 16, 18, 19, 11, 21, 22 or 30 of Table 9; in another preferred embodiment the group of diagnostic biomarkers comprises, preferably consists of, the diagnostic biomarkers of panel, 2, 6, 7, 3, 4, 5, 9, 10, 12, 13, 14, 15, 16, 19, 11, 21 or 30 of Table 9, in an even more preferred embodiment group of diagnostic biomarkers comprises, preferably consists of, the diagnostic biomarkers of panel of 2, 7, 4, 12, 13, 14, 16, 19, 21 or 30 Table 9.

Further preferred are groups of diagnostic biomarkers comprising proline, preferably the diagnostic biomarkers of a panel selected from the list consisting of panels 1-21, 38-55, and 104 of Table 9. Further preferred are groups of diagnostic biomarkers comprising ceramide (d18:1, C24:0), preferably the diagnostic biomarkers of a panel selected from the list consisting of panels 1-11, 16-18, 22-25, 30-33, 38-46, 56-67, 80-91, and 104 of Table 9. Further preferred are groups of diagnostic biomarkers comprising proline and ceramide (d18:1, C24:0), preferably the diagnostic biomarkers of a panel selected from the list consisting of panels 1-11, 16-18, 38-46, and 104 of Table 9. In a preferred embodiment, the subject to be tested is above 40 years of age and the diagnostic biomarkers of a panel selected from the list consisting of panels 1-21, 38-55, and 104 of Table 9 or from the list consisting of panels 1-11, 16-18, 22-25, 30-33, 38-46, 56-67, 80-91, and 104 of Table 9 or from the list consisting of panels 1-11, 16-18, 38-46, and 104 of Table 9 are determined.

Further preferred are groups of diagnostic biomarkers comprising ceramide (d18:2, C24:0) preferably the diagnostic biomarkers of a panel selected from the list consisting of panels 12-15, 19-21, 26-29, 34-37, 47-55, 68-79, 92-103, and 104 of Table 9. Further preferred are groups of diagnostic biomarkers comprising proline and ceramide (d18:2, C24:0), preferably the diagnostic biomarkers of a panel selected from the list consisting of panels 12-15, 19-21, 47-55, and 104 of Table 9.

Further preferred are groups of diagnostic biomarkers comprising sphingomyelin (35:1), preferably the diagnostic biomarkers of a panel selected from the list consisting of panels 1-15, 22, 26, 30, 34, 56, 57, 58, 68, 69, 70, 80, 81, 82, 92, 93, 94, and 104 of Table 9. Further preferred are groups of diagnostic biomarkers comprising proline, ceramide (d18:1,C24:0), sphingomyelin (35:1), and CA19-9, preferably the diagnostic biomarkers of a panel selected from the list consisting of panels 1-11 and 104 of Table 9. Further preferred are groups of diagnostic biomarkers comprising proline, ceramide (d18:2,C24:0), sphingomyelin (35:1), and CA19-9, preferably the diagnostic biomarkers of a panel selected from the list consisting of panels 12-15 and 104 of Table 9. In a preferred embodiment, the subject to be tested is above 40 years of age and the diagnostic biomarkers of a panel selected from the list consisting of panels 1-15, 22, 26, 30, 34, 56, 57, 58, 68, 69, 70, 80, 81, 82, 92, 93, 94, and 104 of Table 9 or from the list consisting of panels 1-11 and 104 of Table 9 are determined.

Further preferred are groups of diagnostic biomarkers comprising at least one diagnostic ethanolamine lipid, preferably the diagnostic biomarkers of a panel selected from the list consisting of panels 2-6, 8-11, 13-15, 38-103, and 104 of Table 9. In a preferred embodiment, the subject to be tested is above 40 years of age and the diagnostic biomarkers of a panel selected from the list consisting of panels 2-6, 8-11, 13-15, 38-103, and 104 of Table 9 are determined.

Further preferred are groups of diagnostic biomarkers comprising phosphatidylethanolamine (C18:0,C22:6), preferably the diagnostic biomarkers of a panel selected from the list consisting of panels 2, 9-11, 13, 43, 44, 46-49, 58, 65-67, 70, 71, 78, 79, 82, 88-90, 92, 95-97, and 104 of Table 9. Further preferred are groups of diagnostic biomarkers comprising proline, ceramide (d18:1,C24:0), sphingomyelin (35:1), phosphatidylethanolamine (C18:0,C22:6), and CA19-9, preferably the diagnostic biomarkers of a panel selected from the list consisting of panels 2, 9, 10, 11, and 104 of Table 9. Further preferred are groups of diagnostic biomarkers comprising proline, ceramide (d18:2,C24:0), sphingomyelin (35:1), phosphatidylethanolamine (C18:0,C22:6) and CA19-9, preferably the diagnostic biomarkers of a panel selected from the list consisting of panels 13 and 104 of Table 9.

In a preferred embodiment, the group of diagnostic biomarkers comprising the diagnostic biomarkers of panel 1, 18, 22, 23, 25, or 61 of Table 9 comprises at least one further diagnostic biomarker. Also in a preferred embodiment, the sample is a sample obtained from said subject while said subject was fasting and the group of diagnostic biomarkers comprises, preferably consists of, the diagnostic biomarkers of panel 1, 18, 22, 23, 25, or 61 of Table 9. Also in a preferred embodiment, the subject is a subject at risk of suffering from pancreatic cancer and the group of diagnostic biomarkers comprises, preferably consists of, the diagnostic biomarkers of panel 1, 18, 22, 23, 25, or 61 of Table 9. Also in a preferred embodiment, the subject is a subject with new-onset diabetes and the group of diagnostic biomarkers comprises, preferably consists of, the diagnostic biomarkers of panel 1, 18, 22, 23, 25, or 61 of Table 9. Also in a preferred embodiment, the subject is a subject suffering from chronic pancreatitis and the group of diagnostic biomarkers comprises, preferably consists of, the diagnostic biomarkers of panel 1, 18, 22, 23, 25, or 61 of Table 9. Also in a preferred embodiment, the subject is a subject with a low CA19-9 value and the group of diagnostic biomarkers comprises, preferably consists of, the diagnostic biomarkers of panel 1, 18, 22, 23, 25, or 61 of Table 9. Also in a preferred embodiment, the subject is a subject suspected to suffer from pancreatic cancer and the group of diagnostic biomarkers comprises, preferably consists of, the diagnostic biomarkers of panel 1, 18, 22, 23, 25, or 61 of Table 9. Also in a preferred embodiment, the pancreatic cancer is a pancreatic cancer with a resectable tumor stage and the group of diagnostic biomarkers comprises, preferably consists of, the diagnostic biomarkers of panel 1, 18, 22, 23, 25, or 61 of Table 9.

In a most preferred embodiment, the group of diagnostic biomarkers comprises, preferably consists of, the diagnostic biomarkers CA19-9, Ceramide (d18:1,C24:0), Ceramide (d18:2,C24:0), Histidine, Lysophosphatidylethanolamine (C18:0), Lysophosphatidylethanolamine (C18:2), Phosphatidylethanolamine (C18:0,C22:6), Proline, Sphingomyelin (d17:1,C16:0), Sphingomyelin (35:1), Sphingomyelin (41:2), Sphingomyelin (d18:2,C17:0), and Tryptophan.

In a further preferred embodiment, the group of diagnostic biomarkers comprises a sum parameter, i.e. a parameter obtained by summing up the semi-quantitative or, preferably, quantitative amounts determined for two or more metabolites. A sum parameter as used herein, is indicated as [A+B], i.e. as the sum of A and B.

Thus, in a preferred embodiment, the group of diagnostic biomarkers comprises a sum parameter of more than one amino acid, preferably a sum parameter including the semi-quantitative or, preferably, quantitative amounts of histidine and proline, of histidine and tryptophan, and/or of proline and tryptophan. In a further preferred embodiment, the group of diagnostic biomarkers comprises a sum parameter including, preferably consisting of, the semi-quantitative or, preferably, quantitative amounts of histidine, proline, and tryptophan.

In a further preferred embodiment, the group of diagnostic biomarkers comprises a sum parameter of more than one sphingomyelin, preferably a sum parameter including the semi-quantitative or, preferably, quantitative amounts of (i) sphingomyelin (d17:1,C16:0) and sphingomyelin (d18:2,C17:0), (ii) of sphingomyelin (d17:1,C16:0) and sphingomyelin (35:1), (iii) of sphingomyelin (d17:1,C16:0) and sphingomyelin (41:2), (iv) of sphingomyelin (d18:2,C17:0) and sphingomyelin (35:1), (v) of sphingomyelin (d18:2,C17:0) and sphingomyelin (41:2), (vi) of sphingomyelin (d17:1,C16:0), sphingomyelin (d18:2,C17:0), and sphingomyelin (35:1), (vii) sphingomyelin (d17:1,C16:0), sphingomyelin (d18:2,C17:0), and sphingomyelin (41:2); (viii) sphingomyelin (d17:1,C16:0), sphingomyelin (35:1), and sphingomyelin (41:2), (ix) sphingomyelin (d18:2,C17:0), sphingomyelin (35:1), and sphingomyelin (41:2). In a preferred embodiment, the group of diagnostic biomarkers comprises a sum parameter including, preferably consisting of, the semi-quantitative or, preferably, quantitative amounts of sphingomyelin (d17:1,C16:0), sphingomyelin (d18:2,C17:0), sphingomyelin (35:1), and sphingomyelin (41:2).

In a further preferred embodiment, the group of diagnostic biomarkers comprises a sum parameter of more than one ceramide, preferably a sum parameter including, preferably consisting of, the semi-quantitative or, preferably, quantitative amounts of ceramide (d18:1,C24:0) and ceramide (d18:2,C24:0).

In a further preferred embodiment, the group of diagnostic biomarkers comprises a ratio parameter of lysophosphatidylethanolamines or a ratio of lysophosphatidylethanolamines and phosphatidylethanolamines, preferably a ratio parameter including, preferably consisting of, the semi-quantitative or, preferably, quantitative amounts of lysophosphatidylethanolamine (C18:2) and phosphatidylethanolamine (C18:0,C22:6), i.e., preferably the ratio lysophosphatidylethanolamine (C18:2)/phosphatidylethanolamine (C18:0,C22:6). A ratio parameter, as used herein, is indicated as [NB], i.e. as ratio of A divided by B.

Thus, in a preferred embodiment, the group of diagnostic biomarkers comprises, preferably consists of, CA 19-9, [histidine+proline+tryptophan], [sphingomyelin (d17:1,C16:0)+sphingomyelin (d18:2,C17:0)+sphingomyelin (35:1)+sphingomyelin (41:2)], and [ceramide (d18:1,C24:0)+ceramide (d18:2,C24:0)]. In a further preferred embodiment, the group of diagnostic biomarkers comprises, preferably consists of, CA 19-9, [histidine+proline+tryptophan], [sphingomyelin (d17:1,C16:0)+sphingomyelin (d18:2,C17:0)+sphingomyelin (35:1)+sphingomyelin (41:2)], [ceramide (d18:1,C24:0)+ceramide (d18:2,C24:0)], and [lysophosphatidylethanolamine (C18:2)/phosphatidylethanolamine (C18:0,C22:6)].

As noted above, the method of the present invention may comprise determining further biomarkers as well. As noted above, a further biomarker is not a diagnostic biomarker. In such case, in an embodiment, each further biomarker determined decreases the false-positive rate and/or the false negative rate of the method by at least 0.1%, preferably 1%. In a further embodiment, each further biomarker determined significantly increases the AUC value of the method. Accordingly, the method of the present invention, preferably, avoids determining biomarkers not contributing to improvement of diagnosis. Preferably, the group of diagnostic biomarkers does not comprise sphinganine-1-phosphate (d18:0); and/or, if said group of diagnostic biomarkers comprises histidine, the group of diagnostic biomarkers does not comprise sphingomyelin (d18:2,C17:0).

The term “determining the amount”, in particular of a biomarker, as used herein, refers to determining at least one characteristic feature of a biomarker to be determined a sample. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemiluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the said at least one biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z information, as well as an intensity value being related to the abundance of the said biomarker (i.e. its amount) in the sample.

As discussed before, a biomarker, preferably a diagnostic biomarker, comprised by a sample may be, preferably, determined in accordance with the present invention semi-quantitatively or quantitatively For semi-quantitative determination, preferably, the relative amount of the biomarker is determined based on the value determined for the characteristic feature(s) referred to herein above. The relative amount may be determined in a case were the precise amount of a biomarker can or shall not be determined. In said case, it can be determined whether the amount in which the biomarker is present, is increased or diminished with respect to a second sample comprising said biomarker in a second amount; or it can be determined whether the amount in which the biomarker is present, is increased or diminished with respect to an internal control analyte. Preferably, said second sample comprising said biomarker is a calculated reference as specified elsewhere herein. More preferably, the biomarker, in particular the diagnostic biomarker, is determined quantitatively, i.e. preferably, determining is measuring an absolute amount or a concentration of a biomarker.

A sample is, preferably, pre-treated before it is used for the method of the present invention. As described in more detail below, said pre-treatment may include treatments required to release or separate the compounds or to remove excessive material or waste. Suitable techniques comprise centrifugation, extraction, fractioning, ultrafiltration, separation (e.g. by binding to paramagnetic beads and applying magnetic force), protein precipitation followed by filtration and purification and/or enrichment of compounds. Moreover, other pre-treatments are preferably carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

Preferably, the pre-treatment of the sample allows for a subsequent separation of compounds, in particular of the small molecule diagnostic biomarkers as referred to above, comprised by the sample. Molecules of interest, in particular the biomarkers as referred to above may be extracted in an extraction step which comprises mixing of the sample with a suitable extraction solvent. The extraction solvent shall be capable of precipitating the proteins in a sample, thereby facilitating the, preferably, centrifugation-based, removal of protein contaminants which otherwise would interfere with the subsequent analysis of the biomarkers as referred above. Preferably, at least the small molecule diagnostic biomarkers as referred to herein are soluble in the extraction solvent. More preferably, the extraction solvent is a non-phase separating, i.e., a one phase solvent. Even more preferably, the extraction solvent is a non-phase separating, protein precipitating solution, preferably a mixture comprising a first solvent selected from the group consisting of dichloromethane (DCM), chloroform, tertiary butyl methyl ether (tBME or MTBE, also known as 2-methoxy-2-methylpropane), ethyl ethanoate, and isooctane, and a second solvent selected from the group consisting of methanol, ethanol, isopropanol and dimethyl sulfoxide (DMSO). More preferably, the non-phase separating, protein precipitating solution comprises methanol and DCM, in particular in a ratio of about 2:1 (v/v) to about 3:2 (v/v), preferably in a ratio of about 2:1 (v/v) or about 3:2 (v/v). More preferably, the non-phase separating, protein precipitating solution comprises methanol:dichloromethane in a ratio of 2:1 (v/v).

Preferably, the determination of the amount of a biomarker as referred to herein is achieved by a compound separation step as specified above and a subsequent mass spectrometry step. Thus, determining as used in the method of the present invention, preferably, includes using a compound separation step prior to the analysis step. Preferably, said compound separation step yields a time resolved separation of the metabolites, in particular of the diagnostic biomarkers, comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. Moreover, determination via ion mobility chromatography, preferably in combination with electrospray/MS/MS is envisaged. These techniques are well known in the art and can be applied by the person skilled in the art without further ado. Most preferably, LC and/or HPLC are chromatographic techniques to be envisaged by the method of the present invention. Suitable devices for such determination of biomarkers are well known in the art. Preferably, chromatography is reverse phase chromatography, more preferably reverse phase liquid chromatography, most preferably on a C18 reverse phase column.

Preferably, mass spectrometry is used, in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). More preferably, LC-MS, in particular LC-MS/MS are used as described in detail below. The techniques described above are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference.

As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). In a preferred embodiment, a biomarker may also be determined by its binding to a specific ligand, e.g. to an aptamer, an antibody, and the like. These techniques are well known to the person skilled in the art and can be applied without further ado.

The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

In an embodiment, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once.

More preferably, said mass spectrometry is liquid chromatography (LC) MS, such as high performance liquid chromatography (HPLC) MS, in particular HPLC-MS/MS. Liquid chromatography as used herein refers to all techniques which allow for separation of compounds (i.e. metabolites) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. For examples, HPLC can be carried out with commercially available reversed phase separation columns with e.g. C8, C18 or C30 stationary phases. The person skilled in the art is capable to select suitable solvents for the HPLC or any other chromatography method as described herein. The eluate that emerges from the chromatography device shall comprise the biomarkers as referred to above.

A suitable solvent for elution for lipid chromatography can be determined by the skilled person. In an embodiment, the solvents for gradient elution in the HPLC separation consist of a polar solvent and a lipid solvent. Preferably, the polar solvent is a mixture of water and a water miscible solvent with an acid modifier. Examples of suitable organic solvents which are completely miscible with water include the C1-C3-alkanols, tetrahydrofurane, dioxane, C3-C4-ketones such as acetone and acetonitril and mixtures thereof, with methanol being particularly preferred. Preferably, the lipid solvent is a mixture of at least one of the above mentioned organic solvents together with hydrophobic solvents from the groups consisting of dichloromethane (DCM), chloroform, tertiary butyl methyl ether (tBME or MTBE), ethyl ethanoate, and isooctane. Examples of acidic modifiers are formic acid or acidic acid. Preferred solvents for gradient elution are disclosed in the Examples section.

Gas chromatography, which may be also applied in accordance with the present invention, in principle, operates comparable to liquid chromatography. However, rather than having the compounds (i.e. metabolites) in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time that is required for passing through the column.

Moreover, in the case of gas chromatography, but also in liquid chromatography, in particular in reverse-phase liquid chromatography, it is preferably envisaged that the compounds are derivatized prior to chromatography. Suitable techniques for derivatization are well known in the art. Preferably, derivatization in accordance with the present invention relates to methoxymation and trimethylsilylation of, preferably, polar compounds and transmethylation, methoxymation and trimethylsilylation of, preferably, non-polar (i.e. lipophilic) compounds. More preferably, derivatization comprises, even more preferably consists of, contacting metabolites in the non-proteinaceous fraction with a reagent introducing hydrophobic side chains. Preferably, said reagent introducing hydrophobic side chains is a reagent derivatizing amino groups, preferably primary and secondary amino groups. More preferably, said reagent introducing hydrophobic side chains is 5-(dimethylamino)naphthalene-1-sulfonyl chloride (dansylchloride, CAS Registry No: 605-65-2). Preferably, of the small molecule diagnostic biomarkers, histidine is double-dansylated, and proline, tryptophan, lysophosphatidylethanolamine (C18:0), lysophosphatidylethanolamine (C18:2), and phosphatidylethanolamine (C18:0,C22:6) are monodansylated, respectively.

For mass spectrometry, the analytes in the sample are ionized in order to generate charged molecules or molecule fragments. Afterwards, the mass-to-charge of the ionized analyte, in particular of the ionized biomarkers, or fragments thereof is measured. Thus, the mass spectrometry step preferably comprises an ionization step in which the biomarkers to be determined are ionized. Of course, other compounds present in the sample/eluate are ionized as well. Ionization of the biomarkers can be carried out by any method deemed appropriate, in particular by electron impact ionization, fast atom bombardment, electrospray ionization (ESI), atmospheric pressure chemical ionization (APCI), matrix assisted laser desorption ionization (MALDI). Preferably, the ionization step is carried out by atmospheric pressure chemical ionization (APCI); in a preferred embodiment, the ionization step is carried out by APCI to resolve at least one of the specific sphingomyelins referred to herein under the designations sphingomyelin (35:1), sphingomyelin (41:2) and sphingomyelin (35:2). More preferably, the ionization step (for mass spectrometry) is carried out by electrospray ionization (ESI). Accordingly, the mass spectrometry is preferably ESI-MS (or if tandem MS is carried out: ESI-MS/MS). Electrospray is a soft ionization method which results in the formation of ions without breaking any chemical bonds. Electrospray ionization (ESI) is a technique used in mass spectrometry to produce ions using an electrospray in which a high voltage is applied to the sample to create an aerosol. It is especially useful in producing ions from macromolecules because it overcomes the propensity of these molecules to fragment when ionized. Preferably, the electrospray ionization is positive ion mode electrospray ionization. Thus, the ionization is preferably a protonation (or an adduct formation with positive charged ions such as NH₄⁺, Na⁺, or K⁺, in particular NH₄⁺). According a suitable cation, preferably, a proton (H⁺) is added to the biomarkers to be determined (and of course to any compound in the sample, i.e. in the eluate from the chromatography column). Therefore, the determination of the amounts of the diagnostic biomarkers might be the determination of the amount of protonated biomarkers.

The person skilled in the art knows that the ionization step is carried out at the beginning of the mass spectrometry step. If tandem MS is carried out, the ionization, in particular the electrospray ionization, is carried out in the first mass spectrometry step.

The ionization of the biomarkers can be preferably carried out by feeding the liquid eluting from the chromatography column (in particular from the LC or HPLC column) directly to an electrospray. Alternatively the fractions can be collected and are later analyzed in a classical nanoelectrospray-mass spectrometry setup.

As set forth above, the mass spectrometry step is carried out after the separation step, in particular the chromatography step. In an embodiment, the eluate that emerges from the chromatography column (e.g. the LC or HPLC column) may be pre-treated prior to subjecting it to the mass spectrometry step.

In a preferred embodiment of the present invention, the diagnostic biomarkers, preferably the small molecule diagnostic biomarkers, are determined together in a single measurement. In particular, it is envisaged to determine the amounts together in a single LC-MS (or LC-MS/MS), HPLC-MS (HPLC-MS/MS) measurement (i.e. run). Preferably, the amounts of the diagnostic biomarkers are determined as described in the Examples described elsewhere herein.

For example, a blood, serum, or plasma sample (in particular a plasma sample) can be analyzed. The sample may be a fresh sample or a frozen sample. If frozen, the sample may be thawed at suitable temperature for a suitable time. An aliquot of the sample is then transferred to a microcentrifuge tube and diluted with a suitable diluent as specified elsewhere herein. An internal standard may be added, before, upon, or after dilution. Preferably, in said internal standard only three, more preferably only two standard compounds are comprised. Preferably said internal standard comprises, more preferably consists of, L-Alanine d4 and Ceramide (d18:1,17:0). Afterwards an extraction is done (e.g. for 5 minutes using a vortexer). Afterwards, the sample may be centrifuged (e.g. at about 20.000 g). An aliquot of the supernatant (e.g. 200 μl) can be used for the quantification of the biomarkers.

In a preferred embodiment, at least one internal standard compound can be added to the sample. As used herein, the term “internal standard compound” refers to a compound which is added to the sample and which is determined (i.e. the amount of the internal standard compound is determined). The at least one internal standard compound can be added before, during, or after extraction of the sample to be tested. In an embodiment, the at least one internal standard compound is added before mixing an aliquot of the sample with the extraction solvent. Preferably, the at least one internal standard compound is dissolved separately in a suitable solvent before addition to the sample and, subsequent addition of the extraction solvent. In an embodiment, the extraction solvent is an extraction solvent as described elsewhere herein, such as methanol/dichloromethane (2:1 v/v).

Preferably, the internal standard compound is a compound, in particular a lipid, which is essentially not present or which is not present in the sample to be tested. Thus, the compound is preferably not naturally present in the sample to be tested. Preferably, the internal standard is very similar to a respective lipid biomarker according to the present invention. More preferably, the at least one internal standard compound is L-Alanine d4 or Ceramide (d18:1,17:0). Even more preferably, both L-Alanine d4 and ceramide (d18:1,17:0) are used as internal standard compounds.

As mentioned above, the internal standard compound(s) can be dissolved in a suitable solvent (the solution comprising the internal standard compound and the suitable solvent is herein also referred to as “internal standard solution”). Preferably, the solvent comprises or is dimethyl sulfoxide, methanol, dichloromethane and water. More preferably the solvent is a mixture comprising dimethyl sulfoxide, methanol, dichloromethane and water, in a ratio of about 12:2:1:1, v/v/v/v, even more preferably in a ratio of 12.3:2.2:1.1:1, v/v/v/v. Preferably, the internal standard solution comprises or consists of dimethyl sulfoxide, methanol, dichloromethane and water, in a ratio of about 12:2:1:1, v/v/v/v, even more preferably in a ratio of 12.3:2.2:1.1:1, v/v/v/v, and the internal standard compounds L-Alanine d4 and ceramide (d18:1,17:0) in a range of ratios of about 50/1 to 100/1 (L-Alanine d4/ceramide (d18:1,17:0), w/w), more preferably in a ratio of about 80/1 (L-Alanine d4/ceramide (d18:1,17:0), w/w), most preferably in a ratio of 79.5/1 (L-Alanine d4/ceramide (d18:1,17:0), w/w).

Preferably, the internal standard solution is added to the sample to be tested before extracting the samples with an extraction solvent as described elsewhere herein and removing the proteins from the sample by centrifugation. Thus, the extraction step shall preferably be carried out on a sample to which the internal standard solution was added.

If L-Alanine d4 is used as internal standard compound, the concentration of the internal standard compound in the internal standard solution to be added to the (thus pretreated) sample is preferably within a range of 1 to 200 μg/ml, more preferably 10 to 15 μg/ml, most preferably 12 to 13 μg/ml.

If ceramide (d18:1,17:0) is used as internal standard compound, the concentration of the internal standard compound in the internal standard solution to be added to the (thus pretreated) sample is preferably within a range of 0.05 to 0.5 μg/ml, more preferably 0.1 to 0.2 μg/ml, most preferably 0.150 to 0.160 μg/ml.

In an embodiment, the volume of the internal standard solution to be used is five times the volume of the sample before pretreatment, e.g. 20 μl of plasma and 100 μl internal standard solution are used. In this case, the volume of the extraction solvent is at least five times the volume of the sample pretreated with internal standard solution, e.g. 700 μl.

The determination of the amount of the internal standard shall preferably allow for a normalization of the amounts of the at least three biomarkers as referred to herein. Preferably, the determined peak areas for the diagnostic biomarkers are divided by the peak area of the at least one internal standard compound.

In particular, it is possible to calculate a correction factor for the test samples (samples from subjects to be tested, but also calibration samples). This correction factor can be used in order to correct the peak area of the at least three biomarkers for variations of the devices, inherent system errors, or the like. For each biomarker determined as peak area, the area ratio of the biomarker peak area to the internal standard peak area can be determined.

Preferably, the determination of the at least one standard compound does not interfere with the determination of the amounts of the diagnostic biomarkers. In a preferred embodiment, the internal standard solution is a solution not comprising sample, more preferably is a solution consisting of standard compound(s) and solvent(s) as specified herein above. Thus, in a preferred embodiment, the present invention relates to an internal standard solution as specified herein above.

In a preferred embodiment, quantitative determinations are calibrated using external calibration, preferably including delipidized plasma and preferably extraction solvent as specified herein for the sample. Preferably, an appropriate concentration of a quantification standard compound, preferably a quantification standard compound as described in Table 1, is provided, and a calibration curve is established using appropriate dilution series of said quantification standard. Preferably, the highest concentrations of the quantification standards used are 5.3475 μg/ml for Sphingomyelin (d18:1,C17:0), 12.177 μg/ml for Sphingomyelin (d18:1,C24:1), 3.125 μg/ml for Phosphatidylethanolamine (C18:0,C22:6), 2.932 μg/ml for Ceramide (d18:1,C24:0) 0.912 μg/ml for Ceramide (d18:1,C24:1), 1.24 μg/ml for Lysophosphatidylethanolamine (C18:0), 25.45 μg/ml for L-Proline, 27.5 μg/ml for L-Tryptophan, and/or 12.75 μg/ml for L-Histidine.

Thus, in a preferred embodiment, the present invention also relates to a kit comprising (i) at least one solution comprising at least one quantification standard compound selected from Sphingomyelin (d18:1,C17:0), Sphingomyelin (d18:1,C24:1), Phosphatidylethanolamine (C18:0,C22:6), Ceramide (d18:1,C24:0), Ceramide (d18:1,C24:1), Lysophosphatidylethanolamine (C18:0), L-Proline, L-Tryptophan, and L-Histidine; and (ii) delipidized plasma and/or an internal standard solution as specified herein above. Preferably, the concentration of said quantification standard compound in said solution is the concentration indicated herein above. As will be understood by the skilled person, in case the kit comprises more than one quantification standard compound, the quantification standard compounds are, preferably provided in the kit as separate solutions. In a further preferred embodiment, the present invention relates to the use of the aforesaid kit for diagnosing pancreatic cancer in a subject. Moreover, in a further preferred embodiment, the present invention relates to the use of the aforesaid kit in a method of the present invention.

As indicated above, the determination of the amount of CA19-9 may differ from the determination of the amounts of the other diagnostic biomarkers as referred to in the context of the method of the present invention (since the amounts of the other diagnostic biomarkers are preferably determined by the methods involving chromatography and mass spectrometry, see elsewhere herein). Preferably, the amount of CA19-9 is determined in a blood, serum or plasma sample. Preferably, the amount is determined by using at least one antibody which specifically binds to CA19-9, the at least one antibody forming a complex with the marker to be determined (CA19-9). Afterwards the amount of the formed complex is measured. The complex comprises the marker and the antibody (which might be labelled in order to allow for a detection of the complex).

It is to be understood that the sample in which CA19-9 is determined requires or may require a pretreatment which differs from the pretreatment of the sample in which the other diagnostic biomarkers as referred to herein are determined. For example, the proteins comprised by the sample in which this marker is determined may not be required to be precipitated. This is taken into account by the skilled person. Preferably, however, the amounts of the other diagnostic biomarkers and of CA19-9 are measured in aliquots derived from the same sample. Alternatively, the amounts of the other diagnostic biomarkers and of CA19-9 may be measured in aliquots derived from separate samples from the subject.

In an embodiment, the method of the present invention may further comprise carrying out a correction for confounders. Preferably, the values or ratios determined in a sample of a subject according to the present invention are adjusted for age, body mass index (BMI), gender or pre-existing diseases, e.g., renal and/or liver insufficiency. Alternatively, the references can be derived from values or ratios which have likewise been adjusted for age, BMI, and/or gender (see Examples). Such an adjustment can be made by deriving the references and the underlying values or ratios from a group of subjects the individual subjects of which are essentially identical with respect to these parameters to the subject to be investigated. Alternatively, the adjustment may be done by statistical calculations. Thus, a correction for confounders may be carried out. Preferred confounders are age, BMI (body mass index) and gender.

In another embodiment, a correction for confounders is not carried out. Preferably, no correction for the confounders age, BMI and gender is carried out. This may be advantageous since the diagnosis can be done even without the knowledge of certain patient's characteristics such as body mass index and gender. Thus, in an embodiment, the body mass index and/or gender are not known (and thus are not taken into account for the diagnosis).

The term “reference” in connection with diagnostic methods is well known in the art. The reference in accordance with the present invention shall allow for the diagnosis of pancreatic cancer. A suitable reference may be established by the skilled person without further ado. The term reference, preferably, refers to values of characteristic features which can be correlated to a medical condition, i.e. the presence or absence of the disease, diseases status or an effect referred to herein, or a calculated value or calculated values derived from said values. Preferably, the reference will be stored in a suitable data storage medium such as a database and are, thus, also available for future assessments.

Preferably, the reference is a value determined and/or calculated from a subject or group of subjects known to suffer from pancreatic cancer or is a value determined and/or calculated from an apparently healthy subject or group thereof, i.e. a “reference amount”.

The reference to be applied may be an individual reference for each of the diagnostic biomarkers to be determined in the method of the present invention. Accordingly, the amount of each of the diagnostic biomarkers as referred to in step a) of the method of the present invention is compared to a reference amount for each of the diagnostic biomarkers. For example, if four diagnostic biomarkers are determined in step a), four reference amounts (a reference amount for the first, a reference amount for the second, a reference amount for the third, and a reference amount for the fourth biomarker) are applied in step b). Based on the comparison of the amounts of the diagnostic biomarkers with the reference amounts, a diagnosis of pancreatic cancer, i.e. whether the subject as referred to herein suffers from pancreatic cancer, or not, is established. It is understood by the skilled person that a reference amount is not required to be an amount as determined in the determination step, but may also be a value derived therefrom by mathematical calculations well known to the skilled person. Preferably, a reference amount is a threshold value for a biomarker, preferably, a diagnostic biomarker, as referred to in connection with the present invention, whereby values found in a sample to be investigated which are higher than (or depending on the marker lower than) the threshold are indicative for the presence of pancreatic cancer while those being lower (or depending on the marker higher than) are indicative for the absence of pancreatic cancer.

The diagnostic algorithm, i.e. the specific set of calculation rules applied to obtain the diagnosis, may depend on the reference. If the reference amount is e.g. derived from a subject or group of subjects known to suffer from pancreatic cancer, the presence of pancreatic cancer is preferably indicated by amounts in the test sample which are essentially identical to the reference(s). If the reference amount is e.g. derived from an apparently healthy subject or group thereof, the presence of pancreatic cancer is preferably indicated by amounts of the diagnostic biomarkers in the test sample which are different from (e.g. increased (“up”) or decreased (“down”) as compared to the reference(s).

In accordance with the aforementioned method of the present invention, a reference amount (or reference amounts) is, preferably, a reference amount (or reference amounts) obtained from a sample from a subject or group of subjects known to suffer from pancreatic cancer. In such a case, a value for each of the diagnostic biomarkers found in the at least one test sample being essentially identical is indicative for the presence of the disease, i.e. of pancreatic cancer. Moreover, the reference amount, also preferably, could be from a subject or group of subjects known not to suffer from pancreatic cancer, preferably, an apparently healthy subject or group of subjects. In such a case, a value for each of the diagnostic biomarkers found in the test sample being altered, preferably altered in the direction indicated in Table 1, with respect to the reference amount is indicative for the presence of the disease. Alternatively, a value for each of the diagnostic biomarkers found in the test sample being essentially identical with respect to the reference amount is indicative for the absence of the disease. The same applies mutatis mutandis for a calculated reference, most preferably the average or median, for the relative or absolute value of the diagnostic biomarkers in a population of individuals comprising the subject to be investigated. The absolute or relative values of the biomarkers of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be diagnosed by the method of the present invention and the subjects of the said plurality of subjects are of the same species.

The value for a biomarker of the test sample and the reference amounts are essentially identical if the values for the characteristic features and, in the case of quantitative determination, the intensity values, or values derived therefrom, for the biomarker and the reference are essentially identical. Essentially identical means that the difference between two values is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value. Statistical test for determining whether two amounts or values are essentially identical are well known in the art and are also described elsewhere herein. An observed difference for two values, on the other hand, shall be statistically significant. A difference in the relative or absolute value is, preferably, significant outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference value. In a preferred embodiment, the value for the characteristic feature can also be a calculated output such as score of a classification algorithm like “elastic net” as set forth elsewhere herein.

More preferably, the reference is derived from two subjects or groups of subjects known to differ in a property to be diagnosed, e.g. a subject or group of subjects known to suffer from pancreatic cancer and an apparently healthy subject or group thereof, e.g. as a reference score or as a cutoff value distinguishing between said two subjects or groups, as specified herein below. The skilled person understands that other subject groups which shall be distinguished can be used as well for establishing e.g. a cutoff value. Preferably, a group of subjects known to suffer from pancreatic cancer is distinguished from a group of subjects known to suffer from chronic pancreatitis; or a group of subjects known to suffer from pancreatic cancer is distinguished from non-pancreatic controls; or a group of subjects known to suffer from pancreatic cancer is distinguished from group of subjects known to suffer from chronic pancreatitis and/or non-pancreatic controls; or a group of subjects known to suffer from pancreatic cancer is distinguished from all non-cancer subjects, preferably including chronic pancreatitis patients, non-pancreatic controls, diabetes patients, and/or non-diabetic subjects; or a group of subjects known to suffer from pancreatic cancer is distinguished from diabetic subjects. Moreover, more than one cutoff value may be provided, e.g., two cutoff values may be determined, wherein the interval between the first and the second cutoff may define, preferably diagnosis of increased risk of suffering from a disease to be diagnosed, warranting, e.g., closer monitoring of the patient.

The term “comparing”, preferably, refers to determining whether the determined value of the diagnostic biomarkers, or score (see below) is essentially identical to a reference or differs therefrom. Based on the comparison referred to above, a subject can be assessed to suffer from pancreatic cancer, or not. For the diagnostic biomarkers referred to in this specification, the kind of direction of change (i.e. “up”- or “down” or increase or decrease resulting in a higher or lower relative and/or absolute amount or ratio) are indicated in the Table 1 in the Examples section. It is to be understood that the diagnostic algorithm may be adjusted to the reference or references to be applied. This is taken into account by the skilled person who can establish suitable reference values and/or diagnostic algorithms based on the diagnosis provided herein. The comparison is, preferably, assisted by automation. For example, a suitable computer program comprising algorithms for the comparison of two different data sets (e.g., data sets comprising the values of the characteristic feature(s)) may be used. Such computer programs and algorithms are well known in the art. Notwithstanding the above, a comparison can also be carried out manually.

In the context of step b) of the method of the present invention, the amounts of a group of diagnostic biomarkers as referred to in step a) of the method of the present invention shall be compared to a reference. As used herein, the term “comparing said amounts of the diagnostic biomarkers with a reference” preferably relates to comparing said amounts to corresponding references on a one-to-one basis.

As can be derived from Table 1, an increased amount of phosphatidylethanolamine (C18:0,C22:6), sphingomyelin (d17:1,C16:0), sphingomyelin (35:1), and/or sphingomyelin (41:2) as compared to a reference amount is indicative for the presence of pancreatic cancer (and thus for the diagnosis of pancreatic cancer), whereas a decreased or an essentially identical amount as compared to the reference amount shall be indicative for the absence of pancreatic cancer. Preferably, said reference amount is a reference amount derived from healthy control subjects (i.e. subjects known not to suffer from pancreatic cancer), or from a healthy control subject. In a preferred embodiment, said term relates to comparing one or more calculated value(s) derived from said values to a reference value or reference values, preferably calculated mutatis mutandis from a reference population as specified elsewhere herein. Thereby, the presence or absence of disease as referred to herein is diagnosed.

As can be also derived from Table 1, a decreased amount of histidine, proline, tryptophan, ceramide (d18:1,C24:0), ceramide (d18:2,C24:0), Lysophosphatidylethanolamine (C18:0), and/or Lysophosphatidylethanolamine (C18:2) as compared to the reference amount shall be indicative for the presence of pancreatic cancer (and thus for the diagnosis of pancreatic cancer), whereas an increased or an essentially identical amount as compared to the reference amount shall be indicative for the absence of pancreatic cancer. Preferably, said reference amount is a reference amount derived from healthy control subjects (i.e. subjects known not to suffer from pancreatic cancer), or from a healthy control subject.

For CA19-9, an increased amount of this diagnostic biomarker is indicative for the presence of pancreatic cancer (and thus for the diagnosis of pancreatic cancer), whereas a decreased or an essentially identical amount is indicative for the absence of pancreatic cancer.

More preferably, the term “comparing said amounts of the diagnostic biomarkers with a reference” relates to calculating a score (in particular a single score) based on the amounts of the diagnostic biomarkers as referred to in step a) of the method of the present invention and comparing said score to a reference score or, preferably, to a cutoff. Preferably, the score is based on the amounts of the diagnostic biomarkers in the sample from the subject. The calculated score combines information on the amounts of the diagnostic biomarkers. The score can be regarded as a classifier parameter for diagnosing pancreatic cancer. In particular, it enables the person to provide the diagnosis based on a single score and based on the comparison with a reference score. The reference score is preferably a value, in particular a cutoff value, which allows for differentiating between the presence of pancreatic cancer and the absence of pancreatic cancer in the subject to be tested. Preferably, the reference score is a single value. Thus, the person does not have to interpret the entire information on the amounts of the individual diagnostic biomarkers.

Thus, in a preferred embodiment of the present invention, the comparison of the amounts of the diagnostic biomarkers to a reference as set forth in step b) of the method of the present invention encompasses step b1) of calculating a score based on the determined amounts of the diagnostic biomarkers as referred to in step a), and step b2) of comparing the, thus, calculated score to a reference score. Alternatively, the amount of each of the diagnostic biomarkers is compared to a reference, wherein the result of this comparison is used for the calculation of a score (in particular a single score), and wherein said score is compared to a reference score.

As set forth elsewhere herein, the aforementioned method may further comprise in step

a) the determination of the amount CA19-9. The amount of CA19-9 may contribute to the score calculated in step b). Accordingly, the method preferably comprises the following steps:
a) determining in a sample of a subject as referred to herein the amounts of small molecule diagnostic biomarkers as referred to above and the amount of CA19-9; and
b1) calculating a score based on the determined amounts of the at small molecule diagnostic biomarkers and on the amount of CA19-9 as referred to in step a), and
b2) comparing the, thus, calculated score to a reference score, whereby pancreatic cancer is diagnosed.

Alternatively, the amount of CA19-9 may not contribute to the score calculated in step b1). Accordingly, the method preferably comprises the following steps:

a) determining in a sample of a subject as referred to herein the amounts of small molecule diagnostic biomarkers as referred to above and the amount of CA19-9; and
b1) calculating a score based on the determined amounts of the small molecule diagnostic biomarkers, and
b2) comparing the, thus, calculated score to a reference score, and comparing the amount of CA19-9 to a reference, whereby pancreatic cancer is to be diagnosed.

Preferably, the score is calculated based on a suitable scoring algorithm. Said scoring algorithm, preferably, shall allow for differentiating whether a subject suffers from a disease as referred to herein, or not, based on the amounts of the biomarkers to be determined. Preferably, said scoring algorithm has been previously determined by comparing the information regarding the amounts of the individual biomarkers as referred to in step a) in samples from patients suffering from pancreatic cancer as referred to herein and from patients not suffering from pancreatic cancer. Accordingly, step b) may also comprise step b0) of determining or implementing a scoring algorithm. Preferably, this step is carried out prior steps b1) and b2).

Preferably, the reference score shall allow for differentiating whether a subject suffers from pancreatic cancer as referred to herein, or not. Preferably, the diagnosis is made by assessing whether the score of the test subject is above or below the reference score. Thus, in an embodiment, it is not necessary to provide an exact reference score. Preferably, the reference score refers to the same markers as the score. The reference score may be a “Cutoff” value which allows for differentiating between the presence and the absence of pancreatic cancer in the subject.

A cutoff value which delimitates the group of subjects suffering from pancreatic cancer from those which do not suffer from pancreatic cancer can be calculated by algorithms well known in the art, e.g., on the basis of the amounts of the biomarkers found in either group. Typically, a cutoff value can be, preferably, determined based on sensitivity, specificity and expected, known (e.g., from literature) or estimated (e.g, based on a prospective cohort study) prevalence for the disease in a certain population of subjects to be investigated. Preferably, receiver-operating characteristics (ROC) can be used for determining cutoff values (Zweig 1993, Clin. Chem. 39:561-577). How to apply the ROC technique is well known to the skilled artisan and the cutoff value preferably used in the method of the present invention is a cutoff value which allows discriminating between subjects suffering from the disease and those not suffering from the disease. It will be understood that a reference score resulting in a high sensitivity results in a lower specificity and vice versa; Thus, sensitivity and specificity may be adjusted according to the intended use case of the method of the present invention: In an embodiment, sensitivity and specificity are adjusted such that the group of false negatives is minimal in order to exclude a subject for being at increased risk efficiently (i.e. a rule-out); in another embodiment, sensitivity and specificity are adjusted such that the group of false positives is minimal in order for a subject to be assessed as being at an increased risk efficiently (i.e. a rule-in). In a further embodiment, a reference score for an optimized accuracy can be obtained using methods well known to the person skilled in the art, e.g. by maximizing the “Youden Index”. Moreover, the area under the curve (AUC) values can be derived from the ROC plots giving an indication for the cutoff independent, overall performance of the biomarker. Furthermore, each point of the ROC curve represents a sensitivity and specificity pair at a certain cutoff value.

In an embodiment, the reference score is calculated such that an increased value of the score of the test subject as compared to the reference score is indicative for the presence of pancreatic cancer, and/or a decreased value of the score of the test subject as compared to the reference score is indicative for the absence of pancreatic cancer. In particular, the score may be a cutoff value.

In a preferred embodiment of the present invention (e.g. of the methods, devices, uses etc.), the reference score is a single cutoff value. Preferably, said value allows for allocating the test subject either into a group of subjects suffering from pancreatic cancer or a into a group of subjects not suffering from pancreatic cancer. Preferably, a score for a subject lower than the reference score is indicative for the absence of pancreatic cancer in said subject (and thus can be used for ruling out pancreatic cancer), whereas a score for a subject larger than the reference score is indicative for the presence of pancreatic cancer in said subject (and thus can be used for ruling in pancreatic cancer).

In another preferred embodiment of the present invention (e.g. of the methods, devices, uses etc.), the reference score is a reference score range. In this context, a reference score range indicative for the presence of pancreatic cancer, a reference score range indicative for the absence of pancreatic cancer, or two reference score ranges (i.e. a reference score range indicative for the presence of pancreatic cancer and a reference score range indicative for the absence of pancreatic cancer) can be applied. A reference score range indicative for the absence of pancreatic cancer can be applied, if pancreatic cancer shall be ruled out. A reference score range indicative for the presence of pancreatic cancer can be applied, if pancreatic cancer shall be ruled in. The two reference score ranges as set forth above can be applied, if it should be diagnosed, whether the subject suffers from pancreatic cancer, or not. Preferably, the score of a subject is compared to the reference score range (or ranges). Preferably, the absence of pancreatic cancer is diagnosed, if the score is within the reference score range indicative for the absence of pancreatic cancer. Alternatively, the presence of pancreatic cancer is diagnosed, if the score is within the reference score range indicative for the presence of pancreatic cancer.

A suitable scoring algorithm can be determined with the diagnostic biomarkers referred to in step a) by the skilled person without further ado. E.g., the scoring algorithm may be a mathematical function that uses information regarding the amounts of the diagnostic biomarkers in a cohort of subjects suffering from pancreatic cancer and not suffering from pancreatic cancer. Methods for determining a scoring algorithm are well known in the art and include Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Commit-tee, j48, LMT, NBTree, Part, Random Forest, Ordinal Classifier, Sparse Linear Programming (SPLP), Sparse Logistic Regression (SPLR), Elastic net, Support Vector Machine, Prediction of Residual Error Sum of Squares (PRESS), Penalized Logistic Regression, Mutual Information. Preferably, the scoring algorithm is determined with or without correction for confounders as set forth elsewhere herein. In an embodiment, the scoring algorithm is determined with an elastic net with diagnostic biomarkers.

In a preferred embodiment of the method of the present invention, different reference scores are provided in dependence on the value of the concentration of CA19-9 determined in a sample. Preferably, in case a value of CA19-9 indicating that the subject is a Lewis positive subject, preferably a value of more than 5 U/ml, preferably more than 2 U/ml, is determined, the reference value is determined as specified above; in case a value of CA19-9 indicating that the subject is a Lewis negative subject, preferably a value of less than or equal to 5 U/ml, preferably less than or equal to 2 U/ml, is determined, the reference value is determined attributing significantly less weight to the CA19-9 concentration, preferably a weight of 0. Also preferably, different weights are attributed to the CA19-9 concentration for the following classes of CA19-9 concentrations: 0 U/ml or below detection limit up to 2 U/ml, of from more than 2 U/ml up to 15 U/ml, and more than 15 U/ml. More preferably, different weights are attributed to the CA19-9 concentration for the following classes of CA19-9 concentrations: 0 U/ml or below detection limit up to 2 U/ml, of from more than 2 U/ml up to 12 U/ml, and more than 12 U/ml.

In a further preferred embodiment, the score for a subject is calculated including the subject's data, e.g. age, gender, and the like, and/or clinical parameters, like BMI, weight, blood pressure, blood group, and the like, and/or life style risk factors such as smoking, alcohol consumption, diet, and the like, in addition to the metabolic biomarkers of the invention.

In an embodiment, the score for a subject is calculated with a logistic regression model fitted e.g. by using the elastic net algorithm such as implemented in the R package glmnet (e.g. as disclosed by Zou, H. and Hastie, T., 2003: Regression shrinkage and selection via the elastic net, with applications to microarrays. Journal of the Royal Statistical Society: Series B, 67, 301-320; Friedman, J., Hastie, T., and Tibshirani, R, 2010: Regularization Paths for Generalized Linear Models via Coordinate Descent. J. Stat. Softw. 33).

Preferably, a score is calculated as a prediction score as specified elsewhere herein.

Preferably, a classifier of pancreatic cancer diagnosis is obtained by training the elastic net algorithm on predefined groups of diagnostic biomarkers, preferably as described by Zou and Hastie ((2005) Regularization and variable selection via the elastic net, Journal of the Royal Statistical Society, Series B, 67, 301-320).

In a preferred embodiment, cutoff values are compared to the values obtained from the diagnostic biomarkers; more preferably, said cutoff value is a gender-specific cutoff, as an example, preferably a gender-specific cutoff value according to Table 16. In a further preferred embodiment, comparing of the amounts of the diagnostic biomarkers to a reference comprises the steps of calculating a prediction score for a subject, as specified elsewhere herein; as an example, e.g., preferably, the parameters and diagnostic biomarkers of Tables 13 to 15 may be used.

It will be understood that the method can also be applied to determine whether a subject will benefit from or is in need of a therapy against the aforementioned diseases. Such a method can be applied in therapeutic approaches like “active surveillance”. In this approach, a subject suffering from, e.g., less advanced pancreatitis is subjected to a method for diagnosing pancreatic cancer as set forth above on a regular basis with short intervals in order to early detect pancreatic cancer development. Only after pancreatic cancer is detectable, the subject is treated by a suitable therapy, such as surgery or irradiation, as specified herein below. Thus, “active surveillance” prevents the harmful side effects of a therapy in subjects which are not in an immediate need for a therapy. By avoiding the therapy at this stage, it will be understood that the harmful side effects of the therapy can be avoided as well.

Advantageously, it was found in the work underlying the present invention that combining the biomarker CA19-9 with specific, hand-elected biomarkers improves the diagnostic performance of diagnosing pancreatic cancer. In particular, it was found that groups of biomarkers can be selected, e.g. the diagnostic biomarkers of the present invention, which can, except for CA19-9, be determined in a single LC-MS/MS run, thus decreasing the effort required for reliable diagnosis. Moreover, it was found that with specific groups of biomarkers, prediction of pancreatic cancer in specific subgroups of patients, e.g. patients with low Lewis a/b antigen, in patients with new-onset diabetes, or in patients with chronic pancreatitis, or also the diagnosis of pancreatic cancer in an early stage, where the tumor is still resectable, is possible.

The definitions made above apply mutatis mutandis to the following. Additional definitions and explanations made further below also apply for all embodiments described in this specification mutatis mutandis.

The present invention further relates to a method of treating pancreatic cancer in a subject, comprising diagnosing pancreatic cancer in said subject according to a method of diagnosing pancreas cancer of the present invention, and treating said pancreatic cancer in said subject.

The present invention further relates a method of treating pancreatic cancer in a subject, comprising providing a diagnosis of pancreatic cancer according to according to a method of diagnosing pancreas cancer of the present invention, and treating said pancreatic cancer in said subject.

The term “treating” refers to ameliorating the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that a statistically significant portion of subjects suffering from a disease or disorder referred to herein can be successfully treated. Whether a portion is statistically significant can be determined without further ado by the person skilled in the art using various well known statistic evaluation tools indicated elsewhere herein.

The term “treating pancreas cancer”, as used herein, refers to therapeutic measures aiming to cure or ameliorate pancreatic cancer or aiming at preventing progression of the said disease as well as patient health management measures, such as monitoring, including selection of monitoring measures and monitoring frequency and hospitalization. Preferably, said treating comprises a measure selected from the group consisting of: surgery, administration of cancer therapy, patient monitoring, active surveillance, and hospitalization. More preferably, said treating comprises surgery and/or administration of cancer therapy. Suitable cancer therapies include low- and high-dose irradiation, and systemic chemotherapy, e.g., cytostatic drugs, alone, or in combination with other drugs. Preferred surgery-based therapies include resection of the pancreas or parts thereof, such as pancreaticoduodenectomy, tail pancreatectomy, total or partial pancreatectomy, and palliative bridging procedures. Drug-based therapies, preferably, include the administration of one or more drugs with anti-tumour properties including but not exclusive to platinum derivatives, e.g., oxaliplatin, fluoropyrimidines, pyrimidine analogues, gemcitabine, antimetabolites, alkylating agents, anthracyclines, plant alkaloids, topoisomerase inhibitors, targeted antibodies and tryosine kinase inhibitors. Particular preferred drugs include, but are not limited to, gemcitabine alone or in combination with erlotinib and/or oxaliplatin. In a more preferred embodiment of the method of the present invention, said method also comprises the step of applying the said therapeutic or patient health management measure to the subject.

The present invention further relates to a method of detecting biomarkers, preferably diagnostic biomarkers, more preferably small molecule diagnostic biomarkers, in a sample comprising

(a) adding a non-phase separating, protein precipitating solution to said sample,
(b) removing precipitated protein,
(c) separating said biomarkers in the non-proteinaceous fraction by chromatography, and
(d) detecting the biomarkers.

The method of detecting biomarkers of the present invention, preferably, is an in vitro method. Moreover, it may comprise steps in addition to those explicitly mentioned above. For example, further steps may relate, e.g., obtaining a sample for step (a), or calculating a value derived from the value obtained in step (d). Moreover, one or more of said steps may be performed by automated equipment. Preferably, in said method, at least one diagnostic biomarker of the present invention which is not CA19-9 is determined. More preferably, at least two, at least three, at least four, at least five, at least six, or at least seven diagnostic biomarkers of the present invention, with the exception of CA19-9, preferably, small molecule diagnostic biomarkers of a panel of Table 9, are determined. Preferably, at least the small molecule diagnostic biomarkers of a panel of Table 9 are determined. Preferably, the method for detecting a biomarker is used in the method of diagnosing pancreatic cancer according to the present invention. In a preferred embodiment, the method of detecting biomarkers is a method for detecting amino acids and lipids in a sample.

As indicated herein above, the term “non-phase separating, protein precipitating solution” relates to a non-phase separating, i.e., a one phase solvent, having the additional property of precipitating proteins from a solution. Appropriate solvents, including mixtures of solvents, are known in the art. Preferably, the non-phase separating, protein precipitating solution is a mixture comprising a first solvent selected from the group consisting of dichloromethane (DCM), chloroform, tertiary butyl methyl ether (tBME or MTBE, also known as 2-methoxy-2-methylpropane), ethyl ethanoate, and isooctane, and a second solvent selected from the group consisting of methanol, ethanol, isopropanol and dimethyl sulfoxide (DMSO). More preferably, the non-phase separating, protein precipitating solution comprises methanol and DCM, in particular in a ratio of about 2:1 (v/v) to about 3:2 (v/v), preferably in a ratio of about 2:1 (v/v) or about 3:2 (v/v). More preferably, the non-phase separating, protein precipitating solution comprises methanol:dichloromethane in a ratio of 2:1 (v/v). Preferably, the term “non-phase separating solution” relates to a solution having only one liquid phase, even if a fifth of its volume of water and/or dimethylsulfoxide (DMSO) is added. Preferably, at least three volumes, more preferably at least four volumes, most preferably at least five volumes of non-phase separating, protein precipitating solution are added to a given volume of a sample or to a diluted sample. Preferably, the sample is diluted by at least a factor of four, more preferably a factor of five, before the non-phase separating, protein precipitating solution is added. Preferably, the diluent used in said dilution of the sample is a solution comprising at least 50% (v/v) DMSO, more preferably comprising at least 70% (v/v) DMSO, even more preferably a solution of DMSO:methanol:dichloromethane:water in a ratio of 12.3:2.2:1.1:1 (v/v/v/v).

Methods for removing precipitated protein are known to the skilled person and include, preferably, centrifugation and/or filtration, preferably ultrafiltration.

Thus, in a preferred embodiment, the method of detecting biomarkers of the present invention comprises pre-treating a sample with a non-phase separating, protein precipitating solution as described above. Preferably, said metabolites are determined by a method comprising electrospray-ionization (ESI), more preferably positive-ion ESI in such case. In a further preferred embodiment, the method comprises pre-treating the sample pre-treated with said first non-phase separating, protein precipitating solution with a second non-phase separating, protein precipitating solution, said second non-phase separating, protein precipitating solution comprising the first solvent and the second solvent as described above for the first non-phase separating, protein precipitating solution, however, at a different ratio and/or a suitable dilution, preferably using methanol and/or dichloromethane, preferably dichlorormethane as a diluent; preferably, the extracts obtained with the first non-phase separating, protein precipitating solution and with the second non-phase separating, protein precipitating solution are combined before determining a metabolite in such case. In a further preferred embodiment, after the first extraction as specified above, a first aliquot is obtained, and the residual extract including precipitated proteins is further diluted using the same non-phase separating, protein precipitating solution, and, after removal of precipitated protein, a second aliquot of the sample is obtained; preferably, metabolites expected or known to be present at a high concentration are determined from the second aliquot, and metabolites expected or known to be present in the sample at a low concentration are determined from the first aliquot in such case. In a further preferred embodiment, the sample is diluted at least five-fold (v/v), preferably at least tenfold (v/v), more preferably at least 100 fold (v/v), with said non-phase separating, protein precipitating solution and, after removal of precipitated proteins, a first aliquot is obtained, and at least part, preferably all of the non-phase separating, protein precipitating solution or all liquid is removed, preferably under vacuum, from the residual extract; preferably, metabolites expected or known to be present at a high concentration are determined from the first aliquot, and metabolites expected or known to be present in the sample at a low concentration are determined from the second aliquot in such case. Preferably, said metabolites are determined by a method comprising electrospray-ionization (ESI), more preferably positive-ion and/or negative-ion ESI in such case.

Preferably, the method of detecting biomarkers comprises the further step of contacting biomarkers in the non-proteinaceous fraction with a reagent introducing hydrophobic side chains before separating said biomarkers in the non-proteinaceous fraction by chromatography, i.e. preferably, a step of hydrophobic derivatization. Preferably, said derivatization comprises, even more preferably consists of, contacting biomarkers in the non-proteinaceous fraction with a reagent introducing hydrophobic side chains, even more preferably with only one reagent introducing hydrophobic side chains. Preferably, said reagent introducing hydrophobic side chains is a reagent derivatizing amino groups, preferably primary and secondary amino groups. More preferably, said reagent introducing hydrophobic side chains is 5-(dimethylamino)naphthalene-1-sulfonyl chloride (dansylchloride, CAS Registry No: 605-65-2).

Preferably, in the method of detecting biomarkers, protein precipitation is the only purification step before the step of separating the biomarkers in the non-proteinaceous fraction by chromatography. In another embodiment, in the method of detecting biomarkers, protein precipitation is the only purification step before the step of derivatizing followed by separating the analytes in the non-proteinaceous fraction by chromatography.

Methods of separating biomarkers and of detecting biomarkers are well known in the art and are described herein above. Preferably, the separation step comprises reverse phase chromatography, more preferably reverse phase liquid chromatography. Preferably, detecting the metabolites is effected by mass spectrometry (MS), preferably MS/MS. Thus, steps c) and d) of the method of detecting metabolites are preferably performed with LC-MS/MS.

In an also preferred embodiment, the present invention also relates to a method for diagnosing pancreatic cancer in a subject, wherein the step of determining in at least one sample of said subject the amounts of a group of diagnostic biomarkers is preceded by the steps of the method of detecting biomarkers as described above.

In a preferred embodiment, the method of diagnosing pancreas cancer comprises the steps of
(a) semiquantitatively or, preferably, quantitatively determining the amounts of a group of diagnostic biomarkers according to the present invention in at least one sample of a subject,
(b1) for each amount determined in step (a), calculating a scaled amount by first subtracting a predetermined, diagnostic biomarker-specific subtrahend from said amount and then dividing the resulting value by a predetermined, diagnostic biomarker-specific divisor,
(b2) calculating a prediction score by
   (i) assigning a diagnostic biomarker-specific weight value to each scaled amount of (b1), thereby providing a weighed amount,
   (ii) summing up said weighed amounts for all diagnostic biomarkers, providing a sum of weighted amounts,
   (iii) preferably, assigning a bias value to the sum of weighted amounts of step (ii) to provide a bias-corrected sum,
   (iv) preferably, scaling the bias-corrected sum of step (iii), preferably to a value between 0 and 1, and
(b3) determining the probability for a subject to suffer from pancreatic cancer based on the prediction score determined in step (b2).

Step (a) of the method for determining the probability for a subject to suffer from pancreatic cancer, preferably, corresponds to step (a) of the method for diagnosing pancreatic cancer as specified above. Preferably, the measured values are scaled by first subtracting a predetermined, diagnostic biomarker-specific subtrahend from said amount and then dividing the resulting value by a predetermined, diagnostic biomarker-specific divisor. From the values thus obtained, a prediction score is calculated by weighting the individual biomarkers and summing up the weighted values for all biomarkers determined, preferably, followed by assigning a bias value to said sum and, preferably, scaling the bias-corrected sum, preferably to a value between 0 and 1. Preferably, scaling of measured values comprises log 10 transforming the measured values, preferably after subtracting said predetermined, diagnostic biomarker-specific subtrahend. More preferably, the prediction score is calculated according to the following formula (I)

$$p = \frac{1}{1 + {e}^{- ( {ω}_{0} + {Σ}_{i}^{n} {ω}_{i} {\hat{x}}_{i} )}} , \qquad ( I )$$

wherein
p=prediction score;
e=Euler's number;
ω₀=bias value;
ωᵢ=diagnostic biomarker-specific weight value for diagnostic biomarker i; and
\hat{x}ᵢ=scaled amount for diagnostic biomarker i.

Preferably, \hat{x}ᵢ is calculated by scaling the log₁₀-transformed input data x by first subtracting an analyte-specific constant mᵢ and then dividing by a analyte-specific constant sᵢ, resulting in log₁₀-transformed and scaled input data \hat{x}:

$${\hat{x}}_{i} = \frac{{x}_{i} - {m}_{i}}{{s}_{i}}$$

Preferably, optimized values of the predetermined, diagnostic biomarker-specific subtrahend, the predetermined, diagnostic biomarker-specific divisor, the diagnostic biomarker-specific weight value, and the bias value are determined by optimizing the differentiation between subjects suffering from a specific disease and subjects not suffering from said disease. Thus preferably, the group of diagnostic biomarkers is, preferably, trained on data obtained from two groups of subjects with known disease state. E.g., preferably, one of said groups of subjects is a group of subjects suffering from pancreatitis, and the second group is a group of subjects suffering from pancreatic cancer; this way, optimized parameters for a differentiation between pancreatitis and pancreatic cancer are obtained.

Moreover, the present invention relates to a method for diagnosing pancreatic cancer by determining at least one diagnostic biomarker in a subject comprising the steps of:

(a) determining in a sample of said subject the amount of at least one diagnostic biomarker selected from any one of Tables 2 to 7; and
(b) comparing the said amount of said diagnostic biomarker with a reference, whereby pancreatic cancer is diagnosed.

The method for diagnosing pancreatic cancer by determining at least one diagnostic biomarker, preferably, is an in vitro method. Moreover, it may comprise steps in addition to those explicitly mentioned above. For example, further steps may relate, e.g., to obtaining a sample for step a), or determining at least one further biomarker, preferably at least one diagnostic biomarker of the present invention. Moreover, one or more of said steps may be performed by automated equipment.

Regarding definition of terms not specifically defined with regard to the method for diagnosing pancreatic cancer by determining at least one diagnostic biomarker, reference is made to the definitions provided in the context of the method for diagnosing pancreatic cancer above, which are applicable mutatis mutandis, if not noted otherwise.

In a preferred embodiment, in said method, the at least one diagnostic biomarker is selected from the list consisting of Phosphatidylethanolamine (C18:0,C22:6), Lysophosphatidylethanolamine (C18:0), and Sphingomyelin (35:1).

In a further preferred embodiment, the subject is a subject suffering from chronic pancreatitis and the at least one diagnostic biomarker is selected from the list consisting of Sphingomyelin (35:1), Phosphatidylethanolamine (C18:0,C22:6), and Lysophosphatidylethanolamine (C18:0).

In a further preferred embodiment, the subject is a subject suffering from new-onset diabetes and the at least one diagnostic biomarker is selected from the list consisting of Lysophosphatidylethanolamine (C18:2), Proline, Sphingomyelin (35:1), Sphingomyelin (d18:2,C17:0), Phosphatidylethanolamine (C18:0,C22:6), Sphingomyelin (d17:1,C16:0), Histidine, Sphingomyelin (41:2), Tryptophan, Lysophosphatidylethanolamine (C18:0), Ceramide (d18:1,C24:0), and Ceramide (d18:2,C24:0).

In a further preferred embodiment, the pancreatic cancer is a resectable pancreatic cancer and the at least one diagnostic biomarker is selected from the list consisting of Proline, Ceramide (d18:2,C24:0), Ceramide (d18:1,C24:0), Phosphatidylethanolamine (C18:0,C22:6), Tryptophan, Histidine, Lysophosphatidylethanolamine (C18:0), Sphingomyelin (35:1), Sphingomyelin (d18:2,C17:0), Sphingomyelin (d17:1,C16:0), and Sphingomyelin (41:2).

In a further preferred embodiment, the pancreatic cancer is a resectable pancreatic cancer, the subject is a subject suffering from chronic pancreatitis, and the at least one diagnostic biomarker is selected from the list consisting of is Sphingomyelin (35:1), Ceramide (d18:1,C24:0), Phosphatidylethanolamine (C18:0,C22:6), Ceramide (d18:2,C24:0), Lysophosphatidylethanolamine (C18:0), and Tryptophan.

In a further preferred embodiment, the pancreatic cancer is a resectable pancreatic cancer, the subject is a subject suffering from new-onset diabetes, and the at least one diagnostic biomarker is selected from the list consisting of Lysophosphatidylethanolamine (C18:2), Proline, Sphingomyelin (35:1), Sphingomyelin (d18:2,C17:0), Phosphatidylethanolamine (C18:0,C22:6), Tryptophan, Sphingomyelin (d17:1,C16:0), Ceramide (d18:1,C24:0), Ceramide (d18:2,C24:0), Sphingomyelin (41:2), Lysophosphatidylethanolamine (C18:0) and Histidine.

In a further preferred embodiment, the at least one diagnostic biomarker is selected from the list consisting of Lysophosphatidylethanolamine (C18:0), Phosphatidylethanolamine (C18:0, C22:6), and sphingomyelin (35:1).

In the preferred embodiments of the method for diagnosing pancreatic cancer by determining at least one diagnostic biomarker, sphingomyelin (35:1), preferably, is the sum of the amounts of sphingomyelin (d18:1,C17:0) and sphingomyelin (d17:1,C18:0); or is the amount of sphingomyelin (d18:1,C17:0).

Moreover, the present invention relates to a diagnostic device for carrying out a method for diagnosing pancreatic cancer of the present invention, comprising:

a) an analysing unit comprising at least one detector for at least the small molecule diagnostic biomarkers of a group of diagnostic biomarkers according to the present invention, wherein said analyzing unit is adapted for determining the amounts of at least said small molecule diagnostic biomarkers detected by the at least one detector, and, operatively linked thereto;
b) an evaluation unit comprising a computer comprising tangibly embedded a computer program code for carrying out a comparison of the determined amounts of the small molecule diagnostic biomarkers, and, preferably, CA19-9, with a reference and a data base comprising said reference for said diagnostic biomarkers, whereby it is diagnosed whether a subject suffers from pancreatic cancer.

A “device”, as the term is used herein, comprises at least the aforementioned units. The units of the device are operatively linked to each other. How to link the means in an operating manner will depend on the type of units included into the device. For example, where the detector allows for automatic qualitative or quantitative determination of the biomarker, the data obtained by said automatically operating analyzing unit can be processed by, e.g., a computer program in order to facilitate the assessment in the evaluation unit. Preferably, the units are comprised by a single device in such a case. Preferably, the device includes an analyzing unit for the biomarker and a computer or data processing device as an evaluation unit for processing the resulting data for the assessment and for establishing the output information. Preferably, the analyzing unit comprises at least one detector for at least the diagnostic biomarker or the diagnostic biomarkers of a group according to the present invention, said at least one detector determining the amounts of said markers in said sample. Preferred devices are those which can be applied without the particular knowledge of a specialized clinician, e.g., electronic devices which merely require loading with a sample. The output information of the device, preferably, is a numerical value which allows drawing conclusions on the quality of the sample and, thus, is an aid for the reliability of a diagnosis or for troubleshooting. Preferred references to be used in accordance with the device of the present invention are values for the biomarkers analyzed or values derived therefrom as specified above. Preferably, said device is a device for diagnosing pancreatic cancer. Preferably, the device further comprises an input unit adapted to receive input data, preferably a value of an amount of CA19-9.

The units of the device, also preferably, can be implemented into a system comprising several devices which are operatively linked to each other. Depending on the units to be used for the system of the present invention, said means may be functionally linked by connecting each means with the other by means which allow data transport in between said means, e.g., glass fiber cables, and other cables for high throughput data transport. Nevertheless, wireless data transfer between the means is also envisaged by the present invention, e.g., via LAN (Wireless LAN, W-LAN). A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or LC-MS/MS are used in the system of the present invention as described in detail elsewhere in the specification. Further comprised shall be means for comparing and/or analyzing the results obtained from the means for determination of biomarkers. The means for comparing and/or analyzing the results may comprise at least one databases and an implemented computer program for comparison of the values measured with corresponding references. Preferred embodiments of the aforementioned systems and devices are also described in detail below.

Furthermore, the present invention relates to a data collection comprising characteristic values of at least the markers of at least one panel of Table 9, being indicative for a subject suffering from pancreas cancer, or not.

The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for pancreatic cancer as set forth above (e.g. a query search). Thus, if an identical or similar data set can be identified in the data collection, the test data set will be associated with the presence of disease, or not. Consequently, the information obtained from the data collection can be used, e.g., as a reference for the methods of the present invention described above. More preferably, the data collection comprises characteristic values of all diagnostic biomarkers comprised by any one of the groups recited above.

In light of the foregoing, the present invention encompasses a data storage medium comprising the aforementioned data collection.

The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to the use

(i) of a group of diagnostic biomarkers according to the present invention; or
(ii) of a diagnostic biomarker according to the present invention;
in a sample of a subject for diagnosing pancreatic cancer or for the preparation of a pharmaceutical and/or diagnostic composition for diagnosing pancreatic cancer.

All references cited in this specification are herewith incorporated by reference with respect to their entire disclosure content and the disclosure content specifically mentioned in this specification.

In view of the above, the following embodiments are preferred:

1. A method for diagnosing pancreatic cancer in a subject comprising the steps of:
(a) determining in at least one sample of said subject the amounts of a group of diagnostic biomarkers comprising
(i) at least one diagnostic amino acid, said diagnostic amino acid being proline, histidine or tryptophan, preferably, being proline;
(ii) at least one diagnostic ceramide, said diagnostic ceramide being ceramide (d18:1,C24:0) or ceramide (d18:2,C24:0), preferably being ceramide (d18:1,C24:0);
(iii) at least one diagnostic sphingomyelin, said diagnostic sphingomyelin being sphingomyelin (35:1), sphingomyelin (d17:1,C16:0), sphingomyelin (41:2) or sphingomyelin (d18:2,C17:0), preferably being sphingomyelin (35:1); and

### (iv) CA19-9;

and
(b) comparing said amounts of the diagnostic biomarkers with a reference, whereby pancreatic cancer is diagnosed.
2. The method of embodiment 1, wherein said subject is a subject at least 40 years old, preferably at least 50 years old.
3. The method of embodiment 1 or 2, wherein said sample is a sample obtained from said subject while said subject was fasting, preferably for at least eight hours.
4. The method of any one of embodiments 1 to 3, wherein said subject is a subject at risk of suffering from pancreatic cancer.
5. The method of embodiment 4, wherein said subject at risk of suffering from pancreatic cancer is a subject with new-onset diabetes.
6. The method of embodiment 4, wherein said subject at risk of suffering from pancreatic cancer is a subject suffering from chronic pancreatitis.
7. The method of any one of embodiments 1 to 6, wherein said subject is a subject with a low CA19-9 value.
8. The method of embodiment 7, wherein a low CA19-9 value is a blood CA19-9 value of less than 42 U/mL, preferably less than 37 U/mL.
9. The method of embodiment 7 or 8, wherein said subject is a subject being blood group Lewis a/b negative.
10. The method of any one of embodiments 1 to 9, wherein said subject is a subject suspected to suffer from pancreatic cancer.
11. The method of embodiment 10, wherein said subject suspected to suffer from pancreatic cancer is a subject having at least one clinical symptom of pancreatic cancer, preferably selected from the list consisting of abdominal pain, lower back pain, nausea, vomiting, and jaundice.
12. The method of embodiment 10 or 11, wherein said subject suspected to suffer from pancreatic cancer is a subject suspected to suffer from pancreatic cancer or from chronic pancreatitis.
13. The method of any one of embodiments 1 to 12, wherein said pancreatic cancer is a pancreatic cancer with a resectable tumor stage.
14. The method of any one of embodiments 1 to 13, wherein said diagnostic amino acid is proline.
15. The method of any one of embodiments 1 to 14, wherein said diagnostic ceramide is ceramide (d18:1,C24:0).
16. The method of any one of embodiments 1 to 14, wherein said diagnostic ceramide is ceramide (d18:2,C24:0).
17. The method of any one of embodiments 1 to 16, wherein said diagnostic sphingomyelin is sphingomyelin (35:1).
18. The method of any one of embodiments 1 to 13, wherein said diagnostic amino acid is proline and wherein said diagnostic ceramide is ceramide (d18:2,C24:0).
19. The method of any one of embodiments 1 to 13, wherein said diagnostic amino acid is proline and wherein said diagnostic sphingomyelin is sphingomyelin (35:1) or sphingomyelin (d18:1,C17:0).
20. The method of any one of embodiments 1 to 13, wherein said group of diagnostic biomarkers comprises, preferably consists of, the diagnostic biomarkers proline, ceramide (d18:1,C24:0), sphingomyelin (35:1), and CA19-9.
21. The method of any one of embodiments 1 to 13, wherein said group of diagnostic biomarkers comprises, preferably consists of, the diagnostic biomarkers proline, ceramide (d18:2,C24:0), sphingomyelin (35:1), and CA19-9.
22. The method of any one of embodiments 1 to 13, wherein said group of diagnostic biomarkers further comprises at least one diagnostic ethanolamine lipid, said diagnostic ethanolamine lipid being phosphatidylethanolamine (C18:0,C22:6), lysophosphatidylethanolamine (C18:2) or lysophosphatidylethanolamine (C18:0), preferably being phosphatidylethanolamine (C18:0,C22:6).
23. The method of embodiment 22, wherein said diagnostic ethanolamine lipid is phosphatidylethanolamine (C18:0,C22:6).
24. The method of any one of embodiments 1 to 13, wherein said group of diagnostic biomarkers comprises, preferably consists of, the diagnostic biomarkers proline, ceramide (d18:1,C24:0), sphingomyelin (35:1), phosphatidylethanolamine (C18:0,C22:6), and CA19-9.
25. The method of any one of embodiments 1 to 13, wherein said group of diagnostic biomarkers comprises, preferably consists of, the diagnostic biomarkers proline, ceramide (d18:2,C24:0), sphingomyelin (35:1), phosphatidylethanolamine (C18:0,C22:6) and CA19-9.
26. The method of any one of embodiments 1 to 13, wherein said group of diagnostic biomarkers comprises, preferably consists of, the diagnostic biomarkers of at least one of the panels of Table 9, preferably, the diagnostic biomarkers of panel 1, 2, 6, 7, 11, 82, 9, 89, 44, 10, 58, 46, 66, 13, 31, 30, 92, 86, 48, 81 or 90 of Table 9.
27. The method of any one of embodiments 1 to 3, wherein said subject is a subject suffering from chronic pancreatitis and wherein said group of diagnostic biomarkers comprises, preferably consists of, the diagnostic biomarkers of panel 1, 2, 6, 7, 4, 12, 14, 43, 19, 13, 16, 41, 21, 50, 44, 47, 46, 48, 3, or 5 of Table 9.
28. The method of any one of embodiments 1 to 3, wherein said subject is a subject suffering from new-onset diabetes and wherein said group of diagnostic biomarkers comprises, preferably consists of, the diagnostic biomarkers of panel 1, 2, 6, 7, 13, 9, 43, 12, 10, 11, 47, 21, 14, 49, 48, 4, 19, 46, 82 or 52 of Table 9.
29. The method of any one of embodiments 1 to 3, wherein said subject is a subject with a low CA19-9 value and wherein said group of diagnostic biomarkers comprises, preferably consists of, the diagnostic biomarkers of panel 1, 2, 6, 7, 9, 13, 12, or 3 of Table 9.
30. The method of any one of embodiments 1 to 3, wherein said pancreatic cancer is a resectable pancreatic cancer and wherein said group of diagnostic biomarkers comprises, preferably consists of, the diagnostic biomarkers of panel 1, 2, 6, 7, 3, 4, 5, 9, 10, 12, 13, 14, 15, 16, 18, 19, 11, 21, 22 or 30 of Table 9.
31. The method of any one of embodiments 1 to 30, wherein said group of diagnostic biomarkers comprises, preferably consists of, the diagnostic biomarkers CA19-9, Ceramide (d18:1,C24:0), Ceramide (d18:2,C24:0), Histidine, Lysophosphatidylethanolamine (C18:0), Lysophosphatidylethanolamine (C18:2), Phosphatidylethanolamine (C18:0,C22:6), Proline, Sphingomyelin (d17:1,C16:0), Sphingomyelin (35:1), Sphingomyelin (41:2), Sphingomyelin (d18:2,C17:0), and Tryptophan.
32. The method of any one of embodiments 1 to 31, wherein said sphingomyelin (41:2) represents
sphingomyelin (d18:1,C23:1), sphingomyelin (d17:1,C24:1), and sphingomyelin (d18:2,C23:0);
sphingomyelin (d18:1,C23:1) and sphingomyelin (d17:1,C24:1);
sphingomyelin (d17:1,C24:1) and sphingomyelin (d18:2,C23:0);
sphingomyelin (d18:1,C23:1) and sphingomyelin (d18:2,C23:0);
sphingomyelin (d18:1,C23:1);
sphingomyelin (d17:1,C24:1); or
sphingomyelin (d18:2,C23:0).
33. The method of any one of embodiments 1 to 32, wherein said sphingomyelin (35:1) represents
sphingomyelin (d18:1,C17:0) and sphingomyelin (d17:1,C18:0);
sphingomyelin (d18:1,C17:0); or
sphingomyelin (d17:1,C18:0).
34. The method of any one of embodiments 1 to 33, wherein each further biomarker determined decreases the false-positive rate and/or the false negative rate of the method by at least 0.1% or a significantly increased AUC.
35. The method of any one embodiments 1 to 34, wherein said group of diagnostic biomarkers does not comprise sphinganine-1-phosphate (d18:0).
36. The method of any one of embodiments 1 to 35, wherein, if said group of diagnostic biomarkers comprises histidine, said group of diagnostic biomarkers does not comprise sphingomyelin (d18:2,C17:0).
37. The method of any one of embodiments 1 to 36, wherein determining the amount of a diagnostic biomarker is quantitatively determining the amount of said diagnostic biomarker.
38. The method of any one of embodiments 1 to 37, wherein said sample is a sample of a bodily fluid, preferably, a blood, plasma, serum, or urine sample, more preferably a blood sample, most preferably a plasma sample.
39. The method of any one of embodiments 1 to 38, wherein said comparing amounts of diagnostic biomarkers with references comprises comparing said amounts or a value calculated therefrom to one or more cutoff values.
40. The method of embodiment 39, wherein said cutoff value is calculated according to the method of any one of embodiments 53 to 55.
41. The method of embodiment 40, wherein said cutoff value is a gender-specific cutoff.
42. The method of any one of embodiments 1 to 41, wherein said method further comprises the step of removing proteins from said sample by precipitation preceding step (a).
43. The method of any one of embodiments 1 to 42, wherein said step of removing proteins by precipitation comprises adding a non-phase separating, protein precipitating solution, preferably methanol:dichloromethane in a ratio of 2:1 (v/v) to said sample.
44. The method of any one of embodiments 1 to 43, comprising the further step of separating said at least one diagnostic amino acid from said at least one diagnostic ceramide and, preferably, from said at least one diagnostic sphingomyelin, by chromatography, preferably by reverse phase chromatography, more preferably by reverse phase liquid chromatography, said further step preceding step (a).
45. A method of detecting biomarkers, preferably of detecting diagnostic biomarkers, more preferably of detecting small molecule diagnostic biomarkers, of the present invention, in a sample comprising
(a) adding a non-phase separating, protein precipitating solution to said sample,
(b) removing precipitated protein,
(c) separating said biomarkers in the non-proteinaceous fraction by chromatography, and
(d) detecting the biomarkers.
46. The method of embodiment 45, wherein said sample is a blood, plasma, serum, or urine sample, preferably, is a blood sample, more preferably a plasma sample.
47. The method of embodiment 45 or 46, wherein said non-phase separating, protein precipitating solution is methanol:dichloromethane in a ratio of 2:1 (v/v).
48. The method of any one of embodiments 45 to 47, further comprising the step of diluting said sample with a solution comprising at least 50% (v/v) dimethylsulfoxide (DMSO), preferably comprising at least 70% (v/v) DMSO, more preferably a solution of DMSO:methanol:dichloromethane:water in a ratio of 12.3:2.2:1.1:1 (v/v/v/v).
49. The method of any one of embodiments 45 to 48, comprising the further step of contacting said biomarkers with a reagent introducing hydrophobic side chains before step (c), preferably, wherein said reagent introducing hydrophobic side chains is a reagent derivatizing amino groups, preferably primary and secondary amino groups.
50. The method of any one of embodiments 45 to 49, wherein said reagent introducing hydrophobic side chains is 5-(dimethylamino)naphthalene-1-sulfonyl chloride (dansylchloride, CAS Registry No: 605-65-2).
51. A method of treating pancreatic cancer in a subject, comprising
diagnosing pancreatic cancer in said subject according to any one of embodiments 1 to 44, and
treating said pancreatic cancer in said subject.
52. A method of treating pancreatic cancer in a subject, comprising
providing a diagnosis of pancreatic cancer according to any one of embodiments 1 to 44, and
treating said pancreatic cancer in said subject.
53. The method for diagnosing pancreatic cancer of any one of embodiments 1 to 44, comprising the steps of
(a) semiquantitatively and or quantitatively, preferably, quantitatively, determining the amounts of a group of diagnostic biomarkers according to any one of embodiments 1 to 44 in at least one sample of a subject,
(b1) for each amount determined in step (a), calculating a scaled amount by first subtracting a predetermined, diagnostic biomarker-specific subtrahend from said amount and then dividing the resulting value by a predetermined, diagnostic biomarker-specific divisor,
(b2) calculating a prediction score by
(i) assigning a diagnostic biomarker-specific weight value to each scaled amount of (b1), thereby providing a weighed amount,
(ii) summing up said weighed amounts for all diagnostic biomarkers, providing a sum of weighted amounts,
(iii) preferably, assigning a bias value to the sum of weighted amounts of step (ii) to provide a bias-corrected sum,
(iv) preferably, scaling the bias-corrected sum of step (iii), preferably to a value between 0 and 1, and
(b3) determining the probability for a subject to suffer from pancreatic cancer based on the prediction score determined in step (b2).
54. The method of embodiment 53, wherein said amounts are logo transformed before scaling said amounts in step (b).
55. The method of embodiment 53 or 54, wherein said prediction probability is calculated according to the following formula (I)

$$p = \frac{1}{1 + {e}^{- ( {ω}_{0} + {Σ}_{i}^{n} {ω}_{i} {\hat{x}}_{i} )}} , \qquad ( I )$$

wherein
p=prediction score;
e=Euler's number;
ω₀=bias value;
ωᵢ=diagnostic biomarker-specific weight value for diagnostic biomarker i; and
\hat{x}ᵢ=scaled amount for diagnostic biomarker i.
56. A method for diagnosing pancreatic cancer in a subject comprising the steps of:
(a) determining in a sample of said subject the amount of at least one diagnostic biomarker selected from any one of Tables 2 to 7; and
(b) comparing the said amount of said diagnostic biomarker with a reference, whereby pancreatic cancer is diagnosed.
57. The method of embodiment 56, wherein said at least one diagnostic biomarker is selected from the list consisting of Phosphatidylethanolamine (C18:0,C22:6), Lysophosphatidylethanolamine (C18:0), and Sphingomyelin (35:1).
58. The method of embodiment 56, wherein said subject is a subject suffering from chronic pancreatitis and wherein said at least one diagnostic biomarker is selected from the list consisting of Sphingomyelin (35:1), Phosphatidylethanolamine (C18:0,C22:6), and Lysophosphatidylethanolamine (C18:0).
59. The method of embodiment 56, wherein said subject is a subject suffering from new-onset diabetes and wherein said at least one diagnostic biomarker is selected from the list consisting of Lysophosphatidylethanolamine (C18:2), Proline, Sphingomyelin (35:1), Sphingomyelin (d18:2,C17:0), Phosphatidylethanolamine (C18:0,C22:6), Sphingomyelin (d17:1,C16:0), Histidine, Sphingomyelin (41:2), Tryptophan, Lysophosphatidylethanolamine (C18:0), Ceramide (d18:1,C24:0), and Ceramide (d18:2,C24:0).
60. The method of embodiment 56, wherein said pancreatic cancer is a resectable pancreatic cancer and wherein said at least one diagnostic biomarker is selected from the list consisting of Proline, Ceramide (d18:2,C24:0), Ceramide (d18:1,C24:0), Phosphatidylethanolamine (C18:0,C22:6), Tryptophan, Histidine, Lysophosphatidylethanolamine (C18:0), Sphingomyelin (35:1), Sphingomyelin (d18:2,C17:0), Sphingomyelin (d17:1,C16:0), and Sphingomyelin (41:2).
61. The method of embodiment 56, wherein said pancreatic cancer is a resectable pancreatic cancer, wherein said subject is a subject suffering from chronic pancreatitis, and wherein said at least one diagnostic biomarker is selected from the list consisting of Sphingomyelin (35:1), Ceramide (d18:1,C24:0), Phosphatidylethanolamine (C18:0,C22:6), Ceramide (d18:2,C24:0), Lysophosphatidylethanolamine (C18:0), and Tryptophan.
62. The method of embodiment 56, wherein said pancreatic cancer is a resectable pancreatic cancer, wherein said subject is a subject suffering from new-onset diabetes, and wherein said wherein said at least one diagnostic biomarker is selected from the list consisting of Lysophosphatidylethanolamine (C18:2), Proline, Sphingomyelin (35:1), Sphingomyelin (d18:2,C17:0), Phosphatidylethanolamine (C18:0,C22:6), Tryptophan, Sphingomyelin (d17:1,C16:0), Ceramide (d18:1,C24:0), Ceramide (d18:2,C24:0), Sphingomyelin (41:2), Lysophosphatidylethanolamine (C18:0) and Histidine.
63. The method of any one of embodiments 56 to 62, wherein said at least one diagnostic biomarker is selected from the list consisting of Lysophosphatidylethanolamine (C18:0), Phosphatidylethanolamine (C18:0, C22:6), and sphingomyelin (35:1), preferably, wherein said sphingomyelin (35:1) is the sum of the amounts of sphingomyelin (d18:1,C17:0) and sphingomyelin (d17:1,C18:0); or is the amount of sphingomyelin (d18:1,C17:0).
64. The method of any one of embodiments 56 to 63, further comprising the steps of any one of embodiments 53 to 55.
65. A diagnostic device for carrying out a method according to embodiments 1 to 44 or 53 to 64, comprising:
a) an analysing unit comprising at least one detector for at least the small molecule diagnostic biomarkers of a group of diagnostic biomarkers according to said embodiments, wherein said analyzing unit is adapted for determining the amounts of at least said small molecule diagnostic biomarkers detected by the at least one detector, and, operatively linked thereto;
b) an evaluation unit comprising a computer comprising tangibly embedded a computer program code for carrying out a comparison of the determined amounts of the small molecule diagnostic biomarkers, and, preferably, CA19-9, with a reference and a data base comprising said reference for said diagnostic biomarkers, whereby it is diagnosed whether a subject suffers from pancreatic cancer.

### 66. Use

(i) of a group of diagnostic biomarkers according to any one of embodiments 1 to 44 or 53 to 55; or
(ii) of a diagnostic biomarker according to any one of embodiments 56 to 64; in a sample of a subject for diagnosing pancreatic cancer or for the preparation of a pharmaceutical and/or diagnostic composition for diagnosing pancreatic cancer.
67. Use of a mixture comprising at least one, preferably both of L-Alanine d4 and Ceramide (d18:1,17:0) as a, preferably internal, standard in a method according to any one of embodiments 1 to 44 or according to any one of embodiments 53 to 64.
68. The method of any one of embodiments 1 to 44, wherein said group of diagnostic biomarkers comprises, preferably consists of, the diagnostic biomarkers CA 19-9, [histidine+proline+tryptophan], [sphingomyelin (d17:1,C16:0)+sphingomyelin (d18:2,C17:0)+sphingomyelin (35:1)+sphingomyelin (41:2)], and [ceramide (d18:1,C24:0)+ceramide (d18:2,C24:0)].
69. The method of any one of embodiments 1 to 44, wherein said group of diagnostic biomarkers comprises, preferably consists of, the diagnostic biomarkers CA 19-9, [histidine+proline+tryptophan], [sphingomyelin (d17:1,C16:0)+sphingomyelin (d18:2,C17:0)+sphingomyelin (35:1)+sphingomyelin (41:2)], [ceramide (d18:1,C24:0)+ceramide (d18:2,C24:0)], and [lysophosphatidylethanolamine (C18:2)/phosphatidylethanolamine (C18:0,C22:6)].
70. The method of any one of embodiments 1 to 44 or 68 to 69, wherein comparing said amounts of the diagnostic biomarkers with a reference comprises assigning a smaller weight, preferably a weight of 0, to the amount of CA19-9 in case the amount of CA19-9 determined is less than about 5 U/ml.
71. The method of any one of embodiments 45 to 50, wherein said method comprises additional steps
b1) obtaining a first aliquot of the non-phase separating, protein precipitating solution of step b),
b2) removing at least part of the solvent, preferably of the liquid from the remaining non-phase separating, protein precipitating solution of step b1),
b3) optionally, dissolving a residue obtained in step b2 in an appropriate solvent to yield a second aliquot, and, preferably,
determining metabolites expected or known to be present at a high concentration from the first aliquot, and determining metabolites expected or known to be present in the sample at a low concentration from the second aliquot.
72. The method of any one of embodiments 45 to 50 or 71, further comprising derivatizing, preferably dansylating, said diagnostic biomarkers before step d).
73. The method of any one of embodiments 45 to 50 or 71 to 72, wherein separating said biomarkers in the non-proteinaceous fraction by chromatography comprises separating said biomarkers in the non-proteinaceous fraction by reverse-phase chromatography, preferably by RP18-HPLC or RP18-UPLC.
74. The method of any one of embodiments 45 to 50 or 71 to 73, wherein detecting said biomarkers comprises positive-ion and/or negative-ion ESI, preferably positive-ion ESI.

The following Examples shall merely illustrate the invention. They shall not be construed, whatsoever, to limit the scope of the invention.

### FIGURE LEGENDS

FIG. 1: Legend to the graphical presentation of FIGS. 2 to 5.

FIG. 2: CA19-9 concentration (U/ml) in samples from patients suffering from various diseases: 1: Pancreatic cancer; 2: Chronic pancreatitis; 3: Non-pancreatic control (thyroid resections and hernia repair); 4: Diabetes and other comorbidities; 5: Other comorbidities, but no diabetes; 6: Small cell lung cancer; 7: Non-small cell lung cancer (NSCLC); 8: NSCLC adenocarcinoma; 9: NSCLC large cell carcinoma; 10: NSCLC squamous cell carcinoma; 11: Diabetes and dyslipidemia, most also hypertension; 12: Diabetes but no dyslipidemia, more than half also hypertension; 13: No comorbidities, age 62 or younger; 14: No comorbidities, age 63 or older; 15: Prostate cancer; 16: Cardiovascular diseases; 17: Chronic obstructive pulmonary disease, half also hypertension; 18: Dyslipidemia but no diabetes, more than half also hypertension; 19: Hypertension with other comorbidities, but no diabetes or dyslipidemia; 20: Hypertension only; 21: Other comorbidities, age 62 or younger; 22: Other comorbidities, age 63 or older; 23: Thyroid disorders. Graphical representation as described in FIG. 1.

FIG. 3: Prediction scores of panel 1 with (A) and without (B) CA19-9 for various diseases; graphical representation as described in FIG. 1, diseases as described in legend to FIG. 2.

FIG. 4: Prediction scores of panel 7 with (A) and without (B) CA19-9 for various diseases; graphical representation as described in FIG. 1, diseases as described in legend to FIG. 2.

FIG. 5: Prediction scores of panel 28 with (A) and without (B) CA19-9 for various diseases; graphical representation as described in FIG. 1, diseases as described in legend to FIG. 2.

### EXAMPLE 1: PATIENT CHARACTERISTICS, PLASMA PREPARATION

A total of 235 patients with pancreatic cancer, chronic pancreatitis and non-pancreatic controls (hernia repair and thyroid resections) were enrolled in the clinical study. In this retrospective case control study, samples of 77 patients suffering from pancreatic ductal adenocarcinoma (PDAC) (40 of them in a resectable tumor stage (IA-IIB)), 79 samples of chronic pancreatitis (CP) patients, samples of 79 non-pancreatic control patients matched for age, gender and BMI were included. The mean age of the pancreatic cancer patients was 67 years. The mean age of the chronic pancreatitis patients was 51 years. The mean age of the non-pancreatic control patients was 64 years. Patients were overnight fasted and consecutively recruited from one clinical center. Exclusion criteria were a concomitant malignant disease, curative treatment of malignant disease less than 2 years of recruitment to the trial, concomitant cystic diseases of the pancreas, pregnancy or patients unable to give informed consent.

Additionally, plasma samples of 52 male diabetes patients and 52 male non-diabetic patients, age and BMI matched from five different centers, overnight fasted, were analyzed. The mean age of the diabetic patients was 69 years. The mean age of the non-diabetic patients was 69 years. All patients or their legal representatives gave their written informed consent and the local ethics review boards approved the protocol. After blood drawing and centrifugation according to the blood draw tube manufacturer's instruction, EDTA plasma was collected in Eppendorf tubes and stored at −80° C. for further analysis.

### EXAMPLE 2: BIOMARKER ANALYSIS

The small molecule diagnostic biomarkers of the groups of diagnostic biomarkers listed as panels in Table 9 were analyzed by a one-shot LC-MS/MS measurement described in Example 3, where the analytes are further characterized by multiple reaction monitoring (MRM) transitions. Each analyte may contain more than one metabolite, whereby the metabolites contained in the same analyte have at least identical sum formula parameters and, thus, in the case of, e.g., lipids an identical chain length and identical numbers of double bonds in the fatty acid and/or other long-chain aliphatic moieties, e.g., sphingobase moieties.

Carbohydrate antigen 19-9 (CA 19-9) was analyzed in blood plasma or serum by a radioimmunoassay (RIA) in clinical chemistry laboratories. The normal range of CA 19-9 in the blood of a healthy individual is 0-37 U/mL (Units per milliliter).

### EXAMPLE 3: ANALYTICAL METHOD

Human plasma samples were prepared and subjected to LC-MS/MS analysis as follows: 20 μl human plasma was mixed with 100 μl internal standard mixture (alanine d4: 12.24 μg/ml; ceramide (d18:1,C17:0): 0.154 μg/ml were dissolved in dimethyl sulfoxide, methanol, dichloromethane and water (in a ratio 12.3:2.2:1.1:1, v/v/v/v)) and 700 μl extraction solvent containing methanol and dichloromethane in a ratio of 2:1 (v/v).

After the samples were thoroughly mixed at 20° C. for 5 min, the precipitated proteins were removed by centrifugation for 10 min. 150 μl of the liquid supernatant was transferred to an appropriate glass vial for further derivatization with dansyl chloride, which allows the dansylation of primary and secondary amine groups. For this purpose, 25 μl of 0.2 mol/l sodium bicarbonate buffer (dissolved in water), 25 μl of 4 mg/ml dansyl chloride solution (dissolved in acetonitrile) and 50 μl dimethyl sulfoxide were added. The dansylation was carried out under constant mixing at 35° C. for 150 min. The so-obtained reaction mixtures were analyzed by LC-MS/MS.

The LC-MS/MS systems consisted of an Agilent 1100 LC system (Agilent Technologies, Waldbronn, Germany) coupled with an API 4000 Mass spectrometer (ABSCIEX, Toronto, Canada). HPLC analysis was performed on commercially available reversed phase separation columns with C18 stationary phases (Phenomenex Ascentis Express C18, 2.7 μm, 50×2.1 mm).

Up to 2 μl of the above-mentioned so-obtained reaction mixture was injected and separated by gradient elution using a mixture of solvents consisting of methanol, water, formic acid, 2-propanol and 2-methoxy-2-methylpropane at a flow rate of 600 μl/min (e.g. starting from 0% solvent B to 100% solvent B in 7 min):

Solvent A: 400 g methanol, 400 g water, 1 g formic acid
Solvent B: 400 g 2-methoxy-2-methylpropane, 200 g 2-propanol, 100 g methanol, 1 g formic acid

Mass spectrometry was carried out by electrospray ionization (ESI) in positive ion mode using multiple reaction monitoring (MRM). Using ESI, sphingomyelins with equal numbers of carbons and double bonds were detected together, these isobaric species were not separated chromatographically.

The diagnostic biomarkers listed in Table 1 can be measured with MRM. The respective amino acid analytes were measured with a quantifier and a qualifier MRM transition, whereas the analytes of the diagnostic biomarkers listed in Table 1 are measured with a respective quantifier only.

Quantitative evaluations of all small molecule biomarkers with commercially available quantification standards were achieved by external calibration in delipidized plasma. Delipidized plasma was used to simulate a matrix as close as possible to real plasma.

For small molecule biomarkers without commercially available standards, a commercially available standard of the same lipid class was used for the external calibration.

Reference controls were prepared by lyophilization of different amounts of commercially available human plasma (12 μl, 20 μl, 28 μl) to check the linearity of small molecule biomarkers under real matrix condition. The ratios of the calculated concentrations of 12 μl/20 μl and 28 μl/20 μl of the lyophilized human reference control plasma delivered values between 0.5 and 0.7 and between 1.3 and 1.5, respectively, for all small molecule biomarkers of Table 1.

Recovery controls were prepared by adding a known standard concentration to lyophilized plasma samples of the same commercially available human reference control plasma as used for the reference controls. The lyophilized plasma samples were stored in a freezer until sample preparation.

Inter-day quality controls were prepared by extracting multiple samples of the commercially available human reference control plasma with extraction solvent and internal standard solution followed by dansylation. The dansylated reaction mixtures of all samples were pooled, and stored in aliquots in a freezer until they were used for the daily quality control of the instrument performance and sample preparation.

Quality controls for the method precision were prepared daily by extracting multiple samples of the commercially available human reference control plasma and were measured equally distributed in the sample batch.

TABLE 1
Diagnostic biomarkers used for generating groups of diagnostic biomarkers and their
quantification and internal standards for analysis; for the diagnostic biomarkers
“sphingomyelin (35:1)” and sphingomyelin (41:2)”, see text; the term
“direction” refers to the direction of change of the respective diagnostic
biomarker in samples from pancreatic cancer patients versus the non-pancreatic
cancer samples of the study.

| | | | Transition | | |
|---|---|---|---|---|---|
| | | | (parent/ | | Internal |
| | | | fragment) | Quantification Standard | Standard |
| | Diagnostic biomarker | Direction | (Da) | Compound | Compound |
| Amino acids | Histidine | down | 622.5/170.1 | L-Histidine | L-Alanine |
| | | | | | d4 |
| Amino acids | Proline | down | 349.3/170.1 | L-Proline | L-Alanine |
| | | | | | d4 |
| Amino acids | Tryptophan | down | 438.3/170.1 | L-Tryptophan | L-Alanine |
| | | | | | d4 |
| Ceramides | Ceramide (d18:1, C24:0) | down | 650.6/264.2 | Ceramide (d18:1, C24:0) | Ceramide |
| | | | | | (d18:1, 17:0) |
| Ceramides | Ceramide (d18:2, C24:0) | down | 648.6/262.2 | Ceramide (d18:1, C24:1) | Ceramide |
| | | | | | (d18:1, 17:0) |
| Ethanolamine | Lysophosphatidylethanolamine | down | 715.6/341.4 | Lysophosphatidylethanolamine | L-Alanine |
| lipids | (C18:0) | | | (C18:0) | d4 |
| Ethanolamine | Lysophosphatidylethanolamine | down | 711.6/337.4 | Lysophosphatidylethanolamine | L-Alanine |
| lipids | (C18:2) | | | (C18:0) | d4 |
| Ethanolamine | Phosphatidylethanolamine | up | 1025.8/651.6 | Phosphatidylethanolamine | L-Alanine |
| lipids | (C18:0, C22:6) | | | (C18:0, C22:6) | d4 |
| Sphingomyelins | Sphingomyelin (d17:1, C16:0) | up | 689.5/184.1 | Sphingomyelin (d18:1, C17:0) | Ceramide |
| | | | | | (d18:1, 17:0) |
| Sphingomyelins | Sphingomyelin (35:1) | up | 717.5/184.1 | Sphingomyelin (d18:1, C17:0) | Ceramide |
| | | | | | (d18:1, 17:0) |
| Sphingomyelins | Sphingomyelin (41:2) | up | 799.5/184.1 | Sphingomyelin (d18:1, C24:1) | Ceramide |
| | | | | | (d18:1, 17:0) |
| Sphinomyelins | Sphingomyelin (d18:2, C17:0) | up | 715.5/184.1 | Sphingomyelin (d18:1, C17:0) | Ceramide |
| | | | | | (d18:1, 17:0) |

### EXAMPLE 4: DATA ANALYSIS, NORMALIZATION AND STATISTICAL EVALUATION

For each diagnostic biomarker listed in Table 1, the direction of change in PDAC patients relative to controls consisting of CP patients and non-pancreatic controls was calculated by a simple linear model (ANOVA) with “disease”, “age”, “gender”, “BMI”, and “sample storage time”, if appropriate, as fixed effects. The direction ‘Up’ means that the levels of the biomarker are higher in PDAC patients relative to controls consisting of CP patients or non-pancreatic controls, the direction ‘Down’ means that the levels of the biomarker are lower in PDAC patients relative to CP patients or non-pancreatic controls. Prior to statistical analysis, log 10 transformation of ratios was conducted to assure normal distribution of data. The software R 2.8.1 (package nlme) was used for ANOVA.

The direction of change and ANOVA results of all small molecule biomarkers that were subsequently used for biomarker panel definition are given in the Tables 2 to 7, below:

TABLE 2
List of identified biomarkers in plasma for pancreatic
cancer relative to chronic pancreatitis

| | ANOVA result of pancreatic cancer | | |
|---|---|---|---|
| | relative to chronic pancreatitis | | |
| | Estimated fold | | |
| Diagnostic biomarker | change | p-value | t-value |
| Sphingomyelin (35:1) | 1.261 | 0.00023 | 3.749 |
| Phosphatidylethanolamine | 1.327 | 0.00705 | 2.719 |
| (C18:0, C22:6) | | | |
| Lysophosphatidylethanolamine | 0.879 | 0.05333 | −1.942 |
| (C18:0) | | | |

TABLE 3
List of identified biomarkers in plasma for pancreatic
cancer relative to non-pancreatic controls

| | ANOVA result of pancreatic cancer | | |
|---|---|---|---|
| | relative to non-pancreatic controls | | |
| | Estimated fold | | |
| Diagnostic biomarker | change | p-value | t-value |
| Phosphatidylethanolamine | 1.329 | 0.00142 | 3.231 |
| (C18:0, C22:6) | | | |
| Lysophosphatidylethanolamine | 0.897 | 0.05226 | −1.951 |
| (C18:0) | | | |
| Sphingomyelin (35:1) | 1.096 | 0.08245 | 1.744 |

TABLE 4
List of identified biomarkers in plasma for pancreatic cancer
relative to diabetic subjects (from chronic pancreatitis,
non-pancreatic controls, diabetes group)

| | ANOVA result of pancreatic cancer | | |
|---|---|---|---|
| | relative to diabetic subjects (from | | |
| | chronic pancreatitis, non-pancreatic | | |
| | controls, diabetes group) | | |
| | Estimated fold | | |
| Diagnostic biomarker | change | p-value | t-value |
| Lysophosphatidylethanolamine | 0.663 | 0.0000012 | −4.952 |
| (C18:2) | | | |
| Proline | 0.789 | 0.0000021 | −4.836 |
| Sphingomyelin (35:1) | 1.240 | 0.00005 | 4.117 |
| Sphingomyelin (d18:2, C17:0) | 1.209 | 0.00010 | 3.947 |
| Phosphatidylethanolamine | 1.390 | 0.00032 | 3.634 |
| (C18:0, C22:6) | | | |
| Sphingomyelin (d17:1, C16:0) | 1.212 | 0.00054 | 3.494 |
| Histidine | 0.912 | 0.00178 | −3.151 |
| Sphingomyelin (41:2) | 1.151 | 0.00215 | 3.093 |
| Tryptophan | 0.872 | 0.00219 | −3.088 |
| Lysophosphatidylethanolamine | 0.858 | 0.00426 | −2.879 |
| (C18:0) | | | |
| Ceramide (d18:1, C24:0) | 0.861 | 0.00797 | −2.670 |
| Ceramide (d18:2, C24:0) | 0.822 | 0.00797 | −2.670 |

TABLE 5
List of identified biomarkers in plasma for resectable
pancreatic cancer relative to chronic pancreatitis

| | ANOVA result of resectable pancreatic | | |
|---|---|---|---|
| | cancer relative to chronic pancreatitis | | |
| | Estimated fold | | |
| Diagnostic biomarker | change | p-value | t-value |
| Sphingomyelin (35:1) | 1.271 | 0.00140 | 3.242 |
| Ceramide (d18:1, C24:0) | 0.799 | 0.00930 | −2.628 |
| Phosphatidylethanolamine | 1.363 | 0.01124 | 2.560 |
| (C18:0, C22:6) | | | |
| Ceramide (d18:2, C24:0) | 0.772 | 0.02307 | −2.291 |
| Lysophosphatidylethanolamine | 0.874 | 0.08510 | −1.731 |
| (C18:0) | | | |
| Tryptophan | 0.927 | 0.27129 | −1.103 |

TABLE 6
List of identified biomarkers in plasma for resectable
pancreatic cancer relative to non-pancreatic control

| | ANOVA result of resectable | | |
|---|---|---|---|
| | pancreatic cancer relative | | |
| | to non-pancreatic controls | | |
| | Estimated fold | | |
| Diagnostic biomarker | change | p-value | t-value |
| Proline | 0.805 | 0.00096 | −3.356 |
| Ceramide (d18:2, C24:0) | 0.731 | 0.00163 | −3.197 |
| Ceramide (d18:1, C24:0) | 0.805 | 0.00394 | −2.919 |
| Phosphatidylethanolamine | 1.348 | 0.00496 | 2.843 |
| (C18:0, C22:6) | | | |
| Tryptophan | 0.856 | 0.00932 | −2.627 |
| Histidine | 0.919 | 0.03148 | −2.167 |
| Lysophosphatidylethanolamine | 0.881 | 0.06323 | −1.868 |
| (C18:0) | | | |
| Sphingomyelin (35:1) | 1.118 | 0.08464 | 1.733 |
| Sphingomyelin (d18:2, C17:0) | 1.094 | 0.14099 | 1.478 |
| Sphingomyelin (d17:1, C16:0) | 1.073 | 0.30992 | 1.018 |
| Sphingomyelin (41:2) | 1.019 | 0.75048 | 0.318 |

TABLE 7
List of identified biomarkers in plasma for resectable pancreatic
cancer relative to diabetic subjects (from chronic pancreatitis,
non-pancreatic controls, diabetes group)

| | ANOVA result of resectable pancreatic | | |
|---|---|---|---|
| | cancer relative to diabetic subjects | | |
| | (from chronic pancreatitis, non- | | |
| | pancreatic control, diabetes group) | | |
| | Estimated fold | | |
| Diagnostic biomarker | change | p-value | t-value |
| Lysophosphatidylethanolamine | 0.644 | 0.00001 | −4.541 |
| (C18:2) | | | |
| Proline | 0.797 | 0.00020 | −3.769 |
| Sphingomyelin (35:1) | 1.267 | 0.00025 | 3.712 |
| Sphingomyelin (d18:2, C17:0) | 1.229 | 0.00052 | 3.508 |
| Phosphatidylethanolamine | 1.436 | 0.00094 | 3.343 |
| (C18:0, C22:6) | | | |
| Tryptophan | 0.851 | 0.00238 | −3.066 |
| Sphingomyelin (d17:1, C16:0) | 1.219 | 0.00372 | 2.925 |
| Ceramide (d18:1, C24:0) | 0.822 | 0.00375 | −2.922 |
| Ceramide (d18:2, C24:0) | 0.793 | 0.00809 | −2.667 |
| Sphingomyelin (41:2) | 1.154 | 0.01058 | 2.573 |
| Lysophosphatidylethanolamine | 0.862 | 0.01971 | −2.345 |
| (C18:0) | | | |
| Histidine | 0.929 | 0.03442 | −2.125 |

Classification using the Elastic Net algorithm (Zou and Hastie (2005) Regularization and variable selection via the elastic net, Journal of the Royal Statistical Society, Series B: 67, 301-320) as implemented in the R (version 3.0.1) package glmnet (version 1.9-8) was calculated to obtain a logistic regression model on log 10 transformed data including CA 19-9. The L1 and the L2 penalties were given equal weight. The log-transformed biomarker data including CA19-9 were also centered and scaled to unit variance before the analysis. This logistic regression models allows the calculation of predicted probabilities for each of the patients having pancreatic cancer.

A10-fold cross-validation was used to obtain an unbiased estimate of the area under the curve (AUC) on the remaining fold. The 95% confidence intervals for the AUC were calculated using the binormal model of the receiver operating characteristic (ROC) curve as described in Zhou, Obuchowski and McClish [Statistical Methods in Diagnostic Medicine (2011), 2nd Edition, by Zhou, Obuchowski and McClish]. The assumption of the binormality of the logit-transformed prediction scores was visually checked with a QQ-Plot. Afterwards the final model coefficients were determined by retraining the classifier on the entire data.

### EXAMPLE 5: BIOMARKER PANEL FOR DIAGNOSIS OF PANCREATIC CANCER

The diagnostic biomarkers of the biomarker panels allowing for diagnosis of pancreatic cancer versus chronic pancreatitis, pancreatic cancer versus non-pancreatic control, and pancreatic cancer versus (chronic pancreatitis plus non pancreatic control), were manually selected and optimized according to their discriminating performance both multivariate and univariate and the feasibility of their concomitant analysis in a single analytical approach. These pre-defined panels were then tested for their discrimination performance using Elastic Net algorithm combined with ROC curve analysis.

The biomarker panels that were identified for diagnosis of pancreatic cancer consist of a most preferred core panel that comprises in addition to CA19-9 at least one small molecule biomarker from each of the metabolite classes amino acids, ceramides, and sphingomyelins as shown in Table 8A. Frequently, the biomarker panels comprised in addition to CA19-9 at least one small molecule biomarker from each of the metabolite classes amino acids, ceramides, sphingomyelins, and ethanolamine lipids as shown in Table 8B.

TABLE 8A
Core panel definition for diagnosis of pancreatic cancer

| Panel | CA19-9 | Amino acid | Ceramide | Sphingomyelin |
|---|---|---|---|---|
| Core panel | CA19-9 | x | x | x |

TABLE 8B
Extended core panel definition for diagnosis of pancreatic cancer

| | | Amino | Cera- | | Ethanolamine |
|---|---|---|---|---|---|
| Panel | CA19-9 | acid | mide | Sphingomyelin | lipid |
| Extended | CA19-9 | x | x | x | x |
| core panel | | | | | |

The core panel structure shown in Tables 8A or 8B can be composed of the following biomarkers:
amino acids: proline, and/or tryptophan, and/or histidine
ceramides: ceramide (d18:1,C24:0) and/or ceramide (d18:2,C24:0)
sphingomyelins: Sphingomyelin (35:1), and/or sphingomyelin (d17:1,C16:0), and/or sphingomyelin (d18:2,C17:0), and/or (Sphingomyelin (41:2))
ethanolamine lipids: Phosphatidylethanolamine (C18:0,C22:6), and/or lysophosphatidylethanolamine (C18:2) and/or lysophosphatidylethanolamine (C18:0)

Respective biomarker panels of the invention are shown in Table 9, below:

TABLE 9
Panel composition for diagnosis of pancreatic cancer

| Panel | |
|---|---|
| Number | Panel Composition |
| 1 | CA19-9, Ceramide (d18:1, C24:0), Proline, Sphingomyelin (35:1) |
| 2 | CA19-9, Ceramide (d18:1, C24:0), Phosphatidylethanolamine |
| | (C18:0, C22:6), Proline, Sphingomyelin (35:1) |
| 3 | CA19-9, Ceramide (d18:1, C24:0), Lysophosphatidylethanolamine (C18:2), |
| | Proline, Sphingomyelin (35:1) |
| 4 | CA19-9, Ceramide (d18:1, C24:0), Lysophosphatidylethanolamine (C18:0), |
| | Proline, Sphingomyelin (35:1) |
| 5 | CA19-9, Ceramide (d18:1, C24:0), Lysophosphatidylethanolamine (C18:2), |
| | Proline, Sphingomyelin (35:1) |
| 6 | CA19-9, Ceramide (d18:1, C24:0), Lysophosphatidylethanolamine (C18:2), |
| | Proline, Sphingomyelin (35:1), Sphingomyelin (d18:2, C17:0) |
| 7 | CA19-9, Ceramide (d18:1, C24:0), Proline, Sphingomyelin (35:1), |
| | Sphingomyelin (d18:2, C17:0), Tryptophan |
| 8 | CA19-9, Ceramide (d18:1, C24:0), Lysophosphatidylethanolamine (C18:2), |
| | Proline, Sphingomyelin (35:1), Sphingomyelin (d18:2, C17:0), Tryptophan |
| 9 | CA19-9, Ceramide (d18:1, C24:0), Lysophosphatidylethanolamine (C18:2), |
| | Phosphatidylethanolamine (C18:0, C22:6), Proline, Sphingomyelin (35:1), |
| | Tryptophan |
| 10 | CA19-9, Ceramide (d18:1, C24:0), Lysophosphatidylethanolamine (C18:2), |
| | Phosphatidylethanolamine (C18:0, C22:6), Proline, Sphingomyelin (35:1), |
| | Sphingomyelin (d18:2, C17:0), Tryptophan |
| 11 | CA19-9, Ceramide (d18:1, C24:0), Lysophosphatidylethanolamine (C18:0), |
| | Lysophosphatidylethanolamine (C18:2), Phosphatidylethanolamine |
| | (C18:0, C22:6), Proline, Sphingomyelin (35:1), Sphingomyelin |
| | (d18:2, C17:0), Tryptophan |
| 12 | CA19-9, Ceramide (d18:2, C24:0), Proline, Sphingomyelin (35:1) |
| 13 | CA19-9, Ceramide (d18:2, C24:0), Phosphatidylethanolamine |
| | (C18:0, C22:6), Proline, Sphingomyelin (35:1) |
| 14 | CA19-9, Ceramide (d18:2, C24:0), Lysophosphatidylethanolamine (C18:0), |
| | Proline, Sphingomyelin (35:1) |
| 15 | CA19-9, Ceramide (d18:2, C24:0), Lysophosphatidylethanolamine (C18:2), |
| | Proline, Sphingomyelin (35:1) |
| 16 | CA19-9, Ceramide (d18:1, C24:0), Proline, Sphingomyelin (d17:1, C16:0) |
| 17 | CA19-9, Ceramide (d18:1, C24:0), Proline, Sphingomyelin (41:2) |
| 18 | CA19-9, Ceramide (d18:1, C24:0), Proline, Sphingomyelin (d18:2, C17:0) |
| 19 | CA19-9, Ceramide (d18:2, C24:0), Proline, Sphingomyelin (d17:1, C16:0) |
| 20 | CA19-9, Ceramide (d18:2, C24:0), Proline, Sphingomyelin (41:2) |
| 21 | CA19-9, Ceramide (d18:2, C24:0), Proline, Sphingomyelin (d18:2, C17:0) |
| 22 | CA19-9, Ceramide (d18:1, C24:0), Histidine, Sphingomyelin (35:1) |
| 23 | CA19-9, Ceramide (d18:1, C24:0), Histidine, Sphingomyelin (d17:1, C16:0) |
| 24 | CA19-9, Ceramide (d18:1, C24:0), Histidine, Sphingomyelin (41:2) |
| 25 | CA19-9, Ceramide (d18:1, C24:0), Histidine, Sphingomyelin (d18:2, C17:0) |
| 26 | CA19-9, Ceramide (d18:2, C24:0), Histidine, Sphingomyelin (35:1) |
| 27 | CA19-9, Ceramide (d18:2, C24:0), Histidine, Sphingomyelin (d17:1, C16:0) |
| 28 | CA19-9, Ceramide (d18:2, C24:0), Histidine, Sphingomyelin (41:2) |
| 29 | CA19-9, Ceramide (d18:2, C24:0), Histidine, Sphingomyelin (d18:2, C17:0) |
| 30 | CA19-9, Ceramide (d18:1, C24:0), Sphingomyelin (35:1), Tryptophan |
| 31 | CA19-9, Ceramide (d18:1, C24:0), Sphingomyelin (41:2), Tryptophan |
| 32 | CA19-9, Ceramide (d18:1, C24:0), Sphingomyelin (d18:2, C17:0), |
| | Tryptophan |
| 33 | CA19-9, Ceramide (d18:1, C24:0), Sphingomyelin (d17:1, C16:0), |
| | Tryptophan |
| 34 | CA19-9, Ceramide (d18:2, C24:0), Sphingomyelin (35:1), Tryptophan |
| 35 | CA19-9, Ceramide (d18:2, C24:0), Sphingomyelin (d17:1, C16:0), |
| | Tryptophan |
| 36 | CA19-9, Ceramide (d18:2, C24:0), Sphingomyelin (41:2), Tryptophan |
| 37 | CA19-9, Ceramide (d18:2, C24:0), Sphingomyelin (d18:2, C17:0), |
| | Tryptophan |
| 38 | CA19-9, Ceramide (d18:1, C24:0), Lysophosphatidylethanolamine (C18:2), |
| | Proline, Sphingomyelin (d17:1, C16:0) |
| 39 | CA19-9, Ceramide (d18:1, C24:0), Lysophosphatidylethanolamine (C18:2), |
| | Proline, Sphingomyelin (41:2) |
| 40 | CA19-9, Ceramide (d18:1, C24:0), Lysophosphatidylethanolamine (C18:2), |
| | Proline, Sphingomyelin (d18:2, C17:0) |
| 41 | CA19-9, Ceramide (d18:1, C24:0), Lysophosphatidylethanolamine (C18:0), |
| | Proline, Sphingomyelin (d17:1, C16:0) |
| 42 | CA19-9, Ceramide (d18:1, C24:0), Lysophosphatidylethanolamine (C18:0), |
| | Proline, Sphingomyelin (41:2) |
| 43 | CA19-9, Ceramide (d18:1, C24:0), Phosphatidylethanolamine |
| | (C18:0, C22:6), Proline, Sphingomyelin (d17:1, C16:0) |
| 44 | CA19-9, Ceramide (d18:1, C24:0), Phosphatidylethanolamine |
| | (C18:0, C22:6), Proline, Sphingomyelin (41:2) |
| 45 | CA19-9, Ceramide (d18:1, C24:0), Lysophosphatidylethanolamine (C18:0), |
| | Proline, Sphingomyelin (d18:2, C17:0) |
| 46 | CA19-9, Ceramide (d18:1, C24:0), Phosphatidylethanolamine |
| | (C18:0, C22:6), Proline, Sphingomyelin (d18:2, C17:0) |
| 47 | CA19-9, Ceramide (d18:2, C24:0), Phosphatidylethanolamine |
| | (C18:0, C22:6), Proline, Sphingomyelin (d17:1, C16:0) |
| 48 | CA19-9, Ceramide (d18:2, C24:0), Phosphatidylethanolamine |
| | (C18:0, C22:6), Proline, Sphingomyelin (41:2) |
| 49 | CA19-9, Ceramide (d18:2, C24:0), Phosphatidylethanolamine |
| | (C18:0, C22:6), Proline, Sphingomyelin (d18:2, C17:0) |
| 50 | CA19-9, Ceramide (d18:2, C24:0), Lysophosphatidylethanolamine (C18:0), |
| | Proline, Sphingomyelin (d17:1, C16:0) |
| 51 | CA19-9, Ceramide (d18:2, C24:0), Lysophosphatidylethanolamine (C18:0), |
| | Proline, Sphingomyelin (41:2) |
| 52 | CA19-9, Ceramide (d18:2, C24:0), Lysophosphatidylethanolamine (C18:0), |
| | Proline, Sphingomyelin (d18:2, C17:0) |
| 53 | CA19-9, Ceramide (d18:2, C24:0), Lysophosphatidylethanolamine (C18:2), |
| | Proline, Sphingomyelin (d17:1, C16:0) |
| 54 | CA19-9, Ceramide (d18:2, C24:0), Lysophosphatidylethanolamine (C18:2), |
| | Proline, Sphingomyelin (41:2) |
| 55 | CA19-9, Ceramide (d18:2, C24:0), Lysophosphatidylethanolamine (C18:2), |
| | Proline, Sphingomyelin (d18:2, C17:0) |
| 56 | CA19-9, Ceramide (d18:1, C24:0), Histidine, Lysophosphatidylethanolamine |
| | (C18:0), Sphingomyelin (35:1) |
| 57 | CA19-9, Ceramide (d18:1, C24:0), Histidine, Lysophosphatidylethanolamine |
| | (C18:2), Sphingomyelin (35:1) |
| 58 | CA19-9, Ceramide (d18:1, C24:0), Histidine, Phosphatidylethanolamine |
| | (C18:0, C22:6), Sphingomyelin (35:1) |
| 59 | CA19-9, Ceramide (d18:1, C24:0), Histidine, Lysophosphatidylethanolamine |
| | (C18:0), Sphingomyelin (d17:1, C16:0) |
| 60 | CA19-9, Ceramide (d18:1, C24:0), Histidine, Lysophosphatidylethanolamine |
| | (C18:0), Sphingomyelin (41:2) |
| 61 | CA19-9, Ceramide (d18:1, C24:0), Histidine, Lysophosphatidylethanolamine |
| | (C18:2), Sphingomyelin (d17:1, C16:0) |
| 62 | CA19-9, Ceramide (d18:1, C24:0), Histidine, Lysophosphatidylethanolamine |
| | (C18:2), Sphingomyelin (41:2) |
| 63 | CA19-9, Ceramide (d18:1, C24:0), Histidine, Lysophosphatidylethanolamine |
| | (C18:0), Sphingomyelin (d18:2, C17:0) |
| 64 | CA19-9, Ceramide (d18:1, C24:0), Histidine, Lysophosphatidylethanolamine |
| | (C18:2), Sphingomyelin (d18:2, C17:0) |
| 65 | CA19-9, Ceramide (d18:1, C24:0), Histidine, Phosphatidylethanolamine |
| | (C18:0, C22:6), Sphingomyelin (d17:1, C16:0) |
| 66 | CA19-9, Ceramide (d18:1, C24:0), Histidine, Phosphatidylethanolamine |
| | (C18:0, C22:6), Sphingomyelin (41:2) |
| 67 | CA19-9, Ceramide (d18:1, C24:0), Histidine, Phosphatidylethanolamine |
| | (C18:0, C22:6), Sphingomyelin (d18:2, C17:0) |
| 68 | CA19-9, Ceramide (d18:2, C24:0), Histidine, Lysophosphatidylethanolamine |
| | (C18:0), Sphingomyelin (35:1) |
| 69 | CA19-9, Ceramide (d18:2, C24:0), Histidine, Lysophosphatidylethanolamine |
| | (C18:2), Sphingomyelin (35:1) |
| 70 | CA19-9, Ceramide (d18:2, C24:0), Histidine, Phosphatidylethanolamine |
| | (C18:0, C22:6), Sphingomyelin (35:1) |
| 71 | CA19-9, Ceramide (d18:2, C24:0), Histidine, Phosphatidylethanolamine |
| | (C18:0, C22:6), Sphingomyelin (d18:2, C17:0) |
| 72 | CA19-9, Ceramide (d18:2, C24:0), Histidine, Lysophosphatidylethanolamine |
| | (C18:0), Sphingomyelin (d17:1, C16:0) |
| 73 | CA19-9, Ceramide (d18:2, C24:0), Histidine, Lysophosphatidylethanolamine |
| | (C18:0), Sphingomyelin (41:2) |
| 74 | CA19-9, Ceramide (d18:2, C24:0), Histidine, Lysophosphatidylethanolamine |
| | (C18:0), Sphingomyelin (d18:2, C17:0) |
| 75 | CA19-9, Ceramide (d18:2, C24:0), Histidine, Lysophosphatidylethanolamine |
| | (C18:2), Sphingomyelin (d17:1, C16:0) |
| 76 | CA19-9, Ceramide (d18:2, C24:0), Histidine, Lysophosphatidylethanolamine |
| | (C18:2), Sphingomyelin (41:2) |
| 77 | CA19-9, Ceramide (d18:2, C24:0), Histidine, Lysophosphatidylethanolamine |
| | (C18:2), Sphingomyelin (d18:2, C17:0) |
| 78 | CA19-9, Ceramide (d18:2, C24:0), Histidine, Phosphatidylethanolamine |
| | (C18:0, C22:6), Sphingomyelin (d17:1, C16:0) |
| 79 | CA19-9, Ceramide (d18:2, C24:0), Histidine, Phosphatidylethanolamine |
| | (C18:0, C22:6), Sphingomyelin (41:2) |
| 80 | CA19-9, Ceramide (d18:1, C24:0), Lysophosphatidylethanolamine (C18:0), |
| | Sphingomyelin (35:1), Tryptophan |
| 81 | CA19-9, Ceramide (d18:1, C24:0), Lysophosphatidylethanolamine (C18:2), |
| | Sphingomyelin (35:1), Tryptophan |
| 82 | CA19-9, Ceramide (d18:1, C24:0), Phosphatidylethanolamine |
| | (C18:0, C22:6), Sphingomyelin (35:1), Tryptophan |
| 83 | CA19-9, Ceramide (d18:1, C24:0), Lysophosphatidylethanolamine (C18:0), |
| | Sphingomyelin (41:2), Tryptophan |
| 84 | CA19-9, Ceramide (d18:1, C24:0), Lysophosphatidylethanolamine (C18:0), |
| | Sphingomyelin (d18:2, C17:0), Tryptophan |
| 85 | CA19-9, Ceramide (d18:1, C24:0), Lysophosphatidylethanolamine (C18:2), |
| | Sphingomyelin (d17:1, C16:0), Tryptophan |
| 86 | CA19-9, Ceramide (d18:1, C24:0), Lysophosphatidylethanolamine (C18:2), |
| | Sphingomyelin (41:2), Tryptophan |
| 87 | CA19-9, Ceramide (d18:1, C24:0), Lysophosphatidylethanolamine (C18:2), |
| | Sphingomyelin (d18:2, C17:0), Tryptophan |
| 88 | CA19-9, Ceramide (d18:1, C24:0), Phosphatidylethanolamine |
| | (C18:0, C22:6), Sphingomyelin (d17:1, C16:0), Tryptophan |
| 89 | CA19-9, Ceramide (d18:1, C24:0), Phosphatidylethanolamine |
| | (C18:0, C22:6), Sphingomyelin (41:2), Tryptophan |
| 90 | CA19-9, Ceramide (d18:1, C24:0), Phosphatidylethanolamine |
| | (C18:0, C22:6), Sphingomyelin (d18:2, C17:0), Tryptophan |
| 91 | CA19-9, Ceramide (d18:1, C24:0), Lysophosphatidylethanolamine (C18:0), |
| | Sphingomyelin (d17:1, C16:0), Tryptophan |
| 92 | CA19-9, Ceramide (d18:2, C24:0), Phosphatidylethanolamine |
| | (C18:0, C22:6), Sphingomyelin (35:1), Tryptophan |
| 93 | CA19-9, Ceramide (d18:2, C24:0), Lysophosphatidylethanolamine (C18:0), |
| | Sphingomyelin (35:1), Tryptophan |
| 94 | CA19-9, Ceramide (d18:2, C24:0), Lysophosphatidylethanolamine (C18:2), |
| | Sphingomyelin (35:1), Tryptophan |
| 95 | CA19-9, Ceramide (d18:2, C24:0), Phosphatidylethanolamine |
| | (C18:0, C22:6), Sphingomyelin (d17:1, C16:0), Tryptophan |
| 96 | CA19-9, Ceramide (d18:2, C24:0), Phosphatidylethanolamine |
| | (C18:0, C22:6), Sphingomyelin (41:2), Tryptophan |
| 97 | CA19-9, Ceramide (d18:2, C24:0), Phosphatidylethanolamine |
| | (C18:0, C22:6), Sphingomyelin (d18:2, C17:0), Tryptophan |
| 98 | CA19-9, Ceramide (d18:2, C24:0), Lysophosphatidylethanolamine (C18:0), |
| | Sphingomyelin (d17:1, C16:0), Tryptophan |
| 99 | CA19-9, Ceramide (d18:2, C24:0), Lysophosphatidylethanolamine (C18:0), |
| | Sphingomyelin (41:2), Tryptophan |
| 100 | CA19-9, Ceramide (d18:2, C24:0), Lysophosphatidylethanolamine (C18:0), |
| | Sphingomyelin (d18:2, C17:0), Tryptophan |
| 101 | CA19-9, Ceramide (d18:2, C24:0), Lysophosphatidylethanolamine (C18:2), |
| | Sphingomyelin (d17:1, C16:0), Tryptophan |
| 102 | CA19-9, Ceramide (d18:2, C24:0), Lysophosphatidylethanolamine (C18:2), |
| | Sphingomyelin (41:2), Tryptophan |
| 103 | CA19-9, Ceramide (d18:2, C24:0), Lysophosphatidylethanolamine (C18:2), |
| | Sphingomyelin (d18:2, C17:0), Tryptophan |
| 104 | CA19-9, Ceramide (d18:1, C24:0), Ceramide (d18:2, C24:0), Histidine, |
| | Lysophosphatidylethanolamine (C18:0), Lysophosphatidylethanolamine |
| | (C18:2), Phosphatidylethanolamine (C18:0, C22:6), Proline, Sphingomyelin |
| | (d17:1, C16:0), Sphingomyelin (35:1), Sphingomyelin (41:2), Sphingomyelin |
| | (d18:2, C17:0), Tryptophan |

All panels were analyzed for their discrimination performance of pancreatic cancer versus chronic pancreatitis; pancreatic cancer versus non-pancreatic control; pancreatic cancer versus (chronic pancreatitis plus non-pancreatic control) as described in Example 4. The panels shown in Table 9 performed as listed in Table 10, below. The diagnostic performance is in all cases increased compared to CA19-9 alone: CA19-9 alone resulted in AUC of discrimination performance of pancreatic cancer versus chronic pancreatitis; pancreatic cancer versus non-pancreatic control; pancreatic cancer versus (chronic pancreatitis plus non-pancreatic control) of 0.83, 0.87, and 0.85, respectively. In the resectable pancreatic cancer subgroup, CA19-9 alone resulted in AUC of discrimination performance of pancreatic cancer versus chronic pancreatitis; pancreatic cancer versus non-pancreatic control; pancreatic cancer versus (chronic pancreatitis plus non-pancreatic control) of 0.80, 0.86, and 0.84, respectively. In the low CA19-9 (<37 U/ml) subgroup, CA19-9 alone resulted in AUC of discrimination performance of pancreatic cancer versus chronic pancreatitis; pancreatic cancer versus non-pancreatic control; pancreatic cancer versus (chronic pancreatitis plus non-pancreatic control) of 0.53, 0.56, and 0.54, respectively.

TABLE 10
Diagnostic performance of the panels shown in Table 9
for detection of pancreatic cancer

| | AUC of | AUC of comparison | AUC of comparison of |
|---|---|---|---|
| | comparison of | of pancreatic | pancreatic cancer |
| | pancreatic cancer | cancer relative | relative to (chronic |
| Panel | relative to chronic | to non-pancreatic | pancreatitis and non- |
| Number | pancreatitis | controls | pancreatic controls) |
| 1 | 0.913 | 0.897 | 0.899 |
| 2 | 0.929 | 0.897 | 0.901 |
| 3 | 0.916 | 0.893 | 0.897 |
| 4 | 0.912 | 0.897 | 0.898 |
| 5 | 0.916 | 0.893 | 0.897 |
| 6 | 0.913 | 0.891 | 0.894 |
| 7 | 0.916 | 0.894 | 0.896 |
| 8 | 0.914 | 0.887 | 0.894 |
| 9 | 0.928 | 0.889 | 0.899 |
| 10 | 0.925 | 0.888 | 0.896 |
| 11 | 0.93 | 0.888 | 0.896 |
| 12 | 0.908 | 0.897 | 0.899 |
| 13 | 0.92 | 0.895 | 0.899 |
| 14 | 0.908 | 0.897 | 0.898 |
| 15 | 0.911 | 0.893 | 0.897 |
| 16 | 0.903 | 0.895 | 0.891 |
| 17 | 0.912 | 0.889 | 0.892 |
| 18 | 0.908 | 0.893 | 0.892 |
| 19 | 0.896 | 0.896 | 0.892 |
| 20 | 0.907 | 0.892 | 0.896 |
| 21 | 0.901 | 0.895 | 0.893 |
| 22 | 0.905 | 0.886 | 0.887 |
| 23 | 0.895 | 0.882 | 0.879 |
| 24 | 0.907 | 0.881 | 0.886 |
| 25 | 0.902 | 0.883 | 0.881 |
| 26 | 0.898 | 0.885 | 0.886 |
| 27 | 0.885 | 0.881 | 0.878 |
| 28 | 0.901 | 0.883 | 0.888 |
| 29 | 0.891 | 0.881 | 0.881 |
| 30 | 0.918 | 0.889 | 0.894 |
| 31 | 0.919 | 0.882 | 0.892 |
| 32 | 0.908 | 0.885 | 0.887 |
| 33 | 0.903 | 0.884 | 0.885 |
| 34 | 0.911 | 0.884 | 0.891 |
| 35 | 0.896 | 0.881 | 0.882 |
| 36 | 0.912 | 0.882 | 0.892 |
| 37 | 0.899 | 0.881 | 0.885 |
| 38 | 0.906 | 0.891 | 0.89 |
| 39 | 0.915 | 0.886 | 0.891 |
| 40 | 0.908 | 0.888 | 0.89 |
| 41 | 0.902 | 0.895 | 0.89 |
| 42 | 0.909 | 0.89 | 0.891 |
| 43 | 0.916 | 0.896 | 0.892 |
| 44 | 0.926 | 0.894 | 0.894 |
| 45 | 0.906 | 0.893 | 0.892 |
| 46 | 0.921 | 0.893 | 0.893 |
| 47 | 0.906 | 0.894 | 0.89 |
| 48 | 0.917 | 0.893 | 0.895 |
| 49 | 0.91 | 0.892 | 0.891 |
| 50 | 0.898 | 0.895 | 0.892 |
| 51 | 0.906 | 0.892 | 0.895 |
| 52 | 0.901 | 0.893 | 0.892 |
| 53 | 0.9 | 0.892 | 0.891 |
| 54 | 0.909 | 0.887 | 0.893 |
| 55 | 0.9 | 0.888 | 0.891 |
| 56 | 0.908 | 0.885 | 0.885 |
| 57 | 0.913 | 0.884 | 0.89 |
| 58 | 0.923 | 0.887 | 0.893 |
| 59 | 0.897 | 0.881 | 0.877 |
| 60 | 0.907 | 0.88 | 0.884 |
| 61 | 0.901 | 0.883 | 0.884 |
| 62 | 0.912 | 0.879 | 0.888 |
| 63 | 0.901 | 0.881 | 0.878 |
| 64 | 0.903 | 0.88 | 0.883 |
| 65 | 0.909 | 0.883 | 0.883 |
| 66 | 0.921 | 0.883 | 0.89 |
| 67 | 0.914 | 0.882 | 0.884 |
| 68 | 0.903 | 0.884 | 0.886 |
| 69 | 0.907 | 0.882 | 0.89 |
| 70 | 0.914 | 0.883 | 0.888 |
| 71 | 0.902 | 0.88 | 0.88 |
| 72 | 0.893 | 0.88 | 0.878 |
| 73 | 0.903 | 0.881 | 0.886 |
| 74 | 0.895 | 0.88 | 0.879 |
| 75 | 0.892 | 0.881 | 0.883 |
| 76 | 0.904 | 0.88 | 0.889 |
| 77 | 0.894 | 0.879 | 0.882 |
| 78 | 0.896 | 0.881 | 0.879 |
| 79 | 0.911 | 0.883 | 0.889 |
| 80 | 0.916 | 0.888 | 0.891 |
| 81 | 0.917 | 0.885 | 0.894 |
| 82 | 0.929 | 0.889 | 0.896 |
| 83 | 0.917 | 0.882 | 0.889 |
| 84 | 0.906 | 0.884 | 0.884 |
| 85 | 0.904 | 0.883 | 0.887 |
| 86 | 0.918 | 0.879 | 0.89 |
| 87 | 0.905 | 0.881 | 0.885 |
| 88 | 0.912 | 0.886 | 0.885 |
| 89 | 0.928 | 0.885 | 0.892 |
| 90 | 0.917 | 0.885 | 0.887 |
| 91 | 0.902 | 0.884 | 0.882 |
| 92 | 0.918 | 0.884 | 0.891 |
| 93 | 0.911 | 0.883 | 0.888 |
| 94 | 0.912 | 0.882 | 0.893 |
| 95 | 0.901 | 0.881 | 0.881 |
| 96 | 0.916 | 0.883 | 0.891 |
| 97 | 0.905 | 0.881 | 0.884 |
| 98 | 0.897 | 0.88 | 0.879 |
| 99 | 0.911 | 0.88 | 0.889 |
| 100 | 0.9 | 0.88 | 0.882 |
| 101 | 0.896 | 0.881 | 0.886 |
| 102 | 0.911 | 0.877 | 0.891 |
| 103 | 0.899 | 0.878 | 0.886 |
| 104 | 0.922 | 0.896 | 0.891 |

A classifier of pancreatic cancer diagnosis was obtained by training the elastic net algorithm on the predefined panels as described by Zou and Hastie ((2005) Regularization and variable selection via the elastic net, Journal of the Royal Statistical Society, Series B, 67, 301-320) using the pancreatic cancer group as positive class and the chronic pancreatitis and/or the non-pancreatic controls as negative class, respectively. In order to reduce overfitting effects, tenfold cross-validation was done which is well known to a person skilled in the art. As a result, for each of the classifiers weightings and scalings are obtained that are optimized for the classes they were trained for.

Those classifiers are subsequently tested by the same and further classification tasks, those being pancreatic cancer relative to chronic pancreatitis, pancreatic cancer relative to non-pancreatic controls, pancreatic cancer relative to (chronic pancreatitis and non-pancreatic controls), pancreatic cancer relative to all non-cancer subjects (chronic pancreatitis, non-pancreatic controls, diabetes group, non-diabetes group, and pancreatic cancer relative to diabetic subjects (from chronic pancreatitis, non-pancreatic controls, diabetes group). Each of those comparisons were applied on either the entire data set, or the resectable pancreatic cancer group, or the low CA19-9 group.

After training the panels on the differentiation of pancreatic cancer versus chronic pancreatitis, and pancreatic cancer versus non-pancreatic controls, and pancreatic cancer versus (chronic pancreatitis plus non-pancreatic controls), the resulting biomarkers were analyzed for their performance in the training groups, diabetic subgroups and all non-cancer subjects including the 104 extra diabetic and non-diabetic samples (see Example 1). In addition, these biomarker panel were evaluated on the samples from patients with resectable pancreatic cancer and to samples from patients with low (<37 U/ml) CA19-9 values in order to analyze their performance on less progressive cancer states and for potential lewis a/b negative subjects that will give false-negative CA19-9 levels. The AUC subgroup performance of the panels from Table 9 is shown in Table 11, below:

TABLE 11
Subgroup performance of the diagnostic biomarker panels shown in Table
9 including diabetes, resectable pancreatic cancer and low CA19-9.

| Panel Number | Training of panel on | A | B | C | D | E | F |
|---|---|---|---|---|---|---|---|
| 1 | PDCA vs (CP + | all | 0.902 | 0.895 | 0.899 | 0.906 | 0.908 |
| | non-pancreatic ctrl) | | | | | | |
| 1 | PDCA vs (CP + | low CA19-9 | 0.756 | 0.678 | 0.714 | 0.700 | 0.659 |
| | non-pancreatic ctrl) | | | | | | |
| 1 | PDCA vs (CP + | resectable | 0.890 | 0.883 | 0.887 | 0.896 | 0.898 |
| | non-pancreatic ctrl) | | | | | | |
| 1 | PDCA vs CP | all | 0.913 | 0.896 | 0.904 | 0.913 | 0.915 |
| 1 | PDCA vs CP | low CA19-9 | 0.829 | 0.734 | 0.777 | 0.771 | 0.736 |
| 1 | PDCA vs CP | resectable | 0.909 | 0.892 | 0.900 | 0.911 | 0.914 |
| 1 | PDCA vs non- | all | 0.897 | 0.897 | 0.897 | 0.905 | 0.910 |
| | pancreatic ctrl | | | | | | |
| 1 | PDCA vs non- | low CA19-9 | 0.749 | 0.675 | 0.708 | 0.687 | 0.643 |
| | pancreatic ctrl | | | | | | |
| 1 | PDCA vs non- | resectable | 0.886 | 0.886 | 0.886 | 0.897 | 0.904 |
| | pancreatic ctrl | | | | | | |
| 2 | PDCA vs (CP + | all | 0.911 | 0.890 | 0.901 | 0.909 | 0.910 |
| | non-pancreatic ctrl) | | | | | | |
| 2 | PDCA vs (CP + | low CA19-9 | 0.792 | 0.694 | 0.738 | 0.727 | 0.690 |
| | non-pancreatic ctrl) | | | | | | |
| 2 | PDCA vs (CP + | resectable | 0.896 | 0.872 | 0.884 | 0.893 | 0.894 |
| | non-pancreatic ctrl) | | | | | | |
| 2 | PDCA vs CP | all | 0.929 | 0.892 | 0.911 | 0.919 | 0.921 |
| 2 | PDCA vs CP | low CA19-9 | 0.879 | 0.771 | 0.820 | 0.816 | 0.789 |
| 2 | PDCA vs CP | resectable | 0.920 | 0.878 | 0.899 | 0.908 | 0.910 |
| 2 | PDCA vs non- | all | 0.906 | 0.897 | 0.902 | 0.909 | 0.914 |
| | pancreatic ctrl | | | | | | |
| 2 | PDCA vs non- | low CA19-9 | 0.777 | 0.683 | 0.724 | 0.703 | 0.661 |
| | pancreatic ctrl | | | | | | |
| 2 | PDCA vs non- | resectable | 0.896 | 0.885 | 0.891 | 0.900 | 0.907 |
| | pancreatic ctrl | | | | | | |
| 3 | PDCA vs (CP + | all | 0.902 | 0.892 | 0.897 | 0.904 | 0.902 |
| | non-pancreatic ctrl) | | | | | | |
| 3 | PDCA vs (CP + | low CA19-9 | 0.757 | 0.676 | 0.713 | 0.695 | 0.642 |
| | non-pancreatic ctrl) | | | | | | |
| 3 | PDCA vs (CP + | resectable | 0.891 | 0.880 | 0.885 | 0.893 | 0.891 |
| | non-pancreatic ctrl) | | | | | | |
| 3 | PDCA vs CP | all | 0.916 | 0.895 | 0.905 | 0.913 | 0.913 |
| 3 | PDCA vs CP | low CA19-9 | 0.837 | 0.744 | 0.787 | 0.779 | 0.737 |
| 3 | PDCA vs CP | resectable | 0.911 | 0.888 | 0.899 | 0.908 | 0.909 |
| 3 | PDCA vs non- | all | 0.896 | 0.893 | 0.894 | 0.903 | 0.907 |
| | pancreatic ctrl | | | | | | |
| 3 | PDCA vs non- | low CA19-9 | 0.747 | 0.670 | 0.704 | 0.683 | 0.635 |
| | pancreatic ctrl | | | | | | |
| 3 | PDCA vs non- | resectable | 0.886 | 0.882 | 0.884 | 0.895 | 0.901 |
| | pancreatic ctrl | | | | | | |
| 4 | PDCA vs (CP + | all | 0.902 | 0.894 | 0.898 | 0.904 | 0.904 |
| | non-pancreatic ctrl) | | | | | | |
| 4 | PDCA vs (CP + | low CA19-9 | 0.756 | 0.676 | 0.713 | 0.694 | 0.646 |
| | non-pancreatic ctrl) | | | | | | |
| 4 | PDCA vs (CP + | resectable | 0.890 | 0.881 | 0.886 | 0.893 | 0.893 |
| | non-pancreatic ctrl) | | | | | | |
| 4 | PDCA vs CP | all | 0.912 | 0.893 | 0.902 | 0.900 | 0.890 |
| 4 | PDCA vs CP | low CA19-9 | 0.827 | 0.725 | 0.772 | 0.735 | 0.664 |
| 4 | PDCA vs CP | resectable | 0.909 | 0.888 | 0.898 | 0.896 | 0.886 |
| 4 | PDCA vs non- | all | 0.896 | 0.897 | 0.896 | 0.905 | 0.910 |
| | pancreatic ctrl | | | | | | |
| 4 | PDCA vs non- | low CA19-9 | 0.746 | 0.673 | 0.705 | 0.685 | 0.641 |
| | pancreatic ctrl | | | | | | |
| 4 | PDCA vs non- | resectable | 0.886 | 0.887 | 0.887 | 0.897 | 0.905 |
| | pancreatic ctrl | | | | | | |
| 5 | PDCA vs (CP + | all | 0.902 | 0.892 | 0.897 | 0.904 | 0.902 |
| | non-pancreatic ctrl) | | | | | | |
| 5 | PDCA vs (CP + | low CA19-9 | 0.757 | 0.676 | 0.713 | 0.695 | 0.642 |
| | non-pancreatic ctrl) | | | | | | |
| 5 | PDCA vs (CP + | resectable | 0.891 | 0.880 | 0.885 | 0.893 | 0.891 |
| | non-pancreatic ctrl) | | | | | | |
| 5 | PDCA vs CP | all | 0.916 | 0.895 | 0.905 | 0.913 | 0.913 |
| 5 | PDCA vs CP | low CA19-9 | 0.837 | 0.744 | 0.787 | 0.779 | 0.737 |
| 5 | PDCA vs CP | resectable | 0.911 | 0.888 | 0.899 | 0.908 | 0.909 |
| 5 | PDCA vs non- | all | 0.896 | 0.893 | 0.894 | 0.903 | 0.907 |
| | pancreatic ctrl | | | | | | |
| 5 | PDCA vs non- | low CA19-9 | 0.747 | 0.670 | 0.704 | 0.683 | 0.635 |
| | pancreatic ctrl | | | | | | |
| 5 | PDCA vs non- | resectable | 0.886 | 0.882 | 0.884 | 0.895 | 0.901 |
| | pancreatic ctrl | | | | | | |
| 6 | PDCA vs (CP + | all | 0.900 | 0.888 | 0.894 | 0.901 | 0.901 |
| | non-pancreatic ctrl) | | | | | | |
| 6 | PDCA vs (CP + | low CA19-9 | 0.757 | 0.669 | 0.709 | 0.693 | 0.643 |
| | non-pancreatic ctrl) | | | | | | |
| 6 | PDCA vs (CP + | resectable | 0.888 | 0.873 | 0.881 | 0.889 | 0.889 |
| | non-pancreatic ctrl) | | | | | | |
| 6 | PDCA vs CP | all | 0.913 | 0.890 | 0.901 | 0.910 | 0.912 |
| 6 | PDCA vs CP | low CA19-9 | 0.836 | 0.741 | 0.784 | 0.780 | 0.745 |
| 6 | PDCA vs CP | resectable | 0.906 | 0.881 | 0.893 | 0.903 | 0.906 |
| 6 | PDCA vs non- | all | 0.894 | 0.891 | 0.893 | 0.901 | 0.905 |
| | pancreatic ctrl | | | | | | |
| 6 | PDCA vs non- | low CA19-9 | 0.744 | 0.666 | 0.700 | 0.680 | 0.631 |
| | pancreatic ctrl | | | | | | |
| 6 | PDCA vs non- | resectable | 0.883 | 0.879 | 0.881 | 0.892 | 0.898 |
| | pancreatic ctrl | | | | | | |
| 7 | PDCA vs (CP + | all | 0.901 | 0.890 | 0.896 | 0.906 | 0.912 |
| | non-pancreatic ctrl) | | | | | | |
| 7 | PDCA vs (CP + | low CA19-9 | 0.758 | 0.676 | 0.713 | 0.709 | 0.681 |
| | non-pancreatic ctrl) | | | | | | |
| 7 | PDCA vs (CP + | resectable | 0.885 | 0.872 | 0.879 | 0.891 | 0.898 |
| | non-pancreatic ctrl) | | | | | | |
| 7 | PDCA vs CP | all | 0.916 | 0.890 | 0.903 | 0.916 | 0.925 |
| 7 | PDCA vs CP | low CA19-9 | 0.843 | 0.748 | 0.791 | 0.801 | 0.790 |
| 7 | PDCA vs CP | resectable | 0.907 | 0.878 | 0.893 | 0.907 | 0.917 |
| 7 | PDCA vs non- | all | 0.891 | 0.894 | 0.893 | 0.904 | 0.913 |
| | pancreatic ctrl | | | | | | |
| 7 | PDCA vs non- | low CA19-9 | 0.738 | 0.670 | 0.700 | 0.690 | 0.659 |
| | pancreatic ctrl | | | | | | |
| 7 | PDCA vs non- | resectable | 0.873 | 0.876 | 0.875 | 0.888 | 0.900 |
| | pancreatic ctrl | | | | | | |
| 8 | PDCA vs (CP + | all | 0.900 | 0.887 | 0.894 | 0.902 | 0.905 |
| | non-pancreatic ctrl) | | | | | | |
| 8 | PDCA vs (CP + | low CA19-9 | 0.756 | 0.670 | 0.710 | 0.699 | 0.656 |
| | non-pancreatic ctrl) | | | | | | |
| 8 | PDCA vs (CP + | resectable | 0.885 | 0.870 | 0.878 | 0.888 | 0.890 |
| | non-pancreatic ctrl) | | | | | | |
| 8 | PDCA vs CP | all | 0.914 | 0.888 | 0.901 | 0.912 | 0.918 |
| 8 | PDCA vs CP | low CA19-9 | 0.840 | 0.744 | 0.788 | 0.793 | 0.771 |
| 8 | PDCA vs CP | resectable | 0.906 | 0.877 | 0.891 | 0.904 | 0.911 |
| 8 | PDCA vs non- | all | 0.888 | 0.887 | 0.888 | 0.899 | 0.908 |
| | pancreatic ctrl | | | | | | |
| 8 | PDCA vs non- | low CA19-9 | 0.728 | 0.658 | 0.689 | 0.676 | 0.640 |
| | pancreatic ctrl | | | | | | |
| 8 | PDCA vs non- | resectable | 0.870 | 0.868 | 0.869 | 0.883 | 0.893 |
| | pancreatic ctrl | | | | | | |
| 9 | PDCA vs (CP + | all | 0.910 | 0.887 | 0.899 | 0.907 | 0.909 |
| | non-pancreatic ctrl) | | | | | | |
| 9 | PDCA vs (CP + | low CA19-9 | 0.782 | 0.685 | 0.729 | 0.719 | 0.681 |
| | non-pancreatic ctrl) | | | | | | |
| 9 | PDCA vs (CP + | resectable | 0.893 | 0.866 | 0.880 | 0.889 | 0.890 |
| | non-pancreatic ctrl) | | | | | | |
| 9 | PDCA vs CP | all | 0.928 | 0.889 | 0.908 | 0.919 | 0.922 |
| 9 | PDCA vs CP | low CA19-9 | 0.873 | 0.765 | 0.814 | 0.815 | 0.791 |
| 9 | PDCA vs CP | resectable | 0.918 | 0.875 | 0.896 | 0.908 | 0.911 |
| 9 | PDCA vs non- | all | 0.898 | 0.889 | 0.894 | 0.904 | 0.912 |
| | pancreatic ctrl | | | | | | |
| 9 | PDCA vs non- | low CA19-9 | 0.752 | 0.665 | 0.703 | 0.688 | 0.653 |
| | pancreatic ctrl | | | | | | |
| 9 | PDCA vs non- | resectable | 0.881 | 0.871 | 0.877 | 0.889 | 0.899 |
| | pancreatic ctrl | | | | | | |
| 10 | PDCA vs (CP + | all | 0.908 | 0.884 | 0.896 | 0.905 | 0.907 |
| | non-pancreatic ctrl) | | | | | | |
| 10 | PDCA vs (CP + | low CA19-9 | 0.781 | 0.682 | 0.727 | 0.718 | 0.681 |
| | non-pancreatic ctrl) | | | | | | |
| 10 | PDCA vs (CP + | resectable | 0.890 | 0.862 | 0.876 | 0.886 | 0.888 |
| | non-pancreatic ctrl) | | | | | | |
| 10 | PDCA vs CP | all | 0.925 | 0.886 | 0.905 | 0.917 | 0.922 |
| 10 | PDCA vs CP | low CA19-9 | 0.872 | 0.764 | 0.813 | 0.817 | 0.797 |
| 10 | PDCA vs CP | resectable | 0.914 | 0.870 | 0.892 | 0.904 | 0.909 |
| 10 | PDCA vs non- | all | 0.897 | 0.888 | 0.893 | 0.903 | 0.911 |
| | pancreatic ctrl | | | | | | |
| 10 | PDCA vs non- | low CA19-9 | 0.751 | 0.662 | 0.701 | 0.687 | 0.653 |
| | pancreatic ctrl | | | | | | |
| 10 | PDCA vs non- | resectable | 0.879 | 0.869 | 0.874 | 0.887 | 0.897 |
| | pancreatic ctrl | | | | | | |
| 11 | PDCA vs (CP + | all | 0.908 | 0.883 | 0.896 | 0.903 | 0.904 |
| | non-pancreatic ctrl) | | | | | | |
| 11 | PDCA vs (CP + | low CA19-9 | 0.782 | 0.681 | 0.727 | 0.714 | 0.673 |
| | non-pancreatic ctrl) | | | | | | |
| 11 | PDCA vs (CP + | resectable | 0.890 | 0.861 | 0.876 | 0.885 | 0.885 |
| | non-pancreatic ctrl) | | | | | | |
| 11 | PDCA vs CP | all | 0.930 | 0.881 | 0.906 | 0.899 | 0.883 |
| 11 | PDCA vs CP | low CA19-9 | 0.892 | 0.768 | 0.824 | 0.786 | 0.718 |
| 11 | PDCA vs CP | resectable | 0.921 | 0.866 | 0.894 | 0.886 | 0.868 |
| 11 | PDCA vs non- | all | 0.897 | 0.888 | 0.893 | 0.903 | 0.911 |
| | pancreatic ctrl | | | | | | |
| 11 | PDCA vs non- | low CA19-9 | 0.751 | 0.662 | 0.701 | 0.687 | 0.653 |
| | pancreatic ctrl | | | | | | |
| 11 | PDCA vs non- | resectable | 0.879 | 0.869 | 0.874 | 0.887 | 0.897 |
| | pancreatic ctrl | | | | | | |
| 12 | PDCA vs (CP + | all | 0.900 | 0.897 | 0.899 | 0.905 | 0.907 |
| | non-pancreatic ctrl) | | | | | | |
| 12 | PDCA vs (CP + | low CA19-9 | 0.761 | 0.693 | 0.724 | 0.707 | 0.664 |
| | non-pancreatic ctrl) | | | | | | |
| 12 | PDCA vs (CP + | resectable | 0.884 | 0.881 | 0.883 | 0.890 | 0.893 |
| | non-pancreatic ctrl) | | | | | | |
| 12 | PDCA vs CP | all | 0.908 | 0.891 | 0.899 | 0.906 | 0.907 |
| 12 | PDCA vs CP | low CA19-9 | 0.821 | 0.734 | 0.774 | 0.762 | 0.725 |
| 12 | PDCA vs CP | resectable | 0.902 | 0.883 | 0.892 | 0.901 | 0.902 |
| 12 | PDCA vs non- | all | 0.892 | 0.897 | 0.895 | 0.904 | 0.911 |
| | pancreatic ctrl | | | | | | |
| 12 | PDCA vs non- | low CA19-9 | 0.744 | 0.686 | 0.712 | 0.694 | 0.654 |
| | pancreatic ctrl | | | | | | |
| 12 | PDCA vs non- | resectable | 0.878 | 0.885 | 0.882 | 0.894 | 0.903 |
| | pancreatic ctrl | | | | | | |
| 13 | PDCA vs (CP + | all | 0.907 | 0.891 | 0.899 | 0.906 | 0.907 |
| | non-pancreatic ctrl) | | | | | | |
| 13 | PDCA vs (CP + | low CA19-9 | 0.787 | 0.701 | 0.740 | 0.724 | 0.687 |
| | non-pancreatic ctrl) | | | | | | |
| 13 | PDCA vs (CP + | resectable | 0.888 | 0.869 | 0.879 | 0.886 | 0.887 |
| | non-pancreatic ctrl) | | | | | | |
| 13 | PDCA vs CP | all | 0.920 | 0.886 | 0.903 | 0.910 | 0.911 |
| 13 | PDCA vs CP | low CA19-9 | 0.868 | 0.769 | 0.814 | 0.804 | 0.776 |
| 13 | PDCA vs CP | resectable | 0.908 | 0.870 | 0.889 | 0.896 | 0.897 |
| 13 | PDCA vs non- | all | 0.898 | 0.895 | 0.897 | 0.906 | 0.913 |
| | pancreatic ctrl | | | | | | |
| 13 | PDCA vs non- | low CA19-9 | 0.766 | 0.689 | 0.722 | 0.704 | 0.665 |
| | pancreatic ctrl | | | | | | |
| 13 | PDCA vs non- | resectable | 0.886 | 0.882 | 0.884 | 0.895 | 0.905 |
| | pancreatic ctrl | | | | | | |
| 14 | PDCA vs (CP + | all | 0.900 | 0.895 | 0.898 | 0.902 | 0.900 |
| | non-pancreatic ctrl) | | | | | | |
| 14 | PDCA vs (CP + | low CA19-9 | 0.758 | 0.687 | 0.720 | 0.695 | 0.642 |
| | non-pancreatic ctrl) | | | | | | |
| 14 | PDCA vs (CP + | resectable | 0.883 | 0.878 | 0.881 | 0.886 | 0.884 |
| | non-pancreatic ctrl) | | | | | | |
| 14 | PDCA vs CP | all | 0.908 | 0.887 | 0.897 | 0.891 | 0.876 |
| 14 | PDCA vs CP | low CA19-9 | 0.820 | 0.723 | 0.768 | 0.719 | 0.638 |
| 14 | PDCA vs CP | resectable | 0.903 | 0.881 | 0.892 | 0.885 | 0.869 |
| 14 | PDCA vs non- | all | 0.891 | 0.897 | 0.894 | 0.904 | 0.911 |
| | pancreatic ctrl | | | | | | |
| 14 | PDCA vs non- | low CA19-9 | 0.741 | 0.684 | 0.709 | 0.691 | 0.651 |
| | pancreatic ctrl | | | | | | |
| 14 | PDCA vs non- | resectable | 0.877 | 0.885 | 0.881 | 0.893 | 0.903 |
| | pancreatic ctrl | | | | | | |
| 15 | PDCA vs (CP + | all | 0.901 | 0.894 | 0.897 | 0.903 | 0.901 |
| | non-pancreatic ctrl) | | | | | | |
| 15 | PDCA vs (CP + | low CA19-9 | 0.756 | 0.683 | 0.717 | 0.695 | 0.641 |
| | non-pancreatic ctrl) | | | | | | |
| 15 | PDCA vs (CP + | resectable | 0.886 | 0.877 | 0.882 | 0.888 | 0.886 |
| | non-pancreatic ctrl) | | | | | | |
| 15 | PDCA vs CP | all | 0.911 | 0.890 | 0.900 | 0.906 | 0.904 |
| 15 | PDCA vs CP | low CA19-9 | 0.822 | 0.736 | 0.776 | 0.761 | 0.714 |
| 15 | PDCA vs CP | resectable | 0.903 | 0.879 | 0.891 | 0.898 | 0.896 |
| 15 | PDCA vs non- | all | 0.890 | 0.893 | 0.891 | 0.901 | 0.908 |
| | pancreatic ctrl | | | | | | |
| 15 | PDCA vs non- | low CA19-9 | 0.737 | 0.677 | 0.704 | 0.686 | 0.642 |
| | pancreatic ctrl | | | | | | |
| 15 | PDCA vs non- | resectable | 0.878 | 0.881 | 0.880 | 0.892 | 0.901 |
| | pancreatic ctrl | | | | | | |
| 16 | PDCA vs (CP + | all | 0.891 | 0.890 | 0.891 | 0.897 | 0.901 |
| | non-pancreatic ctrl) | | | | | | |
| 16 | PDCA vs (CP + | low CA19-9 | 0.731 | 0.658 | 0.691 | 0.675 | 0.637 |
| | non-pancreatic ctrl) | | | | | | |
| 16 | PDCA vs (CP + | resectable | 0.877 | 0.875 | 0.876 | 0.884 | 0.889 |
| | non-pancreatic ctrl) | | | | | | |
| 16 | PDCA vs CP | all | 0.903 | 0.892 | 0.897 | 0.905 | 0.909 |
| 16 | PDCA vs CP | low CA19-9 | 0.810 | 0.714 | 0.758 | 0.749 | 0.718 |
| 16 | PDCA vs CP | resectable | 0.893 | 0.881 | 0.887 | 0.896 | 0.901 |
| 16 | PDCA vs non- | all | 0.886 | 0.895 | 0.891 | 0.899 | 0.906 |
| | pancreatic ctrl | | | | | | |
| 16 | PDCA vs non- | low CA19-9 | 0.720 | 0.659 | 0.686 | 0.664 | 0.624 |
| | pancreatic ctrl | | | | | | |
| 16 | PDCA vs non- | resectable | 0.870 | 0.881 | 0.876 | 0.887 | 0.897 |
| | pancreatic ctrl | | | | | | |
| 17 | PDCA vs (CP + | all | 0.892 | 0.892 | 0.892 | 0.901 | 0.910 |
| | non-pancreatic ctrl) | | | | | | |
| 17 | PDCA vs (CP + | low CA19-9 | 0.719 | 0.643 | 0.677 | 0.667 | 0.640 |
| | non-pancreatic ctrl) | | | | | | |
| 17 | PDCA vs (CP + | resectable | 0.878 | 0.878 | 0.878 | 0.890 | 0.900 |
| | non-pancreatic ctrl) | | | | | | |
| 17 | PDCA vs CP | all | 0.912 | 0.893 | 0.902 | 0.915 | 0.927 |
| 17 | PDCA vs CP | low CA19-9 | 0.809 | 0.692 | 0.746 | 0.750 | 0.741 |
| 17 | PDCA vs CP | resectable | 0.901 | 0.878 | 0.889 | 0.904 | 0.919 |
| 17 | PDCA vs non- | all | 0.865 | 0.889 | 0.877 | 0.886 | 0.897 |
| | pancreatic ctrl | | | | | | |
| 17 | PDCA vs non- | low CA19-9 | 0.659 | 0.633 | 0.644 | 0.622 | 0.585 |
| | pancreatic ctrl | | | | | | |
| 17 | PDCA vs non- | resectable | 0.844 | 0.873 | 0.859 | 0.871 | 0.885 |
| | pancreatic ctrl | | | | | | |
| 18 | PDCA vs (CP + | all | 0.894 | 0.891 | 0.892 | 0.901 | 0.909 |
| | non-pancreatic ctrl) | | | | | | |
| 18 | PDCA vs (CP + | low CA19-9 | 0.738 | 0.660 | 0.696 | 0.688 | 0.660 |
| | non-pancreatic ctrl) | | | | | | |
| 18 | PDCA vs (CP + | resectable | 0.878 | 0.874 | 0.876 | 0.887 | 0.896 |
| | non-pancreatic ctrl) | | | | | | |
| 18 | PDCA vs CP | all | 0.908 | 0.893 | 0.901 | 0.912 | 0.920 |
| 18 | PDCA vs CP | low CA19-9 | 0.819 | 0.721 | 0.766 | 0.768 | 0.751 |
| 18 | PDCA vs CP | resectable | 0.897 | 0.880 | 0.889 | 0.902 | 0.912 |
| 18 | PDCA vs non- | all | 0.884 | 0.893 | 0.889 | 0.898 | 0.907 |
| | pancreatic ctrl | | | | | | |
| 18 | PDCA vs non- | low CA19-9 | 0.714 | 0.655 | 0.681 | 0.663 | 0.627 |
| | pancreatic ctrl | | | | | | |
| 18 | PDCA vs non- | resectable | 0.868 | 0.879 | 0.874 | 0.886 | 0.897 |
| | pancreatic ctrl | | | | | | |
| 19 | PDCA vs (CP + | all | 0.890 | 0.894 | 0.892 | 0.898 | 0.902 |
| | non-pancreatic ctrl) | | | | | | |
| 19 | PDCA vs (CP + | low CA19-9 | 0.736 | 0.676 | 0.704 | 0.683 | 0.645 |
| | non-pancreatic ctrl) | | | | | | |
| 19 | PDCA vs (CP + | resectable | 0.872 | 0.876 | 0.874 | 0.880 | 0.885 |
| | non-pancreatic ctrl) | | | | | | |
| 19 | PDCA vs CP | all | 0.896 | 0.887 | 0.891 | 0.897 | 0.898 |
| 19 | PDCA vs CP | low CA19-9 | 0.797 | 0.714 | 0.752 | 0.737 | 0.704 |
| 19 | PDCA vs CP | resectable | 0.883 | 0.873 | 0.878 | 0.885 | 0.887 |
| 19 | PDCA vs non- | all | 0.882 | 0.896 | 0.889 | 0.899 | 0.909 |
| | pancreatic ctrl | | | | | | |
| 19 | PDCA vs non- | low CA19-9 | 0.716 | 0.673 | 0.692 | 0.674 | 0.640 |
| | pancreatic ctrl | | | | | | |
| 19 | PDCA vs non- | resectable | 0.863 | 0.881 | 0.872 | 0.885 | 0.898 |
| | pancreatic ctrl | | | | | | |
| 20 | PDCA vs (CP + | all | 0.895 | 0.897 | 0.896 | 0.906 | 0.915 |
| | non-pancreatic ctrl) | | | | | | |
| 20 | PDCA vs (CP + | low CA19-9 | 0.739 | 0.670 | 0.702 | 0.691 | 0.666 |
| | non-pancreatic ctrl) | | | | | | |
| 20 | PDCA vs (CP + | resectable | 0.876 | 0.879 | 0.877 | 0.889 | 0.900 |
| | non-pancreatic ctrl) | | | | | | |
| 20 | PDCA vs CP | all | 0.907 | 0.890 | 0.898 | 0.910 | 0.920 |
| 20 | PDCA vs CP | low CA19-9 | 0.803 | 0.701 | 0.748 | 0.748 | 0.737 |
| 20 | PDCA vs CP | resectable | 0.891 | 0.870 | 0.880 | 0.894 | 0.907 |
| 20 | PDCA vs non- | all | 0.867 | 0.892 | 0.880 | 0.892 | 0.905 |
| | pancreatic ctrl | | | | | | |
| 20 | PDCA vs non- | low CA19-9 | 0.674 | 0.656 | 0.664 | 0.649 | 0.618 |
| | pancreatic ctrl | | | | | | |
| 20 | PDCA vs non- | resectable | 0.845 | 0.876 | 0.861 | 0.876 | 0.893 |
| | pancreatic ctrl | | | | | | |
| 21 | PDCA vs (CP + | all | 0.892 | 0.893 | 0.893 | 0.901 | 0.910 |
| | non-pancreatic ctrl) | | | | | | |
| 21 | PDCA vs (CP + | low CA19-9 | 0.744 | 0.677 | 0.708 | 0.697 | 0.670 |
| | non-pancreatic ctrl) | | | | | | |
| 21 | PDCA vs (CP + | resectable | 0.871 | 0.873 | 0.872 | 0.882 | 0.892 |
| | non-pancreatic ctrl) | | | | | | |
| 21 | PDCA vs CP | all | 0.901 | 0.889 | 0.895 | 0.904 | 0.912 |
| 21 | PDCA vs CP | low CA19-9 | 0.806 | 0.722 | 0.760 | 0.757 | 0.738 |
| 21 | PDCA vs CP | resectable | 0.886 | 0.872 | 0.879 | 0.890 | 0.899 |
| 21 | PDCA vs non- | all | 0.882 | 0.895 | 0.889 | 0.900 | 0.911 |
| | pancreatic ctrl | | | | | | |
| 21 | PDCA vs non- | low CA19-9 | 0.719 | 0.673 | 0.693 | 0.679 | 0.649 |
| | pancreatic ctrl | | | | | | |
| 21 | PDCA vs non- | resectable | 0.864 | 0.879 | 0.872 | 0.886 | 0.900 |
| | pancreatic ctrl | | | | | | |
| 22 | PDCA vs (CP + | all | 0.892 | 0.881 | 0.887 | 0.892 | 0.884 |
| | non-pancreatic ctrl) | | | | | | |
| 22 | PDCA vs (CP + | low CA19-9 | 0.719 | 0.642 | 0.677 | 0.659 | 0.604 |
| | non-pancreatic ctrl) | | | | | | |
| 22 | PDCA vs (CP + | resectable | 0.886 | 0.873 | 0.880 | 0.887 | 0.878 |
| | non-pancreatic ctrl) | | | | | | |
| 22 | PDCA vs CP | all | 0.905 | 0.882 | 0.894 | 0.900 | 0.894 |
| 22 | PDCA vs CP | low CA19-9 | 0.809 | 0.704 | 0.752 | 0.742 | 0.687 |
| 22 | PDCA vs CP | resectable | 0.906 | 0.882 | 0.893 | 0.902 | 0.896 |
| 22 | PDCA vs non- | all | 0.881 | 0.886 | 0.884 | 0.888 | 0.880 |
| | pancreatic ctrl | | | | | | |
| 22 | PDCA vs non- | low CA19-9 | 0.694 | 0.636 | 0.662 | 0.632 | 0.561 |
| | pancreatic ctrl | | | | | | |
| 22 | PDCA vs non- | resectable | 0.873 | 0.879 | 0.877 | 0.883 | 0.875 |
| | pancreatic ctrl | | | | | | |
| 23 | PDCA vs (CP + | all | 0.880 | 0.877 | 0.879 | 0.883 | 0.876 |
| | non-pancreatic ctrl) | | | | | | |
| 23 | PDCA vs (CP + | low CA19-9 | 0.688 | 0.623 | 0.652 | 0.631 | 0.577 |
| | non-pancreatic ctrl) | | | | | | |
| 23 | PDCA vs (CP + | resectable | 0.871 | 0.866 | 0.869 | 0.874 | 0.866 |
| | non-pancreatic ctrl) | | | | | | |
| 23 | PDCA vs CP | all | 0.895 | 0.877 | 0.886 | 0.891 | 0.884 |
| 23 | PDCA vs CP | low CA19-9 | 0.789 | 0.685 | 0.733 | 0.718 | 0.664 |
| 23 | PDCA vs CP | resectable | 0.887 | 0.868 | 0.877 | 0.884 | 0.877 |
| 23 | PDCA vs non- | all | 0.867 | 0.882 | 0.875 | 0.879 | 0.873 |
| | pancreatic ctrl | | | | | | |
| 23 | PDCA vs non- | low CA19-9 | 0.657 | 0.617 | 0.635 | 0.604 | 0.536 |
| | pancreatic ctrl | | | | | | |
| 23 | PDCA vs non- | resectable | 0.852 | 0.870 | 0.862 | 0.868 | 0.861 |
| | pancreatic ctrl | | | | | | |
| 24 | PDCA vs (CP + | all | 0.889 | 0.883 | 0.886 | 0.893 | 0.892 |
| | non-pancreatic ctrl) | | | | | | |
| 24 | PDCA vs (CP + | low CA19-9 | 0.704 | 0.626 | 0.661 | 0.648 | 0.605 |
| | non-pancreatic ctrl) | | | | | | |
| 24 | PDCA vs (CP + | resectable | 0.879 | 0.872 | 0.876 | 0.885 | 0.883 |
| | non-pancreatic ctrl) | | | | | | |
| 24 | PDCA vs CP | all | 0.907 | 0.882 | 0.895 | 0.905 | 0.907 |
| 24 | PDCA vs CP | low CA19-9 | 0.803 | 0.679 | 0.736 | 0.736 | 0.704 |
| 24 | PDCA vs CP | resectable | 0.899 | 0.870 | 0.884 | 0.896 | 0.900 |
| 24 | PDCA vs non- | all | 0.862 | 0.881 | 0.872 | 0.877 | 0.873 |
| | pancreatic ctrl | | | | | | |
| 24 | PDCA vs non- | low CA19-9 | 0.644 | 0.614 | 0.627 | 0.599 | 0.535 |
| | pancreatic ctrl | | | | | | |
| 24 | PDCA vs non- | resectable | 0.846 | 0.869 | 0.858 | 0.865 | 0.861 |
| | pancreatic ctrl | | | | | | |
| 25 | PDCA vs (CP + | all | 0.885 | 0.878 | 0.881 | 0.888 | 0.887 |
| | non-pancreatic ctrl) | | | | | | |
| 25 | PDCA vs (CP + | low CA19-9 | 0.705 | 0.629 | 0.663 | 0.651 | 0.609 |
| | non-pancreatic ctrl) | | | | | | |
| 25 | PDCA vs (CP + | resectable | 0.874 | 0.866 | 0.870 | 0.879 | 0.878 |
| | non-pancreatic ctrl) | | | | | | |
| 25 | PDCA vs CP | all | 0.902 | 0.882 | 0.892 | 0.901 | 0.902 |
| 25 | PDCA vs CP | low CA19-9 | 0.806 | 0.701 | 0.749 | 0.749 | 0.714 |
| 25 | PDCA vs CP | resectable | 0.895 | 0.873 | 0.884 | 0.895 | 0.897 |
| 25 | PDCA vs non- | all | 0.872 | 0.883 | 0.878 | 0.883 | 0.878 |
| | pancreatic ctrl | | | | | | |
| 25 | PDCA vs non- | low CA19-9 | 0.676 | 0.625 | 0.648 | 0.619 | 0.554 |
| | pancreatic ctrl | | | | | | |
| 25 | PDCA vs non- | resectable | 0.859 | 0.872 | 0.866 | 0.873 | 0.868 |
| | pancreatic ctrl | | | | | | |
| 26 | PDCA vs (CP + | all | 0.889 | 0.882 | 0.886 | 0.890 | 0.883 |
| | non-pancreatic ctrl) | | | | | | |
| 26 | PDCA vs (CP + | low CA19-9 | 0.715 | 0.647 | 0.678 | 0.656 | 0.600 |
| | non-pancreatic ctrl) | | | | | | |
| 26 | PDCA vs (CP + | resectable | 0.878 | 0.870 | 0.874 | 0.879 | 0.871 |
| | non-pancreatic ctrl) | | | | | | |
| 26 | PDCA vs CP | all | 0.898 | 0.875 | 0.887 | 0.891 | 0.884 |
| 26 | PDCA vs CP | low CA19-9 | 0.796 | 0.702 | 0.745 | 0.730 | 0.675 |
| 26 | PDCA vs CP | resectable | 0.894 | 0.869 | 0.882 | 0.888 | 0.880 |
| 26 | PDCA vs non- | all | 0.874 | 0.885 | 0.879 | 0.885 | 0.878 |
| | pancreatic ctrl | | | | | | |
| 26 | PDCA vs non- | low CA19-9 | 0.682 | 0.637 | 0.657 | 0.627 | 0.557 |
| | pancreatic ctrl | | | | | | |
| 26 | PDCA vs non- | resectable | 0.861 | 0.874 | 0.868 | 0.875 | 0.869 |
| | pancreatic ctrl | | | | | | |
| 27 | PDCA vs (CP + | all | 0.878 | 0.878 | 0.878 | 0.881 | 0.874 |
| | non-pancreatic ctrl) | | | | | | |
| 27 | PDCA vs (CP + | low CA19-9 | 0.683 | 0.629 | 0.653 | 0.627 | 0.572 |
| | non-pancreatic ctrl) | | | | | | |
| 27 | PDCA vs (CP + | resectable | 0.863 | 0.863 | 0.863 | 0.867 | 0.859 |
| | non-pancreatic ctrl) | | | | | | |
| 27 | PDCA vs CP | all | 0.885 | 0.870 | 0.878 | 0.881 | 0.874 |
| 27 | PDCA vs CP | low CA19-9 | 0.773 | 0.683 | 0.724 | 0.704 | 0.649 |
| 27 | PDCA vs CP | resectable | 0.874 | 0.857 | 0.865 | 0.869 | 0.861 |
| 27 | PDCA vs non- | all | 0.859 | 0.881 | 0.870 | 0.876 | 0.872 |
| | pancreatic ctrl | | | | | | |
| 27 | PDCA vs non- | low CA19-9 | 0.642 | 0.619 | 0.629 | 0.599 | 0.534 |
| | pancreatic ctrl | | | | | | |
| 27 | PDCA vs non- | resectable | 0.840 | 0.867 | 0.854 | 0.861 | 0.859 |
| | pancreatic ctrl | | | | | | |
| 28 | PDCA vs (CP + | all | 0.889 | 0.886 | 0.888 | 0.895 | 0.894 |
| | non-pancreatic ctrl) | | | | | | |
| 28 | PDCA vs (CP + | low CA19-9 | 0.711 | 0.641 | 0.673 | 0.657 | 0.614 |
| | non-pancreatic ctrl) | | | | | | |
| 28 | PDCA vs (CP + | resectable | 0.873 | 0.870 | 0.872 | 0.880 | 0.880 |
| | non-pancreatic ctrl) | | | | | | |
| 28 | PDCA vs CP | all | 0.901 | 0.879 | 0.890 | 0.899 | 0.901 |
| 28 | PDCA vs CP | low CA19-9 | 0.796 | 0.687 | 0.737 | 0.735 | 0.702 |
| 28 | PDCA vs CP | resectable | 0.886 | 0.860 | 0.873 | 0.884 | 0.887 |
| 28 | PDCA vs non- | all | 0.861 | 0.883 | 0.872 | 0.879 | 0.878 |
| | pancreatic ctrl | | | | | | |
| 28 | PDCA vs non- | low CA19-9 | 0.646 | 0.627 | 0.635 | 0.610 | 0.548 |
| | pancreatic ctrl | | | | | | |
| 28 | PDCA vs non- | resectable | 0.841 | 0.868 | 0.855 | 0.865 | 0.863 |
| | pancreatic ctrl | | | | | | |
| 29 | PDCA vs (CP + | all | 0.882 | 0.879 | 0.881 | 0.887 | 0.887 |
| | non-pancreatic ctrl) | | | | | | |
| 29 | PDCA vs (CP + | low CA19-9 | 0.704 | 0.636 | 0.667 | 0.652 | 0.609 |
| | non-pancreatic ctrl) | | | | | | |
| 29 | PDCA vs (CP + | resectable | 0.866 | 0.862 | 0.864 | 0.872 | 0.871 |
| | non-pancreatic ctrl) | | | | | | |
| 29 | PDCA vs CP | all | 0.891 | 0.875 | 0.883 | 0.891 | 0.892 |
| 29 | PDCA vs CP | low CA19-9 | 0.787 | 0.696 | 0.738 | 0.733 | 0.697 |
| 29 | PDCA vs CP | resectable | 0.880 | 0.861 | 0.871 | 0.880 | 0.882 |
| 29 | PDCA vs non- | all | 0.865 | 0.881 | 0.874 | 0.880 | 0.877 |
| | pancreatic ctrl | | | | | | |
| 29 | PDCA vs non- | low CA19-9 | 0.664 | 0.629 | 0.644 | 0.618 | 0.554 |
| | pancreatic ctrl | | | | | | |
| 29 | PDCA vs non- | resectable | 0.849 | 0.868 | 0.859 | 0.867 | 0.865 |
| | pancreatic ctrl | | | | | | |
| 30 | PDCA vs (CP + | all | 0.901 | 0.886 | 0.894 | 0.904 | 0.907 |
| | non-pancreatic ctrl) | | | | | | |
| 30 | PDCA vs (CP + | low CA19-9 | 0.744 | 0.664 | 0.700 | 0.697 | 0.664 |
| | non-pancreatic ctrl) | | | | | | |
| 30 | PDCA vs (CP + | resectable | 0.891 | 0.874 | 0.883 | 0.895 | 0.899 |
| | non-pancreatic ctrl) | | | | | | |
| 30 | PDCA vs CP | all | 0.918 | 0.886 | 0.902 | 0.915 | 0.919 |
| 30 | PDCA vs CP | low CA19-9 | 0.838 | 0.733 | 0.781 | 0.790 | 0.767 |
| 30 | PDCA vs CP | resectable | 0.914 | 0.880 | 0.897 | 0.912 | 0.916 |
| 30 | PDCA vs non- | all | 0.884 | 0.889 | 0.886 | 0.898 | 0.905 |
| | pancreatic ctrl | | | | | | |
| 30 | PDCA vs non- | low CA19-9 | 0.700 | 0.646 | 0.670 | 0.662 | 0.624 |
| | pancreatic ctrl | | | | | | |
| 30 | PDCA vs non- | resectable | 0.872 | 0.878 | 0.875 | 0.890 | 0.898 |
| | pancreatic ctrl | | | | | | |
| 31 | PDCA vs (CP + | all | 0.897 | 0.886 | 0.892 | 0.905 | 0.917 |
| | non-pancreatic ctrl) | | | | | | |
| 31 | PDCA vs (CP + | low CA19-9 | 0.729 | 0.644 | 0.681 | 0.687 | 0.675 |
| | non-pancreatic ctrl) | | | | | | |
| 31 | PDCA vs (CP + | resectable | 0.883 | 0.870 | 0.877 | 0.893 | 0.907 |
| | non-pancreatic ctrl) | | | | | | |
| 31 | PDCA vs CP | all | 0.919 | 0.885 | 0.902 | 0.920 | 0.934 |
| 31 | PDCA vs CP | low CA19-9 | 0.832 | 0.703 | 0.762 | 0.784 | 0.789 |
| 31 | PDCA vs CP | resectable | 0.908 | 0.870 | 0.889 | 0.908 | 0.925 |
| 31 | PDCA vs non- | all | 0.862 | 0.882 | 0.872 | 0.887 | 0.901 |
| | pancreatic ctrl | | | | | | |
| 31 | PDCA vs non- | low CA19-9 | 0.645 | 0.620 | 0.631 | 0.629 | 0.606 |
| | pancreatic ctrl | | | | | | |
| 31 | PDCA vs non- | resectable | 0.841 | 0.866 | 0.854 | 0.872 | 0.889 |
| | pancreatic ctrl | | | | | | |
| 32 | PDCA vs (CP + | all | 0.891 | 0.881 | 0.887 | 0.898 | 0.908 |
| | non-pancreatic ctrl) | | | | | | |
| 32 | PDCA vs (CP + | low CA19-9 | 0.728 | 0.649 | 0.684 | 0.686 | 0.667 |
| | non-pancreatic ctrl) | | | | | | |
| 32 | PDCA vs (CP + | resectable | 0.877 | 0.865 | 0.871 | 0.885 | 0.897 |
| | non-pancreatic ctrl) | | | | | | |
| 32 | PDCA vs CP | all | 0.908 | 0.883 | 0.895 | 0.909 | 0.919 |
| 32 | PDCA vs CP | low CA19-9 | 0.821 | 0.716 | 0.764 | 0.778 | 0.768 |
| 32 | PDCA vs CP | resectable | 0.898 | 0.869 | 0.884 | 0.899 | 0.910 |
| 32 | PDCA vs non- | all | 0.873 | 0.885 | 0.879 | 0.892 | 0.903 |
| | pancreatic ctrl | | | | | | |
| 32 | PDCA vs non- | low CA19-9 | 0.678 | 0.634 | 0.653 | 0.649 | 0.622 |
| | pancreatic ctrl | | | | | | |
| 32 | PDCA vs non- | resectable | 0.855 | 0.869 | 0.862 | 0.879 | 0.892 |
| | pancreatic ctrl | | | | | | |
| 33 | PDCA vs (CP + | all | 0.889 | 0.881 | 0.885 | 0.895 | 0.900 |
| | non-pancreatic ctrl) | | | | | | |
| 33 | PDCA vs (CP + | low CA19-9 | 0.713 | 0.644 | 0.674 | 0.669 | 0.641 |
| | non-pancreatic ctrl) | | | | | | |
| 33 | PDCA vs (CP + | resectable | 0.876 | 0.868 | 0.872 | 0.884 | 0.890 |
| | non-pancreatic ctrl) | | | | | | |
| 33 | PDCA vs CP | all | 0.903 | 0.879 | 0.891 | 0.903 | 0.907 |
| 33 | PDCA vs CP | low CA19-9 | 0.807 | 0.701 | 0.749 | 0.754 | 0.731 |
| 33 | PDCA vs CP | resectable | 0.894 | 0.867 | 0.881 | 0.894 | 0.899 |
| 33 | PDCA vs non- | all | 0.869 | 0.884 | 0.877 | 0.890 | 0.900 |
| | pancreatic ctrl | | | | | | |
| 33 | PDCA vs non- | low CA19-9 | 0.663 | 0.627 | 0.643 | 0.636 | 0.606 |
| | pancreatic ctrl | | | | | | |
| 33 | PDCA vs non- | resectable | 0.851 | 0.869 | 0.860 | 0.876 | 0.889 |
| | pancreatic ctrl | | | | | | |
| 34 | PDCA vs (CP + | all | 0.897 | 0.885 | 0.891 | 0.900 | 0.903 |
| | non-pancreatic ctrl) | | | | | | |
| 34 | PDCA vs (CP + | low CA19-9 | 0.738 | 0.666 | 0.698 | 0.690 | 0.655 |
| | non-pancreatic ctrl) | | | | | | |
| 34 | PDCA vs (CP + | resectable | 0.884 | 0.870 | 0.877 | 0.887 | 0.891 |
| | non-pancreatic ctrl) | | | | | | |
| 34 | PDCA vs CP | all | 0.911 | 0.879 | 0.895 | 0.907 | 0.910 |
| 34 | PDCA vs CP | low CA19-9 | 0.826 | 0.726 | 0.771 | 0.777 | 0.752 |
| 34 | PDCA vs CP | resectable | 0.905 | 0.869 | 0.887 | 0.901 | 0.904 |
| 34 | PDCA vs non- | all | 0.875 | 0.884 | 0.880 | 0.892 | 0.899 |
| | pancreatic ctrl | | | | | | |
| 34 | PDCA vs non- | low CA19-9 | 0.683 | 0.640 | 0.659 | 0.650 | 0.613 |
| | pancreatic ctrl | | | | | | |
| 34 | PDCA vs non- | resectable | 0.861 | 0.872 | 0.867 | 0.882 | 0.891 |
| | pancreatic ctrl | | | | | | |
| 35 | PDCA vs (CP + | all | 0.884 | 0.881 | 0.882 | 0.891 | 0.896 |
| | non-pancreatic ctrl) | | | | | | |
| 35 | PDCA vs (CP + | low CA19-9 | 0.702 | 0.644 | 0.670 | 0.660 | 0.630 |
| | non-pancreatic ctrl) | | | | | | |
| 35 | PDCA vs (CP + | resectable | 0.867 | 0.864 | 0.866 | 0.876 | 0.882 |
| | non-pancreatic ctrl) | | | | | | |
| 35 | PDCA vs CP | all | 0.896 | 0.875 | 0.885 | 0.895 | 0.899 |
| 35 | PDCA vs CP | low CA19-9 | 0.794 | 0.699 | 0.742 | 0.742 | 0.718 |
| 35 | PDCA vs CP | resectable | 0.883 | 0.859 | 0.871 | 0.883 | 0.887 |
| 35 | PDCA vs non- | all | 0.861 | 0.881 | 0.871 | 0.884 | 0.895 |
| | pancreatic ctrl | | | | | | |
| 35 | PDCA vs non- | low CA19-9 | 0.646 | 0.624 | 0.633 | 0.626 | 0.595 |
| | pancreatic ctrl | | | | | | |
| 35 | PDCA vs non- | resectable | 0.840 | 0.865 | 0.853 | 0.869 | 0.883 |
| | pancreatic ctrl | | | | | | |
| 36 | PDCA vs (CP + | all | 0.897 | 0.888 | 0.892 | 0.905 | 0.917 |
| | non-pancreatic ctrl) | | | | | | |
| 36 | PDCA vs (CP + | low CA19-9 | 0.736 | 0.655 | 0.691 | 0.694 | 0.681 |
| | non-pancreatic ctrl) | | | | | | |
| 36 | PDCA vs (CP + | resectable | 0.878 | 0.868 | 0.873 | 0.888 | 0.903 |
| | non-pancreatic ctrl) | | | | | | |
| 36 | PDCA vs CP | all | 0.912 | 0.881 | 0.897 | 0.913 | 0.926 |
| 36 | PDCA vs CP | low CA19-9 | 0.821 | 0.702 | 0.755 | 0.775 | 0.777 |
| 36 | PDCA vs CP | resectable | 0.896 | 0.860 | 0.878 | 0.897 | 0.912 |
| 36 | PDCA vs non- | all | 0.859 | 0.882 | 0.871 | 0.887 | 0.901 |
| | pancreatic ctrl | | | | | | |
| 36 | PDCA vs non- | low CA19-9 | 0.644 | 0.627 | 0.634 | 0.633 | 0.608 |
| | pancreatic ctrl | | | | | | |
| 36 | PDCA vs non- | resectable | 0.837 | 0.864 | 0.850 | 0.870 | 0.888 |
| | pancreatic ctrl | | | | | | |
| 37 | PDCA vs (CP + | all | 0.888 | 0.881 | 0.885 | 0.895 | 0.905 |
| | non-pancreatic ctrl) | | | | | | |
| 37 | PDCA vs (CP + | low CA19-9 | 0.720 | 0.652 | 0.682 | 0.680 | 0.660 |
| | non-pancreatic ctrl) | | | | | | |
| 37 | PDCA vs (CP + | resectable | 0.869 | 0.862 | 0.866 | 0.879 | 0.889 |
| | non-pancreatic ctrl) | | | | | | |
| 37 | PDCA vs CP | all | 0.899 | 0.877 | 0.888 | 0.901 | 0.911 |
| 37 | PDCA vs CP | low CA19-9 | 0.806 | 0.711 | 0.754 | 0.764 | 0.754 |
| 37 | PDCA vs CP | resectable | 0.885 | 0.860 | 0.873 | 0.887 | 0.898 |
| 37 | PDCA vs non- | all | 0.867 | 0.881 | 0.874 | 0.888 | 0.899 |
| | pancreatic ctrl | | | | | | |
| 37 | PDCA vs non- | low CA19-9 | 0.668 | 0.632 | 0.647 | 0.642 | 0.614 |
| | pancreatic ctrl | | | | | | |
| 37 | PDCA vs non- | resectable | 0.847 | 0.865 | 0.856 | 0.873 | 0.887 |
| | pancreatic ctrl | | | | | | |
| 38 | PDCA vs (CP + | all | 0.892 | 0.888 | 0.890 | 0.896 | 0.895 |
| | non-pancreatic ctrl) | | | | | | |
| 38 | PDCA vs (CP + | low CA19-9 | 0.736 | 0.660 | 0.694 | 0.673 | 0.622 |
| | non-pancreatic ctrl) | | | | | | |
| 38 | PDCA vs (CP + | resectable | 0.879 | 0.874 | 0.877 | 0.883 | 0.883 |
| | non-pancreatic ctrl) | | | | | | |
| 38 | PDCA vs CP | all | 0.906 | 0.888 | 0.897 | 0.903 | 0.904 |
| 38 | PDCA vs CP | low CA19-9 | 0.821 | 0.723 | 0.768 | 0.755 | 0.714 |
| 38 | PDCA vs CP | resectable | 0.898 | 0.878 | 0.888 | 0.896 | 0.897 |
| 38 | PDCA vs non- | all | 0.886 | 0.891 | 0.889 | 0.897 | 0.904 |
| | pancreatic ctrl | | | | | | |
| 38 | PDCA vs non- | low CA19-9 | 0.721 | 0.657 | 0.685 | 0.663 | 0.617 |
| | pancreatic ctrl | | | | | | |
| 38 | PDCA vs non- | resectable | 0.871 | 0.877 | 0.875 | 0.886 | 0.895 |
| | pancreatic ctrl | | | | | | |
| 39 | PDCA vs (CP + | all | 0.893 | 0.889 | 0.891 | 0.900 | 0.905 |
| | non-pancreatic ctrl) | | | | | | |
| 39 | PDCA vs (CP + | low CA19-9 | 0.720 | 0.641 | 0.677 | 0.662 | 0.622 |
| | non-pancreatic ctrl) | | | | | | |
| 39 | PDCA vs (CP + | resectable | 0.879 | 0.874 | 0.877 | 0.887 | 0.893 |
| | non-pancreatic ctrl) | | | | | | |
| 39 | PDCA vs CP | all | 0.915 | 0.888 | 0.901 | 0.914 | 0.923 |
| 39 | PDCA vs CP | low CA19-9 | 0.820 | 0.701 | 0.755 | 0.758 | 0.738 |
| 39 | PDCA vs CP | resectable | 0.902 | 0.872 | 0.887 | 0.901 | 0.913 |
| 39 | PDCA vs non- | all | 0.865 | 0.886 | 0.876 | 0.885 | 0.895 |
| | pancreatic ctrl | | | | | | |
| 39 | PDCA vs non- | low CA19-9 | 0.660 | 0.631 | 0.644 | 0.620 | 0.579 |
| | pancreatic ctrl | | | | | | |
| 39 | PDCA vs non- | resectable | 0.844 | 0.869 | 0.857 | 0.869 | 0.883 |
| | pancreatic ctrl | | | | | | |
| 40 | PDCA vs (CP + | all | 0.892 | 0.887 | 0.890 | 0.898 | 0.904 |
| | non-pancreatic ctrl) | | | | | | |
| 40 | PDCA vs (CP + | low CA19-9 | 0.736 | 0.654 | 0.691 | 0.679 | 0.641 |
| | non-pancreatic ctrl) | | | | | | |
| 40 | PDCA vs (CP + | resectable | 0.876 | 0.869 | 0.873 | 0.882 | 0.889 |
| | non-pancreatic ctrl) | | | | | | |
| 40 | PDCA vs CP | all | 0.908 | 0.890 | 0.899 | 0.910 | 0.917 |
| 40 | PDCA vs CP | low CA19-9 | 0.821 | 0.726 | 0.770 | 0.770 | 0.746 |
| 40 | PDCA vs CP | resectable | 0.898 | 0.878 | 0.888 | 0.900 | 0.908 |
| 40 | PDCA vs non- | all | 0.882 | 0.888 | 0.886 | 0.895 | 0.904 |
| | pancreatic ctrl | | | | | | |
| 40 | PDCA vs non- | low CA19-9 | 0.711 | 0.651 | 0.677 | 0.658 | 0.617 |
| | pancreatic ctrl | | | | | | |
| 40 | PDCA vs non- | resectable | 0.867 | 0.873 | 0.870 | 0.882 | 0.894 |
| | pancreatic ctrl | | | | | | |
| 41 | PDCA vs (CP + | all | 0.891 | 0.889 | 0.890 | 0.895 | 0.896 |
| | non-pancreatic ctrl) | | | | | | |
| 41 | PDCA vs (CP + | low CA19-9 | 0.727 | 0.653 | 0.687 | 0.665 | 0.620 |
| | non-pancreatic ctrl) | | | | | | |
| 41 | PDCA vs (CP + | resectable | 0.875 | 0.873 | 0.874 | 0.880 | 0.882 |
| | non-pancreatic ctrl) | | | | | | |
| 41 | PDCA vs CP | all | 0.902 | 0.889 | 0.895 | 0.893 | 0.885 |
| 41 | PDCA vs CP | low CA19-9 | 0.807 | 0.704 | 0.752 | 0.712 | 0.648 |
| 41 | PDCA vs CP | resectable | 0.894 | 0.879 | 0.886 | 0.884 | 0.876 |
| 41 | PDCA vs non- | all | 0.885 | 0.895 | 0.890 | 0.899 | 0.906 |
| | pancreatic ctrl | | | | | | |
| 41 | PDCA vs non- | low CA19-9 | 0.718 | 0.659 | 0.685 | 0.663 | 0.623 |
| | pancreatic ctrl | | | | | | |
| 41 | PDCA vs non- | resectable | 0.870 | 0.882 | 0.876 | 0.887 | 0.898 |
| | pancreatic ctrl | | | | | | |
| 42 | PDCA vs (CP + | all | 0.890 | 0.891 | 0.891 | 0.900 | 0.908 |
| | non-pancreatic ctrl) | | | | | | |
| 42 | PDCA vs (CP + | low CA19-9 | 0.714 | 0.640 | 0.674 | 0.662 | 0.632 |
| | non-pancreatic ctrl) | | | | | | |
| 42 | PDCA vs (CP + | resectable | 0.876 | 0.877 | 0.876 | 0.887 | 0.897 |
| | non-pancreatic ctrl) | | | | | | |
| 42 | PDCA vs CP | all | 0.909 | 0.887 | 0.898 | 0.904 | 0.909 |
| 42 | PDCA vs CP | low CA19-9 | 0.800 | 0.678 | 0.734 | 0.717 | 0.684 |
| 42 | PDCA vs CP | resectable | 0.899 | 0.873 | 0.886 | 0.892 | 0.899 |
| 42 | PDCA vs non- | all | 0.867 | 0.890 | 0.878 | 0.888 | 0.898 |
| | pancreatic ctrl | | | | | | |
| 42 | PDCA vs non- | low CA19-9 | 0.662 | 0.636 | 0.648 | 0.627 | 0.590 |
| | pancreatic ctrl | | | | | | |
| 42 | PDCA vs non- | resectable | 0.847 | 0.875 | 0.861 | 0.873 | 0.887 |
| | pancreatic ctrl | | | | | | |
| 43 | PDCA vs (CP + | all | 0.899 | 0.886 | 0.892 | 0.900 | 0.903 |
| | non-pancreatic ctrl) | | | | | | |
| 43 | PDCA vs (CP + | low CA19-9 | 0.767 | 0.677 | 0.717 | 0.704 | 0.669 |
| | non-pancreatic ctrl) | | | | | | |
| 43 | PDCA vs (CP + | resectable | 0.881 | 0.865 | 0.873 | 0.882 | 0.885 |
| | non-pancreatic ctrl) | | | | | | |
| 43 | PDCA vs CP | all | 0.916 | 0.888 | 0.902 | 0.910 | 0.914 |
| 43 | PDCA vs CP | low CA19-9 | 0.856 | 0.750 | 0.798 | 0.792 | 0.768 |
| 43 | PDCA vs CP | resectable | 0.903 | 0.870 | 0.887 | 0.896 | 0.900 |
| 43 | PDCA vs non- | all | 0.895 | 0.896 | 0.896 | 0.904 | 0.911 |
| | pancreatic ctrl | | | | | | |
| 43 | PDCA vs non- | low CA19-9 | 0.750 | 0.671 | 0.705 | 0.684 | 0.646 |
| | pancreatic ctrl | | | | | | |
| 43 | PDCA vs non- | resectable | 0.882 | 0.882 | 0.882 | 0.892 | 0.903 |
| | pancreatic ctrl | | | | | | |
| 44 | PDCA vs (CP + | all | 0.901 | 0.886 | 0.894 | 0.904 | 0.911 |
| | non-pancreatic ctrl) | | | | | | |
| 44 | PDCA vs (CP + | low CA19-9 | 0.759 | 0.662 | 0.706 | 0.699 | 0.672 |
| | non-pancreatic ctrl) | | | | | | |
| 44 | PDCA vs (CP + | resectable | 0.882 | 0.864 | 0.873 | 0.885 | 0.893 |
| | non-pancreatic ctrl) | | | | | | |
| 44 | PDCA vs CP | all | 0.926 | 0.890 | 0.908 | 0.921 | 0.931 |
| 44 | PDCA vs CP | low CA19-9 | 0.860 | 0.738 | 0.793 | 0.798 | 0.789 |
| 44 | PDCA vs CP | resectable | 0.912 | 0.870 | 0.891 | 0.905 | 0.916 |
| 44 | PDCA vs non- | all | 0.883 | 0.894 | 0.889 | 0.897 | 0.907 |
| | pancreatic ctrl | | | | | | |
| 44 | PDCA vs non- | low CA19-9 | 0.712 | 0.658 | 0.682 | 0.660 | 0.625 |
| | pancreatic ctrl | | | | | | |
| 44 | PDCA vs non- | resectable | 0.865 | 0.878 | 0.872 | 0.883 | 0.895 |
| | pancreatic ctrl | | | | | | |
| 45 | PDCA vs (CP + | all | 0.893 | 0.890 | 0.892 | 0.899 | 0.906 |
| | non-pancreatic ctrl) | | | | | | |
| 45 | PDCA vs (CP + | low CA19-9 | 0.735 | 0.657 | 0.693 | 0.681 | 0.648 |
| | non-pancreatic ctrl) | | | | | | |
| 45 | PDCA vs (CP + | resectable | 0.877 | 0.873 | 0.875 | 0.884 | 0.892 |
| | non-pancreatic ctrl) | | | | | | |
| 45 | PDCA vs CP | all | 0.906 | 0.889 | 0.897 | 0.900 | 0.901 |
| 45 | PDCA vs CP | low CA19-9 | 0.813 | 0.710 | 0.757 | 0.735 | 0.692 |
| 45 | PDCA vs CP | resectable | 0.896 | 0.877 | 0.886 | 0.889 | 0.890 |
| 45 | PDCA vs non- | all | 0.884 | 0.893 | 0.889 | 0.898 | 0.907 |
| | pancreatic ctrl | | | | | | |
| 45 | PDCA vs non- | low CA19-9 | 0.714 | 0.655 | 0.681 | 0.663 | 0.627 |
| | pancreatic ctrl | | | | | | |
| 45 | PDCA vs non- | resectable | 0.868 | 0.879 | 0.874 | 0.886 | 0.897 |
| | pancreatic ctrl | | | | | | |
| 46 | PDCA vs (CP + | all | 0.901 | 0.885 | 0.893 | 0.902 | 0.910 |
| | non-pancreatic ctrl) | | | | | | |
| 46 | PDCA vs (CP + | low CA19-9 | 0.768 | 0.674 | 0.717 | 0.710 | 0.684 |
| | non-pancreatic ctrl) | | | | | | |
| 46 | PDCA vs (CP + | resectable | 0.881 | 0.862 | 0.872 | 0.882 | 0.890 |
| | non-pancreatic ctrl) | | | | | | |
| 46 | PDCA vs CP | all | 0.921 | 0.891 | 0.906 | 0.917 | 0.926 |
| 46 | PDCA vs CP | low CA19-9 | 0.862 | 0.757 | 0.805 | 0.807 | 0.794 |
| 46 | PDCA vs CP | resectable | 0.907 | 0.872 | 0.890 | 0.902 | 0.911 |
| 46 | PDCA vs non- | all | 0.893 | 0.893 | 0.893 | 0.902 | 0.909 |
| | pancreatic ctrl | | | | | | |
| 46 | PDCA vs non- | low CA19-9 | 0.744 | 0.665 | 0.700 | 0.681 | 0.643 |
| | pancreatic ctrl | | | | | | |
| 46 | PDCA vs non- | resectable | 0.878 | 0.877 | 0.878 | 0.889 | 0.899 |
| | pancreatic ctrl | | | | | | |
| 47 | PDCA vs (CP + | all | 0.894 | 0.886 | 0.890 | 0.896 | 0.899 |
| | non-pancreatic ctrl) | | | | | | |
| 47 | PDCA vs (CP + | low CA19-9 | 0.761 | 0.685 | 0.719 | 0.702 | 0.666 |
| | non-pancreatic ctrl) | | | | | | |
| 47 | PDCA vs (CP + | resectable | 0.873 | 0.863 | 0.868 | 0.875 | 0.878 |
| | non-pancreatic ctrl) | | | | | | |
| 47 | PDCA vs CP | all | 0.906 | 0.882 | 0.894 | 0.900 | 0.902 |
| 47 | PDCA vs CP | low CA19-9 | 0.845 | 0.751 | 0.794 | 0.781 | 0.756 |
| 47 | PDCA vs CP | resectable | 0.891 | 0.863 | 0.877 | 0.884 | 0.887 |
| 47 | PDCA vs non- | all | 0.889 | 0.894 | 0.892 | 0.901 | 0.911 |
| | pancreatic ctrl | | | | | | |
| 47 | PDCA vs non- | low CA19-9 | 0.742 | 0.678 | 0.706 | 0.687 | 0.654 |
| | pancreatic ctrl | | | | | | |
| 47 | PDCA vs non- | resectable | 0.873 | 0.879 | 0.876 | 0.888 | 0.901 |
| | pancreatic ctrl | | | | | | |
| 48 | PDCA vs (CP + | all | 0.899 | 0.890 | 0.895 | 0.904 | 0.912 |
| | non-pancreatic ctrl) | | | | | | |
| 48 | PDCA vs (CP + | low CA19-9 | 0.766 | 0.682 | 0.720 | 0.710 | 0.686 |
| | non-pancreatic ctrl) | | | | | | |
| 48 | PDCA vs (CP + | resectable | 0.877 | 0.865 | 0.871 | 0.882 | 0.891 |
| | non-pancreatic ctrl) | | | | | | |
| 48 | PDCA vs CP | all | 0.917 | 0.886 | 0.901 | 0.913 | 0.923 |
| 48 | PDCA vs CP | low CA19-9 | 0.852 | 0.744 | 0.793 | 0.794 | 0.785 |
| 48 | PDCA vs CP | resectable | 0.898 | 0.861 | 0.880 | 0.892 | 0.903 |
| 48 | PDCA vs non- | all | 0.879 | 0.893 | 0.887 | 0.897 | 0.910 |
| | pancreatic ctrl | | | | | | |
| 48 | PDCA vs non- | low CA19-9 | 0.715 | 0.670 | 0.690 | 0.673 | 0.644 |
| | pancreatic ctrl | | | | | | |
| 48 | PDCA vs non- | resectable | 0.861 | 0.878 | 0.869 | 0.883 | 0.899 |
| | pancreatic ctrl | | | | | | |
| 49 | PDCA vs (CP + | all | 0.896 | 0.886 | 0.891 | 0.900 | 0.908 |
| | non-pancreatic ctrl) | | | | | | |
| 49 | PDCA vs (CP + | low CA19-9 | 0.766 | 0.684 | 0.721 | 0.711 | 0.685 |
| | non-pancreatic ctrl) | | | | | | |
| 49 | PDCA vs (CP + | resectable | 0.872 | 0.861 | 0.867 | 0.876 | 0.885 |
| | non-pancreatic ctrl) | | | | | | |
| 49 | PDCA vs CP | all | 0.910 | 0.885 | 0.898 | 0.907 | 0.915 |
| 49 | PDCA vs CP | low CA19-9 | 0.848 | 0.756 | 0.798 | 0.795 | 0.780 |
| 49 | PDCA vs CP | resectable | 0.891 | 0.863 | 0.877 | 0.887 | 0.897 |
| 49 | PDCA vs non- | all | 0.888 | 0.892 | 0.890 | 0.901 | 0.910 |
| | pancreatic ctrl | | | | | | |
| 49 | PDCA vs non- | low CA19-9 | 0.741 | 0.675 | 0.704 | 0.688 | 0.655 |
| | pancreatic ctrl | | | | | | |
| 49 | PDCA vs non- | resectable | 0.871 | 0.876 | 0.874 | 0.887 | 0.899 |
| | pancreatic ctrl | | | | | | |
| 50 | PDCA vs (CP + | all | 0.890 | 0.893 | 0.892 | 0.895 | 0.896 |
| | non-pancreatic ctrl) | | | | | | |
| 50 | PDCA vs (CP + | low CA19-9 | 0.736 | 0.673 | 0.702 | 0.674 | 0.625 |
| | non-pancreatic ctrl) | | | | | | |
| 50 | PDCA vs (CP + | resectable | 0.871 | 0.873 | 0.872 | 0.876 | 0.877 |
| | non-pancreatic ctrl) | | | | | | |
| 50 | PDCA vs CP | all | 0.898 | 0.885 | 0.891 | 0.883 | 0.871 |
| 50 | PDCA vs CP | low CA19-9 | 0.799 | 0.705 | 0.748 | 0.694 | 0.619 |
| 50 | PDCA vs CP | resectable | 0.887 | 0.873 | 0.880 | 0.871 | 0.858 |
| 50 | PDCA vs non- | all | 0.880 | 0.895 | 0.888 | 0.898 | 0.907 |
| | pancreatic ctrl | | | | | | |
| 50 | PDCA vs non- | low CA19-9 | 0.712 | 0.670 | 0.688 | 0.671 | 0.635 |
| | pancreatic ctrl | | | | | | |
| 50 | PDCA vs non- | resectable | 0.862 | 0.880 | 0.871 | 0.884 | 0.897 |
| | pancreatic ctrl | | | | | | |
| 51 | PDCA vs (CP + | all | 0.893 | 0.896 | 0.895 | 0.903 | 0.911 |
| | non-pancreatic ctrl) | | | | | | |
| 51 | PDCA vs (CP + | low CA19-9 | 0.731 | 0.664 | 0.694 | 0.680 | 0.649 |
| | non-pancreatic ctrl) | | | | | | |
| 51 | PDCA vs (CP + | resectable | 0.873 | 0.877 | 0.875 | 0.885 | 0.894 |
| | non-pancreatic ctrl) | | | | | | |
| 51 | PDCA vs CP | all | 0.906 | 0.887 | 0.896 | 0.898 | 0.900 |
| 51 | PDCA vs CP | low CA19-9 | 0.797 | 0.687 | 0.738 | 0.710 | 0.669 |
| 51 | PDCA vs CP | resectable | 0.891 | 0.868 | 0.879 | 0.882 | 0.883 |
| 51 | PDCA vs non- | all | 0.867 | 0.892 | 0.880 | 0.892 | 0.905 |
| | pancreatic ctrl | | | | | | |
| 51 | PDCA vs non- | low CA19-9 | 0.672 | 0.655 | 0.662 | 0.647 | 0.616 |
| | pancreatic ctrl | | | | | | |
| 51 | PDCA vs non- | resectable | 0.845 | 0.876 | 0.861 | 0.876 | 0.893 |
| | pancreatic ctrl | | | | | | |
| 52 | PDCA vs (CP + | all | 0.891 | 0.892 | 0.892 | 0.898 | 0.904 |
| | non-pancreatic ctrl) | | | | | | |
| 52 | PDCA vs (CP + | low CA19-9 | 0.740 | 0.671 | 0.702 | 0.684 | 0.648 |
| | non-pancreatic ctrl) | | | | | | |
| 52 | PDCA vs (CP + | resectable | 0.871 | 0.871 | 0.871 | 0.879 | 0.885 |
| | non-pancreatic ctrl) | | | | | | |
| 52 | PDCA vs CP | all | 0.901 | 0.887 | 0.894 | 0.892 | 0.890 |
| 52 | PDCA vs CP | low CA19-9 | 0.804 | 0.712 | 0.754 | 0.720 | 0.668 |
| 52 | PDCA vs CP | resectable | 0.887 | 0.872 | 0.879 | 0.877 | 0.875 |
| 52 | PDCA vs non- | all | 0.880 | 0.893 | 0.886 | 0.898 | 0.908 |
| | pancreatic ctrl | | | | | | |
| 52 | PDCA vs non- | low CA19-9 | 0.713 | 0.667 | 0.687 | 0.673 | 0.640 |
| | pancreatic ctrl | | | | | | |
| 52 | PDCA vs non- | resectable | 0.862 | 0.877 | 0.870 | 0.884 | 0.897 |
| | pancreatic ctrl | | | | | | |
| 53 | PDCA vs (CP + | all | 0.892 | 0.891 | 0.891 | 0.896 | 0.896 |
| | non-pancreatic ctrl) | | | | | | |
| 53 | PDCA vs (CP + | low CA19-9 | 0.739 | 0.672 | 0.703 | 0.678 | 0.626 |
| | non-pancreatic ctrl) | | | | | | |
| 53 | PDCA vs (CP + | resectable | 0.875 | 0.874 | 0.875 | 0.880 | 0.880 |
| | non-pancreatic ctrl) | | | | | | |
| 53 | PDCA vs CP | all | 0.900 | 0.883 | 0.891 | 0.896 | 0.894 |
| 53 | PDCA vs CP | low CA19-9 | 0.804 | 0.717 | 0.757 | 0.738 | 0.693 |
| 53 | PDCA vs CP | resectable | 0.889 | 0.870 | 0.880 | 0.885 | 0.883 |
| 53 | PDCA vs non- | all | 0.881 | 0.892 | 0.887 | 0.897 | 0.907 |
| | pancreatic ctrl | | | | | | |
| 53 | PDCA vs non- | low CA19-9 | 0.716 | 0.668 | 0.689 | 0.671 | 0.632 |
| | pancreatic ctrl | | | | | | |
| 53 | PDCA vs non- | resectable | 0.865 | 0.878 | 0.872 | 0.885 | 0.898 |
| | pancreatic ctrl | | | | | | |
| 54 | PDCA vs (CP + | all | 0.894 | 0.893 | 0.893 | 0.902 | 0.908 |
| | non-pancreatic ctrl) | | | | | | |
| 54 | PDCA vs (CP + | low CA19-9 | 0.731 | 0.660 | 0.692 | 0.677 | 0.639 |
| | non-pancreatic ctrl) | | | | | | |
| 54 | PDCA vs (CP + | resectable | 0.875 | 0.874 | 0.874 | 0.885 | 0.892 |
| | non-pancreatic ctrl) | | | | | | |
| 54 | PDCA vs CP | all | 0.909 | 0.886 | 0.898 | 0.909 | 0.917 |
| 54 | PDCA vs CP | low CA19-9 | 0.808 | 0.704 | 0.752 | 0.749 | 0.727 |
| 54 | PDCA vs CP | resectable | 0.892 | 0.865 | 0.879 | 0.891 | 0.901 |
| 54 | PDCA vs non- | all | 0.865 | 0.887 | 0.876 | 0.889 | 0.902 |
| | pancreatic ctrl | | | | | | |
| 54 | PDCA vs non- | low CA19-9 | 0.667 | 0.647 | 0.656 | 0.641 | 0.606 |
| | pancreatic ctrl | | | | | | |
| 54 | PDCA vs non- | resectable | 0.845 | 0.872 | 0.859 | 0.874 | 0.891 |
| | pancreatic ctrl | | | | | | |
| 55 | PDCA vs (CP + | all | 0.891 | 0.890 | 0.891 | 0.898 | 0.904 |
| | non-pancreatic ctrl) | | | | | | |
| 55 | PDCA vs (CP + | low CA19-9 | 0.739 | 0.669 | 0.701 | 0.685 | 0.647 |
| | non-pancreatic ctrl) | | | | | | |
| 55 | PDCA vs (CP + | resectable | 0.872 | 0.871 | 0.872 | 0.880 | 0.887 |
| | non-pancreatic ctrl) | | | | | | |
| 55 | PDCA vs CP | all | 0.900 | 0.885 | 0.892 | 0.901 | 0.907 |
| 55 | PDCA vs CP | low CA19-9 | 0.802 | 0.717 | 0.756 | 0.750 | 0.722 |
| 55 | PDCA vs CP | resectable | 0.887 | 0.869 | 0.878 | 0.888 | 0.896 |
| 55 | PDCA vs non- | all | 0.878 | 0.888 | 0.884 | 0.895 | 0.906 |
| | pancreatic ctrl | | | | | | |
| 55 | PDCA vs non- | low CA19-9 | 0.712 | 0.663 | 0.684 | 0.670 | 0.635 |
| | pancreatic ctrl | | | | | | |
| 55 | PDCA vs non- | resectable | 0.862 | 0.874 | 0.868 | 0.883 | 0.897 |
| | pancreatic ctrl | | | | | | |
| 56 | PDCA vs (CP + | all | 0.891 | 0.878 | 0.885 | 0.886 | 0.871 |
| | non-pancreatic ctrl) | | | | | | |
| 56 | PDCA vs (CP + | low CA19-9 | 0.719 | 0.636 | 0.673 | 0.642 | 0.569 |
| | non-pancreatic ctrl) | | | | | | |
| 56 | PDCA vs (CP + | resectable | 0.885 | 0.870 | 0.878 | 0.879 | 0.863 |
| | non-pancreatic ctrl) | | | | | | |
| 56 | PDCA vs CP | all | 0.908 | 0.882 | 0.895 | 0.885 | 0.859 |
| 56 | PDCA vs CP | low CA19-9 | 0.817 | 0.704 | 0.756 | 0.702 | 0.598 |
| 56 | PDCA vs CP | resectable | 0.909 | 0.881 | 0.894 | 0.884 | 0.856 |
| 56 | PDCA vs non- | all | 0.880 | 0.885 | 0.883 | 0.886 | 0.877 |
| | pancreatic ctrl | | | | | | |
| 56 | PDCA vs non- | low CA19-9 | 0.693 | 0.632 | 0.659 | 0.626 | 0.552 |
| | pancreatic ctrl | | | | | | |
| 56 | PDCA vs non- | resectable | 0.872 | 0.877 | 0.875 | 0.880 | 0.871 |
| | pancreatic ctrl | | | | | | |
| 57 | PDCA vs (CP + | all | 0.897 | 0.883 | 0.890 | 0.895 | 0.886 |
| | non-pancreatic ctrl) | | | | | | |
| 57 | PDCA vs (CP + | low CA19-9 | 0.742 | 0.656 | 0.695 | 0.673 | 0.609 |
| | non-pancreatic ctrl) | | | | | | |
| 57 | PDCA vs (CP + | resectable | 0.891 | 0.876 | 0.884 | 0.890 | 0.880 |
| | non-pancreatic ctrl) | | | | | | |
| 57 | PDCA vs CP | all | 0.913 | 0.889 | 0.901 | 0.907 | 0.901 |
| 57 | PDCA vs CP | low CA19-9 | 0.830 | 0.734 | 0.778 | 0.767 | 0.710 |
| 57 | PDCA vs CP | resectable | 0.912 | 0.887 | 0.899 | 0.907 | 0.901 |
| 57 | PDCA vs non- | all | 0.885 | 0.884 | 0.885 | 0.889 | 0.882 |
| | pancreatic ctrl | | | | | | |
| 57 | PDCA vs non- | low CA19-9 | 0.711 | 0.645 | 0.674 | 0.641 | 0.565 |
| | pancreatic ctrl | | | | | | |
| 57 | PDCA vs non- | resectable | 0.877 | 0.876 | 0.877 | 0.883 | 0.876 |
| | pancreatic ctrl | | | | | | |
| 58 | PDCA vs (CP + | all | 0.905 | 0.880 | 0.893 | 0.899 | 0.894 |
| | non-pancreatic ctrl) | | | | | | |
| 58 | PDCA vs (CP + | low CA19-9 | 0.766 | 0.668 | 0.712 | 0.697 | 0.649 |
| | non-pancreatic ctrl) | | | | | | |
| 58 | PDCA vs (CP + | resectable | 0.893 | 0.864 | 0.879 | 0.886 | 0.880 |
| | non-pancreatic ctrl) | | | | | | |
| 58 | PDCA vs CP | all | 0.923 | 0.882 | 0.903 | 0.910 | 0.907 |
| 58 | PDCA vs CP | low CA19-9 | 0.867 | 0.755 | 0.806 | 0.800 | 0.761 |
| 58 | PDCA vs CP | resectable | 0.915 | 0.869 | 0.892 | 0.900 | 0.896 |
| 58 | PDCA vs non- | all | 0.894 | 0.887 | 0.890 | 0.895 | 0.889 |
| | pancreatic ctrl | | | | | | |
| 58 | PDCA vs non- | low CA19-9 | 0.737 | 0.651 | 0.688 | 0.659 | 0.593 |
| | pancreatic ctrl | | | | | | |
| 58 | PDCA vs non- | resectable | 0.885 | 0.876 | 0.880 | 0.886 | 0.881 |
| | pancreatic ctrl | | | | | | |
| 59 | PDCA vs (CP + | all | 0.880 | 0.874 | 0.877 | 0.876 | 0.862 |
| | non-pancreatic ctrl) | | | | | | |
| 59 | PDCA vs (CP + | low CA19-9 | 0.688 | 0.616 | 0.648 | 0.613 | 0.541 |
| | non-pancreatic ctrl) | | | | | | |
| 59 | PDCA vs (CP + | resectable | 0.870 | 0.863 | 0.867 | 0.866 | 0.850 |
| | non-pancreatic ctrl) | | | | | | |
| 59 | PDCA vs CP | all | 0.897 | 0.876 | 0.887 | 0.876 | 0.851 |
| 59 | PDCA vs CP | low CA19-9 | 0.797 | 0.683 | 0.735 | 0.677 | 0.577 |
| 59 | PDCA vs CP | resectable | 0.892 | 0.870 | 0.881 | 0.869 | 0.840 |
| 59 | PDCA vs non- | all | 0.867 | 0.881 | 0.874 | 0.878 | 0.871 |
| | pancreatic ctrl | | | | | | |
| 59 | PDCA vs non- | low CA19-9 | 0.657 | 0.615 | 0.634 | 0.601 | 0.530 |
| | pancreatic ctrl | | | | | | |
| 59 | PDCA vs non- | resectable | 0.852 | 0.871 | 0.862 | 0.867 | 0.861 |
| | pancreatic ctrl | | | | | | |
| 60 | PDCA vs (CP + | all | 0.887 | 0.881 | 0.884 | 0.889 | 0.883 |
| | non-pancreatic ctrl) | | | | | | |
| 60 | PDCA vs (CP + | low CA19-9 | 0.702 | 0.620 | 0.657 | 0.634 | 0.578 |
| | non-pancreatic ctrl) | | | | | | |
| 60 | PDCA vs (CP + | resectable | 0.877 | 0.870 | 0.873 | 0.879 | 0.873 |
| | non-pancreatic ctrl) | | | | | | |
| 60 | PDCA vs CP | all | 0.907 | 0.879 | 0.892 | 0.891 | 0.880 |
| 60 | PDCA vs CP | low CA19-9 | 0.800 | 0.668 | 0.729 | 0.696 | 0.629 |
| 60 | PDCA vs CP | resectable | 0.899 | 0.867 | 0.883 | 0.881 | 0.869 |
| 60 | PDCA vs non- | all | 0.861 | 0.880 | 0.870 | 0.875 | 0.870 |
| | pancreatic ctrl | | | | | | |
| 60 | PDCA vs non- | low CA19-9 | 0.642 | 0.611 | 0.625 | 0.594 | 0.527 |
| | pancreatic ctrl | | | | | | |
| 60 | PDCA vs non- | resectable | 0.843 | 0.867 | 0.855 | 0.862 | 0.857 |
| | pancreatic ctrl | | | | | | |
| 61 | PDCA vs (CP + | all | 0.888 | 0.880 | 0.884 | 0.887 | 0.879 |
| | non-pancreatic ctrl) | | | | | | |
| 61 | PDCA vs (CP + | low CA19-9 | 0.718 | 0.641 | 0.676 | 0.649 | 0.585 |
| | non-pancreatic ctrl) | | | | | | |
| 61 | PDCA vs (CP + | resectable | 0.880 | 0.871 | 0.876 | 0.880 | 0.871 |
| | non-pancreatic ctrl) | | | | | | |
| 61 | PDCA vs CP | all | 0.901 | 0.881 | 0.891 | 0.895 | 0.888 |
| 61 | PDCA vs CP | low CA19-9 | 0.813 | 0.713 | 0.759 | 0.742 | 0.683 |
| 61 | PDCA vs CP | resectable | 0.897 | 0.876 | 0.886 | 0.892 | 0.885 |
| 61 | PDCA vs non- | all | 0.874 | 0.883 | 0.879 | 0.883 | 0.879 |
| | pancreatic ctrl | | | | | | |
| 61 | PDCA vs non- | low CA19-9 | 0.684 | 0.633 | 0.655 | 0.623 | 0.552 |
| | pancreatic ctrl | | | | | | |
| 61 | PDCA vs non- | resectable | 0.864 | 0.874 | 0.870 | 0.876 | 0.873 |
| | pancreatic ctrl | | | | | | |
| 62 | PDCA vs (CP + | all | 0.892 | 0.883 | 0.888 | 0.894 | 0.892 |
| | non-pancreatic ctrl) | | | | | | |
| 62 | PDCA vs (CP + | low CA19-9 | 0.719 | 0.634 | 0.672 | 0.654 | 0.601 |
| | non-pancreatic ctrl) | | | | | | |
| 62 | PDCA vs (CP + | resectable | 0.881 | 0.872 | 0.877 | 0.885 | 0.883 |
| | non-pancreatic ctrl) | | | | | | |
| 62 | PDCA vs CP | all | 0.912 | 0.884 | 0.898 | 0.908 | 0.912 |
| 62 | PDCA vs CP | low CA19-9 | 0.818 | 0.699 | 0.754 | 0.752 | 0.717 |
| 62 | PDCA vs CP | resectable | 0.902 | 0.871 | 0.887 | 0.899 | 0.903 |
| 62 | PDCA vs non- | all | 0.861 | 0.879 | 0.870 | 0.876 | 0.874 |
| | pancreatic ctrl | | | | | | |
| 62 | PDCA vs non- | low CA19-9 | 0.646 | 0.619 | 0.631 | 0.600 | 0.532 |
| | pancreatic ctrl | | | | | | |
| 62 | PDCA vs non- | resectable | 0.846 | 0.868 | 0.857 | 0.865 | 0.864 |
| | pancreatic ctrl | | | | | | |
| 63 | PDCA vs (CP + | all | 0.882 | 0.874 | 0.878 | 0.881 | 0.875 |
| | non-pancreatic ctrl) | | | | | | |
| 63 | PDCA vs (CP + | low CA19-9 | 0.700 | 0.618 | 0.655 | 0.631 | 0.574 |
| | non-pancreatic ctrl) | | | | | | |
| 63 | PDCA vs (CP + | resectable | 0.870 | 0.861 | 0.866 | 0.869 | 0.862 |
| | non-pancreatic ctrl) | | | | | | |
| 63 | PDCA vs CP | all | 0.901 | 0.880 | 0.890 | 0.886 | 0.873 |
| 63 | PDCA vs CP | low CA19-9 | 0.805 | 0.694 | 0.745 | 0.708 | 0.635 |
| 63 | PDCA vs CP | resectable | 0.894 | 0.871 | 0.882 | 0.878 | 0.864 |
| 63 | PDCA vs non- | all | 0.869 | 0.881 | 0.875 | 0.880 | 0.874 |
| | pancreatic ctrl | | | | | | |
| 63 | PDCA vs non- | low CA19-9 | 0.668 | 0.618 | 0.640 | 0.610 | 0.542 |
| | pancreatic ctrl | | | | | | |
| 63 | PDCA vs non- | resectable | 0.856 | 0.870 | 0.863 | 0.869 | 0.864 |
| | pancreatic ctrl | | | | | | |
| 64 | PDCA vs (CP + | all | 0.888 | 0.879 | 0.883 | 0.890 | 0.888 |
| | non-pancreatic ctrl) | | | | | | |
| 64 | PDCA vs (CP + | low CA19-9 | 0.720 | 0.637 | 0.674 | 0.658 | 0.608 |
| | non-pancreatic ctrl) | | | | | | |
| 64 | PDCA vs (CP + | resectable | 0.877 | 0.867 | 0.872 | 0.880 | 0.879 |
| | non-pancreatic ctrl) | | | | | | |
| 64 | PDCA vs CP | all | 0.903 | 0.884 | 0.893 | 0.902 | 0.904 |
| 64 | PDCA vs CP | low CA19-9 | 0.813 | 0.716 | 0.761 | 0.758 | 0.721 |
| 64 | PDCA vs CP | resectable | 0.897 | 0.876 | 0.886 | 0.897 | 0.900 |
| 64 | PDCA vs non- | all | 0.873 | 0.880 | 0.877 | 0.882 | 0.879 |
| | pancreatic ctrl | | | | | | |
| 64 | PDCA vs non- | low CA19-9 | 0.684 | 0.631 | 0.654 | 0.624 | 0.556 |
| | pancreatic ctrl | | | | | | |
| 64 | PDCA vs non- | resectable | 0.861 | 0.870 | 0.866 | 0.873 | 0.871 |
| | pancreatic ctrl | | | | | | |
| 65 | PDCA vs (CP + | all | 0.892 | 0.874 | 0.883 | 0.888 | 0.884 |
| | non-pancreatic ctrl) | | | | | | |
| 65 | PDCA vs (CP + | low CA19-9 | 0.735 | 0.650 | 0.688 | 0.670 | 0.623 |
| | non-pancreatic ctrl) | | | | | | |
| 65 | PDCA vs (CP + | resectable | 0.877 | 0.856 | 0.867 | 0.873 | 0.868 |
| | non-pancreatic ctrl) | | | | | | |
| 65 | PDCA vs CP | all | 0.909 | 0.876 | 0.893 | 0.899 | 0.897 |
| 65 | PDCA vs CP | low CA19-9 | 0.842 | 0.733 | 0.782 | 0.773 | 0.735 |
| 65 | PDCA vs CP | resectable | 0.896 | 0.859 | 0.878 | 0.885 | 0.882 |
| 65 | PDCA vs non- | all | 0.880 | 0.883 | 0.882 | 0.886 | 0.882 |
| | pancreatic ctrl | | | | | | |
| 65 | PDCA vs non- | low CA19-9 | 0.697 | 0.632 | 0.661 | 0.632 | 0.570 |
| | pancreatic ctrl | | | | | | |
| 65 | PDCA vs non- | resectable | 0.864 | 0.868 | 0.866 | 0.872 | 0.869 |
| | pancreatic ctrl | | | | | | |
| 66 | PDCA vs (CP + | all | 0.901 | 0.879 | 0.890 | 0.898 | 0.899 |
| | non-pancreatic ctrl) | | | | | | |
| 66 | PDCA vs (CP + | low CA19-9 | 0.751 | 0.651 | 0.695 | 0.686 | 0.648 |
| | non-pancreatic ctrl) | | | | | | |
| 66 | PDCA vs (CP + | resectable | 0.884 | 0.858 | 0.871 | 0.881 | 0.882 |
| | non-pancreatic ctrl) | | | | | | |
| 66 | PDCA vs CP | all | 0.921 | 0.882 | 0.902 | 0.913 | 0.919 |
| 66 | PDCA vs CP | low CA19-9 | 0.858 | 0.734 | 0.790 | 0.793 | 0.773 |
| 66 | PDCA vs CP | resectable | 0.907 | 0.861 | 0.884 | 0.896 | 0.902 |
| 66 | PDCA vs non- | all | 0.877 | 0.883 | 0.881 | 0.886 | 0.885 |
| | pancreatic ctrl | | | | | | |
| 66 | PDCA vs non- | low CA19-9 | 0.693 | 0.635 | 0.661 | 0.634 | 0.576 |
| | pancreatic ctrl | | | | | | |
| 66 | PDCA vs non- | resectable | 0.862 | 0.869 | 0.866 | 0.873 | 0.873 |
| | pancreatic ctrl | | | | | | |
| 67 | PDCA vs (CP + | all | 0.894 | 0.874 | 0.884 | 0.892 | 0.893 |
| | non-pancreatic ctrl) | | | | | | |
| 67 | PDCA vs (CP + | low CA19-9 | 0.745 | 0.652 | 0.693 | 0.683 | 0.646 |
| | non-pancreatic ctrl) | | | | | | |
| 67 | PDCA vs (CP + | resectable | 0.877 | 0.854 | 0.866 | 0.875 | 0.875 |
| | non-pancreatic ctrl) | | | | | | |
| 67 | PDCA vs CP | all | 0.914 | 0.881 | 0.898 | 0.907 | 0.912 |
| 67 | PDCA vs CP | low CA19-9 | 0.854 | 0.746 | 0.795 | 0.796 | 0.772 |
| 67 | PDCA vs CP | resectable | 0.901 | 0.864 | 0.883 | 0.893 | 0.898 |
| 67 | PDCA vs non- | all | 0.884 | 0.882 | 0.883 | 0.888 | 0.885 |
| | pancreatic ctrl | | | | | | |
| 67 | PDCA vs non- | low CA19-9 | 0.714 | 0.639 | 0.672 | 0.645 | 0.584 |
| | pancreatic ctrl | | | | | | |
| 67 | PDCA vs non- | resectable | 0.870 | 0.868 | 0.869 | 0.876 | 0.873 |
| | pancreatic ctrl | | | | | | |
| 68 | PDCA vs (CP + | all | 0.891 | 0.881 | 0.886 | 0.883 | 0.866 |
| | non-pancreatic ctrl) | | | | | | |
| 68 | PDCA vs (CP + | low CA19-9 | 0.723 | 0.645 | 0.680 | 0.639 | 0.558 |
| | non-pancreatic ctrl) | | | | | | |
| 68 | PDCA vs (CP + | resectable | 0.880 | 0.869 | 0.875 | 0.872 | 0.852 |
| | non-pancreatic ctrl) | | | | | | |
| 68 | PDCA vs CP | all | 0.903 | 0.875 | 0.889 | 0.874 | 0.844 |
| 68 | PDCA vs CP | low CA19-9 | 0.809 | 0.700 | 0.750 | 0.684 | 0.573 |
| 68 | PDCA vs CP | resectable | 0.903 | 0.873 | 0.887 | 0.872 | 0.838 |
| 68 | PDCA vs non- | all | 0.873 | 0.884 | 0.878 | 0.883 | 0.877 |
| | pancreatic ctrl | | | | | | |
| 68 | PDCA vs non- | low CA19-9 | 0.675 | 0.633 | 0.651 | 0.620 | 0.550 |
| | pancreatic ctrl | | | | | | |
| 68 | PDCA vs non- | resectable | 0.861 | 0.874 | 0.868 | 0.874 | 0.869 |
| | pancreatic ctrl | | | | | | |
| 69 | PDCA vs (CP + | all | 0.896 | 0.884 | 0.890 | 0.893 | 0.884 |
| | non-pancreatic ctrl) | | | | | | |
| 69 | PDCA vs (CP + | low CA19-9 | 0.736 | 0.658 | 0.694 | 0.667 | 0.602 |
| | non-pancreatic ctrl) | | | | | | |
| 69 | PDCA vs (CP + | resectable | 0.886 | 0.873 | 0.880 | 0.883 | 0.873 |
| | non-pancreatic ctrl) | | | | | | |
| 69 | PDCA vs CP | all | 0.907 | 0.883 | 0.894 | 0.899 | 0.891 |
| 69 | PDCA vs CP | low CA19-9 | 0.814 | 0.724 | 0.765 | 0.748 | 0.687 |
| 69 | PDCA vs CP | resectable | 0.903 | 0.877 | 0.890 | 0.895 | 0.887 |
| 69 | PDCA vs non- | all | 0.878 | 0.882 | 0.880 | 0.886 | 0.881 |
| | pancreatic ctrl | | | | | | |
| 69 | PDCA vs non- | low CA19-9 | 0.697 | 0.644 | 0.667 | 0.637 | 0.562 |
| | pancreatic ctrl | | | | | | |
| 69 | PDCA vs non- | resectable | 0.869 | 0.874 | 0.872 | 0.879 | 0.874 |
| | pancreatic ctrl | | | | | | |
| 70 | PDCA vs (CP + | all | 0.898 | 0.878 | 0.888 | 0.892 | 0.887 |
| | non-pancreatic ctrl) | | | | | | |
| 70 | PDCA vs (CP + | low CA19-9 | 0.749 | 0.664 | 0.703 | 0.683 | 0.635 |
| | non-pancreatic ctrl) | | | | | | |
| 70 | PDCA vs (CP + | resectable | 0.881 | 0.858 | 0.870 | 0.875 | 0.868 |
| | non-pancreatic ctrl) | | | | | | |
| 70 | PDCA vs CP | all | 0.914 | 0.876 | 0.895 | 0.900 | 0.896 |
| 70 | PDCA vs CP | low CA19-9 | 0.855 | 0.752 | 0.798 | 0.787 | 0.746 |
| 70 | PDCA vs CP | resectable | 0.902 | 0.858 | 0.880 | 0.886 | 0.881 |
| 70 | PDCA vs non- | all | 0.884 | 0.883 | 0.884 | 0.888 | 0.884 |
| | pancreatic ctrl | | | | | | |
| 70 | PDCA vs non- | low CA19-9 | 0.717 | 0.647 | 0.677 | 0.647 | 0.582 |
| | pancreatic ctrl | | | | | | |
| 70 | PDCA vs non- | resectable | 0.870 | 0.869 | 0.870 | 0.876 | 0.873 |
| | pancreatic ctrl | | | | | | |
| 71 | PDCA vs (CP + | all | 0.887 | 0.873 | 0.880 | 0.887 | 0.888 |
| | non-pancreatic ctrl) | | | | | | |
| 71 | PDCA vs (CP + | low CA19-9 | 0.731 | 0.650 | 0.686 | 0.672 | 0.635 |
| | non-pancreatic ctrl) | | | | | | |
| 71 | PDCA vs (CP + | resectable | 0.866 | 0.849 | 0.857 | 0.865 | 0.866 |
| | non-pancreatic ctrl) | | | | | | |
| 71 | PDCA vs CP | all | 0.902 | 0.875 | 0.888 | 0.896 | 0.901 |
| 71 | PDCA vs CP | low CA19-9 | 0.838 | 0.744 | 0.787 | 0.783 | 0.758 |
| 71 | PDCA vs CP | resectable | 0.884 | 0.853 | 0.869 | 0.877 | 0.882 |
| 71 | PDCA vs non- | all | 0.875 | 0.880 | 0.877 | 0.883 | 0.882 |
| | pancreatic ctrl | | | | | | |
| 71 | PDCA vs non- | low CA19-9 | 0.699 | 0.638 | 0.665 | 0.637 | 0.577 |
| | pancreatic ctrl | | | | | | |
| 71 | PDCA vs non- | resectable | 0.857 | 0.863 | 0.860 | 0.868 | 0.867 |
| | pancreatic ctrl | | | | | | |
| 72 | PDCA vs (CP + | all | 0.879 | 0.876 | 0.878 | 0.873 | 0.856 |
| | non-pancreatic ctrl) | | | | | | |
| 72 | PDCA vs (CP + | low CA19-9 | 0.692 | 0.626 | 0.656 | 0.609 | 0.528 |
| | non-pancreatic ctrl) | | | | | | |
| 72 | PDCA vs (CP + | resectable | 0.865 | 0.862 | 0.864 | 0.859 | 0.839 |
| | non-pancreatic ctrl) | | | | | | |
| 72 | PDCA vs CP | all | 0.893 | 0.873 | 0.883 | 0.866 | 0.836 |
| 72 | PDCA vs CP | low CA19-9 | 0.788 | 0.681 | 0.730 | 0.657 | 0.547 |
| 72 | PDCA vs CP | resectable | 0.885 | 0.863 | 0.874 | 0.855 | 0.820 |
| 72 | PDCA vs non- | all | 0.858 | 0.880 | 0.869 | 0.874 | 0.871 |
| | pancreatic ctrl | | | | | | |
| 72 | PDCA vs non- | low CA19-9 | 0.637 | 0.616 | 0.625 | 0.594 | 0.528 |
| | pancreatic ctrl | | | | | | |
| 72 | PDCA vs non- | resectable | 0.839 | 0.866 | 0.853 | 0.860 | 0.857 |
| | pancreatic ctrl | | | | | | |
| 73 | PDCA vs (CP + | all | 0.888 | 0.884 | 0.886 | 0.888 | 0.881 |
| | non-pancreatic ctrl) | | | | | | |
| 73 | PDCA vs (CP + | low CA19-9 | 0.710 | 0.633 | 0.668 | 0.638 | 0.577 |
| | non-pancreatic ctrl) | | | | | | |
| 73 | PDCA vs (CP + | resectable | 0.872 | 0.867 | 0.869 | 0.872 | 0.864 |
| | non-pancreatic ctrl) | | | | | | |
| 73 | PDCA vs CP | all | 0.903 | 0.877 | 0.890 | 0.885 | 0.871 |
| 73 | PDCA vs CP | low CA19-9 | 0.799 | 0.676 | 0.733 | 0.690 | 0.615 |
| 73 | PDCA vs CP | resectable | 0.891 | 0.861 | 0.876 | 0.870 | 0.854 |
| 73 | PDCA vs non- | all | 0.858 | 0.881 | 0.869 | 0.876 | 0.874 |
| | pancreatic ctrl | | | | | | |
| 73 | PDCA vs non- | low CA19-9 | 0.639 | 0.619 | 0.628 | 0.600 | 0.536 |
| | pancreatic ctrl | | | | | | |
| 73 | PDCA vs non- | resectable | 0.837 | 0.865 | 0.851 | 0.861 | 0.859 |
| | pancreatic ctrl | | | | | | |
| 74 | PDCA vs (CP + | all | 0.882 | 0.877 | 0.879 | 0.879 | 0.871 |
| | non-pancreatic ctrl) | | | | | | |
| 74 | PDCA vs (CP + | low CA19-9 | 0.704 | 0.629 | 0.662 | 0.629 | 0.566 |
| | non-pancreatic ctrl) | | | | | | |
| 74 | PDCA vs (CP + | resectable | 0.866 | 0.859 | 0.863 | 0.863 | 0.853 |
| | non-pancreatic ctrl) | | | | | | |
| 74 | PDCA vs CP | all | 0.895 | 0.874 | 0.885 | 0.876 | 0.859 |
| 74 | PDCA vs CP | low CA19-9 | 0.793 | 0.689 | 0.737 | 0.687 | 0.604 |
| 74 | PDCA vs CP | resectable | 0.886 | 0.863 | 0.874 | 0.865 | 0.846 |
| 74 | PDCA vs non- | all | 0.864 | 0.880 | 0.872 | 0.878 | 0.875 |
| | pancreatic ctrl | | | | | | |
| 74 | PDCA vs non- | low CA19-9 | 0.659 | 0.624 | 0.639 | 0.610 | 0.544 |
| | pancreatic ctrl | | | | | | |
| 74 | PDCA vs non- | resectable | 0.847 | 0.866 | 0.857 | 0.864 | 0.861 |
| | pancreatic ctrl | | | | | | |
| 75 | PDCA vs (CP + | all | 0.886 | 0.881 | 0.883 | 0.885 | 0.876 |
| | non-pancreatic ctrl) | | | | | | |
| 75 | PDCA vs (CP + | low CA19-9 | 0.712 | 0.644 | 0.674 | 0.643 | 0.578 |
| | non-pancreatic ctrl) | | | | | | |
| 75 | PDCA vs (CP + | resectable | 0.874 | 0.869 | 0.872 | 0.874 | 0.864 |
| | non-pancreatic ctrl) | | | | | | |
| 75 | PDCA vs CP | all | 0.892 | 0.874 | 0.883 | 0.885 | 0.876 |
| 75 | PDCA vs CP | low CA19-9 | 0.790 | 0.701 | 0.742 | 0.718 | 0.655 |
| 75 | PDCA vs CP | resectable | 0.887 | 0.867 | 0.877 | 0.880 | 0.870 |
| 75 | PDCA vs non- | all | 0.867 | 0.881 | 0.874 | 0.880 | 0.877 |
| | pancreatic ctrl | | | | | | |
| 75 | PDCA vs non- | low CA19-9 | 0.667 | 0.631 | 0.647 | 0.616 | 0.547 |
| | pancreatic ctrl | | | | | | |
| 75 | PDCA vs non- | resectable | 0.854 | 0.871 | 0.863 | 0.871 | 0.869 |
| | pancreatic ctrl | | | | | | |
| 76 | PDCA vs (CP + | all | 0.892 | 0.887 | 0.889 | 0.895 | 0.893 |
| | non-pancreatic ctrl) | | | | | | |
| 76 | PDCA vs (CP + | low CA19-9 | 0.723 | 0.646 | 0.681 | 0.660 | 0.606 |
| | non-pancreatic ctrl) | | | | | | |
| 76 | PDCA vs (CP + | resectable | 0.877 | 0.871 | 0.874 | 0.881 | 0.879 |
| | non-pancreatic ctrl) | | | | | | |
| 76 | PDCA vs CP | all | 0.904 | 0.880 | 0.892 | 0.901 | 0.903 |
| 76 | PDCA vs CP | low CA19-9 | 0.803 | 0.696 | 0.745 | 0.739 | 0.701 |
| 76 | PDCA vs CP | resectable | 0.891 | 0.863 | 0.877 | 0.887 | 0.889 |
| 76 | PDCA vs non- | all | 0.859 | 0.880 | 0.870 | 0.877 | 0.877 |
| | pancreatic ctrl | | | | | | |
| 76 | PDCA vs non- | low CA19-9 | 0.645 | 0.625 | 0.634 | 0.607 | 0.542 |
| | pancreatic ctrl | | | | | | |
| 76 | PDCA vs non- | resectable | 0.844 | 0.868 | 0.856 | 0.866 | 0.867 |
| | pancreatic ctrl | | | | | | |
| 77 | PDCA vs (CP + | all | 0.885 | 0.879 | 0.882 | 0.888 | 0.886 |
| | non-pancreatic ctrl) | | | | | | |
| 77 | PDCA vs (CP + | low CA19-9 | 0.716 | 0.643 | 0.676 | 0.655 | 0.605 |
| | non-pancreatic ctrl) | | | | | | |
| 77 | PDCA vs (CP + | resectable | 0.871 | 0.865 | 0.869 | 0.875 | 0.874 |
| | non-pancreatic ctrl) | | | | | | |
| 77 | PDCA vs CP | all | 0.894 | 0.878 | 0.886 | 0.893 | 0.895 |
| 77 | PDCA vs CP | low CA19-9 | 0.792 | 0.708 | 0.747 | 0.739 | 0.699 |
| 77 | PDCA vs CP | resectable | 0.885 | 0.867 | 0.876 | 0.884 | 0.887 |
| 77 | PDCA vs non- | all | 0.867 | 0.879 | 0.873 | 0.880 | 0.879 |
| | pancreatic ctrl | | | | | | |
| 77 | PDCA vs non- | low CA19-9 | 0.674 | 0.633 | 0.650 | 0.623 | 0.558 |
| | pancreatic ctrl | | | | | | |
| 77 | PDCA vs non- | resectable | 0.855 | 0.869 | 0.862 | 0.871 | 0.872 |
| | pancreatic ctrl | | | | | | |
| 78 | PDCA vs (CP + | all | 0.885 | 0.873 | 0.879 | 0.883 | 0.879 |
| | non-pancreatic ctrl) | | | | | | |
| 78 | PDCA vs (CP + | low CA19-9 | 0.721 | 0.648 | 0.681 | 0.659 | 0.611 |
| | non-pancreatic ctrl) | | | | | | |
| 78 | PDCA vs (CP + | resectable | 0.865 | 0.851 | 0.858 | 0.863 | 0.857 |
| | non-pancreatic ctrl) | | | | | | |
| 78 | PDCA vs CP | all | 0.896 | 0.867 | 0.882 | 0.886 | 0.882 |
| 78 | PDCA vs CP | low CA19-9 | 0.827 | 0.728 | 0.773 | 0.758 | 0.718 |
| 78 | PDCA vs CP | resectable | 0.881 | 0.848 | 0.865 | 0.869 | 0.865 |
| 78 | PDCA vs non- | all | 0.871 | 0.881 | 0.876 | 0.882 | 0.880 |
| | pancreatic ctrl | | | | | | |
| 78 | PDCA vs non- | low CA19-9 | 0.681 | 0.633 | 0.654 | 0.625 | 0.566 |
| | pancreatic ctrl | | | | | | |
| 78 | PDCA vs non- | resectable | 0.852 | 0.864 | 0.859 | 0.866 | 0.865 |
| | pancreatic ctrl | | | | | | |
| 79 | PDCA vs (CP + | all | 0.897 | 0.880 | 0.889 | 0.896 | 0.898 |
| | non-pancreatic ctrl) | | | | | | |
| 79 | PDCA vs (CP + | low CA19-9 | 0.748 | 0.660 | 0.699 | 0.686 | 0.649 |
| | non-pancreatic ctrl) | | | | | | |
| 79 | PDCA vs (CP + | resectable | 0.876 | 0.856 | 0.866 | 0.875 | 0.876 |
| | non-pancreatic ctrl) | | | | | | |
| 79 | PDCA vs CP | all | 0.911 | 0.876 | 0.894 | 0.903 | 0.909 |
| 79 | PDCA vs CP | low CA19-9 | 0.849 | 0.738 | 0.788 | 0.787 | 0.768 |
| 79 | PDCA vs CP | resectable | 0.892 | 0.851 | 0.872 | 0.882 | 0.888 |
| 79 | PDCA vs non- | all | 0.873 | 0.883 | 0.878 | 0.885 | 0.885 |
| | pancreatic ctrl | | | | | | |
| 79 | PDCA vs non- | low CA19-9 | 0.689 | 0.640 | 0.661 | 0.634 | 0.577 |
| | pancreatic ctrl | | | | | | |
| 79 | PDCA vs non- | resectable | 0.853 | 0.864 | 0.859 | 0.867 | 0.868 |
| | pancreatic ctrl | | | | | | |
| 80 | PDCA vs (CP + | all | 0.899 | 0.882 | 0.891 | 0.898 | 0.897 |
| | non-pancreatic ctrl) | | | | | | |
| 80 | PDCA vs (CP + | low CA19-9 | 0.738 | 0.652 | 0.690 | 0.678 | 0.634 |
| | non-pancreatic ctrl) | | | | | | |
| 80 | PDCA vs (CP + | resectable | 0.888 | 0.869 | 0.879 | 0.887 | 0.886 |
| | non-pancreatic ctrl) | | | | | | |
| 80 | PDCA vs CP | all | 0.916 | 0.884 | 0.900 | 0.902 | 0.895 |
| 80 | PDCA vs CP | low CA19-9 | 0.838 | 0.727 | 0.778 | 0.758 | 0.702 |
| 80 | PDCA vs CP | resectable | 0.912 | 0.877 | 0.895 | 0.898 | 0.890 |
| 80 | PDCA vs non- | all | 0.883 | 0.888 | 0.885 | 0.897 | 0.903 |
| | pancreatic ctrl | | | | | | |
| 80 | PDCA vs non- | low CA19-9 | 0.699 | 0.644 | 0.668 | 0.659 | 0.619 |
| | pancreatic ctrl | | | | | | |
| 80 | PDCA vs non- | resectable | 0.871 | 0.877 | 0.874 | 0.888 | 0.896 |
| | pancreatic ctrl | | | | | | |
| 81 | PDCA vs (CP + | all | 0.903 | 0.886 | 0.894 | 0.902 | 0.900 |
| | non-pancreatic ctrl) | | | | | | |
| 81 | PDCA vs (CP + | low CA19-9 | 0.752 | 0.666 | 0.705 | 0.692 | 0.642 |
| | non-pancreatic ctrl) | | | | | | |
| 81 | PDCA vs (CP + | resectable | 0.894 | 0.875 | 0.885 | 0.894 | 0.892 |
| | non-pancreatic ctrl) | | | | | | |
| 81 | PDCA vs CP | all | 0.917 | 0.888 | 0.902 | 0.913 | 0.915 |
| 81 | PDCA vs CP | low CA19-9 | 0.841 | 0.742 | 0.787 | 0.790 | 0.757 |
| 81 | PDCA vs CP | resectable | 0.913 | 0.883 | 0.898 | 0.910 | 0.913 |
| 81 | PDCA vs non- | all | 0.886 | 0.885 | 0.886 | 0.896 | 0.901 |
| | pancreatic ctrl | | | | | | |
| 81 | PDCA vs non- | low CA19-9 | 0.711 | 0.648 | 0.675 | 0.661 | 0.615 |
| | pancreatic ctrl | | | | | | |
| 81 | PDCA vs non- | resectable | 0.877 | 0.875 | 0.876 | 0.889 | 0.896 |
| | pancreatic ctrl | | | | | | |
| 82 | PDCA vs (CP + | all | 0.910 | 0.882 | 0.896 | 0.906 | 0.908 |
| | non-pancreatic ctrl) | | | | | | |
| 82 | PDCA vs (CP + | low CA19-9 | 0.776 | 0.677 | 0.721 | 0.716 | 0.683 |
| | non-pancreatic ctrl) | | | | | | |
| 82 | PDCA vs (CP + | resectable | 0.896 | 0.865 | 0.881 | 0.892 | 0.893 |
| | non-pancreatic ctrl) | | | | | | |
| 82 | PDCA vs CP | all | 0.929 | 0.883 | 0.906 | 0.918 | 0.923 |
| 82 | PDCA vs CP | low CA19-9 | 0.877 | 0.763 | 0.815 | 0.821 | 0.803 |
| 82 | PDCA vs CP | resectable | 0.921 | 0.871 | 0.897 | 0.910 | 0.914 |
| 82 | PDCA vs non- | all | 0.895 | 0.889 | 0.892 | 0.903 | 0.909 |
| | pancreatic ctrl | | | | | | |
| 82 | PDCA vs non- | low CA19-9 | 0.734 | 0.658 | 0.691 | 0.680 | 0.642 |
| | pancreatic ctrl | | | | | | |
| 82 | PDCA vs non- | resectable | 0.883 | 0.877 | 0.880 | 0.893 | 0.902 |
| | pancreatic ctrl | | | | | | |
| 83 | PDCA vs (CP + | all | 0.895 | 0.883 | 0.889 | 0.902 | 0.912 |
| | non-pancreatic ctrl) | | | | | | |
| 83 | PDCA vs (CP + | low CA19-9 | 0.723 | 0.635 | 0.673 | 0.674 | 0.657 |
| | non-pancreatic ctrl) | | | | | | |
| 83 | PDCA vs (CP + | resectable | 0.880 | 0.866 | 0.873 | 0.888 | 0.901 |
| | non-pancreatic ctrl) | | | | | | |
| 83 | PDCA vs CP | all | 0.917 | 0.883 | 0.900 | 0.912 | 0.921 |
| 83 | PDCA vs CP | low CA19-9 | 0.826 | 0.695 | 0.754 | 0.759 | 0.748 |
| 83 | PDCA vs CP | resectable | 0.906 | 0.868 | 0.887 | 0.900 | 0.911 |
| 83 | PDCA vs non- | all | 0.861 | 0.882 | 0.872 | 0.887 | 0.900 |
| | pancreatic ctrl | | | | | | |
| 83 | PDCA vs non- | low CA19-9 | 0.642 | 0.620 | 0.630 | 0.627 | 0.602 |
| | pancreatic ctrl | | | | | | |
| 83 | PDCA vs non- | resectable | 0.840 | 0.866 | 0.853 | 0.872 | 0.888 |
| | pancreatic ctrl | | | | | | |
| 84 | PDCA vs (CP + | all | 0.889 | 0.879 | 0.884 | 0.894 | 0.900 |
| | non-pancreatic ctrl) | | | | | | |
| 84 | PDCA vs (CP + | low CA19-9 | 0.720 | 0.637 | 0.674 | 0.669 | 0.641 |
| | non-pancreatic ctrl) | | | | | | |
| 84 | PDCA vs (CP + | resectable | 0.874 | 0.861 | 0.868 | 0.879 | 0.886 |
| | non-pancreatic ctrl) | | | | | | |
| 84 | PDCA vs CP | all | 0.906 | 0.881 | 0.894 | 0.899 | 0.900 |
| 84 | PDCA vs CP | low CA19-9 | 0.819 | 0.709 | 0.759 | 0.749 | 0.714 |
| 84 | PDCA vs CP | resectable | 0.896 | 0.869 | 0.882 | 0.888 | 0.890 |
| 84 | PDCA vs non- | all | 0.872 | 0.884 | 0.878 | 0.891 | 0.902 |
| | pancreatic ctrl | | | | | | |
| 84 | PDCA vs non- | low CA19-9 | 0.676 | 0.632 | 0.651 | 0.645 | 0.618 |
| | pancreatic ctrl | | | | | | |
| 84 | PDCA vs non- | resectable | 0.854 | 0.868 | 0.861 | 0.877 | 0.891 |
| | pancreatic ctrl | | | | | | |
| 85 | PDCA vs (CP + | all | 0.891 | 0.882 | 0.887 | 0.894 | 0.893 |
| | non-pancreatic ctrl) | | | | | | |
| 85 | PDCA vs (CP + | low CA19-9 | 0.728 | 0.651 | 0.685 | 0.669 | 0.621 |
| | non-pancreatic ctrl) | | | | | | |
| 85 | PDCA vs (CP + | resectable | 0.882 | 0.872 | 0.877 | 0.885 | 0.885 |
| | non-pancreatic ctrl) | | | | | | |
| 85 | PDCA vs CP | all | 0.904 | 0.882 | 0.893 | 0.902 | 0.902 |
| 85 | PDCA vs CP | low CA19-9 | 0.819 | 0.715 | 0.762 | 0.758 | 0.719 |
| 85 | PDCA vs CP | resectable | 0.899 | 0.874 | 0.887 | 0.897 | 0.897 |
| 85 | PDCA vs non- | all | 0.875 | 0.883 | 0.879 | 0.890 | 0.898 |
| | pancreatic ctrl | | | | | | |
| 85 | PDCA vs non- | low CA19-9 | 0.680 | 0.634 | 0.654 | 0.641 | 0.600 |
| | pancreatic ctrl | | | | | | |
| 85 | PDCA vs non- | resectable | 0.861 | 0.870 | 0.866 | 0.879 | 0.890 |
| | pancreatic ctrl | | | | | | |
| 86 | PDCA vs (CP + | all | 0.896 | 0.884 | 0.890 | 0.901 | 0.908 |
| | non-pancreatic ctrl) | | | | | | |
| 86 | PDCA vs (CP + | low CA19-9 | 0.729 | 0.639 | 0.679 | 0.675 | 0.644 |
| | non-pancreatic ctrl) | | | | | | |
| 86 | PDCA vs (CP + | resectable | 0.883 | 0.870 | 0.876 | 0.889 | 0.898 |
| | non-pancreatic ctrl) | | | | | | |
| 86 | PDCA vs CP | all | 0.918 | 0.886 | 0.902 | 0.917 | 0.930 |
| 86 | PDCA vs CP | low CA19-9 | 0.833 | 0.709 | 0.765 | 0.780 | 0.774 |
| 86 | PDCA vs CP | resectable | 0.907 | 0.871 | 0.889 | 0.906 | 0.920 |
| 86 | PDCA vs non- | all | 0.863 | 0.879 | 0.871 | 0.885 | 0.897 |
| | pancreatic ctrl | | | | | | |
| 86 | PDCA vs non- | low CA19-9 | 0.648 | 0.621 | 0.633 | 0.625 | 0.591 |
| | pancreatic ctrl | | | | | | |
| 86 | PDCA vs non- | resectable | 0.845 | 0.864 | 0.855 | 0.872 | 0.887 |
| | pancreatic ctrl | | | | | | |
| 87 | PDCA vs (CP + | all | 0.890 | 0.879 | 0.885 | 0.894 | 0.900 |
| | non-pancreatic ctrl) | | | | | | |
| 87 | PDCA vs (CP + | low CA19-9 | 0.726 | 0.643 | 0.680 | 0.673 | 0.639 |
| | non-pancreatic ctrl) | | | | | | |
| 87 | PDCA vs (CP + | resectable | 0.877 | 0.865 | 0.871 | 0.882 | 0.889 |
| | non-pancreatic ctrl) | | | | | | |
| 87 | PDCA vs CP | all | 0.905 | 0.883 | 0.894 | 0.906 | 0.913 |
| 87 | PDCA vs CP | low CA19-9 | 0.819 | 0.718 | 0.764 | 0.771 | 0.749 |
| 87 | PDCA vs CP | resectable | 0.897 | 0.872 | 0.884 | 0.898 | 0.906 |
| 87 | PDCA vs non- | all | 0.874 | 0.881 | 0.877 | 0.889 | 0.899 |
| | pancreatic ctrl | | | | | | |
| 87 | PDCA vs non- | low CA19-9 | 0.682 | 0.633 | 0.654 | 0.644 | 0.608 |
| | pancreatic ctrl | | | | | | |
| 87 | PDCA vs non- | resectable | 0.859 | 0.867 | 0.863 | 0.878 | 0.890 |
| | pancreatic ctrl | | | | | | |
| 88 | PDCA vs (CP + | all | 0.894 | 0.876 | 0.885 | 0.895 | 0.898 |
| | non-pancreatic ctrl) | | | | | | |
| 88 | PDCA vs (CP + | low CA19-9 | 0.741 | 0.656 | 0.694 | 0.687 | 0.656 |
| | non-pancreatic ctrl) | | | | | | |
| 88 | PDCA vs (CP + | resectable | 0.878 | 0.858 | 0.868 | 0.879 | 0.883 |
| | non-pancreatic ctrl) | | | | | | |
| 88 | PDCA vs CP | all | 0.912 | 0.877 | 0.895 | 0.906 | 0.911 |
| 88 | PDCA vs CP | low CA19-9 | 0.847 | 0.733 | 0.784 | 0.788 | 0.767 |
| 88 | PDCA vs CP | resectable | 0.902 | 0.863 | 0.883 | 0.896 | 0.901 |
| 88 | PDCA vs non- | all | 0.882 | 0.886 | 0.884 | 0.896 | 0.905 |
| | pancreatic ctrl | | | | | | |
| 88 | PDCA vs non- | low CA19-9 | 0.700 | 0.642 | 0.667 | 0.657 | 0.625 |
| | pancreatic ctrl | | | | | | |
| 88 | PDCA vs non- | resectable | 0.865 | 0.870 | 0.868 | 0.882 | 0.893 |
| | pancreatic ctrl | | | | | | |
| 89 | PDCA vs (CP + | all | 0.903 | 0.880 | 0.892 | 0.905 | 0.914 |
| | non-pancreatic ctrl) | | | | | | |
| 89 | PDCA vs (CP + | low CA19-9 | 0.757 | 0.656 | 0.701 | 0.703 | 0.685 |
| | non-pancreatic ctrl) | | | | | | |
| 89 | PDCA vs (CP + | resectable | 0.884 | 0.858 | 0.871 | 0.886 | 0.897 |
| | non-pancreatic ctrl) | | | | | | |
| 89 | PDCA vs CP | all | 0.928 | 0.883 | 0.906 | 0.922 | 0.937 |
| 89 | PDCA vs CP | low CA19-9 | 0.868 | 0.737 | 0.796 | 0.813 | 0.817 |
| 89 | PDCA vs CP | resectable | 0.916 | 0.865 | 0.891 | 0.909 | 0.925 |
| 89 | PDCA vs non- | all | 0.877 | 0.885 | 0.881 | 0.895 | 0.907 |
| | pancreatic ctrl | | | | | | |
| 89 | PDCA vs non- | low CA19-9 | 0.687 | 0.637 | 0.659 | 0.652 | 0.627 |
| | pancreatic ctrl | | | | | | |
| 89 | PDCA vs non- | resectable | 0.857 | 0.867 | 0.862 | 0.878 | 0.893 |
| | pancreatic ctrl | | | | | | |
| 90 | PDCA vs (CP + | all | 0.897 | 0.877 | 0.887 | 0.899 | 0.907 |
| | non-pancreatic ctrl) | | | | | | |
| 90 | PDCA vs (CP + | low CA19-9 | 0.754 | 0.659 | 0.701 | 0.700 | 0.679 |
| | non-pancreatic ctrl) | | | | | | |
| 90 | PDCA vs (CP + | resectable | 0.879 | 0.855 | 0.867 | 0.880 | 0.889 |
| | non-pancreatic ctrl) | | | | | | |
| 90 | PDCA vs CP | all | 0.917 | 0.880 | 0.899 | 0.912 | 0.923 |
| 90 | PDCA vs CP | low CA19-9 | 0.859 | 0.747 | 0.797 | 0.808 | 0.800 |
| 90 | PDCA vs CP | resectable | 0.906 | 0.865 | 0.886 | 0.900 | 0.912 |
| 90 | PDCA vs non- | all | 0.884 | 0.885 | 0.885 | 0.897 | 0.906 |
| | pancreatic ctrl | | | | | | |
| 90 | PDCA vs non- | low CA19-9 | 0.712 | 0.645 | 0.674 | 0.665 | 0.635 |
| | pancreatic ctrl | | | | | | |
| 90 | PDCA vs non- | resectable | 0.867 | 0.867 | 0.867 | 0.882 | 0.893 |
| | pancreatic ctrl | | | | | | |
| 91 | PDCA vs (CP + | all | 0.886 | 0.878 | 0.882 | 0.888 | 0.889 |
| | non-pancreatic ctrl) | | | | | | |
| 91 | PDCA vs (CP + | low CA19-9 | 0.704 | 0.631 | 0.663 | 0.649 | 0.608 |
| | non-pancreatic ctrl) | | | | | | |
| 91 | PDCA vs (CP + | resectable | 0.872 | 0.863 | 0.867 | 0.875 | 0.876 |
| | non-pancreatic ctrl) | | | | | | |
| 91 | PDCA vs CP | all | 0.902 | 0.879 | 0.891 | 0.891 | 0.884 |
| 91 | PDCA vs CP | low CA19-9 | 0.810 | 0.698 | 0.749 | 0.721 | 0.666 |
| 91 | PDCA vs CP | resectable | 0.893 | 0.867 | 0.880 | 0.881 | 0.873 |
| 91 | PDCA vs non- | all | 0.869 | 0.884 | 0.877 | 0.890 | 0.899 |
| | pancreatic ctrl | | | | | | |
| 91 | PDCA vs non- | low CA19-9 | 0.662 | 0.627 | 0.642 | 0.635 | 0.605 |
| | pancreatic ctrl | | | | | | |
| 91 | PDCA vs non- | resectable | 0.851 | 0.870 | 0.861 | 0.877 | 0.889 |
| | pancreatic ctrl | | | | | | |
| 92 | PDCA vs (CP + | all | 0.902 | 0.880 | 0.891 | 0.900 | 0.901 |
| | non-pancreatic ctrl) | | | | | | |
| 92 | PDCA vs (CP + | low CA19-9 | 0.762 | 0.675 | 0.714 | 0.704 | 0.670 |
| | non-pancreatic ctrl) | | | | | | |
| 92 | PDCA vs (CP + | resectable | 0.886 | 0.860 | 0.873 | 0.882 | 0.884 |
| | non-pancreatic ctrl) | | | | | | |
| 92 | PDCA vs CP | all | 0.918 | 0.875 | 0.897 | 0.908 | 0.911 |
| 92 | PDCA vs CP | low CA19-9 | 0.865 | 0.757 | 0.806 | 0.808 | 0.788 |
| 92 | PDCA vs CP | resectable | 0.908 | 0.860 | 0.885 | 0.896 | 0.900 |
| 92 | PDCA vs non- | all | 0.885 | 0.884 | 0.885 | 0.896 | 0.903 |
| | pancreatic ctrl | | | | | | |
| 92 | PDCA vs non- | low CA19-9 | 0.717 | 0.651 | 0.679 | 0.667 | 0.631 |
| | pancreatic ctrl | | | | | | |
| 92 | PDCA vs non- | resectable | 0.872 | 0.870 | 0.871 | 0.885 | 0.894 |
| | pancreatic ctrl | | | | | | |
| 93 | PDCA vs (CP + | all | 0.894 | 0.881 | 0.888 | 0.891 | 0.887 |
| | non-pancreatic ctrl) | | | | | | |
| 93 | PDCA vs (CP + | low CA19-9 | 0.731 | 0.654 | 0.688 | 0.665 | 0.611 |
| | non-pancreatic ctrl) | | | | | | |
| 93 | PDCA vs (CP + | resectable | 0.881 | 0.865 | 0.873 | 0.878 | 0.872 |
| | non-pancreatic ctrl) | | | | | | |
| 93 | PDCA vs CP | all | 0.911 | 0.879 | 0.895 | 0.893 | 0.881 |
| 93 | PDCA vs CP | low CA19-9 | 0.831 | 0.723 | 0.772 | 0.739 | 0.672 |
| 93 | PDCA vs CP | resectable | 0.907 | 0.871 | 0.889 | 0.887 | 0.874 |
| 93 | PDCA vs non- | all | 0.874 | 0.883 | 0.879 | 0.891 | 0.898 |
| | pancreatic ctrl | | | | | | |
| 93 | PDCA vs non- | low CA19-9 | 0.682 | 0.639 | 0.658 | 0.647 | 0.609 |
| | pancreatic ctrl | | | | | | |
| 93 | PDCA vs non- | resectable | 0.860 | 0.870 | 0.865 | 0.880 | 0.890 |
| | pancreatic ctrl | | | | | | |
| 94 | PDCA vs (CP + | all | 0.900 | 0.885 | 0.893 | 0.899 | 0.896 |
| | non-pancreatic ctrl) | | | | | | |
| 94 | PDCA vs (CP + | low CA19-9 | 0.747 | 0.668 | 0.704 | 0.686 | 0.634 |
| | non-pancreatic ctrl) | | | | | | |
| 94 | PDCA vs (CP + | resectable | 0.889 | 0.872 | 0.881 | 0.888 | 0.885 |
| | non-pancreatic ctrl) | | | | | | |
| 94 | PDCA vs CP | all | 0.912 | 0.883 | 0.897 | 0.906 | 0.908 |
| 94 | PDCA vs CP | low CA19-9 | 0.828 | 0.734 | 0.777 | 0.774 | 0.740 |
| 94 | PDCA vs CP | resectable | 0.906 | 0.874 | 0.890 | 0.900 | 0.902 |
| 94 | PDCA vs non- | all | 0.878 | 0.882 | 0.880 | 0.891 | 0.896 |
| | pancreatic ctrl | | | | | | |
| 94 | PDCA vs non- | low CA19-9 | 0.694 | 0.643 | 0.665 | 0.650 | 0.604 |
| | pancreatic ctrl | | | | | | |
| 94 | PDCA vs non- | resectable | 0.869 | 0.872 | 0.871 | 0.884 | 0.892 |
| | pancreatic ctrl | | | | | | |
| 95 | PDCA vs (CP + | all | 0.886 | 0.875 | 0.881 | 0.889 | 0.892 |
| | non-pancreatic ctrl) | | | | | | |
| 95 | PDCA vs (CP + | low CA19-9 | 0.727 | 0.655 | 0.687 | 0.675 | 0.644 |
| | non-pancreatic ctrl) | | | | | | |
| 95 | PDCA vs (CP + | resectable | 0.867 | 0.854 | 0.861 | 0.870 | 0.874 |
| | non-pancreatic ctrl) | | | | | | |
| 95 | PDCA vs CP | all | 0.901 | 0.870 | 0.886 | 0.896 | 0.900 |
| 95 | PDCA vs CP | low CA19-9 | 0.835 | 0.732 | 0.778 | 0.777 | 0.757 |
| 95 | PDCA vs CP | resectable | 0.888 | 0.853 | 0.871 | 0.882 | 0.887 |
| 95 | PDCA vs non- | all | 0.871 | 0.881 | 0.876 | 0.888 | 0.899 |
| | pancreatic ctrl | | | | | | |
| 95 | PDCA vs non- | low CA19-9 | 0.678 | 0.634 | 0.653 | 0.642 | 0.613 |
| | pancreatic ctrl | | | | | | |
| 95 | PDCA vs non- | resectable | 0.851 | 0.864 | 0.858 | 0.872 | 0.886 |
| | pancreatic ctrl | | | | | | |
| 96 | PDCA vs (CP + | all | 0.900 | 0.882 | 0.891 | 0.903 | 0.913 |
| | non-pancreatic ctrl) | | | | | | |
| 96 | PDCA vs (CP + | low CA19-9 | 0.759 | 0.667 | 0.707 | 0.706 | 0.689 |
| | non-pancreatic ctrl) | | | | | | |
| 96 | PDCA vs (CP + | resectable | 0.878 | 0.857 | 0.867 | 0.881 | 0.893 |
| | non-pancreatic ctrl) | | | | | | |
| 96 | PDCA vs CP | all | 0.916 | 0.876 | 0.896 | 0.912 | 0.926 |
| 96 | PDCA vs CP | low CA19-9 | 0.855 | 0.736 | 0.789 | 0.803 | 0.806 |
| 96 | PDCA vs CP | resectable | 0.899 | 0.854 | 0.877 | 0.894 | 0.909 |
| 96 | PDCA vs non- | all | 0.873 | 0.883 | 0.878 | 0.892 | 0.905 |
| | pancreatic ctrl | | | | | | |
| 96 | PDCA vs non- | low CA19-9 | 0.683 | 0.642 | 0.660 | 0.654 | 0.628 |
| | pancreatic ctrl | | | | | | |
| 96 | PDCA vs non- | resectable | 0.852 | 0.865 | 0.859 | 0.875 | 0.891 |
| | pancreatic ctrl | | | | | | |
| 97 | PDCA vs (CP + | all | 0.892 | 0.876 | 0.884 | 0.894 | 0.902 |
| | non-pancreatic ctrl) | | | | | | |
| 97 | PDCA vs (CP + | low CA19-9 | 0.746 | 0.664 | 0.700 | 0.695 | 0.673 |
| | non-pancreatic ctrl) | | | | | | |
| 97 | PDCA vs (CP + | resectable | 0.871 | 0.853 | 0.862 | 0.874 | 0.883 |
| | non-pancreatic ctrl) | | | | | | |
| 97 | PDCA vs CP | all | 0.905 | 0.874 | 0.890 | 0.902 | 0.913 |
| 97 | PDCA vs CP | low CA19-9 | 0.844 | 0.743 | 0.788 | 0.795 | 0.787 |
| 97 | PDCA vs CP | resectable | 0.890 | 0.855 | 0.873 | 0.886 | 0.898 |
| 97 | PDCA vs non- | all | 0.876 | 0.881 | 0.878 | 0.891 | 0.902 |
| | pancreatic ctrl | | | | | | |
| 97 | PDCA vs non- | low CA19-9 | 0.696 | 0.642 | 0.665 | 0.657 | 0.628 |
| | pancreatic ctrl | | | | | | |
| 97 | PDCA vs non- | resectable | 0.858 | 0.864 | 0.861 | 0.876 | 0.890 |
| | pancreatic ctrl | | | | | | |
| 98 | PDCA vs (CP + | all | 0.881 | 0.877 | 0.879 | 0.882 | 0.880 |
| | non-pancreatic ctrl) | | | | | | |
| 98 | PDCA vs (CP + | low CA19-9 | 0.699 | 0.635 | 0.663 | 0.637 | 0.587 |
| | non-pancreatic ctrl) | | | | | | |
| 98 | PDCA vs (CP + | resectable | 0.865 | 0.860 | 0.863 | 0.867 | 0.864 |
| | non-pancreatic ctrl) | | | | | | |
| 98 | PDCA vs CP | all | 0.897 | 0.874 | 0.885 | 0.880 | 0.868 |
| 98 | PDCA vs CP | low CA19-9 | 0.800 | 0.692 | 0.741 | 0.699 | 0.630 |
| 98 | PDCA vs CP | resectable | 0.887 | 0.860 | 0.874 | 0.868 | 0.854 |
| 98 | PDCA vs non- | all | 0.859 | 0.880 | 0.870 | 0.883 | 0.894 |
| | pancreatic ctrl | | | | | | |
| 98 | PDCA vs non- | low CA19-9 | 0.642 | 0.621 | 0.630 | 0.621 | 0.591 |
| | pancreatic ctrl | | | | | | |
| 98 | PDCA vs non- | resectable | 0.838 | 0.864 | 0.851 | 0.868 | 0.882 |
| | pancreatic ctrl | | | | | | |
| 99 | PDCA vs (CP + | all | 0.893 | 0.885 | 0.889 | 0.900 | 0.909 |
| | non-pancreatic ctrl) | | | | | | |
| 99 | PDCA vs (CP + | low CA19-9 | 0.728 | 0.647 | 0.683 | 0.676 | 0.652 |
| | non-pancreatic ctrl) | | | | | | |
| 99 | PDCA vs (CP + | resectable | 0.874 | 0.865 | 0.870 | 0.882 | 0.893 |
| | non-pancreatic ctrl) | | | | | | |
| 99 | PDCA vs CP | all | 0.911 | 0.880 | 0.896 | 0.903 | 0.909 |
| 99 | PDCA vs CP | low CA19-9 | 0.820 | 0.694 | 0.751 | 0.743 | 0.720 |
| 99 | PDCA vs CP | resectable | 0.897 | 0.860 | 0.879 | 0.887 | 0.893 |
| 99 | PDCA vs non- | all | 0.857 | 0.880 | 0.869 | 0.885 | 0.899 |
| | pancreatic ctrl | | | | | | |
| 99 | PDCA vs non- | low CA19-9 | 0.640 | 0.623 | 0.630 | 0.628 | 0.603 |
| | pancreatic ctrl | | | | | | |
| 99 | PDCA vs non- | resectable | 0.834 | 0.862 | 0.848 | 0.868 | 0.885 |
| | pancreatic ctrl | | | | | | |
| 100 | PDCA vs (CP + | all | 0.886 | 0.878 | 0.882 | 0.889 | 0.892 |
| | non-pancreatic ctrl) | | | | | | |
| 100 | PDCA vs (CP + | low CA19-9 | 0.717 | 0.641 | 0.674 | 0.659 | 0.622 |
| | non-pancreatic ctrl) | | | | | | |
| 100 | PDCA vs (CP + | resectable | 0.868 | 0.858 | 0.863 | 0.871 | 0.875 |
| | non-pancreatic ctrl) | | | | | | |
| 100 | PDCA vs CP | all | 0.900 | 0.876 | 0.888 | 0.889 | 0.886 |
| 100 | PDCA vs CP | low CA19-9 | 0.807 | 0.702 | 0.750 | 0.726 | 0.680 |
| 100 | PDCA vs CP | resectable | 0.888 | 0.860 | 0.874 | 0.875 | 0.872 |
| 100 | PDCA vs non- | all | 0.865 | 0.880 | 0.873 | 0.886 | 0.898 |
| | pancreatic ctrl | | | | | | |
| 100 | PDCA vs non- | low CA19-9 | 0.664 | 0.630 | 0.645 | 0.638 | 0.611 |
| | pancreatic ctrl | | | | | | |
| 100 | PDCA vs non- | resectable | 0.846 | 0.864 | 0.855 | 0.872 | 0.887 |
| | pancreatic ctrl | | | | | | |
| 101 | PDCA vs (CP + | all | 0.889 | 0.882 | 0.886 | 0.891 | 0.890 |
| | non-pancreatic ctrl) | | | | | | |
| 101 | PDCA vs (CP + | low CA19-9 | 0.723 | 0.652 | 0.684 | 0.663 | 0.613 |
| | non-pancreatic ctrl) | | | | | | |
| 101 | PDCA vs (CP + | resectable | 0.876 | 0.868 | 0.873 | 0.879 | 0.878 |
| | non-pancreatic ctrl) | | | | | | |
| 101 | PDCA vs CP | all | 0.896 | 0.876 | 0.886 | 0.893 | 0.891 |
| 101 | PDCA vs CP | low CA19-9 | 0.801 | 0.707 | 0.750 | 0.740 | 0.699 |
| 101 | PDCA vs CP | resectable | 0.889 | 0.866 | 0.878 | 0.885 | 0.884 |
| 101 | PDCA vs non- | all | 0.868 | 0.881 | 0.874 | 0.886 | 0.896 |
| | pancreatic ctrl | | | | | | |
| 101 | PDCA vs non- | low CA19-9 | 0.668 | 0.634 | 0.649 | 0.635 | 0.596 |
| | pancreatic ctrl | | | | | | |
| 101 | PDCA vs non- | resectable | 0.854 | 0.869 | 0.862 | 0.876 | 0.889 |
| | pancreatic ctrl | | | | | | |
| 102 | PDCA vs (CP + | all | 0.896 | 0.886 | 0.891 | 0.902 | 0.909 |
| | non-pancreatic ctrl) | | | | | | |
| 102 | PDCA vs (CP + | low CA19-9 | 0.735 | 0.650 | 0.688 | 0.681 | 0.649 |
| | non-pancreatic ctrl) | | | | | | |
| 102 | PDCA vs (CP + | resectable | 0.879 | 0.868 | 0.874 | 0.886 | 0.895 |
| | non-pancreatic ctrl) | | | | | | |
| 102 | PDCA vs CP | all | 0.911 | 0.881 | 0.896 | 0.910 | 0.921 |
| 102 | PDCA vs CP | low CA19-9 | 0.818 | 0.704 | 0.756 | 0.766 | 0.756 |
| 102 | PDCA vs CP | resectable | 0.896 | 0.862 | 0.879 | 0.895 | 0.907 |
| 102 | PDCA vs non- | all | 0.858 | 0.877 | 0.868 | 0.882 | 0.895 |
| | pancreatic ctrl | | | | | | |
| 102 | PDCA vs non- | low CA19-9 | 0.639 | 0.622 | 0.630 | 0.623 | 0.590 |
| | pancreatic ctrl | | | | | | |
| 102 | PDCA vs non- | resectable | 0.840 | 0.864 | 0.852 | 0.870 | 0.887 |
| | pancreatic ctrl | | | | | | |
| 103 | PDCA vs (CP + | all | 0.889 | 0.882 | 0.886 | 0.894 | 0.899 |
| | non-pancreatic ctrl) | | | | | | |
| 103 | PDCA vs (CP + | low CA19-9 | 0.727 | 0.652 | 0.686 | 0.674 | 0.637 |
| | non-pancreatic ctrl) | | | | | | |
| 103 | PDCA vs (CP + | resectable | 0.874 | 0.865 | 0.870 | 0.880 | 0.886 |
| | non-pancreatic ctrl) | | | | | | |
| 103 | PDCA vs CP | all | 0.899 | 0.879 | 0.889 | 0.899 | 0.906 |
| 103 | PDCA vs CP | low CA19-9 | 0.803 | 0.713 | 0.754 | 0.756 | 0.733 |
| 103 | PDCA vs CP | resectable | 0.887 | 0.865 | 0.876 | 0.888 | 0.896 |
| 103 | PDCA vs non- | all | 0.868 | 0.878 | 0.873 | 0.886 | 0.897 |
| | pancreatic ctrl | | | | | | |
| 103 | PDCA vs non- | low CA19-9 | 0.675 | 0.633 | 0.651 | 0.641 | 0.607 |
| | pancreatic ctrl | | | | | | |
| 103 | PDCA vs non- | resectable | 0.854 | 0.866 | 0.860 | 0.876 | 0.889 |
| | pancreatic ctrl | | | | | | |
| 104 | PDCA vs (CP + | all | 0.902 | 0.879 | 0.891 | 0.899 | 0.900 |
| | non-pancreatic ctrl) | | | | | | |
| 104 | PDCA vs (CP + | low CA19-9 | 0.772 | 0.673 | 0.718 | 0.706 | 0.667 |
| | non-pancreatic ctrl) | | | | | | |
| 104 | PDCA vs (CP + | resectable | 0.883 | 0.856 | 0.870 | 0.879 | 0.879 |
| | non-pancreatic ctrl) | | | | | | |
| 104 | PDCA vs CP | all | 0.922 | 0.873 | 0.898 | 0.894 | 0.883 |
| 104 | PDCA vs CP | low CA19-9 | 0.877 | 0.752 | 0.809 | 0.778 | 0.721 |
| 104 | PDCA vs CP | resectable | 0.909 | 0.854 | 0.882 | 0.877 | 0.863 |
| 104 | PDCA vs non- | all | 0.846 | 0.896 | 0.871 | 0.888 | 0.911 |
| | pancreatic ctrl | | | | | | |
| 104 | PDCA vs non- | low CA19-9 | 0.703 | 0.730 | 0.719 | 0.713 | 0.705 |
| | pancreatic ctrl | | | | | | |
| 104 | PDCA vs non- | resectable | 0.813 | 0.873 | 0.843 | 0.863 | 0.890 |
| | pancreatic ctrl | | | | | | |

PDAC = pancreatic cancer;
CP = chronic pancreatitis;
ctrl = control)
Columns:
A: Sample set for subgroup analysis (“all” refers to the entire data set of pancreatic cancer, chronic pancreatitis, and non-pancreatic control samples)
B: AUC result of pancreatic cancer relative to chronic pancreatitis
C: AUC result of pancreatic cancer relative to non-pancreatic controls
D: AUC result of pancreatic cancer relative to (chronic pancreatitis and non-pancreatic controls)
E: AUC result of pancreatic cancer relative to all non-cancer subjects (chronic pancreatitis, non-pancreatic controls, diabetes group, non-diabetes group)
F: AUC result of pancreatic cancer relative to diabetic subjects (from chronic pancreatitis, non-pancreatic controls, diabetes group)

### EXAMPLE 6: CLASSIFICATION OF PATIENTS USING THE BIOMARKER PANEL

### Log-Transformation and Scaling

Input data:
   Absolute concentration of analytes measured by LC-MS/MS: One value for each analyte and patient sample.
   Absolute concentration of CA19-9 determined by a commercially available radio immunoassay (RIA): One value per patient sample.
Transform all input data by log₁₀. The numerical value for CA19-9 (U/ml) is being treated in the same way as the peak area ratios of the analytes measured by LC-MS/MS.
Scale the log₁₀-transformed input data x by first subtracting an analyte-specific constant mᵢ and then dividing by a analyte-specific constant sᵢ, resulting in log₁₀-transformed and scaled input data \hat{x}:

$${\hat{x}}_{i} = \frac{{x}_{i} - {m}_{i}}{{s}_{i}}$$

For example, scaling parameters for the panel 6 and panel 7 are listed in Tables 12-13.

TABLE 12
Example of scaling parameters of compounds used for the panel 6

| | | | | | Pancreatic | |
|---|---|---|---|---|---|---|
| | | | | | cancer | |
| | | | | | versus | |
| | Pancreatic | | Pancreatic | | (chronic | |
| | cancer | | cancer | | pancreatitis | |
| | versus | | versus non- | | and non- | |
| | chronic | | pancreatic | | pancreatic | |
| | pancreatitis | | control | | control) | |
| Biomarker | mₗ | sₗ | mₗ | sₗ | mₗ | sₗ |
| Proline | 2.096 | 0.147 | 2.094 | 0.150 | 2.112 | 0.15 |
| Ceramide (d18:1,C24:0) | 1.238 | 0.174 | 1.231 | 0.155 | 1.248 | 0.165 |
| Lyso- | 0.611 | 0.265 | 0.597 | 0.257 | 0.633 | 0.26 |
| phosphatidylethanolamine | | | | | | |
| (C18:2) | | | | | | |
| Sphingomyelin (35:1) | 1.376 | 0.179 | 1.435 | 0.135 | 1.388 | 0.163 |
| Sphingomyelin (d18:2,C17:0) | 0.659 | 0.163 | 0.709 | 0.132 | 0.669 | 0.149 |
| CA19-9 | 1.590 | 1.032 | 1.495 | 1.087 | 1.331 | 0.978 |

TABLE 13
Example of scaling parameters of compounds used for the panel 7

| | | | | | Pancreatic cancer | |
|---|---|---|---|---|---|---|
| | Pancreatic | | Pancreatic | | versus (chronic | |
| | cancer versus | | cancer versus | | pancreatitis and | |
| | chronic | | non-pancreatic | | non-pancreatic | |
| | pancreatitis | | control | | control) | |
| Biomarker | mₗ | sₗ | mₗ | sₗ | mₗ | sₗ |
| Proline | 2.096 | 0.147 | 2.094 | 0.150 | 2.112 | 0.150 |
| Tryptophan | 1.767 | 0.140 | 1.775 | 0.136 | 1.781 | 0.136 |
| Ceramide | 1.238 | 0.174 | 1.231 | 0.155 | 1.248 | 0.165 |
| (d18:1,C24:0) | | | | | | |
| Sphingomyelin | 1.376 | 0.179 | 1.435 | 0.135 | 1.388 | 0.163 |
| (35:1) | | | | | | |
| Sphingomyelin | 0.659 | 0.163 | 0.709 | 0.132 | 0.669 | 0.149 |
| (d18:2,C17:0) | | | | | | |
| CA19-9 | 1.590 | 1.032 | 1.495 | 1.087 | 1.331 | 0.978 |

### Calculation of the Prediction Score

The prediction score is calculated for each patient based on the log₁₀-transformed and scaled input data \hat{x} using analyte-specific weights ωᵢ and bias ω₀:

$$p = \frac{1}{1 + {e}^{- ( {ω}_{0} + {Σ}_{i}^{n} {ω}_{i} {\hat{x}}_{i} )}}$$

For example, weights parameters for the biomarker panels 6 and 7 are listed in Tables 14-15.

TABLE 14
Example of weight parameters of compounds used
for the biomarker panel 6 and bias

| | | Pancreatic | |
|---|---|---|---|
| | Pancreatic | cancer | Pancreatic cancer |
| | cancer versus | versus non- | versus (chronic |
| | chronic | pancreatic | pancreatitis and non- |
| | pancreatitis; | control; Bias | pancreatic control); |
| | Bias (ω₀) = −0.017 | (ω₀) = 0.020 | Bias (ω₀) = −0.929 |
| Biomarker | ωₗ | ωₗ | ωₗ |
| Proline | −0.190 | −0.297 | −0.244 |
| Ceramide (d18:1, C24:0) | −0.320 | −0.194 | −0.249 |
| Lysophosphatidylethanolamine | −0.304 | −0.124 | −0.199 |
| (C18:2) | | | |
| Sphingomyelin (35:1) | 0.566 | 0.191 | 0.380 |
| Sphingomyelin (d18:2, C17:0) | 0.264 | 0.000 | 0.123 |
| CA19-9 | 0.910 | 1.143 | 1.079 |

TABLE 15
Example of weight parameters of compounds used
for the biomarker panel 7 and bias

| | Pancreatic | Pancreatic | |
|---|---|---|---|
| | cancer | cancer | Pancreatic cancer |
| | versus | versus non- | versus (chronic |
| | chronic | pancreatic | pancreatitis and |
| | pancreatitis; | control; | non-pancreatic |
| | Bias (ω₀) = | Bias (ω₀) = | control); |
| | −0.009 | 0.021 | Bias (ω₀) = −0.927 |
| Biomarker | ωₗ | ωₗ | ωₗ |
| Proline | −0.206 | −0.318 | −0.272 |
| Tryptophan | −0.243 | −0.131 | −0.131 |
| Ceramide | −0.391 | −0.218 | −0.282 |
| (d18:1, C24:0) | | | |
| Sphingomyelin (35:1) | 0.597 | 0.200 | 0.371 |
| Sphingomyelin | 0.315 | 0.000 | 0.140 |
| (d18:2, C17:0) | | | |
| CA19-9 | 0.971 | 1.198 | 1.094 |

The prediction score can be interpreted as the score that the patient is suffering from PDAC assuming a prevalence of the disease as in the analyzed data set. By adaptation of the bias accordingly, the prediction score can be applied to the targeted patient population with the apparent prevalence. Calculated prediction scores can take on any value between 0 and 1. By comparing the prediction score with a pre-defined cutoff a patient can be classified.

### Determination of the Cutoff

Cutoff values were determined by using two different methods. First, a cutoff was determined at a fixed specificity of 85%. An alternative cutoff was determined with the Youden index method optimizing the accuracy. Both methods were applied on the entire data set or on males or females only, respectively. For example, the cutoff values for biomarker panels 6 and 7 are listed in Table 16.

TABLE 16
Cutoffs for the prediction score allowing a classification
for biomarker panel 6 and biomarker panel 7

| | | Females and | | | | | |
|---|---|---|---|---|---|---|---|
| | | males | | Females | | Males | |
| | | | Spec | | Spec | | Spec |
| Task | Panel | Youden | 85% | Youden | 85% | Youden | 85% |
| Pancreatic | 7 | 0.362 | 0.332 | 0.377 | 0.418 | 0.362 | 0.292 |
| cancer versus | | | | | | | |
| (chronic | | | | | | | |
| pancreatitis and | | | | | | | |
| non-pancreatic | | | | | | | |
| control) | | | | | | | |
| Pancreatic | 7 | 0.568 | 0.502 | 0.568 | 0.742 | 0.466 | 0.466 |
| cancer versus | | | | | | | |
| chronic | | | | | | | |
| pancreatitis | | | | | | | |
| Pancreatic | 7 | 0.510 | 0.455 | 0.510 | 0.503 | 0.445 | 0.405 |
| cancer versus | | | | | | | |
| non-pancreatic | | | | | | | |
| control | | | | | | | |
| Pancreatic | 6 | 0.391 | 0.337 | 0.407 | 0.416 | 0.391 | 0.278 |
| cancer versus | | | | | | | |
| (chronic | | | | | | | |
| pancreatitis and | | | | | | | |
| non-pancreatic | | | | | | | |
| control) | | | | | | | |
| Pancreatic | 6 | 0.502 | 0.502 | 0.611 | 0.714 | 0.502 | 0.424 |
| cancer versus | | | | | | | |
| chronic | | | | | | | |
| pancreatitis | | | | | | | |
| Pancreatic | 6 | 0.523 | 0.457 | 0.581 | 0.503 | 0.425 | 0.423 |
| cancer versus | | | | | | | |
| non-pancreatic | | | | | | | |
| control | | | | | | | |

### Patient/Sample Classification

A positive or negative diagnostic outcome is obtained from comparison of the result (or prediction score) obtained for a sample with the cutoff value. A prediction score greater than or equal to the cutoff value is taken as positive diagnostic outcome, a prediction score smaller than the cutoff value is taken as negative diagnostic outcome.

### EXAMPLE 7: FURTHER BIOMARKER PANELS

Two further biomarker panels (Panel no. 105 and 106) were generated by treating the sum of all representatives of one ontology class together as one feature for the logistic regression model, respectively.

More precisely, the sum of histidine, proline, and tryptophan constitutes one feature, the sum of sphingomyelin (d17:1,C16:0), sphingomyelin (d18:2,C17:0), sphingomyelin (35:1) and sphingomyelin (41:2) constitutes one feature, and the sum of ceramide (d18:1,C24:0), ceramide (d18:2,C24:0) constitutes one feature in both, Panel 105 and Panel 106.

Additionally, the ratio of lysophosphatidylethanolamine (C18:2) to phosphatidylethanolamine (C18:0,C22:6) was used as a feature in Panel 106.

Panels 105 and 106 are constituted as shown in Table 17, below:

TABLE 17

| Panel | |
|---|---|
| Number | Panel Composition |
| 105 | CA 19-9, [histidine + proline + tryptophan], [sphingomyelin |
| | (d17:1, C16:0) + sphingomyelin (d18:2, C17:0) + sphingomyelin |
| | (35:1) + sphingomyelin (41:2)], [ceramide (d18:1, C24:0) + |
| | ceramide (d18:2, C24:0)] |
| 106 | CA 19-9, [histidine + proline + tryptophan], [sphingomyelin |
| | (d17:1, C16:0) + sphingomyelin (d18:2, C17:0) + sphingomyelin |
| | (35:1) + sphingomyelin (41:2)], [ceramide (d18:1, C24:0) + |
| | ceramide (d18:2, C24:0)], [lysophosphatidylethanolamine |
| | (C18:2)/phosphatidylethanolamine (C18:0, C22:6)] |

Performance/classification results for both of the panels are shown in Table 18, below.

TABLE 18
Subgroup performance of the diagnostic biomarker panels shown in Table 17,
including diabetes and resectable pancreatic cancer (PDAC = pancreatic cancer; CP =
chronic pancreatitis; ctrl = control)

| Panel | | | | | | | | | |
|---|---|---|---|---|---|---|---|---|---|
| Number | Training of panel on | A | Cutoff | B | C | D | E | F | G |
| 105 | PDAC vs. CP | all | 0.4901 | 0.914 | 0.915 | 0.82 | 0.81 | 0.83 | 0.89 |
| 105 | PDAC vs. Healthy | all | 0.4965 | 0.865 | 0.914 | 0.79 | 0.81 | 0.81 | 0.89 |
| 105 | PDAC vs. (CP + non-pancreatic ctrl) | all | 0.3095 | 0.900 | 0.919 | 0.82 | 0.81 | 0.83 | 0.89 |
| 105 | PDAC resectable vs. CP | all | 0.3582 | 0.904 | 0.918 | 0.81 | 0.81 | 0.86 | 0.83 |
| 105 | PDAC resectable vs. Healthy | all | 0.3786 | 0.852 | 0.904 | 0.82 | 0.81 | 0.86 | 0.89 |
| 105 | PDAC resectable vs. (CP + non- | all | 0.2065 | 0.890 | 0.923 | 0.84 | 0.81 | 0.86 | 0.83 |
| | pancreatic ctrl) | | | | | | | | |
| 106 | PDAC vs. CP | all | 0.4337 | 0.924 | 0.914 | 0.90 | 0.81 | 0.94 | 0.72 |
| 106 | PDAC vs. Healthy | all | 0.4794 | 0.869 | 0.910 | 0.81 | 0.81 | 0.83 | 0.89 |
| 106 | PDAC vs. (CP + non-pancreatic ctrl) | all | 0.2712 | 0.912 | 0.927 | 0.91 | 0.81 | 0.92 | 0.83 |
| 106 | PDAC resectable vs. CP | all | 0.3305 | 0.918 | 0.920 | 0.91 | 0.81 | 0.94 | 0.83 |
| 106 | PDAC resectable vs. Healthy | all | 0.3807 | 0.860 | 0.892 | 0.81 | 0.81 | 0.83 | 0.89 |
| 106 | PDAC resectable vs. (CP + non- | all | 0.1917 | 0.890 | 0.926 | 0.88 | 0.81 | 0.89 | 0.89 |
| | pancreatic ctrl) | | | | | | | | |
| 105 | PDAC vs. CP | resectable | 0.4901 | 0.898 | 0.904 | 0.80 | 0.81 | 0.78 | 0.89 |
| 105 | PDAC vs. Healthy | resectable | 0.4965 | 0.832 | 0.895 | 0.73 | 0.81 | 0.72 | 0.89 |
| 105 | PDAC vs. (CP + non-pancreatic ctrl) | resectable | 0.3095 | 0.886 | 0.914 | 0.78 | 0.81 | 0.78 | 0.89 |
| 105 | PDAC resectable vs. CP | resectable | 0.3582 | 0.890 | 0.920 | 0.80 | 0.81 | 0.83 | 0.83 |
| 105 | PDAC resectable vs. Healthy | resectable | 0.3786 | 0.823 | 0.898 | 0.80 | 0.81 | 0.83 | 0.89 |
| 105 | PDAC resectable vs. (CP + non- | resectable | 0.2065 | 0.878 | 0.924 | 0.83 | 0.81 | 0.83 | 0.83 |
| | pancreatic ctrl) | | | | | | | | |
| 106 | PDAC vs. CP | resectable | 0.4337 | 0.912 | 0.900 | 0.90 | 0.81 | 0.94 | 0.72 |
| 106 | PDAC vs. Healthy | resectable | 0.4794 | 0.840 | 0.889 | 0.78 | 0.81 | 0.78 | 0.89 |
| 106 | PDAC vs. (CP + non-pancreatic ctrl) | resectable | 0.2712 | 0.898 | 0.914 | 0.90 | 0.81 | 0.94 | 0.83 |
| 106 | PDAC resectable vs. CP | resectable | 0.3305 | 0.897 | 0.902 | 0.90 | 0.81 | 0.94 | 0.83 |
| 106 | PDAC resectable vs. Healthy | resectable | 0.3807 | 0.822 | 0.868 | 0.78 | 0.81 | 0.78 | 0.89 |
| 106 | PDAC resectable vs. (CP + non- | resectable | 0.1917 | 0.876 | 0.922 | 0.88 | 0.81 | 0.89 | 0.89 |
| | pancreatic ctrl) | | | | | | | | |

A: Sample set for subgroup analysis (“all” refers to the entire data set of pancreatic cancer, chronic pancreatitis, and non-pancreatic control samples)
B: AUC result of pancreatic cancer (PDAC) versus chronic pancreatitis
C: AUC result of PDAC versus diabetic controls
D: Sensitivity of PDAC versus chronic pancreatitis
E: Specifity of PDAC versus chronic pancreatitis
F: Sensitivity of PDAC versus diabetic controls
G: Specifity of PDAC versus diabetic controls

### EXAMPLE 8: REFINEMENT OF CLASSIFICATION OF PATIENTS USING THE BIOMARKER PANEL/ADAPTATION OF ALGORITHM

Five to seven percent of the population do not produce CA19-9 due to specific, inherited Lewis a/b antigen negativity (either homo-, or heterozygous). For these patients, CA19-9 always produces a negative test result regardless of whether the patient has cancer or not. The metabolic markers do not have the same problem. To address this issue, it was decided to train both, a logistic regression model including our metabolites and CA19-9, and an additional model using only the respective metabolites (without CA19-9). When optimizing the algorithm for a given panel to obtain a certain specificity, this approach also yields different cut-offs for the two models.

Accordingly, the following rule was used for patient classification: If the patient's CA19-9 value is above a certain threshold, the model including CA19-9 with the respective cutoff was used; otherwise, the model without CA19-9 with its specific cutoff was used. Since the two prediction scores generated by the two models are not directly comparable, calculation of a meaningful AUC would be possible by first aligning the scores in some way. However, calculation of Sensitivity and Specificity is directly possible, and is also an appropriate measure for the application in this case.

In our case the threshold used was a CA19-9 value of 2 U/ml.

The approach can be further refined by first training the model including CA19-9 only on patients which have a CA19-9 measurement above the threshold.

TABLE 19
Effect of using two different, CA 19-9-dependent models (“two-model
algorithm”) per panel instead of just one on sensititvity and specificity; the positive groups
are shown in the “Training”-column, respectively (i.e. PDAC or resectable PDAC):

| Panel | | | | | | | |
|---|---|---|---|---|---|---|---|
| Number | Training of Panel on | A | B | C | D | E | F |
| 1 | PDAC vs. CP | 88.3% | 93.5% | 79.7% | 75.9% | 81.0% | 72.2% |
| 2 | PDAC vs. CP | 89.6% | 94.8% | 79.7% | 75.9% | 75.9% | 63.3% |
| 3 | PDAC vs. CP | 88.3% | 93.5% | 82.3% | 77.2% | 77.2% | 68.4% |
| 4 | PDAC vs. CP | 88.3% | 93.5% | 79.7% | 74.7% | 78.5% | 68.4% |
| 5 | PDAC vs. CP | 88.3% | 93.5% | 82.3% | 77.2% | 77.2% | 68.4% |
| 6 | PDAC vs. CP | 88.3% | 93.5% | 81.0% | 75.9% | 74.7% | 65.8% |
| 7 | PDAC vs. CP | 88.3% | 92.2% | 82.3% | 78.5% | 78.5% | 69.6% |
| 8 | PDAC vs. CP | 89.6% | 94.8% | 79.7% | 75.9% | 78.5% | 70.9% |
| 9 | PDAC vs. CP | 90.9% | 96.1% | 79.7% | 75.9% | 70.9% | 59.5% |
| 10 | PDAC vs. CP | 90.9% | 94.8% | 81.0% | 77.2% | 73.4% | 63.3% |
| 11 | PDAC vs. CP | 90.9% | 96.1% | 82.3% | 78.5% | 72.2% | 62.0% |
| 12 | PDAC vs. CP | 87.0% | 92.2% | 83.5% | 78.5% | 83.5% | 75.9% |
| 13 | PDAC vs. CP | 89.6% | 94.8% | 81.0% | 77.2% | 78.5% | 65.8% |
| 14 | PDAC vs. CP | 87.0% | 92.2% | 81.0% | 77.2% | 81.0% | 73.4% |
| 15 | PDAC vs. CP | 89.6% | 94.8% | 77.2% | 73.4% | 74.7% | 64.6% |
| 16 | PDAC vs. CP | 87.0% | 90.9% | 82.3% | 78.5% | 81.0% | 72.2% |
| 17 | PDAC vs. CP | 84.4% | 89.6% | 81.0% | 77.2% | 79.7% | 69.6% |
| 18 | PDAC vs. CP | 87.0% | 90.9% | 77.2% | 73.4% | 77.2% | 68.4% |
| 19 | PDAC vs. CP | 84.4% | 88.3% | 82.3% | 78.5% | 84.8% | 78.5% |
| 20 | PDAC vs. CP | 83.1% | 88.3% | 79.7% | 74.7% | 82.3% | 72.2% |
| 21 | PDAC vs. CP | 84.4% | 88.3% | 78.5% | 74.7% | 84.8% | 78.5% |
| 22 | PDAC vs. CP | 87.0% | 90.9% | 78.5% | 75.9% | 77.2% | 68.4% |
| 23 | PDAC vs. CP | 85.7% | 89.6% | 79.7% | 77.2% | 78.5% | 69.6% |
| 24 | PDAC vs. CP | 87.0% | 89.6% | 74.7% | 72.2% | 79.7% | 69.6% |
| 25 | PDAC vs. CP | 87.0% | 90.9% | 74.7% | 72.2% | 78.5% | 68.4% |
| 26 | PDAC vs. CP | 87.0% | 92.2% | 78.5% | 74.7% | 82.3% | 74.7% |
| 27 | PDAC vs. CP | 85.7% | 89.6% | 77.2% | 74.7% | 79.7% | 72.2% |
| 28 | PDAC vs. CP | 84.4% | 89.6% | 75.9% | 74.7% | 83.5% | 75.9% |
| 29 | PDAC vs. CP | 84.4% | 88.3% | 75.9% | 73.4% | 83.5% | 77.2% |
| 30 | PDAC vs. CP | 87.0% | 90.9% | 82.3% | 78.5% | 75.9% | 67.1% |
| 31 | PDAC vs. CP | 88.3% | 90.9% | 78.5% | 75.9% | 77.2% | 69.6% |
| 32 | PDAC vs. CP | 88.3% | 90.9% | 81.0% | 75.9% | 73.4% | 64.6% |
| 33 | PDAC vs. CP | 89.6% | 93.5% | 81.0% | 77.2% | 74.7% | 65.8% |
| 34 | PDAC vs. CP | 87.0% | 92.2% | 79.7% | 75.9% | 78.5% | 67.1% |
| 35 | PDAC vs. CP | 84.4% | 88.3% | 78.5% | 74.7% | 81.0% | 70.9% |
| 36 | PDAC vs. CP | 85.7% | 88.3% | 83.5% | 79.7% | 82.3% | 73.4% |
| 37 | PDAC vs. CP | 88.3% | 92.2% | 81.0% | 78.5% | 81.0% | 74.7% |
| 38 | PDAC vs. CP | 88.3% | 93.5% | 83.5% | 79.7% | 74.7% | 65.8% |
| 39 | PDAC vs. CP | 89.6% | 94.8% | 78.5% | 75.9% | 73.4% | 63.3% |
| 40 | PDAC vs. CP | 88.3% | 93.5% | 83.5% | 79.7% | 77.2% | 69.6% |
| 41 | PDAC vs. CP | 85.7% | 89.6% | 82.3% | 77.2% | 81.0% | 70.9% |
| 42 | PDAC vs. CP | 85.7% | 90.9% | 81.0% | 77.2% | 75.9% | 65.8% |
| 43 | PDAC vs. CP | 90.9% | 93.5% | 86.1% | 83.5% | 78.5% | 67.1% |
| 44 | PDAC vs. CP | 90.9% | 93.5% | 79.7% | 77.2% | 77.2% | 65.8% |
| 45 | PDAC vs. CP | 87.0% | 92.2% | 78.5% | 73.4% | 75.9% | 67.1% |
| 46 | PDAC vs. CP | 90.9% | 93.5% | 81.0% | 78.5% | 81.0% | 70.9% |
| 47 | PDAC vs. CP | 90.9% | 93.5% | 81.0% | 77.2% | 77.2% | 65.8% |
| 48 | PDAC vs. CP | 90.9% | 93.5% | 82.3% | 79.7% | 83.5% | 72.2% |
| 49 | PDAC vs. CP | 90.9% | 93.5% | 79.7% | 75.9% | 82.3% | 72.2% |
| 50 | PDAC vs. CP | 84.4% | 88.3% | 82.3% | 78.5% | 83.5% | 77.2% |
| 51 | PDAC vs. CP | 83.1% | 88.3% | 79.7% | 75.9% | 77.2% | 67.1% |
| 52 | PDAC vs. CP | 84.4% | 89.6% | 82.3% | 77.2% | 78.5% | 70.9% |
| 53 | PDAC vs. CP | 88.3% | 93.5% | 82.3% | 78.5% | 74.7% | 65.8% |
| 54 | PDAC vs. CP | 88.3% | 93.5% | 79.7% | 77.2% | 75.9% | 67.1% |
| 55 | PDAC vs. CP | 85.7% | 90.9% | 78.5% | 73.4% | 81.0% | 73.4% |
| 56 | PDAC vs. CP | 87.0% | 92.2% | 77.2% | 72.2% | 78.5% | 70.9% |
| 57 | PDAC vs. CP | 88.3% | 93.5% | 79.7% | 75.9% | 74.7% | 65.8% |
| 58 | PDAC vs. CP | 88.3% | 92.2% | 82.3% | 79.7% | 77.2% | 65.8% |
| 59 | PDAC vs. CP | 85.7% | 89.6% | 81.0% | 77.2% | 81.0% | 72.2% |
| 60 | PDAC vs. CP | 85.7% | 89.6% | 78.5% | 75.9% | 79.7% | 68.4% |
| 61 | PDAC vs. CP | 88.3% | 93.5% | 79.7% | 75.9% | 77.2% | 68.4% |
| 62 | PDAC vs. CP | 88.3% | 92.2% | 77.2% | 74.7% | 81.0% | 69.6% |
| 63 | PDAC vs. CP | 87.0% | 92.2% | 77.2% | 73.4% | 81.0% | 72.2% |
| 64 | PDAC vs. CP | 87.0% | 92.2% | 77.2% | 73.4% | 78.5% | 70.9% |
| 65 | PDAC vs. CP | 88.3% | 93.5% | 79.7% | 77.2% | 77.2% | 67.1% |
| 66 | PDAC vs. CP | 89.6% | 93.5% | 81.0% | 78.5% | 81.0% | 72.2% |
| 67 | PDAC vs. CP | 88.3% | 93.5% | 78.5% | 75.9% | 77.2% | 67.1% |
| 68 | PDAC vs. CP | 85.7% | 90.9% | 78.5% | 74.7% | 82.3% | 75.9% |
| 69 | PDAC vs. CP | 87.0% | 93.5% | 78.5% | 74.7% | 77.2% | 67.1% |
| 70 | PDAC vs. CP | 88.3% | 93.5% | 75.9% | 74.7% | 77.2% | 69.6% |
| 71 | PDAC vs. CP | 87.0% | 90.9% | 79.7% | 78.5% | 79.7% | 72.2% |
| 72 | PDAC vs. CP | 85.7% | 89.6% | 78.5% | 74.7% | 82.3% | 74.7% |
| 73 | PDAC vs. CP | 85.7% | 90.9% | 79.7% | 77.2% | 81.0% | 70.9% |
| 74 | PDAC vs. CP | 84.4% | 89.6% | 77.2% | 73.4% | 82.3% | 74.7% |
| 75 | PDAC vs. CP | 87.0% | 92.2% | 79.7% | 75.9% | 79.7% | 70.9% |
| 76 | PDAC vs. CP | 87.0% | 92.2% | 75.9% | 73.4% | 79.7% | 70.9% |
| 77 | PDAC vs. CP | 85.7% | 90.9% | 77.2% | 73.4% | 83.5% | 75.9% |
| 78 | PDAC vs. CP | 88.3% | 93.5% | 78.5% | 77.2% | 79.7% | 70.9% |
| 79 | PDAC vs. CP | 88.3% | 93.5% | 77.2% | 75.9% | 79.7% | 70.9% |
| 80 | PDAC vs. CP | 88.3% | 93.5% | 82.3% | 78.5% | 74.7% | 64.6% |
| 81 | PDAC vs. CP | 89.6% | 93.5% | 79.7% | 75.9% | 75.9% | 65.8% |
| 82 | PDAC vs. CP | 90.9% | 93.5% | 82.3% | 79.7% | 74.7% | 63.3% |
| 83 | PDAC vs. CP | 87.0% | 90.9% | 82.3% | 78.5% | 77.2% | 67.1% |
| 84 | PDAC vs. CP | 88.3% | 93.5% | 82.3% | 75.9% | 75.9% | 65.8% |
| 85 | PDAC vs. CP | 87.0% | 92.2% | 83.5% | 79.7% | 77.2% | 65.8% |
| 86 | PDAC vs. CP | 88.3% | 93.5% | 79.7% | 75.9% | 77.2% | 65.8% |
| 87 | PDAC vs. CP | 88.3% | 93.5% | 78.5% | 74.7% | 74.7% | 65.8% |
| 88 | PDAC vs. CP | 90.9% | 94.8% | 83.5% | 79.7% | 73.4% | 62.0% |
| 89 | PDAC vs. CP | 89.6% | 92.2% | 79.7% | 77.2% | 72.2% | 59.5% |
| 90 | PDAC vs. CP | 90.9% | 93.5% | 81.0% | 77.2% | 77.2% | 67.1% |
| 91 | PDAC vs. CP | 87.0% | 90.9% | 82.3% | 78.5% | 74.7% | 65.8% |
| 92 | PDAC vs. CP | 90.9% | 94.8% | 81.0% | 78.5% | 74.7% | 64.6% |
| 93 | PDAC vs. CP | 84.4% | 89.6% | 79.7% | 75.9% | 78.5% | 69.6% |
| 94 | PDAC vs. CP | 89.6% | 96.1% | 81.0% | 77.2% | 75.9% | 67.1% |
| 95 | PDAC vs. CP | 89.6% | 93.5% | 79.7% | 77.2% | 75.9% | 65.8% |
| 96 | PDAC vs. CP | 89.6% | 92.2% | 78.5% | 74.7% | 75.9% | 64.6% |
| 97 | PDAC vs. CP | 89.6% | 94.8% | 75.9% | 73.4% | 78.5% | 69.6% |
| 98 | PDAC vs. CP | 83.1% | 87.0% | 82.3% | 78.5% | 81.0% | 74.7% |
| 99 | PDAC vs. CP | 85.7% | 90.9% | 79.7% | 75.9% | 78.5% | 68.4% |
| 100 | PDAC vs. CP | 85.7% | 90.9% | 81.0% | 77.2% | 81.0% | 70.9% |
| 101 | PDAC vs. CP | 88.3% | 93.5% | 82.3% | 78.5% | 77.2% | 68.4% |
| 102 | PDAC vs. CP | 85.7% | 90.9% | 83.5% | 79.7% | 77.2% | 67.1% |
| 103 | PDAC vs. CP | 87.0% | 92.2% | 81.0% | 77.2% | 77.2% | 69.6% |
| 104 | PDAC vs. CP | 89.6% | 94.8% | 81.0% | 77.2% | 75.9% | 65.8% |
| 105 | PDAC vs. CP | 84.4% | 88.3% | 86.1% | 83.5% | 86.1% | 77.2% |
| 105 | PDAC vs. Healthy | 81.8% | 84.4% | 82.3% | 78.5% | 93.7% | 91.1% |
| 105 | PDAC vs. (CP + non-pancreatic ctrl) | 83.1% | 87.0% | 86.1% | 83.5% | 91.1% | 84.8% |
| 105 | PDAC resectable vs. CP | 83.1% | 87.0% | 87.3% | 84.8% | 84.8% | 77.2% |
| 105 | PDAC resectable vs. Healthy | 83.1% | 85.7% | 81.0% | 78.5% | 92.4% | 88.6% |
| 105 | PDAC resectable vs. (CP + non- | 84.4% | 87.0% | 82.3% | 78.5% | 91.1% | 83.5% |
| | pancreatic ctrl) | | | | | | |
| 106 | PDAC vs. CP | 88.3% | 93.5% | 87.3% | 83.5% | 83.5% | 73.4% |
| 106 | PDAC vs. Healthy | 85.7% | 89.6% | 77.2% | 73.4% | 88.6% | 83.5% |
| 106 | PDAC vs. (CP + non-pancreatic ctrl) | 87.0% | 92.2% | 86.1% | 83.5% | 84.8% | 77.2% |
| 106 | PDAC resectable vs. CP | 87.0% | 92.2% | 88.6% | 86.1% | 83.5% | 73.4% |
| 106 | PDAC resectable vs. Healthy | 83.1% | 87.0% | 78.5% | 74.7% | 89.9% | 84.8% |
| 106 | PDAC resectable vs. (CP + non- | 85.7% | 89.6% | 87.3% | 86.1% | 89.9% | 82.3% |
| | pancreatic ctrl) | | | | | | |

A: Sensitivity observed with same model for all samples
B: Sensitivity observed with two-model algorithm according to CA19-9 level
C: Specificity observed with same model for all samples, PDAC versus chronic pancreatitis
D: Specificity observed with two-model algorithm according to CA19-9 level, versus chronic pancreatitis
E: Specificity observed with same model for all samples, versus non-pancreatic control
F: Specificity observed with two-model algorithm according to CA19-9 level, versus non-pancreatic control

### EXAMPLE 9 ANALYSIS OF SPECIFICITY AGAINST OTHER DISEASES

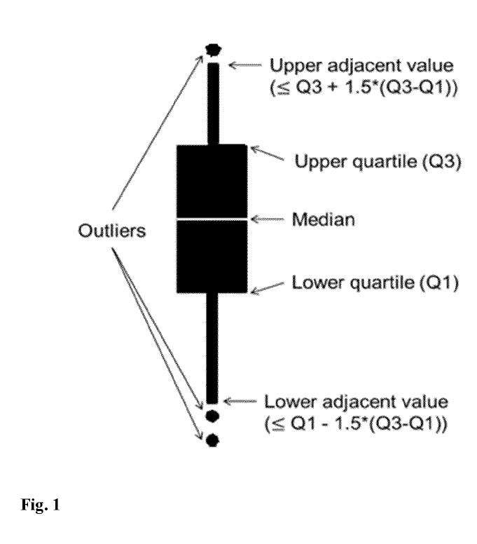

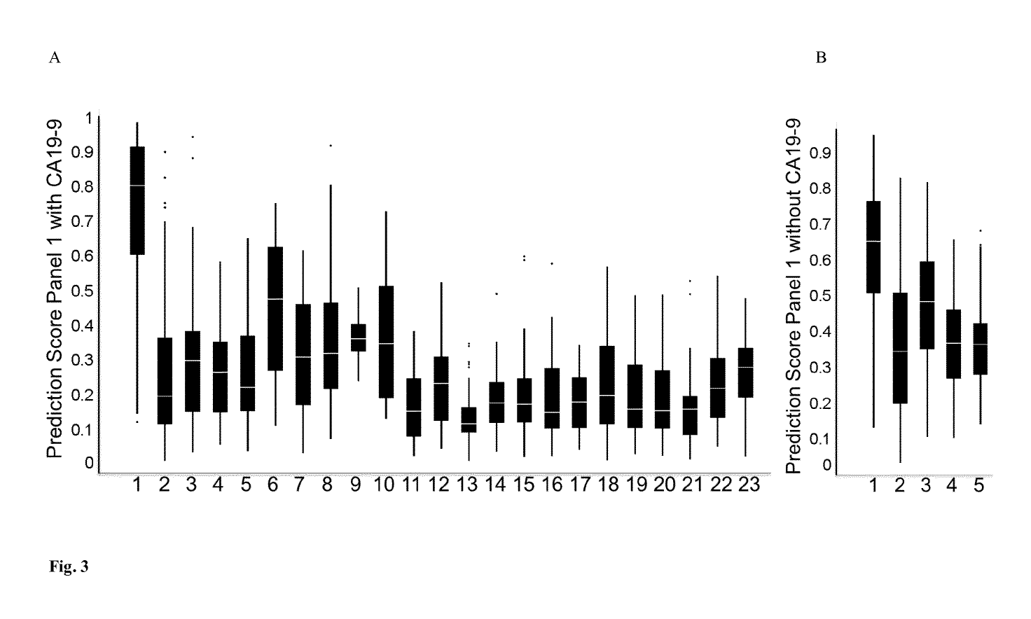

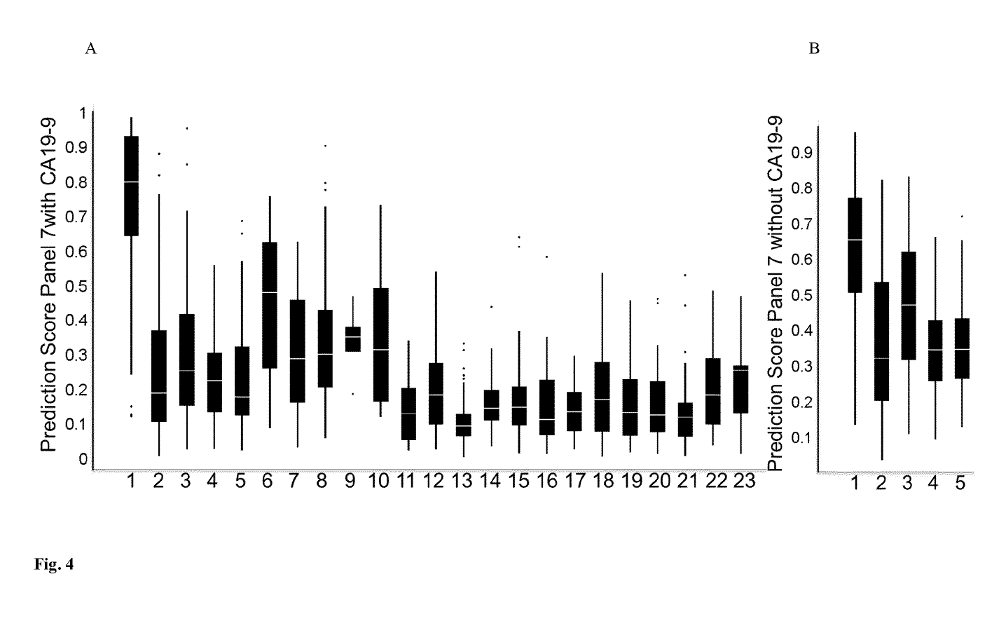

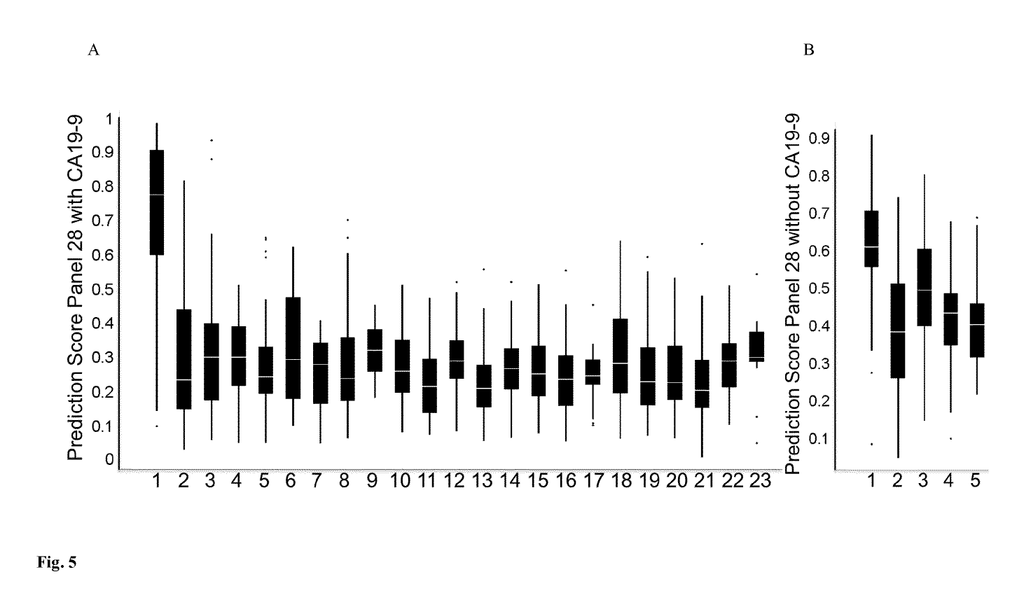

In order to analyze the biomarker specificity against other diseases, two studies were carried out in addition to the studies described in Example 1 (referred to as “PDAC study” and “Diabetes study” in FIGS. 1 to 5 and Table 20). First, a human EDTA plasma sample collection from 97 fasted treatment-naïve lung cancer patients (males and females, age 47-79 years) was analyzed comprising 20 small cell lung cancer cases, 52 non-small cell lung cancer (NSCLC) adeno carcinoma cases, 18 NSCLC squamous cell carcinoma cases, 4 NSCLC large cell carcinoma cases, and 3 NSCLC (not further characterized) cases (referred to as “Lung cancer study” in FIGS. 1 to 5 and Table 20). Second, from a human EDTA plasma sample collection from a prostate cancer diagnosis study, 445 samples were selected according to self-reported comorbidities from fasted male prostate cancer and control patients and analysed (referred to as “Other comorbidity study” in FIGS. 1 to 5 and Table 20).

All patients or their legal representatives gave their written informed consent and the local ethics review boards approved the protocol. After blood drawing and centrifugation according to the blood draw tube manufacturer's instruction, EDTA plasma was collected in Eppendorf tubes and stored at −80° C. for further analysis.

The resulting overall set of samples for the analysis of specificity against other diseases/comorbidities was as shown in Table 20, below:

TABLE 20
overall set of samples for the analysis of specificity against other diseases

| Group | | | |
|---|---|---|---|
| number | Study | Group name | n |
| 1 | PDAC study | Pancreatic cancer | 77 |
| 2 | PDAC study | Chronic pancreatitis | 79 |
| 3 | PDAC study | Non-pancreatic control (thyroid resections and hernia repair) | 79 |
| 4 | Diabetes study | Diabetes and other comorbidities | 51 |
| 5 | Diabetes study | Other comorbidities, but no diabetes | 50 |
| 6 | Lung cancer study | Small cell lung cancer | 20 |
| 7 | Lung cancer study | Non-small cell lung cancer (NSCLC) | 3 |
| 8 | Lung cancer study | NSCLC adenocarcinoma | 52 |
| 9 | Lung cancer study | NSCLC large cell carcinoma | 4 |
| 10 | Lung cancer study | NSCLC squamous cell carcinoma | 18 |
| 11 | Other comorbidities | Diabetes and dyslipidemia, most also hypertension | 12 |
| | study | | |
| 12 | Other comorbidities | Diabetes but no dyslipidemia, more than half also | 34 |
| | study | hypertension | |
| 13 | Other comorbidities | No comorbidities, age 62 or younger | 42 |
| | study | | |
| 14 | Other comorbidities | No comorbidities, age 63 or older | 29 |
| | study | | |
| 15 | Other comorbidities | Prostate cancer | 48 |
| | study | | |
| 16 | Other comorbidities | Cardiovascular diseases | 49 |
| | study | | |
| 17 | Other comorbidities | Chronic obstructive pulmonary disease, half also | 16 |
| | study | hypertension | |
| 18 | Other comorbidities | Dyslipidemia but no diabetes, more than half also | 36 |
| | study | hypertension | |
| 19 | Other comorbidities | Hypertension with other comorbidities, but no diabetes or | 62 |
| | study | dyslipidemia | |
| 20 | Other comorbidities | Hypertension only | 37 |
| | study | | |
| 21 | Other comorbidities | Other comorbidities, age 62 or younger | 35 |
| | study | | |
| 22 | Other comorbidities | Other comorbidities, age 63 or older | 32 |
| | study | | |
| 23 | Other comorbidities | Thyroid disorders | 13 |
| | study | | |

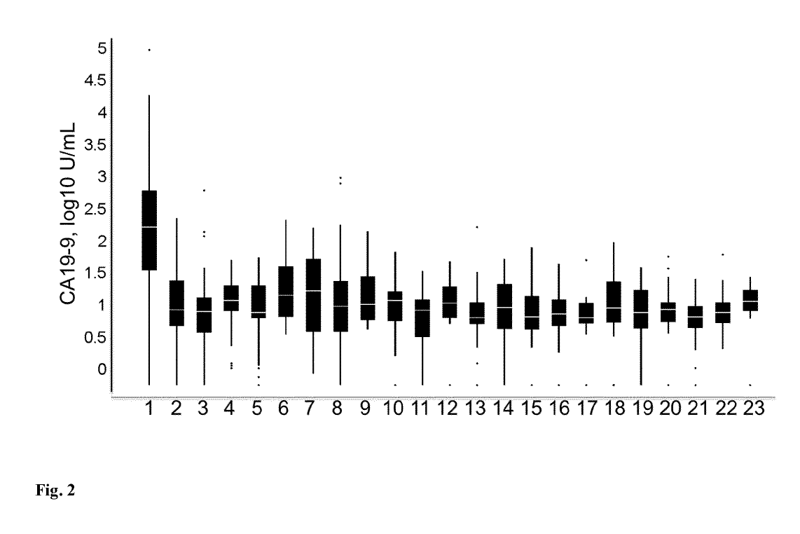

Using the above-described samples in order to assess the disease-specificity of the biomarker panels of the invention, it was found that the classification score showed a remarkably high specificity, also with regard to the panels of the invention in comparison to the CA19-9 as a single marker (see FIGS. 2 to 5). Results of this analysis are shown for all panels of the invention in Table 21, below. Also, prediction scores without CA19-9 were comparable in predictive value to CA19-9 as a single marker, as shown in representative examples in FIGS. 2, 3A, 4A, and 5A.

TABLE 21
Median of prediction score for the disease-/comorbidity groups shown in Table 20.

| Panel | Training of panel | Median of prediction score for group number | | | | | | | | | | |
|---|---|---|---|---|---|---|---|---|---|---|---|---|
| No. | on | 1 | 2 | 3 | 4 | 5 | 6 | 7 | 8 | 9 | 10 | 11 |
| 1 | PDAC vs CP | 0.81 | 0.20 | 0.30 | 0.27 | 0.23 | 0.48 | 0.31 | 0.32 | 0.37 | 0.35 | 0.16 |
| 2 | PDAC vs CP | 0.80 | 0.18 | 0.28 | 0.23 | 0.22 | 0.45 | 0.33 | 0.33 | 0.37 | 0.29 | 0.15 |
| 3 | PDAC vs CP | 0.80 | 0.21 | 0.28 | 0.28 | 0.23 | 0.39 | 0.20 | 0.29 | 0.34 | 0.30 | 0.13 |
| 4 | PDAC vs CP | 0.79 | 0.19 | 0.28 | 0.32 | 0.27 | 0.44 | 0.33 | 0.35 | 0.34 | 0.36 | 0.19 |
| 5 | PDAC vs CP | 0.80 | 0.21 | 0.28 | 0.28 | 0.23 | 0.39 | 0.20 | 0.29 | 0.34 | 0.30 | 0.13 |
| 6 | PDAC vs CP | 0.79 | 0.20 | 0.27 | 0.28 | 0.23 | 0.40 | 0.21 | 0.30 | 0.35 | 0.31 | 0.13 |
| 7 | PDAC vs CP | 0.81 | 0.19 | 0.26 | 0.23 | 0.18 | 0.49 | 0.30 | 0.31 | 0.36 | 0.32 | 0.14 |
| 8 | PDAC vs CP | 0.80 | 0.18 | 0.26 | 0.25 | 0.21 | 0.44 | 0.21 | 0.27 | 0.34 | 0.32 | 0.12 |
| 9 | PDAC vs CP | 0.80 | 0.18 | 0.28 | 0.21 | 0.19 | 0.45 | 0.26 | 0.28 | 0.35 | 0.27 | 0.14 |
| 10 | PDAC vs CP | 0.80 | 0.18 | 0.27 | 0.20 | 0.19 | 0.44 | 0.26 | 0.29 | 0.35 | 0.28 | 0.13 |
| 11 | PDAC vs CP | 0.81 | 0.15 | 0.24 | 0.29 | 0.28 | 0.47 | 0.35 | 0.34 | 0.32 | 0.33 | 0.18 |
| 12 | PDAC vs CP | 0.80 | 0.23 | 0.29 | 0.28 | 0.23 | 0.41 | 0.34 | 0.33 | 0.38 | 0.32 | 0.19 |
| 13 | PDAC vs CP | 0.79 | 0.21 | 0.29 | 0.24 | 0.23 | 0.47 | 0.36 | 0.33 | 0.38 | 0.26 | 0.20 |
| 14 | PDAC vs CP | 0.78 | 0.20 | 0.27 | 0.37 | 0.30 | 0.41 | 0.36 | 0.34 | 0.34 | 0.35 | 0.20 |
| 15 | PDAC vs CP | 0.82 | 0.22 | 0.25 | 0.30 | 0.25 | 0.38 | 0.23 | 0.30 | 0.35 | 0.29 | 0.15 |
| 16 | PDAC vs CP | 0.80 | 0.21 | 0.28 | 0.28 | 0.25 | 0.46 | 0.29 | 0.30 | 0.27 | 0.36 | 0.13 |
| 17 | PDAC vs CP | 0.83 | 0.21 | 0.27 | 0.26 | 0.25 | 0.33 | 0.25 | 0.23 | 0.23 | 0.27 | 0.14 |
| 18 | PDAC vs CP | 0.79 | 0.22 | 0.27 | 0.26 | 0.22 | 0.46 | 0.28 | 0.30 | 0.32 | 0.34 | 0.13 |
| 19 | PDAC vs CP | 0.79 | 0.25 | 0.28 | 0.28 | 0.23 | 0.36 | 0.32 | 0.28 | 0.29 | 0.31 | 0.18 |
| 20 | PDAC vs CP | 0.81 | 0.24 | 0.27 | 0.27 | 0.25 | 0.28 | 0.28 | 0.24 | 0.25 | 0.24 | 0.16 |
| 21 | PDAC vs CP | 0.77 | 0.22 | 0.28 | 0.28 | 0.24 | 0.42 | 0.31 | 0.31 | 0.34 | 0.31 | 0.15 |
| 22 | PDAC vs CP | 0.78 | 0.22 | 0.30 | 0.32 | 0.24 | 0.46 | 0.34 | 0.36 | 0.44 | 0.36 | 0.23 |
| 23 | PDAC vs CP | 0.77 | 0.23 | 0.29 | 0.34 | 0.26 | 0.45 | 0.30 | 0.31 | 0.35 | 0.37 | 0.22 |
| 24 | PDAC vs CP | 0.81 | 0.25 | 0.30 | 0.30 | 0.24 | 0.33 | 0.26 | 0.23 | 0.31 | 0.28 | 0.20 |
| 25 | PDAC vs CP | 0.79 | 0.24 | 0.29 | 0.31 | 0.25 | 0.43 | 0.31 | 0.33 | 0.40 | 0.35 | 0.20 |
| 26 | PDAC vs CP | 0.78 | 0.22 | 0.30 | 0.32 | 0.26 | 0.43 | 0.36 | 0.34 | 0.46 | 0.33 | 0.25 |
| 27 | PDAC vs CP | 0.76 | 0.23 | 0.30 | 0.34 | 0.26 | 0.40 | 0.33 | 0.29 | 0.37 | 0.34 | 0.26 |
| 28 | PDAC vs CP | 0.78 | 0.24 | 0.31 | 0.31 | 0.25 | 0.30 | 0.28 | 0.24 | 0.33 | 0.27 | 0.22 |
| 29 | PDAC vs CP | 0.79 | 0.24 | 0.28 | 0.32 | 0.25 | 0.41 | 0.33 | 0.32 | 0.41 | 0.32 | 0.21 |
| 30 | PDAC vs CP | 0.77 | 0.22 | 0.25 | 0.27 | 0.20 | 0.54 | 0.35 | 0.36 | 0.44 | 0.39 | 0.21 |
| 31 | PDAC vs CP | 0.81 | 0.23 | 0.28 | 0.25 | 0.20 | 0.36 | 0.28 | 0.22 | 0.27 | 0.29 | 0.19 |
| 32 | PDAC vs CP | 0.76 | 0.23 | 0.27 | 0.26 | 0.21 | 0.55 | 0.33 | 0.33 | 0.40 | 0.39 | 0.17 |
| 33 | PDAC vs CP | 0.79 | 0.21 | 0.27 | 0.27 | 0.24 | 0.46 | 0.33 | 0.31 | 0.33 | 0.42 | 0.18 |
| 34 | PDAC vs CP | 0.79 | 0.23 | 0.25 | 0.28 | 0.24 | 0.49 | 0.39 | 0.35 | 0.46 | 0.37 | 0.22 |
| 35 | PDAC vs CP | 0.77 | 0.24 | 0.26 | 0.28 | 0.24 | 0.45 | 0.37 | 0.30 | 0.35 | 0.40 | 0.21 |
| 36 | PDAC vs CP | 0.80 | 0.23 | 0.28 | 0.25 | 0.22 | 0.32 | 0.31 | 0.22 | 0.30 | 0.28 | 0.20 |
| 37 | PDAC vs CP | 0.77 | 0.23 | 0.27 | 0.29 | 0.24 | 0.54 | 0.36 | 0.33 | 0.42 | 0.36 | 0.18 |
| 38 | PDAC vs CP | 0.78 | 0.22 | 0.27 | 0.27 | 0.23 | 0.36 | 0.18 | 0.27 | 0.24 | 0.30 | 0.11 |
| 39 | PDAC vs CP | 0.81 | 0.23 | 0.28 | 0.26 | 0.26 | 0.28 | 0.16 | 0.20 | 0.21 | 0.24 | 0.13 |
| 40 | PDAC vs CP | 0.79 | 0.22 | 0.26 | 0.26 | 0.23 | 0.39 | 0.19 | 0.28 | 0.29 | 0.30 | 0.11 |
| 41 | PDAC vs CP | 0.80 | 0.20 | 0.26 | 0.34 | 0.29 | 0.41 | 0.31 | 0.33 | 0.25 | 0.36 | 0.17 |
| 42 | PDAC vs CP | 0.82 | 0.21 | 0.26 | 0.30 | 0.32 | 0.28 | 0.27 | 0.23 | 0.21 | 0.30 | 0.17 |
| 43 | PDAC vs CP | 0.78 | 0.19 | 0.30 | 0.24 | 0.22 | 0.41 | 0.30 | 0.29 | 0.27 | 0.29 | 0.12 |
| 44 | PDAC vs CP | 0.80 | 0.21 | 0.30 | 0.22 | 0.21 | 0.33 | 0.26 | 0.24 | 0.24 | 0.24 | 0.12 |
| 45 | PDAC vs CP | 0.78 | 0.20 | 0.27 | 0.33 | 0.28 | 0.42 | 0.30 | 0.31 | 0.29 | 0.35 | 0.16 |
| 46 | PDAC vs CP | 0.78 | 0.20 | 0.28 | 0.21 | 0.22 | 0.42 | 0.29 | 0.33 | 0.31 | 0.30 | 0.11 |
| 47 | PDAC vs CP | 0.79 | 0.21 | 0.28 | 0.25 | 0.22 | 0.42 | 0.33 | 0.28 | 0.29 | 0.25 | 0.18 |
| 48 | PDAC vs CP | 0.78 | 0.22 | 0.28 | 0.25 | 0.22 | 0.34 | 0.29 | 0.24 | 0.25 | 0.20 | 0.17 |
| 49 | PDAC vs CP | 0.78 | 0.22 | 0.27 | 0.24 | 0.22 | 0.45 | 0.32 | 0.31 | 0.33 | 0.26 | 0.15 |
| 50 | PDAC vs CP | 0.78 | 0.21 | 0.26 | 0.37 | 0.31 | 0.39 | 0.34 | 0.31 | 0.25 | 0.35 | 0.20 |
| 51 | PDAC vs CP | 0.81 | 0.23 | 0.27 | 0.34 | 0.32 | 0.30 | 0.29 | 0.23 | 0.21 | 0.28 | 0.19 |
| 52 | PDAC vs CP | 0.79 | 0.23 | 0.25 | 0.33 | 0.32 | 0.44 | 0.33 | 0.32 | 0.30 | 0.34 | 0.16 |
| 53 | PDAC vs CP | 0.78 | 0.22 | 0.26 | 0.30 | 0.26 | 0.34 | 0.20 | 0.26 | 0.26 | 0.28 | 0.16 |
| 54 | PDAC vs CP | 0.79 | 0.25 | 0.26 | 0.27 | 0.25 | 0.29 | 0.17 | 0.21 | 0.22 | 0.22 | 0.14 |
| 55 | PDAC vs CP | 0.77 | 0.25 | 0.26 | 0.29 | 0.25 | 0.37 | 0.21 | 0.28 | 0.30 | 0.29 | 0.13 |
| 56 | PDAC vs CP | 0.77 | 0.18 | 0.27 | 0.39 | 0.31 | 0.44 | 0.34 | 0.35 | 0.39 | 0.40 | 0.25 |
| 57 | PDAC vs CP | 0.80 | 0.20 | 0.28 | 0.31 | 0.25 | 0.40 | 0.20 | 0.29 | 0.39 | 0.34 | 0.18 |
| 58 | PDAC vs CP | 0.81 | 0.19 | 0.28 | 0.28 | 0.24 | 0.39 | 0.34 | 0.36 | 0.44 | 0.37 | 0.19 |
| 59 | PDAC vs CP | 0.78 | 0.20 | 0.26 | 0.40 | 0.32 | 0.43 | 0.31 | 0.31 | 0.29 | 0.37 | 0.24 |
| 60 | PDAC vs CP | 0.81 | 0.23 | 0.29 | 0.38 | 0.32 | 0.30 | 0.27 | 0.22 | 0.26 | 0.29 | 0.23 |
| 61 | PDAC vs CP | 0.81 | 0.20 | 0.28 | 0.30 | 0.26 | 0.39 | 0.17 | 0.25 | 0.30 | 0.30 | 0.16 |
| 62 | PDAC vs CP | 0.80 | 0.23 | 0.29 | 0.29 | 0.26 | 0.27 | 0.15 | 0.19 | 0.26 | 0.23 | 0.16 |
| 63 | PDAC vs CP | 0.78 | 0.21 | 0.28 | 0.37 | 0.31 | 0.42 | 0.31 | 0.32 | 0.35 | 0.39 | 0.21 |
| 64 | PDAC vs CP | 0.79 | 0.22 | 0.30 | 0.30 | 0.25 | 0.40 | 0.19 | 0.29 | 0.35 | 0.33 | 0.16 |
| 65 | PDAC vs CP | 0.79 | 0.19 | 0.30 | 0.30 | 0.24 | 0.35 | 0.30 | 0.31 | 0.35 | 0.35 | 0.17 |
| 66 | PDAC vs CP | 0.80 | 0.21 | 0.32 | 0.26 | 0.26 | 0.27 | 0.26 | 0.26 | 0.31 | 0.28 | 0.15 |
| 67 | PDAC vs CP | 0.79 | 0.19 | 0.31 | 0.27 | 0.25 | 0.38 | 0.30 | 0.33 | 0.39 | 0.33 | 0.15 |
| 68 | PDAC vs CP | 0.78 | 0.18 | 0.27 | 0.41 | 0.34 | 0.41 | 0.37 | 0.35 | 0.40 | 0.39 | 0.27 |
| 69 | PDAC vs CP | 0.80 | 0.23 | 0.28 | 0.32 | 0.25 | 0.39 | 0.21 | 0.28 | 0.40 | 0.32 | 0.18 |
| 70 | PDAC vs CP | 0.82 | 0.21 | 0.26 | 0.29 | 0.26 | 0.39 | 0.37 | 0.34 | 0.46 | 0.34 | 0.26 |
| 71 | PDAC vs CP | 0.81 | 0.22 | 0.29 | 0.29 | 0.25 | 0.39 | 0.33 | 0.32 | 0.41 | 0.33 | 0.22 |
| 72 | PDAC vs CP | 0.79 | 0.21 | 0.24 | 0.43 | 0.34 | 0.41 | 0.34 | 0.31 | 0.30 | 0.35 | 0.28 |
| 73 | PDAC vs CP | 0.80 | 0.23 | 0.28 | 0.39 | 0.33 | 0.28 | 0.28 | 0.22 | 0.26 | 0.28 | 0.25 |
| 74 | PDAC vs CP | 0.77 | 0.23 | 0.28 | 0.40 | 0.33 | 0.39 | 0.34 | 0.33 | 0.35 | 0.38 | 0.23 |
| 75 | PDAC vs CP | 0.81 | 0.23 | 0.28 | 0.32 | 0.25 | 0.36 | 0.19 | 0.24 | 0.31 | 0.29 | 0.19 |
| 76 | PDAC vs CP | 0.79 | 0.26 | 0.29 | 0.31 | 0.27 | 0.25 | 0.16 | 0.19 | 0.27 | 0.21 | 0.18 |
| 77 | PDAC vs CP | 0.79 | 0.25 | 0.29 | 0.32 | 0.27 | 0.41 | 0.20 | 0.29 | 0.36 | 0.32 | 0.17 |
| 78 | PDAC vs CP | 0.81 | 0.22 | 0.28 | 0.29 | 0.25 | 0.36 | 0.34 | 0.29 | 0.37 | 0.32 | 0.23 |
| 79 | PDAC vs CP | 0.79 | 0.22 | 0.31 | 0.28 | 0.27 | 0.27 | 0.29 | 0.24 | 0.33 | 0.25 | 0.23 |
| 80 | PDAC vs CP | 0.78 | 0.19 | 0.26 | 0.33 | 0.26 | 0.51 | 0.36 | 0.37 | 0.39 | 0.41 | 0.24 |
| 81 | PDAC vs CP | 0.80 | 0.20 | 0.26 | 0.27 | 0.21 | 0.49 | 0.21 | 0.29 | 0.39 | 0.34 | 0.16 |
| 82 | PDAC vs CP | 0.80 | 0.18 | 0.26 | 0.24 | 0.21 | 0.54 | 0.37 | 0.38 | 0.45 | 0.40 | 0.17 |
| 83 | PDAC vs CP | 0.81 | 0.22 | 0.28 | 0.32 | 0.27 | 0.35 | 0.29 | 0.20 | 0.23 | 0.30 | 0.23 |
| 84 | PDAC vs CP | 0.79 | 0.21 | 0.26 | 0.33 | 0.28 | 0.53 | 0.34 | 0.34 | 0.35 | 0.43 | 0.20 |
| 85 | PDAC vs CP | 0.79 | 0.22 | 0.26 | 0.27 | 0.24 | 0.47 | 0.18 | 0.25 | 0.28 | 0.32 | 0.14 |
| 86 | PDAC vs CP | 0.81 | 0.21 | 0.29 | 0.25 | 0.23 | 0.32 | 0.15 | 0.17 | 0.23 | 0.25 | 0.15 |
| 87 | PDAC vs CP | 0.78 | 0.21 | 0.28 | 0.27 | 0.22 | 0.49 | 0.20 | 0.27 | 0.35 | 0.35 | 0.15 |
| 88 | PDAC vs CP | 0.77 | 0.21 | 0.28 | 0.28 | 0.23 | 0.49 | 0.35 | 0.31 | 0.34 | 0.38 | 0.15 |
| 89 | PDAC vs CP | 0.77 | 0.21 | 0.30 | 0.23 | 0.22 | 0.38 | 0.30 | 0.25 | 0.29 | 0.30 | 0.14 |
| 90 | PDAC vs CP | 0.79 | 0.21 | 0.30 | 0.27 | 0.22 | 0.57 | 0.33 | 0.34 | 0.39 | 0.39 | 0.13 |
| 91 | PDAC vs CP | 0.79 | 0.20 | 0.24 | 0.34 | 0.30 | 0.46 | 0.35 | 0.32 | 0.28 | 0.42 | 0.22 |
| 92 | PDAC vs CP | 0.79 | 0.21 | 0.26 | 0.29 | 0.26 | 0.52 | 0.42 | 0.35 | 0.47 | 0.37 | 0.24 |
| 93 | PDAC vs CP | 0.80 | 0.17 | 0.25 | 0.37 | 0.31 | 0.52 | 0.40 | 0.37 | 0.39 | 0.41 | 0.26 |
| 94 | PDAC vs CP | 0.80 | 0.21 | 0.25 | 0.27 | 0.24 | 0.47 | 0.22 | 0.29 | 0.40 | 0.33 | 0.16 |
| 95 | PDAC vs CP | 0.77 | 0.21 | 0.26 | 0.29 | 0.25 | 0.49 | 0.39 | 0.30 | 0.36 | 0.37 | 0.22 |
| 96 | PDAC vs CP | 0.78 | 0.23 | 0.30 | 0.26 | 0.24 | 0.40 | 0.33 | 0.25 | 0.31 | 0.29 | 0.22 |
| 97 | PDAC vs CP | 0.77 | 0.22 | 0.28 | 0.27 | 0.24 | 0.53 | 0.38 | 0.34 | 0.42 | 0.36 | 0.19 |
| 98 | PDAC vs CP | 0.80 | 0.19 | 0.24 | 0.38 | 0.32 | 0.46 | 0.39 | 0.34 | 0.28 | 0.39 | 0.27 |
| 99 | PDAC vs CP | 0.81 | 0.20 | 0.27 | 0.33 | 0.30 | 0.37 | 0.32 | 0.21 | 0.23 | 0.27 | 0.25 |
| 100 | PDAC vs CP | 0.77 | 0.21 | 0.25 | 0.36 | 0.31 | 0.55 | 0.38 | 0.33 | 0.35 | 0.42 | 0.23 |
| 101 | PDAC vs CP | 0.79 | 0.23 | 0.26 | 0.29 | 0.26 | 0.46 | 0.20 | 0.24 | 0.29 | 0.30 | 0.17 |
| 102 | PDAC vs CP | 0.79 | 0.23 | 0.29 | 0.27 | 0.23 | 0.33 | 0.17 | 0.17 | 0.25 | 0.23 | 0.16 |
| 103 | PDAC vs CP | 0.77 | 0.23 | 0.28 | 0.30 | 0.24 | 0.46 | 0.22 | 0.28 | 0.36 | 0.33 | 0.15 |
| 104 | PDAC vs CP | 0.79 | 0.15 | 0.26 | 0.31 | 0.29 | 0.38 | 0.32 | 0.31 | 0.31 | 0.32 | 0.18 |
| 105 | PDAC vs CP | 0.79 | 0.19 | 0.29 | 0.14 | 0.13 | 0.32 | 0.24 | 0.27 | 0.23 | 0.27 | 0.20 |
| | with two-model | | | | | | | | | | | |
| | algorithm | | | | | | | | | | | |
| | according to | | | | | | | | | | | |
| | CA19-9 level | | | | | | | | | | | |
| 106 | PDAC vs CP | 0.80 | 0.17 | 0.30 | 0.16 | 0.14 | 0.29 | 0.18 | 0.30 | 0.24 | 0.29 | 0.18 |
| | with two-model | | | | | | | | | | | |
| | algorithm | | | | | | | | | | | |
| | according to | | | | | | | | | | | |
| | CA19-9 level | | | | | | | | | | | |
| 105 | PDAC vs CP | 0.79 | 0.17 | 0.24 | 0.21 | 0.17 | 0.32 | 0.24 | 0.21 | 0.23 | 0.24 | 0.14 |
| | with same model | | | | | | | | | | | |
| | for all samples | | | | | | | | | | | |
| 106 | PDAC vs CP | 0.79 | 0.16 | 0.21 | 0.21 | 0.18 | 0.29 | 0.18 | 0.18 | 0.24 | 0.19 | 0.14 |
| | with same model | | | | | | | | | | | |
| | for all samples | | | | | | | | | | | |

| Panel | Median of prediction score for group number | | | | | | | | | | | |
|---|---|---|---|---|---|---|---|---|---|---|---|---|
| No. | 12 | 13 | 14 | 15 | 16 | 17 | 18 | 19 | 20 | 21 | 22 | [symbol] 3 |
| 1 | 0.24 | 0.12 | 0.18 | 0.18 | 0.15 | 0.18 | 0.20 | 0.16 | 0.16 | 0.16 | 0.22 | [symbol] 3 |
| 2 | 0.23 | 0.10 | 0.18 | 0.16 | 0.15 | 0.14 | 0.20 | 0.18 | 0.15 | 0.11 | 0.20 | 0.24 |
| 3 | 0.21 | 0.11 | 0.16 | 0.16 | 0.14 | 0.15 | 0.17 | 0.14 | 0.15 | 0.13 | 0.20 | 0.22 |
| 4 | 0.25 | 0.14 | 0.18 | 0.20 | 0.18 | 0.22 | 0.22 | 0.19 | 0.19 | 0.17 | 0.26 | 0.31 |
| 5 | 0.21 | 0.11 | 0.16 | 0.16 | 0.14 | 0.15 | 0.17 | 0.14 | 0.15 | 0.13 | 0.20 | 0.22 |
| 6 | 0.20 | 0.11 | 0.16 | 0.15 | 0.14 | 0.15 | 0.17 | 0.14 | 0.15 | 0.12 | 0.20 | 0.23 |
| 7 | 0.19 | 0.10 | 0.15 | 0.15 | 0.12 | 0.14 | 0.18 | 0.14 | 0.13 | 0.13 | 0.19 | 0.26 |
| 8 | 0.19 | 0.09 | 0.15 | 0.14 | 0.12 | 0.14 | 0.16 | 0.12 | 0.13 | 0.11 | 0.18 | 0.23 |
| 9 | 0.19 | 0.09 | 0.17 | 0.12 | 0.13 | 0.12 | 0.18 | 0.14 | 0.14 | 0.10 | 0.18 | 0.20 |
| 10 | 0.19 | 0.09 | 0.16 | 0.12 | 0.13 | 0.11 | 0.18 | 0.14 | 0.13 | 0.09 | 0.18 | 0.20 |
| 11 | 0.22 | 0.10 | 0.17 | 0.14 | 0.18 | 0.15 | 0.22 | 0.19 | 0.16 | 0.11 | 0.21 | 0.23 |
| 12 | 0.29 | 0.15 | 0.23 | 0.21 | 0.18 | 0.20 | 0.23 | 0.19 | 0.20 | 0.21 | 0.26 | 0.29 |
| 13 | 0.25 | 0.14 | 0.22 | 0.18 | 0.17 | 0.16 | 0.23 | 0.20 | 0.17 | 0.14 | 0.20 | 0.28 |
| 14 | 0.31 | 0.17 | 0.22 | 0.23 | 0.22 | 0.23 | 0.24 | 0.22 | 0.22 | 0.20 | 0.29 | 0.32 |
| 15 | 0.27 | 0.14 | 0.19 | 0.19 | 0.18 | 0.17 | 0.20 | 0.16 | 0.18 | 0.15 | 0.22 | 0.26 |
| 16 | 0.24 | 0.13 | 0.22 | 0.21 | 0.16 | 0.20 | 0.21 | 0.19 | 0.16 | 0.17 | 0.23 | 0.31 |
| 17 | 0.22 | 0.13 | 0.19 | 0.18 | 0.15 | 0.18 | 0.21 | 0.17 | 0.17 | 0.16 | 0.23 | 0.24 |
| 18 | 0.18 | 0.12 | 0.16 | 0.16 | 0.13 | 0.14 | 0.17 | 0.15 | 0.14 | 0.12 | 0.20 | 0.25 |
| 19 | 0.28 | 0.16 | 0.25 | 0.22 | 0.18 | 0.21 | 0.24 | 0.21 | 0.20 | 0.21 | 0.29 | 0.29 |
| 20 | 0.26 | 0.17 | 0.24 | 0.21 | 0.18 | 0.19 | 0.23 | 0.21 | 0.19 | 0.20 | 0.25 | 0.29 |
| 21 | 0.23 | 0.15 | 0.19 | 0.18 | 0.16 | 0.16 | 0.20 | 0.17 | 0.17 | 0.16 | 0.23 | 0.24 |
| 22 | 0.27 | 0.18 | 0.25 | 0.25 | 0.21 | 0.25 | 0.27 | 0.23 | 0.21 | 0.18 | 0.27 | [symbol] 1 |
| 23 | 0.26 | 0.18 | 0.24 | 0.25 | 0.21 | 0.24 | 0.26 | 0.20 | 0.21 | 0.17 | 0.28 | [symbol] 9 |
| 24 | 0.25 | 0.19 | 0.24 | 0.23 | 0.19 | 0.23 | 0.26 | 0.21 | 0.18 | 0.16 | 0.27 | [symbol] 7 |
| 25 | 0.23 | 0.17 | 0.21 | 0.22 | 0.18 | 0.23 | 0.24 | 0.20 | 0.17 | 0.14 | 0.25 | [symbol] 6 |
| 26 | 0.31 | 0.21 | 0.28 | 0.27 | 0.24 | 0.27 | 0.29 | 0.23 | 0.24 | 0.20 | 0.30 | [symbol] 4 |
| 27 | 0.31 | 0.21 | 0.31 | 0.28 | 0.25 | 0.26 | 0.30 | 0.24 | 0.24 | 0.21 | 0.31 | [symbol] 5 |
| 28 | 0.29 | 0.21 | 0.27 | 0.26 | 0.24 | 0.25 | 0.29 | 0.24 | 0.23 | 0.21 | 0.29 | [symbol] 1 |
| 29 | 0.27 | 0.20 | 0.24 | 0.24 | 0.21 | 0.24 | 0.26 | 0.22 | 0.21 | 0.17 | 0.26 | 0.29 |
| 30 | 0.21 | 0.14 | 0.21 | 0.21 | 0.17 | 0.19 | 0.24 | 0.19 | 0.19 | 0.14 | 0.25 | [symbol] 3 |
| 31 | 0.22 | 0.15 | 0.22 | 0.20 | 0.16 | 0.20 | 0.21 | 0.19 | 0.17 | 0.13 | 0.24 | [symbol] 2 |
| 32 | 0.20 | 0.14 | 0.18 | 0.19 | 0.15 | 0.18 | 0.21 | 0.17 | 0.15 | 0.13 | 0.22 | [symbol] 2 |
| 33 | 0.21 | 0.16 | 0.22 | 0.24 | 0.18 | 0.20 | 0.22 | 0.20 | 0.20 | 0.14 | 0.27 | [symbol] 5 |
| 34 | 0.24 | 0.18 | 0.25 | 0.24 | 0.19 | 0.21 | 0.26 | 0.22 | 0.22 | 0.16 | 0.25 | [symbol] 2 |
| 35 | 0.26 | 0.19 | 0.29 | 0.28 | 0.21 | 0.21 | 0.28 | 0.22 | 0.22 | 0.18 | 0.29 | [symbol] 1 |
| 36 | 0.25 | 0.19 | 0.27 | 0.23 | 0.20 | 0.21 | 0.26 | 0.21 | 0.21 | 0.18 | 0.28 | 0.31 |
| 37 | 0.23 | 0.17 | 0.22 | 0.22 | 0.18 | 0.20 | 0.24 | 0.19 | 0.20 | 0.15 | 0.25 | 0.27 |
| 38 | 0.21 | 0.11 | 0.19 | 0.17 | 0.15 | 0.15 | 0.18 | 0.15 | 0.15 | 0.12 | 0.22 | 0.28 |
| 39 | 0.21 | 0.12 | 0.17 | 0.16 | 0.17 | 0.17 | 0.19 | 0.17 | 0.15 | 0.13 | 0.21 | 0.21 |
| 40 | 0.19 | 0.11 | 0.15 | 0.13 | 0.13 | 0.15 | 0.16 | 0.14 | 0.13 | 0.11 | 0.18 | 0.21 |
| 41 | 0.26 | 0.16 | 0.23 | 0.23 | 0.20 | 0.23 | 0.24 | 0.20 | 0.19 | 0.17 | 0.28 | 0.33 |
| 42 | 0.25 | 0.15 | 0.20 | 0.19 | 0.20 | 0.21 | 0.22 | 0.20 | 0.18 | 0.19 | 0.26 | 0.27 |
| 43 | 0.20 | 0.10 | 0.19 | 0.16 | 0.14 | 0.15 | 0.18 | 0.19 | 0.14 | 0.10 | 0.20 | 0.23 |
| 44 | 0.19 | 0.11 | 0.18 | 0.15 | 0.14 | 0.14 | 0.19 | 0.18 | 0.14 | 0.10 | 0.18 | 0.21 |
| 45 | 0.22 | 0.13 | 0.14 | 0.17 | 0.16 | 0.17 | 0.19 | 0.17 | 0.15 | 0.15 | 0.23 | 0.28 |
| 46 | 0.17 | 0.09 | 0.16 | 0.13 | 0.11 | 0.13 | 0.17 | 0.15 | 0.12 | 0.08 | 0.17 | 0.20 |
| 47 | 0.24 | 0.12 | 0.22 | 0.19 | 0.17 | 0.16 | 0.21 | 0.22 | 0.16 | 0.12 | 0.23 | 0.27 |
| 48 | 0.24 | 0.13 | 0.19 | 0.17 | 0.16 | 0.16 | 0.21 | 0.20 | 0.16 | 0.13 | 0.20 | 0.28 |
| 49 | 0.21 | 0.11 | 0.18 | 0.15 | 0.12 | 0.15 | 0.19 | 0.17 | 0.14 | 0.11 | 0.18 | 0.24 |
| 50 | 0.31 | 0.18 | 0.27 | 0.25 | 0.25 | 0.24 | 0.27 | 0.24 | 0.23 | 0.21 | 0.33 | 0.35 |
| 51 | 0.29 | 0.18 | 0.22 | 0.22 | 0.22 | 0.24 | 0.27 | 0.23 | 0.20 | 0.22 | 0.30 | 0.32 |
| 52 | 0.28 | 0.16 | 0.17 | 0.19 | 0.20 | 0.19 | 0.20 | 0.19 | 0.17 | 0.18 | 0.26 | 0.28 |
| 53 | 0.27 | 0.13 | 0.22 | 0.20 | 0.18 | 0.17 | 0.21 | 0.17 | 0.18 | 0.16 | 0.26 | 0.28 |
| 54 | 0.25 | 0.14 | 0.20 | 0.17 | 0.19 | 0.17 | 0.21 | 0.19 | 0.18 | 0.16 | 0.23 | 0.25 |
| 55 | 0.23 | 0.13 | 0.18 | 0.16 | 0.17 | 0.16 | 0.19 | 0.17 | 0.16 | 0.15 | 0.21 | [symbol] 3 |
| 56 | 0.30 | 0.16 | 0.22 | 0.24 | 0.25 | 0.24 | 0.27 | 0.21 | 0.23 | 0.19 | 0.30 | [symbol] 2 |
| 57 | 0.26 | 0.14 | 0.20 | 0.18 | 0.18 | 0.18 | 0.23 | 0.16 | 0.19 | 0.14 | 0.24 | [symbol] 3 |
| 58 | 0.25 | 0.15 | 0.23 | 0.19 | 0.20 | 0.20 | 0.26 | 0.23 | 0.20 | 0.14 | 0.23 | [symbol] |
| 59 | 0.28 | 0.19 | 0.23 | 0.26 | 0.26 | 0.25 | 0.27 | 0.21 | 0.22 | 0.20 | 0.33 | [symbol] |
| 60 | 0.30 | 0.19 | 0.19 | 0.23 | 0.23 | 0.24 | 0.27 | 0.22 | 0.21 | 0.19 | 0.30 | [symbol] |
| 61 | 0.25 | 0.14 | 0.22 | 0.18 | 0.19 | 0.16 | 0.22 | 0.16 | 0.17 | 0.14 | 0.25 | [symbol] 6 |
| 62 | 0.24 | 0.15 | 0.21 | 0.18 | 0.19 | 0.17 | 0.23 | 0.18 | 0.18 | 0.14 | 0.21 | 0.21 |
| 63 | 0.26 | 0.17 | 0.18 | 0.20 | 0.19 | 0.24 | 0.23 | 0.20 | 0.17 | 0.15 | 0.26 | 0.27 |
| 64 | 0.22 | 0.13 | 0.18 | 0.16 | 0.18 | 0.16 | 0.20 | 0.16 | 0.16 | 0.12 | 0.19 | [symbol] 1 |
| 65 | 0.24 | 0.14 | 0.24 | 0.20 | 0.19 | 0.20 | 0.24 | 0.20 | 0.21 | 0.11 | 0.23 | [symbol] 5 |
| 66 | 0.22 | 0.15 | 0.21 | 0.19 | 0.19 | 0.18 | 0.25 | 0.21 | 0.18 | 0.12 | 0.25 | [symbol] 6 |
| 67 | 0.20 | 0.13 | 0.19 | 0.15 | 0.15 | 0.16 | 0.23 | 0.18 | 0.16 | 0.11 | 0.20 | [symbol] 2 |
| 68 | 0.35 | 0.20 | 0.26 | 0.27 | 0.31 | 0.26 | 0.31 | 0.23 | 0.27 | 0.22 | 0.32 | [symbol] 5 |
| 69 | 0.31 | 0.16 | 0.23 | 0.21 | 0.21 | 0.19 | 0.25 | 0.19 | 0.21 | 0.17 | 0.26 | [symbol] 7 |
| 70 | 0.28 | 0.17 | 0.25 | 0.23 | 0.21 | 0.21 | 0.30 | 0.25 | 0.21 | 0.17 | 0.24 | 0.30 |
| 71 | 0.25 | 0.15 | 0.23 | 0.20 | 0.19 | 0.18 | 0.27 | 0.21 | 0.21 | 0.14 | 0.23 | 0.28 |
| 72 | 0.33 | 0.23 | 0.28 | 0.29 | 0.30 | 0.27 | 0.32 | 0.24 | 0.26 | 0.24 | 0.36 | 0.38 |
| 73 | 0.32 | 0.22 | 0.25 | 0.26 | 0.27 | 0.26 | 0.32 | 0.24 | 0.25 | 0.22 | 0.34 | 0.32 |
| 74 | 0.30 | 0.19 | 0.21 | 0.23 | 0.24 | 0.26 | 0.27 | 0.21 | 0.22 | 0.19 | 0.29 | 0.31 |
| 75 | 0.30 | 0.16 | 0.24 | 0.20 | 0.23 | 0.17 | 0.25 | 0.20 | 0.22 | 0.17 | 0.29 | 0.30 |
| 76 | 0.28 | 0.16 | 0.21 | 0.20 | 0.23 | 0.18 | 0.25 | 0.19 | 0.21 | 0.16 | 0.23 | 0.25 |
| 77 | 0.25 | 0.15 | 0.20 | 0.19 | 0.21 | 0.17 | 0.23 | 0.18 | 0.19 | 0.15 | 0.23 | 0.25 |
| 78 | 0.27 | 0.17 | 0.29 | 0.23 | 0.20 | 0.22 | 0.27 | 0.23 | 0.25 | 0.15 | 0.27 | 0.31 |
| 79 | 0.29 | 0.16 | 0.25 | 0.23 | 0.23 | 0.20 | 0.30 | 0.24 | 0.24 | 0.16 | 0.26 | 0.30 |
| 80 | 0.24 | 0.14 | 0.19 | 0.21 | 0.19 | 0.19 | 0.24 | 0.20 | 0.20 | 0.16 | 0.28 | 0.28 |
| 81 | 0.22 | 0.12 | 0.18 | 0.16 | 0.14 | 0.15 | 0.19 | 0.14 | 0.17 | 0.12 | 0.22 | 0.23 |
| 82 | 0.21 | 0.13 | 0.20 | 0.18 | 0.18 | 0.16 | 0.23 | 0.18 | 0.19 | 0.11 | 0.22 | 0.21 |
| 83 | 0.25 | 0.17 | 0.19 | 0.22 | 0.19 | 0.21 | 0.24 | 0.21 | 0.19 | 0.17 | 0.29 | 0.25 |
| 84 | 0.23 | 0.15 | 0.17 | 0.19 | 0.19 | 0.20 | 0.22 | 0.18 | 0.17 | 0.14 | 0.24 | 0.23 |
| 85 | 0.20 | 0.12 | 0.20 | 0.18 | 0.16 | 0.14 | 0.19 | 0.15 | 0.17 | 0.12 | 0.22 | 0.23 |
| 86 | 0.20 | 0.13 | 0.17 | 0.16 | 0.15 | 0.14 | 0.20 | 0.15 | 0.15 | 0.12 | 0.21 | 0.20 |
| 87 | 0.20 | 0.13 | 0.16 | 0.16 | 0.17 | 0.15 | 0.19 | 0.15 | 0.14 | 0.11 | 0.20 | 0.21 |
| 88 | 0.24 | 0.13 | 0.23 | 0.20 | 0.18 | 0.18 | 0.22 | 0.21 | 0.19 | 0.11 | 0.22 | 0.21 |
| 89 | 0.22 | 0.13 | 0.22 | 0.20 | 0.18 | 0.15 | 0.22 | 0.21 | 0.18 | 0.10 | 0.23 | [symbol] 9 |
| 90 | 0.19 | 0.12 | 0.17 | 0.15 | 0.15 | 0.14 | 0.19 | 0.17 | 0.15 | 0.09 | 0.20 | [symbol] 6 |
| 91 | 0.24 | 0.17 | 0.24 | 0.24 | 0.23 | 0.22 | 0.25 | 0.22 | 0.20 | 0.17 | 0.31 | [symbol] 8 |
| 92 | 0.24 | 0.16 | 0.25 | 0.21 | 0.18 | 0.19 | 0.28 | 0.23 | 0.23 | 0.15 | 0.25 | [symbol] 4 |
| 93 | 0.31 | 0.19 | 0.24 | 0.25 | 0.24 | 0.21 | 0.26 | 0.23 | 0.24 | 0.18 | 0.29 | [symbol] 7 |
| 94 | 0.25 | 0.13 | 0.20 | 0.19 | 0.18 | 0.18 | 0.21 | 0.16 | 0.20 | 0.14 | 0.24 | [symbol] 7 |
| 95 | 0.28 | 0.16 | 0.29 | 0.22 | 0.19 | 0.18 | 0.25 | 0.25 | 0.22 | 0.14 | 0.26 | [symbol] 9 |
| 96 | 0.25 | 0.16 | 0.26 | 0.21 | 0.22 | 0.18 | 0.25 | 0.23 | 0.22 | 0.14 | 0.25 | 0.23 |
| 97 | 0.23 | 0.15 | 0.23 | 0.18 | 0.18 | 0.16 | 0.23 | 0.21 | 0.19 | 0.11 | 0.23 | 0.20 |
| 98 | 0.30 | 0.21 | 0.25 | 0.29 | 0.27 | 0.23 | 0.29 | 0.24 | 0.24 | 0.21 | 0.37 | [symbol] 6 |
| 99 | 0.28 | 0.20 | 0.25 | 0.24 | 0.22 | 0.24 | 0.28 | 0.24 | 0.23 | 0.21 | 0.31 | [symbol] 2 |
| 100 | 0.27 | 0.17 | 0.20 | 0.22 | 0.22 | 0.24 | 0.24 | 0.21 | 0.21 | 0.19 | 0.28 | [symbol] 1 |
| 101 | 0.24 | 0.14 | 0.24 | 0.20 | 0.20 | 0.15 | 0.21 | 0.16 | 0.19 | 0.15 | 0.25 | [symbol] 7 |
| 102 | 0.25 | 0.16 | 0.23 | 0.18 | 0.20 | 0.15 | 0.23 | 0.19 | 0.19 | 0.15 | 0.24 | [symbol] 5 |
| 103 | 0.23 | 0.14 | 0.20 | 0.18 | 0.18 | 0.15 | 0.18 | 0.17 | 0.17 | 0.13 | 0.20 | [symbol] 6 |
| 104 | 0.22 | 0.11 | 0.19 | 0.16 | 0.19 | 0.16 | 0.24 | 0.20 | 0.16 | 0.12 | 0.23 | 0.25 |
| 105 | 0.20 | 0.13 | 0.21 | 0.17 | 0.17 | 0.18 | 0.21 | 0.18 | 0.20 | 0.13 | 0.18 | 0.24 |
| 106 | 0.19 | 0.10 | 0.17 | 0.13 | 0.16 | 0.13 | 0.20 | 0.20 | 0.18 | 0.10 | 0.17 | 0.19 |
| 105 | 0.18 | 0.11 | 0.17 | 0.15 | 0.13 | 0.13 | 0.19 | 0.15 | 0.15 | 0.12 | 0.18 | 0.24 |
| 106 | 0.17 | 0.10 | 0.16 | 0.12 | 0.13 | 0.11 | 0.16 | 0.14 | 0.13 | 0.09 | 0.17 | 0.19 |

[symbol]

indicates data missing or illegible when filed

1. A method for diagnosing pancreatic cancer in a subject comprising the steps of:
(a) determining in at least one sample of said subject the amounts of a group of diagnostic biomarkers comprising
(i) at least one diagnostic amino acid, said diagnostic amino acid being proline, histidine or tryptophan;
(ii) at least one diagnostic ceramide, said diagnostic ceramide being ceramide (d18:1,C24:0) or ceramide (d18:2,C24:0)
(iii) at least one diagnostic sphingomyelin, said diagnostic sphingomyelin being sphingomyelin (35:1), sphingomyelin (d17:1,C16:0), sphingomyelin (41:2) or sphingomyelin (d18:2,C17:0); and
(iv) CA19-9;
and
(b) comparing said amounts of the diagnostic biomarkers with a reference, whereby pancreatic cancer is diagnosed,
wherein said subject is a subject at least 40 years old.

2. The method of claim 1, wherein said subject is a subject at risk of suffering from pancreatic cancer and/or a subject suffering from chronic pancreatitis; or wherein said subject is a subject suspected to suffer from pancreatic cancer.

3. The method of claim 1, wherein said subject is a subject with a low CA19-9 value.

4. The method of claim 1, wherein said pancreatic cancer is a pancreatic cancer with a resectable tumor stage.

5. The method of claim 1, wherein
(i) said diagnostic amino acid is proline;
(ii) said diagnostic ceramide is ceramide (d18:1,C24:0) or ceramide (d18:2,C24:0); and/or
(iii) said diagnostic sphingomyelin is sphingomyelin (35:1).

6. The method of claim 1, wherein said sphingomyelin (35:1) is the sum of sphingomyelin (d18:1,C17:0) and sphingomyelin (d17:1,C18:0);
sphingomyelin (d18:1,C17:0); or
sphingomyelin (d17:1,C18:0).

7. The method of claim 1, wherein said group of diagnostic biomarkers comprises, the diagnostic biomarkers proline, ceramide (d18:2,C24:0), sphingomyelin (35:1), and CA19-9.

8. The method of claim 1, wherein said group of diagnostic biomarkers further comprises at least one diagnostic ethanolamine lipid, said diagnostic ethanolamine lipid being phosphatidylethanolamine (C18:0,C22:6), lysophosphatidylethanolamine (C18:0), or lysophosphatidylethanolamine (C18:2).

9. The method of claim 1, wherein said group of diagnostic biomarkers comprises the diagnostic biomarkers CA19-9, Ceramide (d18:1,C24:0), Ceramide (d18:2,C24:0), Histidine, Lysophosphatidylethanolamine (C18:0), Lysophosphatidylethanolamine (C18:2), Phosphatidylethanolamine (C18:0,C22:6), Proline, Sphingomyelin (d17:1,C16:0), Sphingomyelin (35:1), Sphingomyelin (41:2), Sphingomyelin (d18:2,C17:0), and Tryptophan.

10. The method of claim 1, wherein said comparing amounts of diagnostic biomarkers with references comprises comparing said amounts or a value calculated therefrom to one or more cutoff values.

11. The method of claim 1, wherein said sample is a sample of a bodily fluid.

12. The method of claim 1, comprising the further step of separating said at least one diagnostic amino acid from said at least one diagnostic ceramide, said further step preceding step (a).

13. The method of claim 1, comprising the steps of
(a) quantitatively determining the amounts of a group of diagnostic biomarkers in a sample of a subject,
(b1) for each amount of (a), calculating a scaled amount by first subtracting a predetermined, diagnostic biomarker-specific subtrahend from said amount and then dividing the resulting value by a predetermined, diagnostic biomarker-specific divisor,
(b2) calculating a prediction score by
(i) assigning a diagnostic biomarker-specific weight value to each scaled amount of (b1), thereby providing a weighed amount,
(ii) summing up said weighed amounts for all diagnostic biomarkers, providing a sum of weighted amounts,
and
(b3) determining the probability for a subject to suffer from pancreatic cancer based on the prediction score determined in step (b2).

14. The method of claim 1, wherein comparing said amounts of the diagnostic biomarkers with a reference comprises assigning a smaller weight, to the amount of CA19-9 in case the amount of CA19-9 determined is less than about 5 U/ml.

15. A diagnostic device for carrying out a method according to claim 1, comprising:
a) an analysing unit comprising at least one detector for at least the small molecule diagnostic biomarkers of a group of diagnostic biomarkers according to claim 1, wherein said analyzing unit is adapted for determining the amounts of at least said small molecule diagnostic biomarkers detected by the at least one detector,
and, operatively linked thereto;
b) an evaluation unit comprising a computer comprising tangibly embedded a computer program code for carrying out a comparison of the determined amounts of the small molecule diagnostic biomarkers with a reference and a data base comprising said reference for said diagnostic biomarkers, whereby it is diagnosed whether a subject suffers from pancreatic cancer.

16. (canceled)
